(19) United States
(12) Patent Application Publication
(10) Pub. No.: US 2026/0159556 A1
(43) Pub. Date: Jun. 11, 2026

(54) POLYNUCLEOTIDE AND VECTOR

(71) Applicant: OSPEDALE SAN RAFFAELE S.R.L., Milan (IT)

(72) Inventors: Francesca Maltecca, Milan (IT); Daniele De Ritis, Milan (IT)

(21) Appl. No.: 19/126,994

(22) PCT Filed: Nov. 8, 2023
(86) PCT No.: PCT/EP2023/081198

§ 371 Date: May 2, 2025

(30) Foreign Application Priority Data
Nov. 8, 2022 (EP) 22206245.7

(51) Int. Cl. C07K 14/47; A61K 38/17; A61K 48/00; A61P 25/28; C12N 15/86

(52) U.S. Cl. CPC C07K 14/47; A61K 38/1709; A61K 48/005; A61P 25/28; C12N 15/86; C12N 2750/14143

(57) ABSTRACT

A polynucleotide comprising a nucleotide sequence encoding an engineered sacsin, wherein the engineered sacsin comprises a UbL domain or variant thereof, an SRR domain or variant thereof, a DnaJ domain or variant thereof and an HERN domain or variant thereof, and wherein the nucleotide sequence encoding the engineered sacsin is less than or equal to about 4000 bp in length.

### FIELD OF THE INVENTION

The present invention relates to engineered sacsin proteins and polynucleotides encoding therefor. The invention further relates to vectors encoding engineered sacsin proteins, such as viral vectors, and methods of treatment, in particular of cerebellar ataxias, such as autosomal recessive spastic ataxia of Charlevoix-Saguenay (ARSACS).

### BACKGROUND TO THE INVENTION

Autosomal recessive spastic ataxia of Charlevoix-Saguenay (ARSACS) is a rare cerebellar ataxia characterised by motor-coordination problems from very young ages. ARSACS is the second most common cause of recessive ataxia, with over 200 mutations described worldwide. The first two mutations were identified in the French-Canadian population 20 years ago due to a founder effect, but the disease is now present worldwide with an incidence of 2:100,000 inhabitants. In Quebec the prevalence is 1:1932. As an approximation, with over 300 persons diagnosed with the disease, ARSACS patients in Eastern Canada may represent about a third of all cases worldwide.

ARSACS is caused by mutations in the SACS gene, resulting in the inactivation of the encoded protein, sacsin.

Sacsin is highly expressed in Purkinje cells (PCs) in the cerebellum. Loss of PCs is the main feature of ARSACS patients and of the Sacs^{-/-} mouse model. This model recapitulates human disease progression showing a deficit in motor abilities (at 5 months) preceded by PC degeneration (at 3 months), while motor neuron degeneration occurs at a very late stage.

Sacsin is a very large, multimodular protein of 520 kDa, which contains a combination of domains: a ubiquitin-like domain (UbL), three sacsin repeat regions (SRR), homologous to Hsp90 chaperone, a DnaJ domain homologous to Hsp40 and a nucleotide binding domain (HEPN). Although these domains suggest an involvement in protein quality control, the cellular function of this protein remains largely unknown. Previous studies have indicated a role for sacsin in regulating the cytoskeleton, crucial for cellular functionality. Indeed, cell and mouse models of ARSACS present severe alteration of cytoskeleton. Abnormal bundling and accumulation of neurofilaments were detected in different neurons in brain autopsies of ARSACS patients and in the Sacs^{-/-} mouse at early time points.

There are currently no effective treatments for ARSACS. Accordingly, there remains a significant need for providing effective therapeutic strategies.

### SUMMARY OF THE INVENTION

Gene replacement of mutated genes has shown preclinical and clinical benefits for many inherited human diseases. Such approaches often use viral vectors, and AAV vectors are commonly used for gene transfer, since they don't integrate into the genome and for their safety profile. Furthermore, a promising vector for gene therapy of certain neurological disorders is AAV9, due to its ability to cross the blood-brain barrier and strong neuronal tropism. However, the packaging capacity of AAV is restricted to ˜4.7 kb, making it a substantial challenge to deliver large gene products, such as SACS.

The inventors have developed a miniaturised version of human sacsin protein (minisacsin) that is suitable for a gene replacement therapy using AAV vectors. The SACS minigene is based on a combination of sacsin domains (minisacsin): the N terminal region of sacsin, carrying the Ubiquitin-like (UbL) domain and sacsin repeat region SRR 1 linked in frame to domains from the sacsin C terminal region, DnaJ and HEPN.

The inventors observed abnormal bundling and accumulation of vimentin in ARSACS fibroblasts and neurofilaments in Sacs^{-/-} mice, indicating that sacsin could control in intermediate filament dynamics. They further demonstrated that ARSACS is caused by striking reduction of sacsin regardless of the nature and position of SACS mutations.

The inventors showed that transfection of minisacsin into neuronal-like wild type and SACS^{-/-} SH-SY5Y cells, gave rise to stable, high-level and non-toxic expression of minisacsin, and striking reduction of neurofilament accumulation and bundle volume in SACS cells transfected with minisacsin without affecting neurofilament structure in wild type cells.

The inventors further generated AAV vectors carrying the minisacsin gene and delivered these to mouse models. The mice did not show any sign of toxicity and had normal weight course as monitored after injection, confirming in vivo that minisacsin is not toxic. The inventors further assessed expression of minisacsin in ex vivo PCs, which showed detectable minisacsin expression, and that primary PCs cultures did not suffer from toxicity.

In one aspect, the invention provides a polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof.

In one aspect, the invention provides a polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof, and wherein the nucleotide sequence encoding the engineered sacsin is less than or equal to about 4000 bp in length.

In one aspect, the invention provides a polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof, and wherein the nucleotide sequence encoding the engineered sacsin is less than or equal to about 2700 bp in length.

In one aspect, the invention provides a polynucleotide comprising a nucleotide sequence encoding an engineered sacsin, wherein the engineered sacsin comprises a UbL domain, an SRR domain, a DnaJ domain and an HEPN domain.

In one aspect, the invention provides a polynucleotide comprising a nucleotide sequence encoding an engineered sacsin, wherein the engineered sacsin comprises a UbL domain, an SRR domain, a DnaJ domain and an HEPN domain, and wherein the nucleotide sequence encoding the engineered sacsin is less than or equal to about 4000 bp in length.

In one aspect, the invention provides a polynucleotide comprising a nucleotide sequence encoding an engineered sacsin, wherein the engineered sacsin comprises a UbL domain, an SRR domain, a DnaJ domain and an HEPN domain, and wherein the nucleotide sequence encoding the engineered sacsin is less than or equal to about 2700 bp in length.

In some embodiments, the nucleotide sequence encoding the engineered sacsin is less than or equal to about 3900 bp, 3800 bp, 3700 bp, 3600 bp, 3500 bp, 3400 bp, 3300 bp, 3200 bp, 3100 bp, 3000 bp, 2900 bp, 2800 bp, 2700 bp, 2600 bp, 2500 bp or 2400 bp in length.

In some embodiments, the nucleotide sequence encoding the engineered sacsin is about 2300-4000 bp, 2300-3900 bp, 2300-3800 bp, 2300-3700 bp, 2300-3600 bp, 2300-3500 bp, 2300-3400 bp, 2300-3300 bp, 2300-3200 bp, 2300-3100 bp, 2300-3000 bp, 2300-2900 bp, 2300-2800 bp, 2300-2700 bp, 2300-2600 bp, 2300-2500 bp or 2300-2400 bp in length.

In some embodiments, the nucleotide sequence encoding the engineered sacsin is less than or equal to about 2500 bp in length. In some embodiments, the nucleotide sequence encoding the engineered sacsin is less than or equal to about 2400 bp in length.

In some embodiments, the nucleotide sequence encoding the engineered sacsin is about 2300-2700 bp, 2300-2600 bp, 2300-2500 bp or 2300-2400 bp in length.

In some embodiments, the nucleotide sequence encoding the engineered sacsin is about 2300-2400 bp in length.

In some embodiments, the SRR domain is an SRR1 domain. In some embodiments, the SRR domain is an SRR2 domain. In some embodiments, the SRR domain is an SRR3 domain.

In preferred embodiments, the SRR domain is an SRR1 domain.

In some embodiments, the engineered sacsin comprises one or more SRR domain. In some embodiments, the engineered sacsin comprises two SRR domains. In some embodiments, the engineered sacsin comprises only one SRR domain.

In some embodiments, the engineered sacsin comprises a UbL domain, an SRR1 domain, a DnaJ domain and an HEPN domain. In some embodiments, the engineered sacsin comprises a UbL domain, an SRR2 domain, a DnaJ domain and an HEPN domain. In some embodiments, the engineered sacsin comprises a UbL domain, an SRR3 domain, a DnaJ domain and an HEPN domain. Preferably, the engineered sacsin comprises the domains in the order listed from N- to C-terminus.

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 1. In some embodiments, the nucleotide sequence encoding the SRR domain comprises or consists of the nucleotide sequence of SEQ ID NO: 1.

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 84. In some embodiments, the nucleotide sequence encoding the SRR domain comprises or consists of the nucleotide sequence of SEQ ID NO: 84.

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 2. In some embodiments, the nucleotide sequence encoding the SRR domain comprises or consists of the nucleotide sequence of SEQ ID NO: 2.

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 3. In some embodiments, the nucleotide sequence encoding the SRR domain comprises or consists of the nucleotide sequence of SEQ ID NO: 3.

In some embodiments, the nucleotide sequence encoding the UbL domain comprises or consists of a nucleotide sequence having at least 70%, 75%, 80%, 85%, 90%, 95%, 96%, 97%, 98% or 99% sequence identity to SEQ ID NO: 4. In some embodiments, the nucleotide sequence encoding the UbL domain comprises or consists of the nucleotide sequence of SEQ ID NO: 4.

In some embodiments, the nucleotide sequence encoding the UbL domain comprises or consists of a nucleotide sequence having at least 70%, 75%, 80%, 85%, 90%, 95%, 96%, 97%, 98% or 99% sequence identity to SEQ ID NO: 85. In some embodiments, the nucleotide sequence encoding the UbL domain comprises or consists of the nucleotide sequence of SEQ ID NO: 85.

In some embodiments, the nucleotide sequence encoding the DnaJ domain comprises or consists of a nucleotide sequence having at least 70%, 75%, 80%, 85%, 90%, 95%, 96%, 97%, 98% or 99% sequence identity to SEQ ID NO: 5. In some embodiments, the nucleotide sequence encoding the DnaJ domain comprises or consists of the nucleotide sequence of SEQ ID NO: 5.

In some embodiments, the nucleotide sequence encoding the DnaJ domain comprises or consists of a nucleotide sequence having at least 70%, 75%, 80%, 85%, 90%, 95%, 96%, 97%, 98% or 99% sequence identity to SEQ ID NO: 86. In some embodiments, the nucleotide sequence encoding the DnaJ domain comprises or consists of the nucleotide sequence of SEQ ID NO: 86.

In some embodiments, the nucleotide sequence encoding the HEPN domain comprises or consists of a nucleotide sequence having at least 70%, 75%, 80%, 85%, 90%, 95%, 96%, 97%, 98% or 99% sequence identity to SEQ ID NO: 6. In some embodiments, the nucleotide sequence encoding the HEPN domain comprises or consists of the nucleotide sequence of SEQ ID NO: 6.

In some embodiments, the nucleotide sequence encoding the HEPN domain comprises or consists of a nucleotide sequence having at least 70%, 75%, 80%, 85%, 90%, 95%, 96%, 97%, 98% or 99% sequence identity to SEQ ID NO: 87. In some embodiments, the nucleotide sequence encoding the HEPN domain comprises or consists of the nucleotide sequence of SEQ ID NO: 87.

In some embodiments, the SRR domain is joined to the DnaJ domain via a linker. In some embodiments, the SRR domain is directly joined to the DnaJ domain.

In some embodiments, the UbL domain is directly joined to the SRR domain. In some embodiments, the UbL domain is joined to the SRR domain via a linker.

In some embodiments, the DnaJ domain is directly joined to the HEPN domain. In some embodiments, the DnaJ domain is joined to the HEPN domain via a linker.

In some embodiments, the linker is a polypeptide linker. Preferably, the linker is a GS linker. In some embodiments, the linker is a GGGGS (SEQ ID NO: 46) linker. In some embodiments, the linker is a AEAAAKALEAEAAAKA (SEQ ID NO: 47) linker.

In some embodiments, the DnaJ domain is joined to the HEPN domain via a FQRFYTSWNQEATSHKSERQQQNKEKCPPSAGQTYSQRFFVPPTFKS (SEQ ID NO: 50) linker, or a linker with at least 70%, 75%, 80%, 85%, 90%, 95%, 96%, 97%, 98% or 99% sequence identity thereto.

In some embodiments, the nucleotide sequence encoding an engineered sacsin comprises or consists of a nucleotide sequence having at least 70%, 75%, 80%, 85%, 90%, 95%, 96%, 97%, 98% or 99% sequence identity to SEQ ID NO: 8, 10, 12 or 14. In some embodiments, the nucleotide sequence encoding an engineered sacsin comprises or consists of the nucleotide sequence of SEQ ID NO: 8, 10, 12 or 14.

In some embodiments, the nucleotide sequence encoding an engineered sacsin comprises or consists of a nucleotide sequence having at least 70%, 75%, 80%, 85%, 90%, 95%, 96%, 97%, 98% or 99% sequence identity to SEQ ID NO: 8, 10, 12, 14 or 88. In some embodiments, the nucleotide sequence encoding an engineered sacsin comprises or consists of the nucleotide sequence of SEQ ID NO: 8, 10, 12, 14 or 88.

In some embodiments, the polynucleotide is less than or equal to about 5200 bp in length.

In some embodiments, the polynucleotide is less than or equal to about 5100 bp, 5000 bp, 4900 bp or 4800 bp in length.

In some embodiments, the polynucleotide is about 3200-5200 bp, 3200-5100 bp, 3200-5000 bp, 3200-4900 or 3200-4800 bp in length.

In some embodiments, the polynucleotide is about 3300-5200 bp, 3300-5100 bp, 3300-5000 bp, 3300-4900 bp or 3300-4800 bp in length.

In some embodiments, the polynucleotide is less than or equal to about 3700 bp in length.

In some embodiments, the polynucleotide is less than or equal to about 3600 bp, 3500 bp or 3400 bp in length.

In some embodiments, the polynucleotide is about 3200-3700 bp, 3200-3600 bp, 3200-3500 bp, 3200-3400 or 3200-3350 bp in length.

In some embodiments, the polynucleotide is about 3300-3700 bp, 3300-3600 bp, 3300-3500 bp or 3300-3400 bp in length.

In some embodiments, the nucleotide sequence encoding an engineered sacsin is operably linked to one or more expression control sequence.

In some embodiments, the nucleotide sequence encoding an engineered sacsin is operably linked to one or more promoter.

In some embodiments, the promoter is a constitutive promoter, a tissue-specific promoter or an inducible promoter.

In some embodiments, the promoter is a CBA promoter, a CMV promoter, CAG promoter, an L7-Pcp2 promoter, a human synapsin 1 (hSyn1) promoter or a neuronal specific enolase promoter. In some embodiments, the promoter is a CAG promoter.

In some embodiments, the promoter is a CBA promoter, a CMV promoter or an L7-Pcp2 promoter. In some embodiments, the promoter is a CBA promoter.

In some embodiments, the nucleotide sequence encoding an engineered sacsin is operably linked to one or more enhancer.

In some embodiments, the enhancer is a CMV enhancer.

In some embodiments, the nucleotide sequence encoding an engineered sacsin is operably linked to a CMV enhancer and a CBA promoter. In some embodiments, the nucleotide sequence encoding an engineered sacsin is operably linked to a CMV enhancer, a CBA promoter and a human beta globin intron.

In some embodiments, the nucleotide sequence encoding an engineered sacsin is operably linked to a polyadenylation sequence.

In some embodiments, the polyadenylation sequence is a human growth hormone (hGh) polyadenylation sequence, bovine growth hormone (bGH) polyadenylation sequence, a SV40 polyadenylation sequence, or a rabbit beta-globin polyadenylation sequence. In some embodiments, the polyadenylation sequence is a human growth hormone (hGh) polyadenylation sequence.

In some embodiments, the polynucleotide comprises a Kozak sequence.

In some embodiments, the polynucleotide comprises or consists of a nucleotide sequence having at least 70%, 75%, 80%, 85%, 90%, 95%, 96%, 97%, 98% or 99% sequence identity to any one of SEQ ID NOs: 25-38. In some embodiments, the polynucleotide comprises or consists of the nucleotide sequence of any one of SEQ ID NOs: 25-38.

In one aspect, the invention provides a polypeptide encoded by the polynucleotide of the invention.

In one aspect, the invention provides a polypeptide that comprises or consists of an amino acid sequence having at least 70%, 75%, 80%, 85%, 90%, 95%, 96%, 97%, 98% or 99% sequence identity to any one of SEQ ID NOs: 9, 11, 13 or 15. In one aspect, the invention provides a polypeptide that comprises or consists of the amino acid sequence of any one of SEQ ID NOs: 9, 11, 13 or 15.

In one aspect, the invention provides a vector comprising the polynucleotide of the invention.

In some embodiments, the vector is a viral vector.

In some embodiments, the vector is an AAV, retroviral or lentiviral vector.

In preferred embodiments, wherein the vector is an AAV vector.

In some embodiments, the vector comprises or consists of a nucleotide sequence having at least 70%, 75%, 80%, 85%, 90%, 95%, 96%, 97%, 98% or 99% sequence identity to any one of SEQ ID NOs: 73-80. In some embodiments, the vector comprises or consists of the nucleotide sequence of any one of SEQ ID NOs: 73-80.

In some embodiments, the vector is in the form of a viral vector particle.

In some embodiments, the vector is in the form of an AAV vector particle.

In one aspect, the invention provides a viral vector particle comprising the polynucleotide of the invention.

In one aspect, the invention provides an AAV vector particle comprising the polynucleotide of the invention.

In some embodiments, the AAV vector particle comprises a capsid selected from the group consisting of an AAV9 PHP.eB; AAV-Se2w; AAV9; AAV9 PHP.B; and AAVrh10 capsid. In some embodiments, the AAV vector particle comprises a AAV9 PHP.eB capsid. In some embodiments, the AAV vector particle comprises a AAV-Se2w capsid.

In one aspect, the invention provides a cell comprising the polynucleotide or vector of the invention.

In one aspect, the invention provides a method of producing the vector of the invention, comprising culturing the cell of the invention under conditions for the production of the vector.

In one aspect, the invention provides a pharmaceutical composition comprising the polynucleotide, vector, polypeptide or cell of the invention and a pharmaceutically-acceptable carrier, diluent or excipient.

In some embodiments, the pharmaceutical composition is formulated for systemic or local delivery.

In some embodiments, the pharmaceutical composition is formulated for intravascular, intravenous, intra-arterial, intracranial or intraparenchymal brain delivery.

In one aspect, the invention provides the polynucleotide, vector, polypeptide or cell of the invention for use in medicine.

In one aspect, the invention provides the polynucleotide, vector, polypeptide or cell of the invention for use in treating a cerebellar ataxia.

In one aspect, the invention provides the polynucleotide, vector, polypeptide or cell of the invention for use in treating autosomal recessive spastic ataxia of Charlevoix-Saguenay (ARSACS).

In some embodiments, the polynucleotide, vector, polypeptide or cell is administered to a subject systemically or locally.

In some embodiments, the polynucleotide, vector, polypeptide or cell is administered to a subject intracranially or intraparenchymally.

### DESCRIPTION OF THE DRAWINGS

FIG. 1

Minisacsin gene expression and functionality in human neuronal like cells. A. Top: scheme of sacsin full length structure. Grey bars represent the corresponding regions whose cDNAs were cloned into minisacsin. Bottom: scheme of minisacsin protein and gene structure. NheI and XhoI enzymes sites were used for cloning into pcDNA3.1 between CMV universal promoter and polyadenylation signal (PolyA). A Kozak sequence was put immediately before the AUG site and after NheI. GS-linker indicates a short spacer that was put in frame between sacsin N- and C-terminal regions. Red arrows represent four primers used for sequencing the final construct. B. Western blot (WB) analysis of minisacsin ectopic expression in SH-SYWY cells 48 h after transfection, both with sacsin-specific AbN (left) and V5-tag Ab (right). Stars represent aspecific bands. C. Immunofluorescence followed by widefield deconvolution microscopy (60×) analysis of minisacsin ectopic expression and NFH bundling recovery in SH-SY5Y cells 48 h after transfection. All cells were stained for V5 (green) and NFH (red). GFP-V5 transfection was used as a control. White arrows indicate examples of NFH bundling in SACS/cells; white head-arrows indicate examples of resolved NFH bundles, with more diffused filamentous structure, in SACS^{-/-} cells transfected with minisacsin. Both minisacsin and GFP-V5 transfected the same % of cells (˜10%). D. Quantification graphs, relative to SACS^{-/-} #1 in C, of mean NFH intensity (left) and volume of maximum NFH object (right) as reconstructed by deconvolution analysis, which reflects the presence of big accumulation and bundles in SACS/cells. Bars represent mean±SEM (30 images per sample); Student's t-test: *p<0.05, ***p<0.001, ****p<0.0001. E. Quantification of average maximum NFH volume per cell after deconvolution and 3D-reconstruction, relative to two SACS/clones. Data shown as mean±SEM; n=2; at least 20 cells quantified for each experiment; *p<0.05, **p<0.01 (two-way ANOVA with Tuckey correction).

FIG. 2

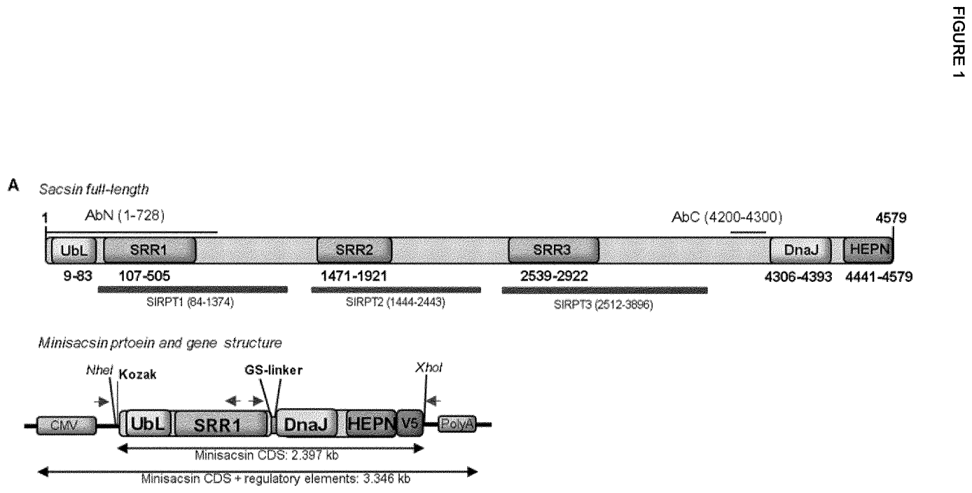

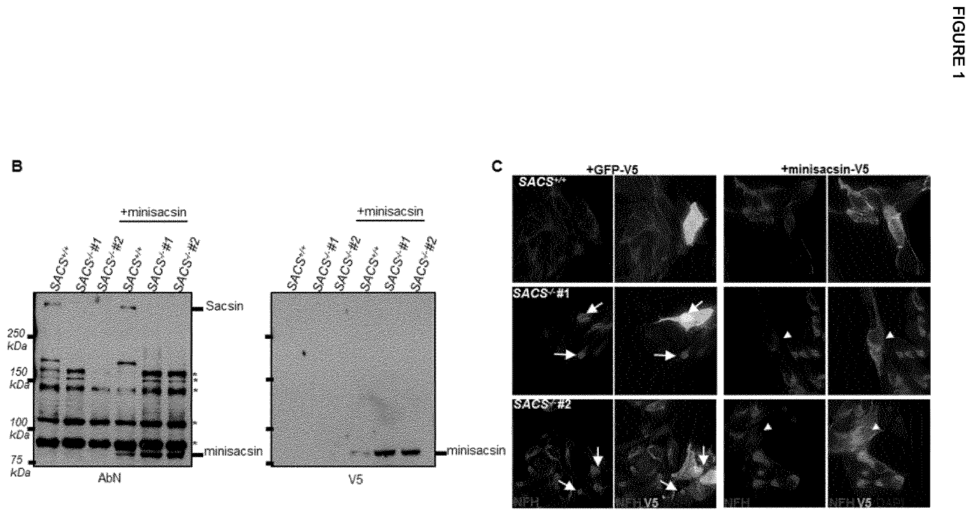

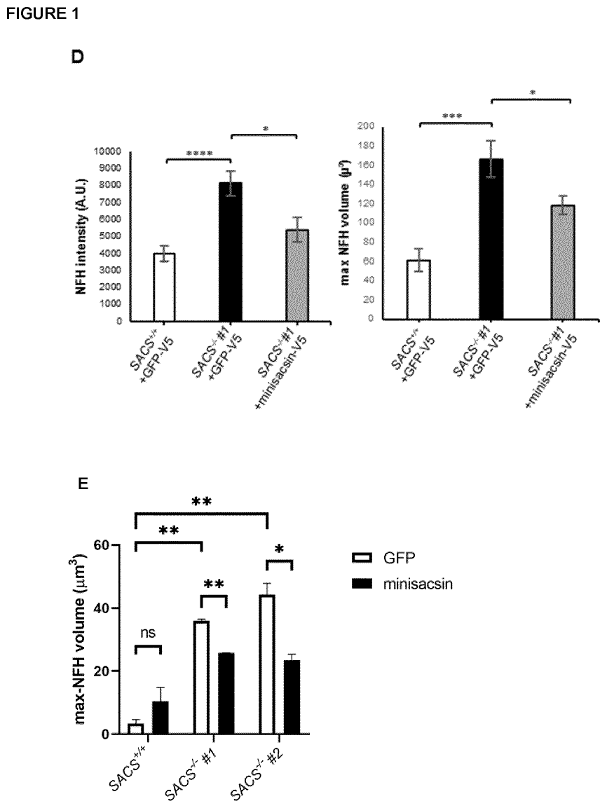

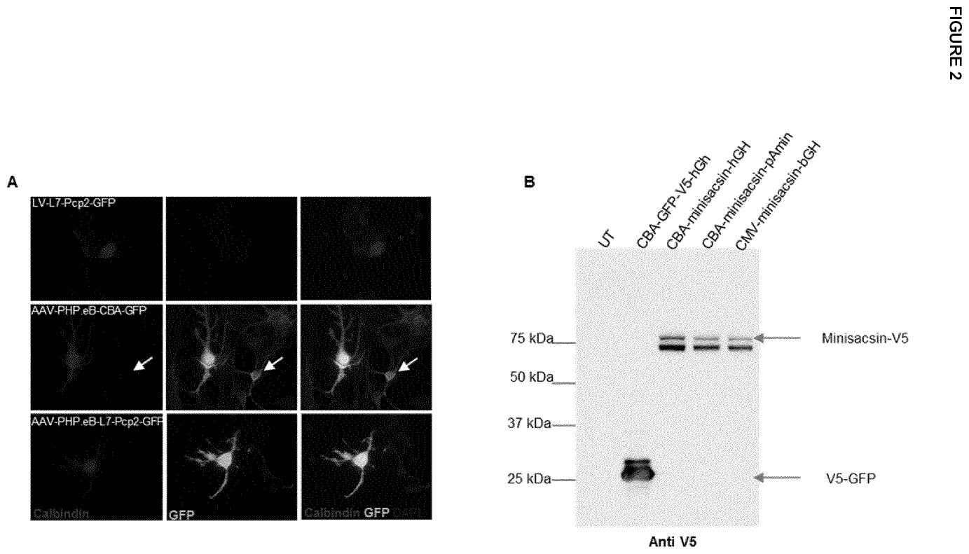

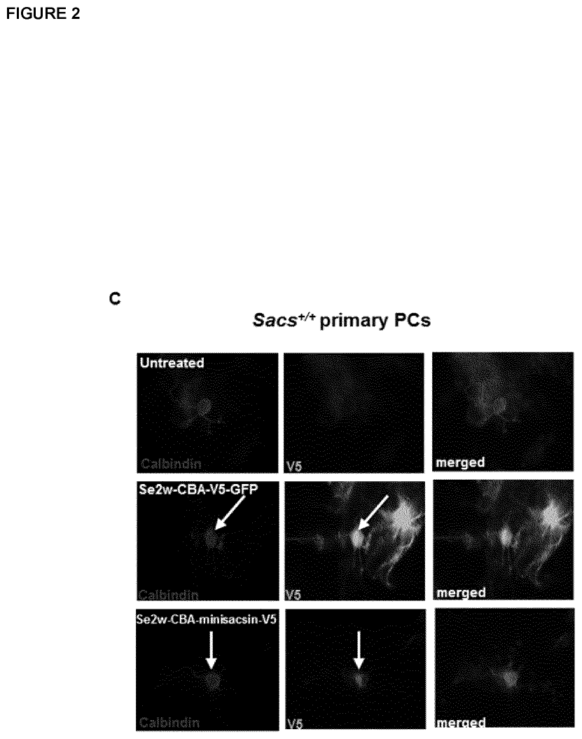

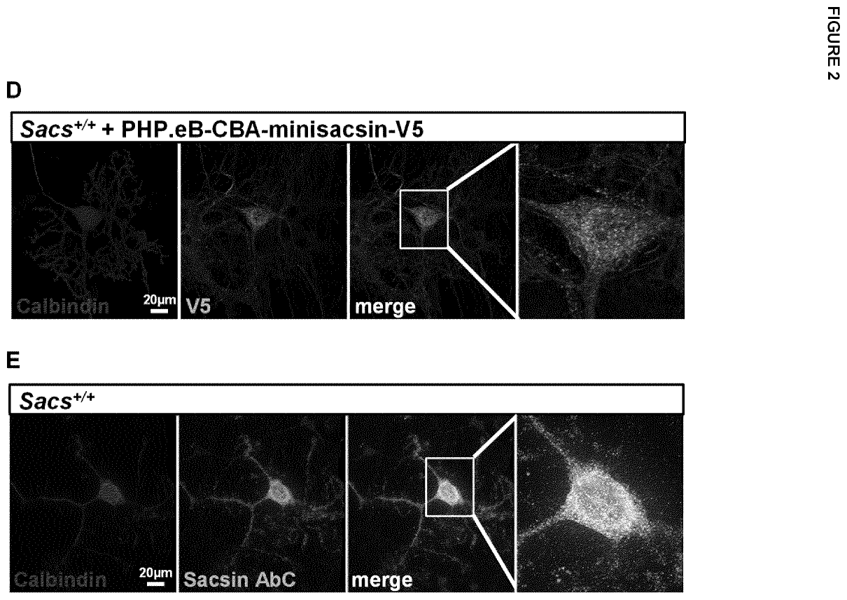

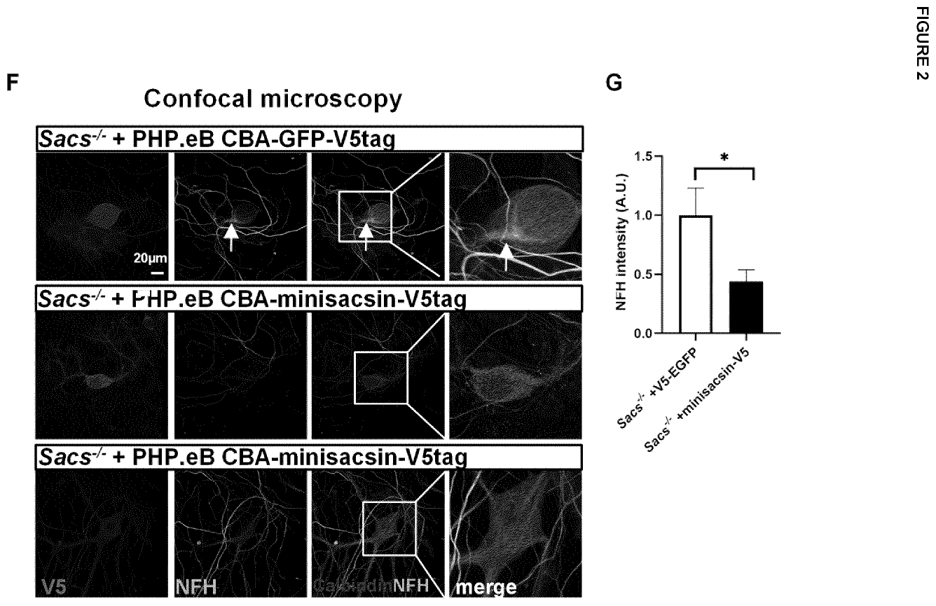

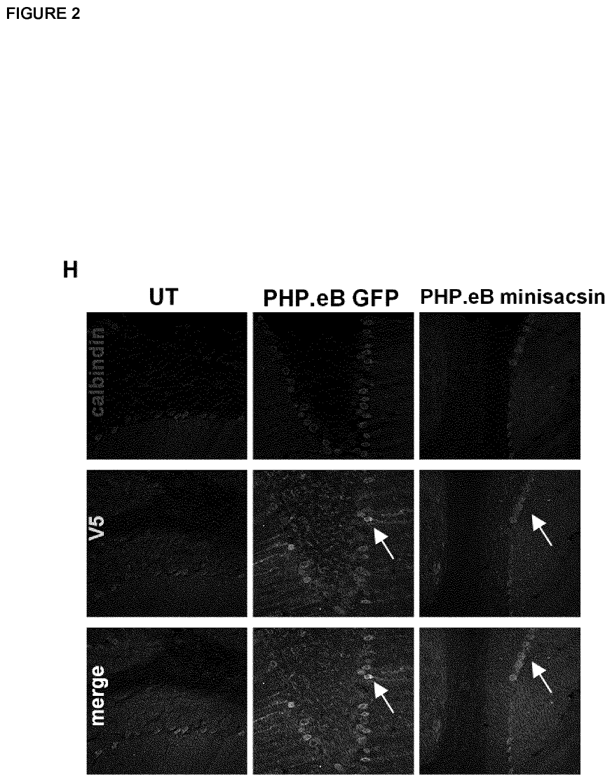

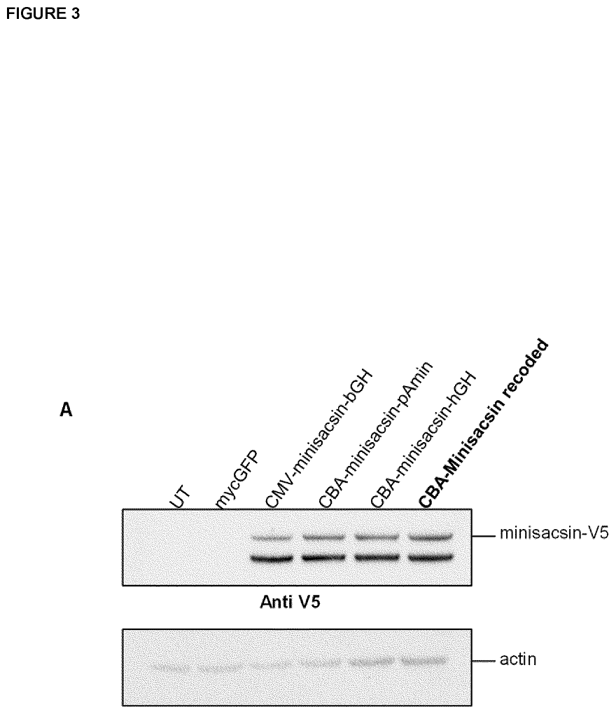

Minisacsin AAV-mediated expression in cerebellum and PCs. A. Representative images at widefield epifluorescence (60×) of DIV11 PCs infected at DIVO with AAV PHP.eB-CBA-GFP or AAV PHP.eB-L7-Pcp2-GFP, stained for calbindin (red). Infection with Lentivirus (LV)-L7-Pcp2-GFP was used as negative control, as they inefficiently transduce PCs in vitro. White arrow indicates examples of cell types other than PCs infected by PHP.eB-CBA-GFP. B. WB analysis of minisacsin ectopic expression in vitro human HEK293T cells 48 h after transfection, with different promoters and PolyA signals: CBA-GFP-hGh, CBA-minisacsin-hGh and CBA-minisacsin-Pamin were cloned into pAAV vectors; CMV-minisacsin-bGh is the same as in FIG. 1B; UT=untransfected. C. Immunofluorescence showing expression of minisacsin ex vivo in cultured PCs at DIV8 after transduction with 10\hat{ }8 vg of Se2w carrying minisacsin or GFP control at DIVO; white arrows indicate calbindin positive PCs. D. Confocal microscopy (63×) of DIV11 control Sacs^{+/+} PCs infected at DIVO with 10\hat{ }10 vg of AAV PHP.eB-CBA-minisacsin, stained for calbindin (red) and V5-Tag (green). Scale bars=20 μm. E. Widefield deconvolution microscopy (63×) of endogenous sacsin (green) and calbindin (red) on DIV15 control Sacs^{+/+} PCs. Scale bars=20 μm. F. Confocal microscopy (63×) with V5-tag antibody (colorized in magenta), calbindin (colorized in red) and total NFH (colorized in green) on DIV15 Sacs^{-/-} PCs infected at DIVO with AAV-PHP.eB-CBA-V5-EGFP-hGh (above) or AAV-PHP.eB-CBA-minisacsin-V5-pAmin (below). The white arrows above indicate NF bundles in proximal dendrites; such bundles are not observed in Sacs^{-/-} with minisacsin. G. Quantification of average NFH intensity peak across each proximal dendrite, showing a significative reduction in Sacs^{-/-} PCs with minisacsin. Data shown as mean±SEM; n=3 biologically independent experiments; at least 10 dendrites were quantified for each experiment; *p<0.05 (Student's t-test with Welch correction). Scale bars=20 μm. H. Confocal microscopy (63×) on sagittal cerebellar slices from wild-type mice at two months of age uninjected or systemically injected with PHP.eB EGFP-V5 or PHP.eB minisacsin-V5 at one month. Immunofluorescence was performed with V5 (green) and calbindin (red). White arrows indicate examples of PCs expressing EGFP or minisacsin.

FIG. 3

The codon-optimized version of minisacsin exhibits stability and high expression in vitro. A. WB analysis of recoded minisacsin ectopic expression in vitro human HeLa cells 48 h after transfection, compared to expression of original minisacsin CDS with different promoters and PolyA signals: CBA-minisacsin-hGh, CBA-minisacsin-Pamin and CBA-recoded minisacsin-hGh were cloned into pAAV vectors; CMV-minisacsin-bGh is the same as in FIG. 1B; UT=untransfected. Cells transfected with pcDNA3.1 myc-GFP were used as control.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### Autosomal Recessive Spastic Ataxia of Charlevoix-Saguenay (ARSACS)

Autosomal recessive spastic ataxia of Charlevoix-Saguenay (ARSACS) is a rare cerebellar ataxia characterised by motor-coordination problems from very young ages. ARSACS is the second most common cause of recessive ataxia, with over 200 mutations described worldwide. The first two mutations were identified in the French-Canadian population 20 years ago due to a founder effect, but the disease is now present worldwide with an incidence of 2:100,000 inhabitants. In Quebec the prevalence is 1:1932. As an approximation, with over 300 persons diagnosed with the disease, ARSACS patients in Eastern Canada may represent about a third of all cases worldwide.

ARSACS is caused by mutations in the SACS gene, resulting in the inactivation of the encoded protein, sacsin.

### Sacsin

Sacsin is a very large, multimodular protein of 520 kDa, which contains a combination of domains: a ubiquitin-like domain (UbL), three sacsin repeat regions (SRR), homologous to Hsp90 chaperone, a DnaJ domain homologous to Hsp 40 and a nucleotide binding domain (HEPN). Although these domains suggest an involvement in protein quality control, the cellular function of this protein remains largely unknown. Previous studies have indicated a role for sacsin in regulating the cytoskeleton, crucial for cellular functionality. Indeed, cell and mouse models of ARSACS present severe alteration of cytoskeleton.

An example nucleotide sequence encoding full length sacsin is NCBI RefSeq NM_014363.6.

### Sacsin Repeat Regions (SRR)

Sacsin comprises three sacsin repeat region (SRR) domains, termed SRR1, SRR2 and SRR3. SRR1 contains regions of homology with the Hsp90 chaperone family.

An example nucleotide sequence encoding the SRR1 domain is:

(SEQ ID NO: 1)
AAAGGGGGAGGTCGATTTGGTCAGACAACGCCACCACTTGTTGAT
TTTCTCAAGGACATTTTGAGAAGATATCCAGAAGGAGGACAGATT
CTTAAGGAATTAATTCAGAATGCAGAAGATGCTGGGGCGACAGAA
GTTAAATTTTTATATGATGAAACTCAATACGGAACAGAGACTCTT
TGGTCAAAAGATATGGCGCCATATCAGGGGCCAGCTCTCTATGTG
TACAACAACGCGGTTTTCACCCCAGAGGACTGGCACGGCATTCAA
GAAATAGCAAGAAGCAGGAAAAAGGATGATCCTCTGAAGGTCGGA
AGATTTGGAATTGGGTTTAATTCTGTCTATCATATAACAGATGTT
CCTTGTATCTTTAGTGGTGACCAAATCGGGATGCTAGATCCTCAT
CAAACACTTTTTGGCCCACATGAATCAGGCCAATGTTGGAATCTC
AAAGATGACAGCAAAGAAATTAGTGAACTTTCAGACCAGTTTGCA
CCATTTGTTGGCATTTTTGGAAGCACCAAGGAAACATTTATAAAC
GGCAATTTTCCAGGAACATTTTTCCGTTTCCCTCTTCGCCTACAA
CCTTCACAACTTAGTAGTAACCTCTACAATAAGCAGAAGGTTCTT
GAGTTGTTTGAGTCTTTTAGGGCAGATGCAGACACAGTGCTGCTC
TTTCTGAAAAGTGTGCAGGATGTTTCCTTATATGTCCGAGAGGCT
GACGGAACAGAGAAACTGGTGTTTAGAGTGACTTCGAGTGAGAGT
AAGGCACTGAAACATGAGCGGCCGAATTCTATAAAGATTCTGGGA
ACTGCTATAAGTAACTATTGTAAAAAGACTCCAAGCAATAACATC
ACCTGTGTAACATATCACGTAAATATTGTTTTAGAAGAGGAGAGT
ACTAAGGATGCACAGAAAACATCTTGGTTGGTGTGTAACAGTGTG
GGTGGGCGAGGGATCAGTAGTAAGCTTGACTCTTTAGCTGATGAA
CTGAAATTTGTCCCAATCATTGGAATAGCCATGCCTTTATCAAGC
AGAGATGATGAAGCAAAAGGAGCAACGTCTGATTTCTCAGGAAAA
GCATTTTGTTTCCTTCCTTTACCACCTGGTGAGGAAAGCAGCACA
GGCCTCCCAGTTCACATCAGTGGGTTCTTTGGCCTTACTGATAAC
CGCAGGAGCATAAAATGGAGAGAGCTGGACCAGTGGAGAGACCCG
GCAGCCTTATGGAATGAGTTTCTTGTCATGAATGTTGTCCCCAAA
GCTTATGCTACTCTG

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 1, preferably wherein the nucleotide sequence encoding the SRR domain maintains the function of SEQ D NO: 1.

In some embodiments, the nucleotide sequence encoding the SRR domain comprises or consists of the nucleotide sequence of SEQ ID NO: 1.

An example codon optimised nucleotide sequence encoding the SRR1 domain is:

(SEQ ID NO: 84)
AAGGGCGGCGGCCGGTTCGGCCAAACAACCCCGCCACTGGTGGAC
TTCCTGAAGGACATCCTGAGAAGATACCCCGAGGGCGGGCAGATA
CTGAAAGAGCTTATTCAGAACGCCGAAGACGCCGGCGCCACCGAG
GTGAAGTTCCTGTACGACGAGACACAGTACGGCACCGAGACTCTG
TGGAGCAAGGACATGGCCCCCTACCAAGGCCCCGCCCTGTACGTG
TACAACAACGCCGTGTTCACCCCCGAGGACTGGCACGGCATCCAA
GAGATCGCTAGAAGCAGAAAGAAGGACGACCCCCTGAAGGTGGGC
AGATTCGGCATCGGCTTCAACAGCGTGTACCACATCACCGACGTG
CCCTGCATCTTCAGCGGCGATCAGATCGGCATGCTGGACCCCCAC
CAAACACTGTTCGGCCCCCACGAGAGCGGGCAGTGCTGGAACCTG
AAGGACGACAGCAAGGAGATCAGCGAACTGTCCGATCAGTTCGCC
CCCTTCGTGGGCATCTTCGGCAGCACCAAGGAGACCTTCATCAAC
GGCAACTTCCCCGGCACCTTCTTCAGATTCCCCCTGAGACTGCAG
CCTTCTCAGCTGAGCAGCAACCTGTACAACAAGCAGAAGGTGCTG
GAGCTGTTCGAGAGCTTCAGAGCCGACGCCGACACCGTGCTGCTG
TTCCTGAAGAGCGTGCAAGACGTGAGCCTGTACGTGAGAGAGGCC
GACGGTACGGAGAAACTGGTGTTCAGAGTGACAAGCAGCGAGAGC
AAGGCCCTGAAGCACGAGAGACCCAACAGCATCAAGATCCTGGGC
ACCGCCATCAGCAACTACTGCAAGAAGACCCCTAGCAACAACATC
ACCTGCGTGACCTACCACGTGAACATCGTGCTGGAGGAGGAGAGC
ACCAAGGACGCTCAGAAGACAAGCTGGCTGGTGTGCAACAGCGTG
GGCGGCAGAGGCATCAGCAGCAAGCTGGACAGCCTGGCCGACGAG
CTGAAGTTCGTGCCCATCATCGGCATCGCCATGCCCCTGAGCAGC
AGAGACGACGAGGCCAAGGGCGCCACAAGCGACTTTAGTGGTAAA
GCCTTCTGCTTCCTGCCACTGCCACCCGGAGAGGAGAGCAGCACC
GGCCTGCCCGTGCACATCAGCGGCTTCTTCGGCCTGACCGACAAC
AGAAGAAGCATCAAGTGGAGAGAGCTGGATCAGTGGAGAGACCCC
GCCGCCCTGTGGAACGAGTTCCTGGTGATGAACGTGGTGCCCAAG
GCCTACGCCACCCTG

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 84, preferably wherein the nucleotide sequence encoding the SRR domain maintains the function of SEQ D NO: 84.

In some embodiments, the nucleotide sequence encoding the SRR domain comprises or consists of the nucleotide sequence of SEQ ID NO: 84.

An example nucleotide sequence encoding the SRR2 domain is:

(SEQ ID NO: 2)
ATAAATCCTGAAAACATGGGATTTGAGCAGTCAGGACAAAGAGAG
CCACTTACTGTAAGAATTAAAAATATTCTGGAAGAATACCCTTCA
GTGTCAGATATTTTTAAAGAACTACTTCAAAACGCTGATGATGCA
AATGCAACAGAATGCAGTTTCTTGATTGATATGAGAAGAAATATG
GACATAAGAGAGAATCTCCTAGACCCAGGGATGGCAGCTTGTCAT
GGACCTGCTTTGTGGTCATTCAACAATTCTCAATTCTCAGATTCA
GATTTTGTGAACATAACTAGGTTAGGAGAATCTTTAAAAAGGGGA
GAAGTTGACAAAGTTGGAAAATTTGGTCTTGGATTTAATTCTGTG
TACCATATCACTGACATTCCCATCATTATGAGTCGGGAATTCATG
ATAATGTTCGATCCAAACATAAATCATATCAGTAAACACATTAAA
GACAAATCCAATCCTGGGATCAAAATTAATTGGAGTAAACAACAG
AAAAGACTTAGAAAATTTCCTAATCAGTTCAAACCATTTATAGAT
GTATTTGGCTGTCAGTTACCTTTGACTGTAGAAGCACCTTACAGC
TATAATGGAACCCTTTTCCGACTGTCCTTTAGAACTCAACAGGAA
GCAAAAGTGAGTGAAGTTAGTAGTACGTGCTACAATACAGCAGAT
ATTTATTCTCTTGTGGATGAATTTAGTCTCTGTGGACACAGGCTT
ATCATTTTCACTCAGAGTGTAAAGTCAATGTATTTGAAGTACTTG
AAAATTGAGGAAACCAACCCCAGTTTAGCACAAGATACAGTAATA
ATTAAAAAAAAATCCTGCTCTTCCAAAGCATTGAACACACCTGTC
TTAAGTGTTTTAAAAGAGGCTGCTAAGCTCATGAAGACTTGCAGC
AGCAGTAATAAAAAGCTTCCCAGTGATGAACCAAAGTCATCTTGC
ATTCTTCAGATCACAGTGGAAGAATTTCACCATGTGTTCAGAAGG
ATTGCTGATTTACAGTCGCCACTTTTTAGAGGTCCAGATGATGAC
CCAGCTGCTCTCTTTGAAATGGCTAAGTCTGGCCAATCAAAAAAG
CCATCAGATGAGTTGTCACAGAAAACAGTAGAGTGTACCACGTGG
CTTCTGTGTACTTGCATGGACACAGGAGAGGCTCTGAAGTTTTCC
CTGAGTGAGAGTGGAAGAAGACTAGGACTGGTTCCATGTGGGGCA
GTAGGAGTTCAGCTGTCAGAAATCCAGGACCAGAAGTGGACAGTG
AAACCACACATTGGAGAGGTGTTTTGCTATTTACCTTTACGAATA
AAAACAGGCTTGCCAGTTCATATCAATGGGTGCTTTGCTGTTACA
TCAAATAGGAAAGAAATCTGGAAAACAGATACAAAAGGACGATGG
AATACCACGTTCATGAGACATGTTATTGTGAAAGCTTACTTACAG
GTACTGAGTGTCTTACGGGACCTGGCCACTAGTGGGGAGCTAATG
GATTATACTTACTATGCAGTATGGCCCGATCCTGATTTAGTTCAT
GATGATTTTTCTGTAATTTGCCAAGGATTTTATGAAGATATAGCT
CATGGA

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 2, preferably wherein the nucleotide sequence encoding the SRR domain maintains the function of SEQ D NO: 2.

In some embodiments, the nucleotide sequence encoding the SRR domain comprises or consists of the nucleotide sequence of SEQ ID NO: 2.

An example nucleotide sequence encoding the SRR3 domain is:

(SEQ ID NO: 3)
AATGTCTGTTTTACAACACTTGGCACAGAATTTGGGCAGAAAGAA
AAATTGACCAGCAGAATTAAGAGCATCCTTAATGCATATCCTTCT
GAAAAGGAAATGTTGAAAGAGCTTCTTCAAAATGCTGATGATGCA
AAGGCGACAGAAATCTGTTTTGTGTTTGATCCTAGACAGCATCCA
GTTGATAGAATATTTGATGATAAGTGGGCCCCATTGCAAGGGCCA
GCACTTTGTGTGTACAACAACCAGCCATTTACAGAAGATGATGTT
AGAGGAATTCAGAATCTTGGAAAAGGCACGAAAGAGGGAAATCCT
TATAAAACTGGACAGTATGGAATAGGATTCAATTCTGTGTATCAT
ATCACAGACTGCCCATCTTTTATTTCTGGCAATGACATCCTGTGT
ATTTTTGATCCTCATGCCAGATATGCACCAGGGGCCACATCCATT
AGTCCCGGACGCATGTTTAGAGATTTGGATGCAGATTTTAGGACA
CAGTTCTCAGATGTTCTGGATCTTTATCTGGGAACCCATTTTAAA
CTGGATAATTGCACAATGTTCAGATTTCCTCTTCGTAATGCAGAA
ATGGCAAAAGTTTCGGAAATTTCGTCTGTTCCAGCATCAGACAGA
ATGGTCCAGAATCTTTTGGACAAACTGCGCTCAGATGGGGCAGAA
CTTCTAATGTTTCTTAATCACATGGAAAAAATTTCTATTTGTGAA
ATAGATAAGAGTACTGGAGCTCTAAATGTGCTGTATTCAGTAAAG
GGCAAAATCACAGATGGAGACAGATTGAAAAGGAAACAATTTCAT
GCATCTGTAATTGATAGTGTTACTAAAAAGAGGCAGCTCAAAGAC
ATACCAGTTCAACAAATAACCTATACTATGGATACTGAGGACTCT
GAAGGAAATCTTACTACGTGGCTAATTTGTAATAGATCAGGCTTT
TCAAGTATGGAGAAAGTATCTAAAAGTGTCATATCAGCTCACAAG
AACCAAGATATTACTCTTTTCCCACGTGGTGGAGTAGCTGCCTGC
ATTACTCACAACTATAAAAAACCCCATAGGGCCTTCTGTTTTTTG
CCTCTTTCTTTGGAGACTGGGCTGCCATTTCATGTGAATGGCCAC
TTTGCACTGGATTCAGCCAGAAGGAACCTGTGGCGTGATGATAAT
GGAGTTGGTGTTCGAAGTGACTGGAATAACAGTTTAATGACAGCA
TTAATAGCTCCTGCATATGTTGAATTGCTAATACAGTTAAAAAAA
CGGTATTTCCCTGGTTCTGATCCAACATTATCAGTGTTACAGAAC
ACCCCTATTCATGTTGTAAAGGACACTTTAAAGAAGTTTTTATCG

In some embodiments, the nucleotide sequence encoding the SRR domain comprises or consists of a nucleotide sequence having at least 70%, 75%, 80%, 85%, 90%, 95%, 96%, 97%, 98% or 99% sequence identity to SEQ ID NO: 3, preferably wherein the nucleotide sequence encoding the SRR domain maintains the function of SEQ D NO: 3.

In some embodiments, the nucleotide sequence encoding the SRR domain comprises or consists of the nucleotide sequence of SEQ ID NO: 3.

### Ubiquitin-Like Domain (UbL)

The ubiquitin-like (UbL) domain has been indicated to be involved in proteasome binding.

An example nucleotide sequence encoding the UbL domain is:

(SEQ ID NO: 4)
ATGGAGACCAAGGAGAACAGGTGGGTCCCGGTGACCGTGCTCCCCGGCTGCGTGGGCTGCAGGACCGTCGCGGCG
CTGGCGTCCTGGACCGTGCGCGATGTGAAGGAACGTATCTTCGCGGAGACTGGCTTCCCGGTGTCGGAGCAGCGG
CTGTGGCGCGGCGGCCGCGAGTTATCTGACTGGATCAAGATTGGAGATCTGACTTCCAAAAATTGTCATCTTTTT
GTAAACCTTCAATCAAAAGGCTTA

In some embodiments, the nucleotide sequence encoding the UbL domain comprises or consists of a nucleotide sequence having at least 70%, 75%, 80%, 85%, 90%, 95%, 96%, 97%, 98% or 99% sequence identity to SEQ ID NO: 4, preferably wherein the nucleotide sequence encoding the UbL domain maintains the function of SEQ D NO: 4.

In some embodiments, the nucleotide sequence encoding the UbL domain comprises or consists of the nucleotide sequence of SEQ ID NO: 4.

An example codon optimised nucleotide sequence encoding the UbL domain is:

(SEQ ID NO: 85)
GAGACCAAGGAGAACAGATGGGTACCCGTGACCGTTCTGCCCGGCTGTGTGGGCTGTCGTACGGTGGCAGCCCTG
GCAAGCTGGACCGTGAGAGACGTGAAGGAGAGAATCTTCGCCGAGACCGGCTTCCCCGTGAGCGAGCAGAGACTG
TGGAGAGGCGGCAGAGAACTGTCCGACTGGATCAAGATCGGCGACCTGACAAGCAAGAACTGCCACCTGTTCGTG
AACCTGCAGAGCAAGGGCCTG

In some embodiments, the nucleotide sequence encoding the UbL domain comprises or consists of a nucleotide sequence having at least 70%, 75%, 80%, 85%, 90%, 95%, 96%, 97%, 98% or 99% sequence identity to SEQ ID NO: 85, preferably wherein the nucleotide sequence encoding the UbL domain maintains the function of SEQ ID NO: 85.

In some embodiments, the nucleotide sequence encoding the UbL domain comprises or consists of the nucleotide sequence of SEQ ID NO: 85.

### DnaJ Domain

The DnaJ is homologous to the Hsp40 co-chaperone.

An example nucleotide sequence encoding the DnaJ domain is:

(SEQ ID NO: 5)
ATCTTAAAAGAAGTGACATCTGTGGTGGAGCAAGCATGGAAGCTTCCAGAATCGGAACGAAAAAAGATTATTAGG
CGGTTGTATTTGAAATGGCATCCTGACAAAAATCCAGAGAACCATGACATTGCCAATGAAGTTTTTAAACATTTG
CAGAATGAAATCAACAGATTAGAAAAACAGGCTTTTCTAGATCAAAATGCAGACAGGGCCTCCAGACGAACATTT
TCAACCTCAGCATCCCGATTTCAGTCAGACAAATACTCA

In some embodiments, the nucleotide sequence encoding the DnaJ domain comprises or consists of a nucleotide sequence having at least 70%, 75%, 80%, 85%, 90%, 95%, 96%, 97%, 98% or 99% sequence identity to SEQ ID NO: 5, preferably wherein the nucleotide sequence encoding the DnaJ domain maintains the function of SEQ D NO: 5.

In some embodiments, the nucleotide sequence encoding the DnaJ domain comprises or consists of the nucleotide sequence of SEQ ID NO: 5.

An example codon optimised nucleotide sequence encoding the DnaJ domain is:

(SEQ ID NO: 86)
ATCCTGAAAGAGGTGACGAGCGTGGTGGAGCAAGCCTGGAAGCTGCCCGAGAGCGAGAGAAAGAAGATCATCAGA
AGACTGTACCTGAAGTGGCACCCCGACAAGAACCCCGAGAACCACGACATCGCCAACGAGGTGTTCAAGCACCTG
CAGAACGAGATCAACAGACTGGAGAAGCAAGCCTTCCTGGACCAAAATGCCGACAGAGCAAGCAGAAGAACCTTC
AGCACAAGCGCAAGCAGATTTCAGAGCGACAAGTACAGC

In some embodiments, the nucleotide sequence encoding the DnaJ domain comprises or consists of a nucleotide sequence having at least 70%, 75%, 80%, 85%, 90%, 95%, 96%, 97%, 98% or 99% sequence identity to SEQ ID NO: 86, preferably wherein the nucleotide sequence encoding the DnaJ domain maintains the function of SEQ D NO: 86.

In some embodiments, the nucleotide sequence encoding the DnaJ domain comprises or consists of the nucleotide sequence of SEQ ID NO: 86.

### HEPN Domain

The HEPN domain is believed to be involved in mediating sacsin dimerisation and in binding nucleotides, such as GTP and ATP.

An example nucleotide sequence encoding the HEPN domain is:

(SEQ ID NO: 6)
GTTGGCAATCCAGTGGAAGCACGCAGATGGCTAAGACAAGCCAGAGCAAACTTCTCAGCTGCCAGGAATGACCTT
CATAAAAATGCCAATGAGTGGGTGTGCTTTAAATGTTACCTTTCTACCAAGTTAGCTTTGATTGCAGCTGACTAT
GCTGTGAGGGGAAAGTCTGATAAAGATGTAAAACCAACTGCACTTGCTCAGAAAATAGAGGAATATAGTCAGCAA
CTTGAAGGACTGACAAATGATGTTCACACATTGGAAGCTTATGGTGTAGACAGTTTAAAAACAAGATACCCTGAT
TTGCTTCCCTTTCCTCAGATCCCAAATGACAGGTTCACTTCTGAGGTTGCTATGAGGGTGATGGAATGTACTGCC
TGTATCATAATAAAACTTGAAAATTTTATGCAACAAAAAGTG

In some embodiments, the nucleotide sequence encoding the HEPN domain comprises or consists of a nucleotide sequence having at least 70%, 75%, 80%, 85%, 90%, 95%, 96%, 97%, 98% or 99% sequence identity to SEQ ID NO: 6, preferably wherein the nucleotide sequence encoding the HEPN domain maintains the function of SEQ D NO: 6.

In some embodiments, the nucleotide sequence encoding the HEPN domain comprises or consists of the nucleotide sequence of SEQ ID NO: 6.

An example codon optimised nucleotide sequence encoding the HEPN domain is:

(SEQ ID NO: 87)
GTGGGCAACCCCGTGGAGGCTAGAAGATGGCTGAGACAAGCTAGAGCCAACTTCAGCGCCGCTAGAAACGACCTG
CACAAGAACGCCAACGAGTGGGTGTGCTTCAAGTGCTACCTGAGCACCAAGCTGGCCCTGATCGCCGCCGACTAC
GCCGTGAGAGGCAAGAGCGACAAGGACGTGAAGCCCACCGCCCTGGCTCAGAAGATCGAGGAGTACTCTCAGCAG
CTGGAGGGCCTGACCAACGACGTGCACACCCTGGAGGCCTACGGCGTGGACAGCCTGAAGACAAGATACCCCGAC
CTGCTGCCCTTCCCTCAGATCCCCAACGACAGATTCACAAGCGAGGTGGCCATGAGAGTGATGGAGTGCACCGCC
TGCATCATCATCAAGCTGGAGAACTTCATGCAGCAGAAGGTG

In some embodiments, the nucleotide sequence encoding the HEPN domain comprises or consists of a nucleotide sequence having at least 70%, 75%, 80%, 85%, 90%, 95%, 96%, 97%, 98% or 99% sequence identity to SEQ ID NO: 87, preferably wherein the nucleotide sequence encoding the HEPN domain maintains the function of SEQ D NO: 87.

In some embodiments, the nucleotide sequence encoding the HEPN domain comprises or consists of the nucleotide sequence of SEQ ID NO: 87.

### Sacsin Internal Repeats (SIRPTs)

Sacsin Internal Repeats (SIRPTs) are three sacsin repeating regions within the full length sacsin, termed SIRPT1, SIRPT2 and SIRPT3. Each SIRPT comprises three sub-repeats in the same order, namely sr1, sr2 and sr3. SIRPT1 and SIRPT3 also have a fourth sub-repeat called srX between their sr2 and sr3. Regions in each SIRPT which include sr1 and sr2 correspond to the respective SRR domain (Romano et al. (2013) Human Mutation 34:525-537).

In one aspect, the invention provides a polynucleotide comprising a nucleotide sequence encoding an engineered sacsin, wherein the engineered sacsin comprises a UbL domain or variant thereof, a SIRPT domain or fragment thereof, a DnaJ domain or variant thereof and an HEPN domain or variant thereof.

In one aspect, the invention provides a polynucleotide comprising a nucleotide sequence encoding an engineered sacsin, wherein the engineered sacsin comprises a UbL domain or variant thereof, a SIRPT domain or fragment thereof, a DnaJ domain or variant thereof and an HEPN domain or variant thereof, and wherein the nucleotide sequence encoding the engineered sacsin is less than or equal to about 5200 bp in length.

In one aspect, the invention provides a polynucleotide comprising a nucleotide sequence encoding an engineered sacsin, wherein the engineered sacsin comprises a UbL domain, a SIRPT domain, a DnaJ domain and an HEPN domain.

In one aspect, the invention provides a polynucleotide comprising a nucleotide sequence encoding an engineered sacsin, wherein the engineered sacsin comprises a UbL domain, a SIRPT domain, a DnaJ domain and an HEPN domain, and wherein the nucleotide sequence encoding the engineered sacsin is less than or equal to about 5200 bp in length.

In some embodiments, the SIRPT domain is a SIRPT1 domain. In some embodiments, the SIRPT domain is a SIRPT2 domain. In some embodiments, the SIRPT domain is a SIRPT3 domain. In some embodiments, the engineered sacsin comprises a fragment of SIRPT1 and/or SIRPT2 and/or SIRPT3.

An example nucleotide sequence encoding a SIRPT1 domain is:

(SEQ ID NO: 81)
AAAGGGGGAGGTCGATTTGGTCAGACAACGCCACCACTTGTTGATTTTCTCAAGGACATTTTGAGAAGATATCCA
GAAGGAGGACAGATTCTTAAGGAATTAATTCAGAATGCAGAAGATGCTGGGGCGACAGAAGTTAAATTTTTATAT
GATGAAACTCAATACGGAACAGAGACTCTTTGGTCAAAAGATATGGCGCCATATCAGGGGCCAGCTCTCTATGTG
TACAACAACGCGGTTTTCACCCCAGAGGACTGGCACGGCATTCAAGAAATAGCAAGAAGCAGGAAAAAGGATGAT
CCTCTGAAGGTCGGAAGATTTGGAATTGGGTTTAATTCTGTCTATCATATAACAGATGTTCCTTGTATCTTTAGT
GGTGACCAAATCGGGATGCTAGATCCTCATCAAACACTTTTTGGCCCACATGAATCAGGCCAATGTTGGAATCTC
AAAGATGACAGCAAAGAAATTAGTGAACTTTCAGACCAGTTTGCACCATTTGTTGGCATTTTTGGAAGCACCAAG
GAAACATTTATAAACGGCAATTTTCCAGGAACATTTTTCCGTTTCCCTCTTCGCCTACAACCTTCACAACTTAGT
AGTAACCTCTACAATAAGCAGAAGGTTCTTGAGTTGTTTGAGTCTTTTAGGGCAGATGCAGACACAGTGCTGCTC
TTTCTGAAAAGTGTGCAGGATGTTTCCTTATATGTCCGAGAGGCTGACGGAACAGAGAAACTGGTGTTTAGAGTG
ACTTCGAGTGAGAGTAAGGCACTGAAACATGAGCGGCCGAATTCTATAAAGATTCTGGGAACTGCTATAAGTAAC
TATTGTAAAAAGACTCCAAGCAATAACATCACCTGTGTAACATATCACGTAAATATTGTTTTAGAAGAGGAGAGT
ACTAAGGATGCACAGAAAACATCTTGGTTGGTGTGTAACAGTGTGGGTGGGCGAGGGATCAGTAGTAAGCTTGAC
TCTTTAGCTGATGAACTGAAATTTGTCCCAATCATTGGAATAGCCATGCCTTTATCAAGCAGAGATGATGAAGCA
AAAGGAGCAACGTCTGATTTCTCAGGAAAAGCATTTTGTTTCCTTCCTTTACCACCTGGTGAGGAAAGCAGCACA
GGCCTCCCAGTTCACATCAGTGGGTTCTTTGGCCTTACTGATAACCGCAGGAGCATAAAATGGAGAGAGCTGGAC
CAGTGGAGAGACCCGGCAGCCTTATGGAATGAGTTTCTTGTCATGAATGTTGTCCCCAAAGCTTATGCTACTCTG
ATCTTAGATTCAATAAAACGTCTGGAGATGGAAAAGAGCTCTGATTTCCCCTTGTCAGTTGATGTTATCTATAAG
CTTTGGCCGGAGGCGAGCAAAGTCAAGGTGCACTGGCAACCGGTGTTAGAGCCTCTATTCAGCGAGCTGTTGCAG
AATGCAGTGATTTATTCAATTAGCTGTGACTGGGTCAGGTTGGAGCAGGTGTACTTCTCAGAACTTGATGAAAAT
TTAGAATACACAAAAACTGTGCTCAACTACCTCCAGAGCTCAGGGAAGCAGATTGCCAAGGTACCAGGGAATGTG
GATGCTGCTGTTCAGCTCACAGCTGCCTCTGGCACAACACCTGTGAGGAAGGTGACGCCCGCGTGGGTGCGGCAG
GTGCTGCGGAAGTGTGCACACCTGGGCTGTGCTGAAGAAAAGCTTCACCTTCTAGAATTTGTGCTTTCTGACCAA
GCCTACAGTGAGCTGCTTGGGCTGGAGCTGCTCCCTTTACAAAATGGCAATTTTGTCCCCTTCTCCTCATCTGTA
TCAGACCAAGATGTCATTTATATTACCTCAGCAGAATATCCAAGGTCCCTTTTCCCAAGTCTTGAAGGAAGATTT
ATTTTGGATAACTTGAAACCTCACCTTGTGGCTGCTTTAAAGGAAGCTGCCCAAACCCGAGGAAGACCATGTACT
CAGCTGCAGCTTCTAAATCCAGAACGATTTGCACGTCTTATCAAGGAAGTAATGAATACATTCTGGCCTGGCAGA
GAATTGATTGTTCAATGGTATCCATTTGATGAAAACAGAAATCACCCATCTGTTTCATGGCTTAAGATGGTTTGG
AAAAATCTTTATATACATTTTTCAGAGGATTTGACTTTATTTGATGAGATGCCACTTATCCCCAGAACTATACTA
GAGGAAGGTCAGACATGTGTGGAACTCATTAGACTCAGGATTCCATCGTTAGTCATTTTAGACGATGAATCTGAA
GCACAGCTTCCAGAATTTTTAGCAGACATTGTACAAAAACTTGGAGGGTTTGTCCTTAAAAAATTAGATGCATCT
ATACAACATCCGCTTATTAAAAAATATATTCATTCACCATTACCAAGTGCTGTTTTGCAGATAATGGAGAAGATG
CCATTGCAGAAATTGTGTAATCAAATAACTTCGCTACTTCCAACACACAAAGATGCCCTGAGGAAGTTCTTGGCT
AGTTTAACCGATAGCAGTGAGAAAGAGAAAAGAATTATTCAAGAATTGGCAATATTCAAGCGCATTAACCATTCT
TCTGATCAGGGAATTTCCTCTTATACAAAATTGAAAGGTTGTAAAGTCTTACACCATACTGCCAAACTCCCAGCA
GATCTGCGACTTTCTATTTCAGTAATAGACAGTAGTGATGAAGCTACTATTCGTCTGGCAAACATGTTGAAAATA
GAACAGTTAAAGACCACTAGCTGCTTAAAGCTTGTTTTAAAAGATATTGAAAATGCATTTTATTCACATGAAGAG
GTAACACAGCTTATGTTATGGGTCCTTGAGAATCTATCTTCTCTTAAAAATGAGAATCCAAATGTGCTTGAGTGG
TTAACACCATTAAAATTCATCCAGATATCACAGGAACAGATGGTATCAGCTGGTGAACTCTTTGACCCTGATATA
GAAGTACTAAAGGATCTCTTTTGTAATGAAGAAGGAACCTATTTCCCACCCTCAGTTTTTACCTCACCAGATATT
CTTCACTCCTTAAGACAGATTGGTTTAAAAAACGAAGCCAGTCTCAAAGAAAAGGATGTTGTGCAAGTGGCAAAA
AAAATTGAAGCCTTACAGGTCGGTGCTTGTCCTGATCAAGATGTTCTTCTGAAGAAAGCCAAAACCCTCTTACTG
GTTTTAAATAAGAATCACACACTGTTGCAATCATCTGAAGGAAAGATGACATTGAAGAAAATAAAATGGGTTCCA
GCCTGCAAGGAAAGGCCTCCAAATTATCCAGGCTCTTTGGTCTGGAAAGGAGATCTCTGTAATCTCTGTGCACCA
CCAGATATGTGTGATGTAGGCCATGCAATTCTCATTGGCTCCTCACTTCCTCTTGTTGAAAGTATCCATGTAAAC
CTGGAAAAAGCATTAGGGATCTTCACAAAACCTAGCCTTAGTGCTGTCTTAAAACACTTTAAAATTGTTGTTGAT
TGGTATTCTTCAAAAACCTTTAGTGATGAAGACTACTATCAATTCCAGCATATTTTGCTTGAGATTTACGGATTC
ATGCATGATCATCTAAATGAAGGGAAAGATTCTTTTAGAGCCTTAAAATTTCCATGGGTTTGGACTGGCAAAAAG
TTTTGTCCACTTGCCCAGGCTGTGATTAAACCAATCCATGATCTTGACCTTCAGCCTTATTTGCATAATGTACCT
AAAACCATGGCAAAATTCCACCAACTATTTAAGGTCTGTGGTTCAATAGAGGAGTTGACATCAGATCATATTTCC
ATGGTTATTCAGAAGATATATCTCAAAAGTGACCAAGATCTCAGTGAACAAGAAAGCAAACAAAATCTTCATCTT
ATGTTGAATATTATCAGATGGCTGTATAGCAATCAGATTCCAGCAAGC

An example nucleotide sequence encoding a SIRPT2 domain is:

(SEQ ID NO: 82)
ATAAATCCTGAAAACATGGGATTTGAGCAGTCAGGACAAAGAGAGCCACTTACTGTAAGAATTAAAAATATTCTG
GAAGAATACCCTTCAGTGTCAGATATTTTTAAAGAACTACTTCAAAACGCTGATGATGCAAATGCAACAGAATGC
AGTTTCTTGATTGATATGAGAAGAAATATGGACATAAGAGAGAATCTCCTAGACCCAGGGATGGCAGCTTGTCAT
GGACCTGCTTTGTGGTCATTCAACAATTCTCAATTCTCAGATTCAGATTTTGTGAACATAACTAGGTTAGGAGAA
TCTTTAAAAAGGGGAGAAGTTGACAAAGTTGGAAAATTTGGTCTTGGATTTAATTCTGTGTACCATATCACTGAC
ATTCCCATCATTATGAGTCGGGAATTCATGATAATGTTCGATCCAAACATAAATCATATCAGTAAACACATTAAA
GACAAATCCAATCCTGGGATCAAAATTAATTGGAGTAAACAACAGAAAAGACTTAGAAAATTTCCTAATCAGTTC
AAACCATTTATAGATGTATTTGGCTGTCAGTTACCTTTGACTGTAGAAGCACCTTACAGCTATAATGGAACCCTT
TTCCGACTGTCCTTTAGAACTCAACAGGAAGCAAAAGTGAGTGAAGTTAGTAGTACGTGCTACAATACAGCAGAT
ATTTATTCTCTTGTGGATGAATTTAGTCTCTGTGGACACAGGCTTATCATTTTCACTCAGAGTGTAAAGTCAATG
TATTTGAAGTACTTGAAAATTGAGGAAACCAACCCCAGTTTAGCACAAGATACAGTAATAATTAAAAAAAAATCC
TGCTCTTCCAAAGCATTGAACACACCTGTCTTAAGTGTTTTAAAAGAGGCTGCTAAGCTCATGAAGACTTGCAGC
AGCAGTAATAAAAAGCTTCCCAGTGATGAACCAAAGTCATCTTGCATTCTTCAGATCACAGTGGAAGAATTTCAC
CATGTGTTCAGAAGGATTGCTGATTTACAGTCGCCACTTTTTAGAGGTCCAGATGATGACCCAGCTGCTCTCTTT
GAAATGGCTAAGTCTGGCCAATCAAAAAAGCCATCAGATGAGTTGTCACAGAAAACAGTAGAGTGTACCACGTGG
CTTCTGTGTACTTGCATGGACACAGGAGAGGCTCTGAAGTTTTCCCTGAGTGAGAGTGGAAGAAGACTAGGACTG
GTTCCATGTGGGGCAGTAGGAGTTCAGCTGTCAGAAATCCAGGACCAGAAGTGGACAGTGAAACCACACATTGGA
GAGGTGTTTTGCTATTTACCTTTACGAATAAAAACAGGCTTGCCAGTTCATATCAATGGGTGCTTTGCTGTTACA
TCAAATAGGAAAGAAATCTGGAAAACAGATACAAAAGGACGATGGAATACCACGTTCATGAGACATGTTATTGTG
AAAGCTTACTTACAGGTACTGAGTGTCTTACGGGACCTGGCCACTAGTGGGGAGCTAATGGATTATACTTACTAT
GCAGTATGGCCCGATCCTGATTTAGTTCATGATGATTTTTCTGTAATTTGCCAAGGATTTTATGAAGATATAGCT
CATGGAAAAGGGAAAGAACTGACCAAAGTCTTCTCTGATGGATCTACTTGGGTTTCCATGAAGAACGTAAGATTT
CTAGATGACTCTATACTTAAAAGAAGAGATGTTGGTTCAGCAGCCTTCAAGATATTTTTGAAATACCTCAAGAAG
ACTGGGTCCAAAAACCTTTGTGCTGTTGAACTTCCTTCTTCGGTAAAATTAGGATTTGAAGAAGCTGGCTGCAAA
CAGATACTACTTGAAAACACATTTTCAGAGAAACAGTTTTTTTCTGAAGTGTTTTTTCCAAATATTCAAGAAATT
GAAGCAGAACTTAGAGATCCTTTAATGATCTTTGTTCTAAATGAAAAAGTTGATGAGTTCTCGGGAGTTCTTCGT
GTTACTCCATGTATTCCTTGTTCCTTGGAGGGGCATCCTTTGGTTTTGCCATCAAGATTGATCCACCCCGAAGGA
CGAGTTGCAAAGTTATTTGATATTAAAGATGGGAGATTCCCTTATGGTTCTACTCAGGATTATCTCAATCCTATT
ATTTTGATTAAACTAGTTCAGTTAGGTATGGCAAAAGATGATATTTTATGGGATGATATGCTAGAACGTGCAGTG
TCAGTAGCTGAAATTAATAAAAGTGATCATGTTGCTGCATGCCTAAGAAGTAGTATCTTATTGAGTCTTATCGAT
GAGAAACTAAAAATAAGGGATCCTAGAGCAAAGGATTTTGCTGCAAAATATCAAACAATCCGCTTCCTTCCATTT
CTGACAAAACCAGCAGGTTTTTCTTTGGACTGGAAAGGCAACAGTTTTAAGCCTGAAACCATGTTTGCAGCAACT
GACCTTTATACAGCTGAACATCAAGATATAGTTTGTCTTTTGCAACCAATTCTAAATGAAAATTCCCATTCTTTT
AGAGGTTGTGGTTCAGTGTCATTGGCTGTTAAAGAGTTTTTGGGATTACTCAAGAAGCCAACAGTTGATCTGGTT
ATAAACCAATTGAAAGAAGTAGCAAAATCAGTTGATGATGGAATTACACTGTACCAGGAGAATATCACCAATGCT
TGCTACAAATACCTTCATGAAGCCTTGATGCAAAATGAAATCACTAAGATGTCAATTATTGATAAGTTAAAACCC
TTTAGCTTCATTCTAGTTGAGAATGCATATGTTGACTCAGAAAAGGTTTCTTTTCATTTAAATTTTGAGGCGGCA
CCATACCTTTATCAGTTGCCTAATAAGTATAAAAATAATTTCCGCGAACTTTTTGAAACCGTGGGTGTGAGGCAG
TCATGCACTGTTGAAGATTTTGCTCTTGTTTTGGAATCTATTGATCAAGAAAGAGGAACAAAGCAAATAACAGAA
GAGAATTTTCAGCTTTGCCGACGAATAATCAGTGAAGGAATATGGAGTCTCATTAGAGAAAAGAAACAAGAATTT

An example nucleotide sequence encoding a SIRPT3 domain is:

(SEQ ID NO: 83)
AATGTCTGTTTTACAACACTTGGCACAGAATTTGGGCAGAAAGAAAAATTGACCAGCAGAATTAAGAGCATCCTT
AATGCATATCCTTCTGAAAAGGAAATGTTGAAAGAGCTTCTTCAAAATGCTGATGATGCAAAGGCGACAGAAATC
TGTTTTGTGTTTGATCCTAGACAGCATCCAGTTGATAGAATATTTGATGATAAGTGGGCCCCATTGCAAGGGCCA
GCACTTTGTGTGTACAACAACCAGCCATTTACAGAAGATGATGTTAGAGGAATTCAGAATCTTGGAAAAGGCACG
AAAGAGGGAAATCCTTATAAAACTGGACAGTATGGAATAGGATTCAATTCTGTGTATCATATCACAGACTGCCCA
TCTTTTATTTCTGGCAATGACATCCTGTGTATTTTTGATCCTCATGCCAGATATGCACCAGGGGCCACATCCATT
AGTCCCGGACGCATGTTTAGAGATTTGGATGCAGATTTTAGGACACAGTTCTCAGATGTTCTGGATCTTTATCTG
GGAACCCATTTTAAACTGGATAATTGCACAATGTTCAGATTTCCTCTTCGTAATGCAGAAATGGCAAAAGTTTCG
GAAATTTCGTCTGTTCCAGCATCAGACAGAATGGTCCAGAATCTTTTGGACAAACTGCGCTCAGATGGGGCAGAA
CTTCTAATGTTTCTTAATCACATGGAAAAAATTTCTATTTGTGAAATAGATAAGAGTACTGGAGCTCTAAATGTG
CTGTATTCAGTAAAGGGCAAAATCACAGATGGAGACAGATTGAAAAGGAAACAATTTCATGCATCTGTAATTGAT
AGTGTTACTAAAAAGAGGCAGCTCAAAGACATACCAGTTCAACAAATAACCTATACTATGGATACTGAGGACTCT
GAAGGAAATCTTACTACGTGGCTAATTTGTAATAGATCAGGCTTTTCAAGTATGGAGAAAGTATCTAAAAGTGTC
ATATCAGCTCACAAGAACCAAGATATTACTCTTTTCCCACGTGGTGGAGTAGCTGCCTGCATTACTCACAACTAT
AAAAAACCCCATAGGGCCTTCTGTTTTTTGCCTCTTTCTTTGGAGACTGGGCTGCCATTTCATGTGAATGGCCAC
TTTGCACTGGATTCAGCCAGAAGGAACCTGTGGCGTGATGATAATGGAGTTGGTGTTCGAAGTGACTGGAATAAC
AGTTTAATGACAGCATTAATAGCTCCTGCATATGTTGAATTGCTAATACAGTTAAAAAAACGGTATTTCCCTGGT
TCTGATCCAACATTATCAGTGTTACAGAACACCCCTATTCATGTTGTAAAGGACACTTTAAAGAAGTTTTTATCG
TTTTTCCCAGTTAACCGTCTTGATCTACAGCCAGATTTATATTGTCTAGTGAAAGCACTTTACAATTGCATTCAC
GAAGACATGAAACGTCTTTTACCTGTTGTGCGGGCTCCAAATATTGATGGCTCTGACTTGCACTCTGCAGTTATA
ATTACTTGGATCAATATGTCTACTTCTAATAAAACTAGACCATTTTTTGACAATTTACTACAGGATGAATTACAA
CACCTTAAAAATGCAGATTATAATATCACCACACGCAAAACAGTAGCAGAGAATGTCTATAGGCTGAAACATCTC
CTTTTAGAAATTGGTTTCAACTTGGTTTATAACTGTGATGAAACTGCTAATCTTTACCACTGTCTTATAGATGCA
GATATTCCTGTTAGTTATGTGACCCCTGCTGATATCAGATCTTTTTTAATGACATTTTCCTCTCCTGACACTAAT
TGCCATATTGGGAAGCTGCCTTGTCGTCTGCAGCAGACTAATCTAAAACTTTTTCATAGTTTAAAACTTTTAGTT
GATTATTGTTTTAAAGATGCAGAAGAAAATGAGATTGAAGTTGAGGGATTGCCCCTTCTCATCACACTGGACAGT
GTTTTGCAAACTTTTGATGCAAAACGACCCAAGTTTCTAACAACATATCATGAATTGATTCCATCCCGCAAAGAC
TTGTTTATGAATACATTATATTTGAAATATAGTAATATTTTATTGAACTGTAAAGTTGCAAAAGTGTTTGACATT
TCCAGCTTTGCTGATTTGTTATCCTCTGTGTTGCCTCGAGAATATAAGACCAAAAGTTGCACAAAGTGGAAAGAC
AATTTTGCAAGTGAGTCTTGGCTTAAGAATGCATGGCATTTTATTAGTGAATCTGTAAGTGTGAAAGAAGATCAG
GAAGAAACAAAACCAACATTTGACATTGTTGTTGATACTCTAAAAGACTGGGCATTGCTTCCAGGAACAAAGTTT
ACTGTTTCAGCCAACCAGCTTGTGGTTCCTGAAGGAGATGTTCTGCTTCCTCTCAGCCTTATGCACATTGCAGTT
TTTCCAAATGCCCAGAGTGATAAAGTTTTTCATGCTCTAATGAAAGCTGGCTGTATTCAGCTTGCTTTGAACAAA
ATCTGTTCCAAAGACAGTGCATTTGTTCCTTTGTTGTCATGTCACACAGCAAATATAGAGAGCCCCACAAGCATC
TTGAAGGCTCTACATTATATGGTCCAAACTTCAACATTTAGAGCAGAAAAATTAGTAGAAAATGATTTTGAGGCA
CTTTTGATGTATTTCAACTGCAATTTGAATCATTTGATGTCCCAAGATGATATAAAAATTCTAAAGTCACTTCCG
TGCTATAAATCCATCAGTGGCCGCTATGTAAGCATTGGAAAATTTGGAACATGCTACGTACTTACAAAAAGTATC
CCTTCAGCTGAAGTGGAGAAATGGACACAGTCATCATCATCTGCATTTCTTGAAGAAAAAATACACTTAAAAGAA
CTATATGAGGTGATTGGTTGTGTACCTGTAGATGATCTTGAGGTATATTTGAAACACCTCTTACCAAAAATTGAA
AATCTCTCTTATGATGCAAAATTAGAGCACTTGATCTACCTTAAGAATAGATTATCAAGTGCTGAGGAATTATCA
GAGATTAAGGAACAACTTTTTGAAAAACTGGAAAGTTTATTGATAATCCATGATGCTAACAGTAGACTAAAGCAA
GCAAAGCATTTCTATGATAGAACTGTGAGAGTTTTTGAAGTTATGCTTCCTGAAAAATTGTTTATTCCTAATGAT
TTCTTTAAGAAATTGGAACAACTTATAAAACCCAAAAATCATGTTACATTTATGACATCCTGGGTGGAATTCTTA
AGAAATATTGGACTAAAATACATACTTTCTCAGCAGCAGTTGTTACAGTTTGCTAAGGAAATCAGTGTGAGGGCT
AATACAGAAAACTGGTCCAAAGAAACATTGCAAAATACAGTTGATATCCTTCTGCATCATATATTCCAAGAACGA
ATGGATTTGTTATCTGGAAATTTTCTGAAAGAACTATCTTTAATACCATTCTTATGTCCTGAGCGGGCCCCCGCG
GAATTCATTAGATTTCATCCTCAATATCAAGAGGTAAATGGAACACTTCCTCTTATAAAGTTCAATGGAGCACAG
GTAAATCCAAAATTCAAGCAATGTGATGTACTCCAGCTGTTATGGACATCCTGCCCTATTCTTCCAGAGAAAGCT
ACACCCTTAAGCATTAAAGAACAAGAAGGTAGTGACCTTGGTCCACAAGAACAGCTTGAACAAGTTTTAAATATG
CTTAATGTTAACCTGGATCCTCCTCTTGATAAGGTAATCAATAACTGCAGAAACATATGCAACATAACGACGTTG
GATGAAGAAATGGTAAAAACTAGAGCAAAAGTCTTAAGGAGCATATATGAATTCCTCAGTGCAGAAAAAAGGGAA
TTTCGTTTTCAGTTGCGAGGGGTTGCTTTTGTGATGGTAGAAGATGGTTGGAAACTTCTGAAGCCTGAGGAGGTA
GTCATAAACCTAGAATATGAATCTGATTTTAAACCTTATTTGTACAAGCTACCTTTAGAACTTGGCACATTTCAC
CAGTTGTTCAAACACTTAGGTACTGAAGATATTATTTCAACTAAGCAATATGTTGAAGTGTTGAGCCGCATATTT
AAAAATTCTGAGGGCAAACAATTAGATCCTAATGAAATGCGTACAGTTAAGAGAGTAGTTTCTGGTCTGTTCAGG
AGTCTACAGAATGATTCAGTCAAGGTGAGG

In some embodiments, the nucleotide sequence encoding the SIRPT domain comprises or consists of a nucleotide sequence having at least 70%, 75%, 80%, 85%, 90%, 95%, 96%, 97%, 98% or 99% sequence identity to any one of SEQ ID NOs: 81-83, preferably wherein the nucleotide sequence encoding the SIRPT domain maintains the function of SEQ ID NO: 81-83, respectively.

In some embodiments, the nucleotide sequence encoding the SIRPT domain comprises or consists of the nucleotide sequence of any one of SEQ ID NOs: 81-83.

### Engineered Sacsin

The engineered sacsin may be a sacsin that is smaller than the full length sacsin protein (e.g. may be a truncated or miniaturised sacsin), for example the full length protein encoded by the nucleotide sequence of NCBI RefSeq NM_014363.6, while preferably retaining the activity of the full length sacsin protein, for example the full length protein encoded by the nucleotide sequence of NCBI RefSeq NM_014363.6. Preferably, the engineered sacsin is sufficiently small for its encoding nucleotide sequence to be packaged in an AAV vector when operably linked to one or more expression control sequence. The engineered (e.g. truncated) sacsin may be smaller than the full length sacsin protein due to the deletion of amino acids internal to the full length sacsin protein and/or from the N- and/or C-terminus of the full length sacsin protein. Preferably, the engineered sacsin is smaller than the full length sacsin protein due to the deletion of amino acids internal to the full length sacsin protein, for example amino acids between the UbL domain and the SRR domain, amino acids between the SRR domain and the DnaJ domain, and/or amino acids between the DnaJ domain and the HEPN domain of the full length sacsin protein.

In some embodiments, the nucleotide sequence encoding the engineered sacsin is less than or equal to about 2700 bp in length. In some embodiments, the nucleotide sequence encoding the engineered sacsin is less than or equal to about 2500 bp in length. In some embodiments, the nucleotide sequence encoding the engineered sacsin is less than or equal to about 2400 bp in length.

In some embodiments, the nucleotide sequence encoding the engineered sacsin is about 2300-2700 bp, 2300-2600 bp, 2300-2500 bp or 2300-2400 bp in length.

In some embodiments, the nucleotide sequence encoding the engineered sacsin is about 2300-2400 bp in length.

In another aspect, the invention provides a polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof, and wherein the nucleotide sequence encoding the engineered sacsin is less than or equal to about 5200 bp in length.

In one aspect, the invention provides a polynucleotide comprising a nucleotide sequence encoding an engineered sacsin, wherein the engineered sacsin comprises a UbL domain, an SRR domain, a DnaJ domain and an HEPN domain, and wherein the nucleotide sequence encoding the engineered sacsin is less than or equal to about 5200 bp in length.

In some embodiments, the nucleotide sequence encoding the engineered sacsin is less than or equal to about 5100 bp, 5000 bp, 4900 bp, 4800 bp, 4700 bp, 4600 bp, 4500 bp, 4400 bp, 4300 bp, 4200 bp, 4100 bp, 4000 bp, 3900 bp, 3800 bp, 3700 bp, 3600 bp, 3500 bp, 3400 bp, 3300 bp, 3200 bp, 3100 bp, 3000 bp, 2900 bp, 2800 bp, 2700 bp, 2600 bp, 2500 bp or 2400 bp in length.

In some embodiments, the nucleotide sequence encoding the engineered sacsin is about 2300-5200 bp, 2300-5100 bp, 2300-5000 bp, 2300-4900 bp, 2300-4800 bp, 2300-4700 bp, 2300-4600 bp, 2300-4500 bp, 2300-4400 bp, 2300-4300 bp, 2300-4200 bp, 2300-4100 bp, 2300-4000 bp, 2300-3900 bp, 2300-3800 bp, 2300-3700 bp, 2300-3600 bp, 2300-3500 bp, 2300-3400 bp, 2300-3300 bp, 2300-3200 bp, 2300-3100 bp, 2300-3000 bp, 2300-2900 bp, 2300-2800 bp, 2300-2700 bp, 2300-2600 bp, 2300-2500 bp or 2300-2400 bp in length.

In some embodiments, the SRR domain is joined to the DnaJ domain via a linker. In some embodiments, the SRR domain is directly joined to the DnaJ domain. In some embodiments, the UbL domain is directly joined to the SRR domain. In some embodiments, the UbL domain is joined to the SRR domain via a linker. In some embodiments, the DnaJ domain is directly joined to the HEPN domain. In some embodiments, the DnaJ domain is joined to the HEPN domain via a linker.

Linkers typically comprise a short polynucleotide or polypeptide sequence. Linkers may be used to physically separate functional sequences in order, e.g., to improve the functionality of said sequences.

In some embodiments, the linker is a polypeptide linker. Preferably, the linker is a GS linker. In some embodiments, the linker is a GGGGS (SEQ ID NO: 46) linker. In some embodiments, the linker is a AEAAAKALEAEAAAKA (SEQ ID NO: 47) linker.

In some embodiments, the DnaJ domain is joined to the HEPN domain via a FQRFYTSWNQEATSHKSERQQQNKEKCPPSAGQTYSQRFFVPPTFKS (SEQ ID NO: 50) linker, or a linker with at least 70%, 75%, 80%, 85%, 90%, 95%, 96%, 97%, 98% or 99% sequence identity thereto.

In some embodiments, the polynucleotide comprises a nucleotide sequence encoding a linker, such as the nucleotide sequence of any one of SEQ ID NOs: 7, 48, 49 or 51, or a nucleotide sequence with at least 70%, 75%, 80%, 85%, 90%, 95%, 96%, 97%, 98% or 99% sequence identity thereto. In some embodiments, the polynucleotide comprises a nucleotide sequence encoding a linker between the nucleotide sequences encoding the SRR domain and the DnaJ domain, such as the nucleotide sequence of any one of SEQ ID NOs: 7, 48 or 49, or a nucleotide sequence with at least 70%, 75%, 80%, 85%, 90%, 95%, 96%, 97%, 98% or 99% sequence identity thereto. In some embodiments, the polynucleotide comprises a nucleotide sequence encoding a linker between the nucleotide sequences encoding the DnaJ domain and the HEPN domain, such as the nucleotide sequence of any one of SEQ ID NOs: 7, 48, 49 or 51 (preferably SEQ ID NO: 51), or a nucleotide sequence with at least 70%, 75%, 80%, 85%, 90%, 95%, 96%, 97%, 98% or 99% sequence identity thereto.

Example nucleotide sequences encoding a linker are:

(SEQ ID NO: 7)
GGATCC
(SEQ ID NO: 48)
GGGGGTGGGGGCAGT
(SEQ ID NO: 49)
GCGGAAGCTGCGGCTAAAGCCCTCGAAGCTGAGGCTGCTGCCAAAGCC
(SEQ ID NO: 51)
TTTCAGAGATTCTATACTTCATGGAATCAAGAAGCAACGAGCCATAAAT
CTGAAAGACAGCAACAGAACAAAGAAAAATGCCCCCCTTCAGCCGGACA
GACTTACTCTCAAAGGTTCTTTGTTCCTCCCACTTTCAAGTCG

An example nucleotide sequence encoding an engineered sacsin is:

(SEQ ID NO: 8)
ATGGAGACCAAGGAGAACAGGTGGGTCCCGGTGACCGTGCTCCCCGGCTGCGTGGGCTGCAGGACCGTCGCGGCG
CTGGCGTCCTGGACCGTGCGCGATGTGAAGGAACGTATCTTCGCGGAGACTGGCTTCCCGGTGTCGGAGCAGCGG
CTGTGGCGCGGCGGCCGCGAGTTATCTGACTGGATCAAGATTGGAGATCTGACTTCCAAAAATTGTCATCTTTTT
GTAAACCTTCAATCAAAAGGCTTAAAAGGGGGAGGTCGATTTGGTCAGACAACGCCACCACTTGTTGATTTTCTC
AAGGACATTTTGAGAAGATATCCAGAAGGAGGACAGATTCTTAAGGAATTAATTCAGAATGCAGAAGATGCTGGG
GCGACAGAAGTTAAATTTTTATATGATGAAACTCAATACGGAACAGAGACTCTTTGGTCAAAAGATATGGCGCCA
TATCAGGGGCCAGCTCTCTATGTGTACAACAACGCGGTTTTCACCCCAGAGGACTGGCACGGCATTCAAGAAATA
GCAAGAAGCAGGAAAAAGGATGATCCTCTGAAGGTCGGAAGATTTGGAATTGGGTTTAATTCTGTCTATCATATA
ACAGATGTTCCTTGTATCTTTAGTGGTGACCAAATCGGGATGCTAGATCCTCATCAAACACTTTTTGGCCCACAT
GAATCAGGCCAATGTTGGAATCTCAAAGATGACAGCAAAGAAATTAGTGAACTTTCAGACCAGTTTGCACCATTT
GTTGGCATTTTTGGAAGCACCAAGGAAACATTTATAAACGGCAATTTTCCAGGAACATTTTTCCGTTTCCCTCTT
CGCCTACAACCTTCACAACTTAGTAGTAACCTCTACAATAAGCAGAAGGTTCTTGAGTTGTTTGAGTCTTTTAGG
GCAGATGCAGACACAGTGCTGCTCTTTCTGAAAAGTGTGCAGGATGTTTCCTTATATGTCCGAGAGGCTGACGGA
ACAGAGAAACTGGTGTTTAGAGTGACTTCGAGTGAGAGTAAGGCACTGAAACATGAGCGGCCGAATTCTATAAAG
ATTCTGGGAACTGCTATAAGTAACTATTGTAAAAAGACTCCAAGCAATAACATCACCTGTGTAACATATCACGTA
AATATTGTTTTAGAAGAGGAGAGTACTAAGGATGCACAGAAAACATCTTGGTTGGTGTGTAACAGTGTGGGTGGG
CGAGGGATCAGTAGTAAGCTTGACTCTTTAGCTGATGAACTGAAATTTGTCCCAATCATTGGAATAGCCATGCCT
TTATCAAGCAGAGATGATGAAGCAAAAGGAGCAACGTCTGATTTCTCAGGAAAAGCATTTTGTTTCCTTCCTTTA
CCACCTGGTGAGGAAAGCAGCACAGGCCTCCCAGTTCACATCAGTGGGTTCTTTGGCCTTACTGATAACCGCAGG
AGCATAAAATGGAGAGAGCTGGACCAGTGGAGAGACCCGGCAGCCTTATGGAATGAGTTTCTTGTCATGAATGTT
GTCCCCAAAGCTTATGCTACTCTGGGATCCATCTTAAAAGAAGTGACATCTGTGGTGGAGCAAGCATGGAAGCTT
CCAGAATCGGAACGAAAAAAGATTATTAGGCGGTTGTATTTGAAATGGCATCCTGACAAAAATCCAGAGAACCAT
GACATTGCCAATGAAGTTTTTAAACATTTGCAGAATGAAATCAACAGATTAGAAAAACAGGCTTTTCTAGATCAA
AATGCAGACAGGGCCTCCAGACGAACATTTTCAACCTCAGCATCCCGATTTCAGTCAGACAAATACTCATTTCAG
AGATTCTATACTTCATGGAATCAAGAAGCAACGAGCCATAAATCTGAAAGACAGCAACAGAACAAAGAAAAATGC
CCCCCTTCAGCCGGACAGACTTACTCTCAAAGGTTCTTTGTTCCTCCCACTTTCAAGTCGGTTGGCAATCCAGTG
GAAGCACGCAGATGGCTAAGACAAGCCAGAGCAAACTTCTCAGCTGCCAGGAATGACCTTCATAAAAATGCCAAT
GAGTGGGTGTGCTTTAAATGTTACCTTTCTACCAAGTTAGCTTTGATTGCAGCTGACTATGCTGTGAGGGGAAAG
TCTGATAAAGATGTAAAACCAACTGCACTTGCTCAGAAAATAGAGGAATATAGTCAGCAACTTGAAGGACTGACA
AATGATGTTCACACATTGGAAGCTTATGGTGTAGACAGTTTAAAAACAAGATACCCTGATTTGCTTCCCTTTCCT
CAGATCCCAAATGACAGGTTCACTTCTGAGGTTGCTATGAGGGTGATGGAATGTACTGCCTGTATCATAATAAAA
CTTGAAAATTTTATGCAACAAAAAGTG

An example amino acid sequence of an engineered sacsin is:

(SEQ ID NO: 9)
METKENRWVPVTVLPGCVGCRTVAALASWTVRDVKERIFAETGFPVSEQRLWRGGRELSDWIKIGDLTSKNCHLF
VNLQSKGLKGGGRFGQTTPPLVDFLKDILRRYPEGGQILKELIQNAEDAGATEVKFLYDETQYGTETLWSKDMAP
YQGPALYVYNNAVFTPEDWHGIQEIARSRKKDDPLKVGRFGIGFNSVYHITDVPCIFSGDQIGMLDPHQTLFGPH
ESGQCWNLKDDSKEISELSDQFAPFVGIFGSTKETFINGNFPGTFFRFPLRLQPSQLSSNLYNKQKVLELFESFR
ADADTVLLFLKSVQDVSLYVREADGTEKLVFRVTSSESKALKHERPNSIKILGTAISNYCKKTPSNNITCVTYHV
NIVLEEESTKDAQKTSWLVCNSVGGRGISSKLDSLADELKFVPIIGIAMPLSSRDDEAKGATSDFSGKAFCFLPL
PPGEESSTGLPVHISGFFGLTDNRRSIKWRELDQWRDPAALWNEFLVMNVVPKAYATLGSILKEVTSVVEQAWKL
PESERKKIIRRLYLKWHPDKNPENHDIANEVFKHLQNEINRLEKQAFLDQNADRASRRTFSTSASRFQSDKYSFQ
RFYTSWNQEATSHKSERQQQNKEKCPPSAGQTYSQRFFVPPTFKSVGNPVEARRWLRQARANFSAARNDLHKNAN
EWVCFKCYLSTKLALIAADYAVRGKSDKDVKPTALAQKIEEYSQQLEGLTNDVHTLEAYGVDSLKTRYPDLLPFP
QIPNDRFTSEVAMRVMECTACIIIKLENFMQQKV

An example codon optimised nucleotide sequence encoding an engineered sacsin is:

(SEQ ID NO: 10)
ATGGAAACTAAGGAGAATAGATGGGTTCCTGTGACTGTCCTCCCGGGATGCGTTGGGTGTAGGACAGTGGCTGCC
TTGGCGAGTTGGACGGTTCGGGATGTAAAGGAGAGAATATTCGCGGAAACTGGGTTCCCCGTTAGTGAGCAACGA
TTGTGGAGAGGTGGAAGGGAGCTGAGTGATTGGATCAAGATAGGGGATCTTACTTCCAAAAACTGTCATCTTTTC
GTAAATCTGCAGTCTAAAGGTCTCAAAGGCGGTGGGCGCTTCGGTCAAACGACGCCTCCGCTCGTTGATTTTCTC
AAAGACATACTTCGCAGATACCCTGAAGGAGGGCAGATCCTCAAGGAACTTATCCAAAATGCCGAGGACGCTGGC
GCTACTGAGGTAAAATTCCTTTATGATGAAACACAGTATGGCACCGAAACCCTGTGGAGCAAGGATATGGCTCCC
TATCAGGGCCCGGCTCTCTACGTCTATAACAACGCGGTTTTCACCCCGGAAGACTGGCATGGTATCCAGGAAATA
GCGAGATCCAGGAAAAAGGACGATCCGCTGAAGGTTGGGCGGTTCGGAATAGGTTTTAATAGCGTTTATCATATT
ACTGATGTGCCGTGCATCTTCTCTGGCGACCAGATAGGTATGCTGGACCCCCATCAGACCCTGTTCGGTCCCCAC
GAGTCTGGGCAGTGTTGGAACTTGAAAGATGATAGTAAGGAAATAAGTGAGTTGAGCGACCAATTTGCGCCATTC
GTAGGTATATTTGGCTCAACGAAAGAGACCTTTATTAACGGAAACTTCCCCGGTACCTTCTTCCGATTCCCTCTC
CGACTCCAACCCTCACAGTTGAGTAGCAATCTGTACAATAAACAGAAGGTATTGGAACTTTTCGAATCCTTTCGA
GCAGATGCGGACACTGTGCTTTTGTTCCTCAAGAGCGTACAAGATGTCTCACTGTATGTACGAGAGGCCGATGGC
ACTGAGAAATTGGTATTTAGAGTAACCAGCTCCGAATCTAAAGCGCTCAAACATGAGCGACCCAACAGTATTAAG
ATTCTTGGGACGGCGATTTCAAACTACTGCAAAAAGACCCCTTCCAACAACATAACTTGTGTAACCTATCATGTT
AACATTGTTCTTGAGGAGGAAAGTACCAAGGACGCTCAAAAGACGTCTTGGCTGGTCTGCAACTCTGTCGGCGGG
CGAGGCATCAGCTCAAAACTGGACTCCTTGGCGGACGAGCTCAAATTCGTCCCCATAATCGGGATCGCAATGCCC
CTGAGTTCCCGGGATGATGAGGCAAAGGGCGCCACAAGTGACTTTAGCGGAAAAGCATTTTGCTTTCTTCCGCTG
CCACCTGGAGAAGAATCATCTACAGGTCTCCCAGTACACATATCAGGGTTTTTTGGCCTCACGGATAATAGAAGG
TCCATAAAGTGGCGAGAACTGGACCAATGGCGGGATCCGGCTGCATTGTGGAACGAGTTCCTGGTAATGAATGTG
GTACCAAAAGCTTACGCCACGCTGGGGTCAATACTCAAGGAGGTAACTTCTGTAGTAGAGCAAGCGTGGAAGCTT
CCGGAATCTGAAAGAAAGAAAATCATACGACGACTGTATCTCAAATGGCATCCCGATAAAAACCCTGAGAACCAT
GATATCGCGAATGAAGTGTTCAAGCATCTGCAAAACGAGATTAACCGACTCGAAAAGCAAGCTTTTCTCGACCAA
AATGCAGATCGCGCATCTCGGAGGACCTTTAGCACCTCTGCCAGTCGCTTTCAGTCCGACAAGTACTCATTCCAA
CGATTTTATACATCCTGGAATCAAGAGGCAACATCCCATAAATCAGAGAGACAGCAACAGAATAAAGAAAAGTGC
CCACCTAGCGCGGGACAGACGTATAGTCAACGCTTTTTTGTTCCACCTACATTCAAGTCTGTTGGTAACCCAGTG
GAGGCCCGAAGATGGCTGAGGCAAGCTCGCGCAAATTTTAGTGCTGCACGCAATGACCTTCACAAGAATGCCAAT
GAGTGGGTGTGTTTTAAATGCTATTTGAGTACAAAACTCGCGTTGATTGCGGCTGACTATGCAGTTCGCGGCAAA
AGTGACAAGGATGTTAAGCCTACGGCTTTGGCGCAGAAGATAGAAGAATACTCCCAACAACTTGAGGGTCTCACT
AATGATGTTCACACCTTGGAAGCGTATGGGGTAGATAGCCTTAAAACGAGATACCCTGATCTGTTGCCATTCCCT
CAAATTCCGAACGACCGCTTCACTAGTGAGGTCGCAATGAGGGTAATGGAGTGCACCGCGTGTATCATTATAAAG
TTGGAGAATTTTATGCAACAGAAGGTA

An example amino acid sequence of an engineered sacsin is:

(SEQ ID NO: 11)
METKENRWVPVTVLPGCVGCRTVAALASWTVRDVKERIFAETGFPVSEQRLWRGGRELSDWIKIGDLTSKNCHLF
VNLQSKGLKGGGRFGQTTPPLVDFLKDILRRYPEGGQILKELIQNAEDAGATEVKFLYDETQYGTETLWSKDMAP
YQGPALYVYNNAVFTPEDWHGIQEIARSRKKDDPLKVGRFGIGFNSVYHITDVPCIFSGDQIGMLDPHQTLFGPH
ESGQCWNLKDDSKEISELSDQFAPFVGIFGSTKETFINGNFPGTFFRFPLRLQPSQLSSNLYNKQKVLELFESFR
ADADTVLLFLKSVQDVSLYVREADGTEKLVFRVTSSESKALKHERPNSIKILGTAISNYCKKTPSNNITCVTYHV
NIVLEEESTKDAQKTSWLVCNSVGGRGISSKLDSLADELKFVPIIGIAMPLSSRDDEAKGATSDFSGKAFCFLPL
PPGEESSTGLPVHISGFFGLTDNRRSIKWRELDQWRDPAALWNEFLVMNVVPKAYATLGSILKEVTSVVEQAWKL
PESERKKIIRRLYLKWHPDKNPENHDIANEVFKHLQNEINRLEKQAFLDQNADRASRRTFSTSASRFQSDKYSFQ
RFYTSWNQEATSHKSERQQQNKEKCPPSAGQTYSQRFFVPPTFKSVGNPVEARRWLRQARANFSAARNDLHKNAN
EWVCFKCYLSTKLALIAADYAVRGKSDKDVKPTALAQKIEEYSQQLEGLTNDVHTLEAYGVDSLKTRYPDLLPFP
QIPNDRFTSEVAMRVMECTACIIIKLENFMQQKV

A further example nucleotide sequence encoding an engineered sacsin is:

(SEQ ID NO: 12)
ATGGAGACCAAGGAGAACAGGTGGGTCCCGGTGACCGTGCTCCCCGGCTGCGTGGGCTGCAGGACCGTCGCGGCG
CTGGCGTCCTGGACCGTGCGCGATGTGAAGGAACGTATCTTCGCGGAGACTGGCTTCCCGGTGTCGGAGCAGCGG
CTGTGGCGCGGCGGCCGCGAGTTATCTGACTGGATCAAGATTGGAGATCTGACTTCCAAAAATTGTCATCTTTTT
GTAAACCTTCAATCAAAAGGCTTAATAAATCCTGAAAACATGGGATTTGAGCAGTCAGGACAAAGAGAGCCACTT
ACTGTAAGAATTAAAAATATTCTGGAAGAATACCCTTCAGTGTCAGATATTTTTAAAGAACTACTTCAAAACGCT
GATGATGCAAATGCAACAGAATGCAGTTTCTTGATTGATATGAGAAGAAATATGGACATAAGAGAGAATCTCCTA
GACCCAGGGATGGCAGCTTGTCATGGACCTGCTTTGTGGTCATTCAACAATTCTCAATTCTCAGATTCAGATTTT
GTGAACATAACTAGGTTAGGAGAATCTTTAAAAAGGGGAGAAGTTGACAAAGTTGGAAAATTTGGTCTTGGATTT
AATTCTGTGTACCATATCACTGACATTCCCATCATTATGAGTCGGGAATTCATGATAATGTTCGATCCAAACATA
AATCATATCAGTAAACACATTAAAGACAAATCCAATCCTGGGATCAAAATTAATTGGAGTAAACAACAGAAAAGA
CTTAGAAAATTTCCTAATCAGTTCAAACCATTTATAGATGTATTTGGCTGTCAGTTACCTTTGACTGTAGAAGCA
CCTTACAGCTATAATGGAACCCTTTTCCGACTGTCCTTTAGAACTCAACAGGAAGCAAAAGTGAGTGAAGTTAGT
AGTACGTGCTACAATACAGCAGATATTTATTCTCTTGTGGATGAATTTAGTCTCTGTGGACACAGGCTTATCATT
TTCACTCAGAGTGTAAAGTCAATGTATTTGAAGTACTTGAAAATTGAGGAAACCAACCCCAGTTTAGCACAAGAT
ACAGTAATAATTAAAAAAAAATCCTGCTCTTCCAAAGCATTGAACACACCTGTCTTAAGTGTTTTAAAAGAGGCT
GCTAAGCTCATGAAGACTTGCAGCAGCAGTAATAAAAAGCTTCCCAGTGATGAACCAAAGTCATCTTGCATTCTT
CAGATCACAGTGGAAGAATTTCACCATGTGTTCAGAAGGATTGCTGATTTACAGTCGCCACTTTTTAGAGGTCCA
GATGATGACCCAGCTGCTCTCTTTGAAATGGCTAAGTCTGGCCAATCAAAAAAGCCATCAGATGAGTTGTCACAG
AAAACAGTAGAGTGTACCACGTGGCTTCTGTGTACTTGCATGGACACAGGAGAGGCTCTGAAGTTTTCCCTGAGT
GAGAGTGGAAGAAGACTAGGACTGGTTCCATGTGGGGCAGTAGGAGTTCAGCTGTCAGAAATCCAGGACCAGAAG
TGGACAGTGAAACCACACATTGGAGAGGTGTTTTGCTATTTACCTTTACGAATAAAAACAGGCTTGCCAGTTCAT
ATCAATGGGTGCTTTGCTGTTACATCAAATAGGAAAGAAATCTGGAAAACAGATACAAAAGGACGATGGAATACC
ACGTTCATGAGACATGTTATTGTGAAAGCTTACTTACAGGTACTGAGTGTCTTACGGGACCTGGCCACTAGTGGG
GAGCTAATGGATTATACTTACTATGCAGTATGGCCCGATCCTGATTTAGTTCATGATGATTTTTCTGTAATTTGC
CAAGGATTTTATGAAGATATAGCTCATGGAGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTG

An example amino acid sequence of an engineered sacsin is:

(SEQ ID NO: 13)
METKENRWVPVTVLPGCVGCRTVAALASWTVRDVKERIFAETGFPVSEQRLWRGGRELSDWIKIGDLTSKNCHLF
VNLQSKGLINPENMGFEQSGQREPLTVRIKNILEEYPSVSDIFKELLQNADDANATECSFLIDMRRNMDIRENLL
DPGMAACHGPALWSFNNSQFSDSDFVNITRLGESLKRGEVDKVGKFGLGFNSVYHITDIPIIMSREFMIMFDPNI
NHISKHIKDKSNPGIKINWSKQQKRLRKFPNQFKPFIDVFGCQLPLTVEAPYSYNGTLFRLSFRTQQEAKVSEVS
STCYNTADIYSLVDEFSLCGHRLIIFTQSVKSMYLKYLKIEETNPSLAQDTVIIKKKSCSSKALNTPVLSVLKEA
AKLMKTCSSSNKKLPSDEPKSSCILQITVEEFHHVFRRIADLQSPLFRGPDDDPAALFEMAKSGQSKKPSDELSQ
KTVECTTWLLCTCMDTGEALKFSLSESGRRLGLVPCGAVGVQLSEIQDQKWTVKPHIGEVFCYLPLRIKTGLPVH
INGCFAVTSNRKEIWKTDTKGRWNTTFMRHVIVKAYLQVLSVLRDLATSGELMDYTYYAVWPDPDLVHDDFSVIC
QGFYEDIAHGGSILKEVTSVVEQAWKLPESERKKIIRRLYLKWHPDKNPENHDIANEVFKHLQNEINRLEKQAFL
DQNADRASRRTFSTSASRFQSDKYSFQRFYTSWNQEATSHKSERQQQNKEKCPPSAGQTYSQRFFVPPTFKSVGN
PVEARRWLRQARANFSAARNDLHKNANEWVCFKCYLSTKLALIAADYAVRGKSDKDVKPTALAQKIEEYSQQLEG
LTNDVHTLEAYGVDSLKTRYPDLLPFPQIPNDRFTSEVAMRVMECTACIIIKLENFMQQKV

A further example nucleotide sequence encoding an engineered sacsin is:

(SEQ ID NO: 14)
ATGGAGACCAAGGAGAACAGGTGGGTCCCGGTGACCGTGCTCCCCGGCTGCGTGGGCTGCAGGACCGTCGCGGCG
CTGGCGTCCTGGACCGTGCGCGATGTGAAGGAACGTATCTTCGCGGAGACTGGCTTCCCGGTGTCGGAGCAGCGG
CTGTGGCGCGGCGGCCGCGAGTTATCTGACTGGATCAAGATTGGAGATCTGACTTCCAAAAATTGTCATCTTTTT
GTAAACCTTCAATCAAAAGGCTTAAATGTCTGTTTTACAACACTTGGCACAGAATTTGGGCAGAAAGAAAAATTG
ACCAGCAGAATTAAGAGCATCCTTAATGCATATCCTTCTGAAAAGGAAATGTTGAAAGAGCTTCTTCAAAATGCT
GATGATGCAAAGGCGACAGAAATCTGTTTTGTGTTTGATCCTAGACAGCATCCAGTTGATAGAATATTTGATGAT
AAGTGGGCCCCATTGCAAGGGCCAGCACTTTGTGTGTACAACAACCAGCCATTTACAGAAGATGATGTTAGAGGA
ATTCAGAATCTTGGAAAAGGCACGAAAGAGGGAAATCCTTATAAAACTGGACAGTATGGAATAGGATTCAATTCT
GTGTATCATATCACAGACTGCCCATCTTTTATTTCTGGCAATGACATCCTGTGTATTTTTGATCCTCATGCCAGA
TATGCACCAGGGGCCACATCCATTAGTCCCGGACGCATGTTTAGAGATTTGGATGCAGATTTTAGGACACAGTTC
TCAGATGTTCTGGATCTTTATCTGGGAACCCATTTTAAACTGGATAATTGCACAATGTTCAGATTTCCTCTTCGT
AATGCAGAAATGGCAAAAGTTTCGGAAATTTCGTCTGTTCCAGCATCAGACAGAATGGTCCAGAATCTTTTGGAC
AAACTGCGCTCAGATGGGGCAGAACTTCTAATGTTTCTTAATCACATGGAAAAAATTTCTATTTGTGAAATAGAT
AAGAGTACTGGAGCTCTAAATGTGCTGTATTCAGTAAAGGGCAAAATCACAGATGGAGACAGATTGAAAAGGAAA
CAATTTCATGCATCTGTAATTGATAGTGTTACTAAAAAGAGGCAGCTCAAAGACATACCAGTTCAACAAATAACC
TATACTATGGATACTGAGGACTCTGAAGGAAATCTTACTACGTGGCTAATTTGTAATAGATCAGGCTTTTCAAGT
ATGGAGAAAGTATCTAAAAGTGTCATATCAGCTCACAAGAACCAAGATATTACTCTTTTCCCACGTGGTGGAGTA
GCTGCCTGCATTACTCACAACTATAAAAAACCCCATAGGGCCTTCTGTTTTTTGCCTCTTTCTTTGGAGACTGGG
CTGCCATTTCATGTGAATGGCCACTTTGCACTGGATTCAGCCAGAAGGAACCTGTGGCGTGATGATAATGGAGTT
GGTGTTCGAAGTGACTGGAATAACAGTTTAATGACAGCATTAATAGCTCCTGCATATGTTGAATTGCTAATACAG
TTAAAAAAACGGTATTTCCCTGGTTCTGATCCAACATTATCAGTGTTACAGAACACCCCTATTCATGTTGTAAAG
GACACTTTAAAGAAGTTTTTATCGGGATCCATCTTAAAAGAAGTGACATCTGTGGTGGAGCAAGCATGGAAGCTT
CCAGAATCGGAACGAAAAAAGATTATTAGGCGGTTGTATTTGAAATGGCATCCTGACAAAAATCCAGAGAACCAT
GACATTGCCAATGAAGTTTTTAAACATTTGCAGAATGAAATCAACAGATTAGAAAAACAGGCTTTTCTAGATCAA
AATGCAGACAGGGCCTCCAGACGAACATTTTCAACCTCAGCATCCCGATTTCAGTCAGACAAATACTCATTTCAG
AGATTCTATACTTCATGGAATCAAGAAGCAACGAGCCATAAATCTGAAAGACAGCAACAGAACAAAGAAAAATGC
CCCCCTTCAGCCGGACAGACTTACTCTCAAAGGTTCTTTGTTCCTCCCACTTTCAAGTCGGTTGGCAATCCAGTG
GAAGCACGCAGATGGCTAAGACAAGCCAGAGCAAACTTCTCAGCTGCCAGGAATGACCTTCATAAAAATGCCAAT
GAGTGGGTGTGCTTTAAATGTTACCTTTCTACCAAGTTAGCTTTGATTGCAGCTGACTATGCTGTGAGGGGAAAG
TCTGATAAAGATGTAAAACCAACTGCACTTGCTCAGAAAATAGAGGAATATAGTCAGCAACTTGAAGGACTGACA
AATGATGTTCACACATTGGAAGCTTATGGTGTAGACAGTTTAAAAACAAGATACCCTGATTTGCTTCCCTTTCCT
CAGATCCCAAATGACAGGTTCACTTCTGAGGTTGCTATGAGGGTGATGGAATGTACTGCCTGTATCATAATAAAA
CTTGAAAATTTTATGCAACAAAAAGTG

An example amino acid sequence of an engineered sacsin is:

(SEQ ID NO: 15)
METKENRWVPVTVLPGCVGCRTVAALASWTVRDVKERIFAETGFPVSEQRLWRGGRELSDWIKIGDLTSKNCHLF
VNLQSKGLNVCFTTLGTEFGQKEKLTSRIKSILNAYPSEKEMLKELLQNADDAKATEICFVFDPRQHPVDRIFDD
KWAPLQGPALCVYNNQPFTEDDVRGIQNLGKGTKEGNPYKTGQYGIGFNSVYHITDCPSFISGNDILCIFDPHAR
YAPGATSISPGRMFRDLDADFRTQFSDVLDLYLGTHFKLDNCTMFRFPLRNAEMAKVSEISSVPASDRMVQNLLD
KLRSDGAELLMFLNHMEKISICEIDKSTGALNVLYSVKGKITDGDRLKRKQFHASVIDSVTKKRQLKDIPVQQIT
YTMDTEDSEGNLTTWLICNRSGFSSMEKVSKSVISAHKNQDITLFPRGGVAACITHNYKKPHRAFCFLPLSLETG
LPFHVNGHFALDSARRNLWRDDNGVGVRSDWNNSLMTALIAPAYVELLIQLKKRYFPGSDPTLSVLQNTPIHVVK
DTLKKFLSGSILKEVTSVVEQAWKLPESERKKIIRRLYLKWHPDKNPENHDIANEVFKHLQNEINRLEKQAFLDQ
NADRASRRTFSTSASRFQSDKYSFQRFYTSWNQEATSHKSERQQQNKEKCPPSAGQTYSQRFFVPPTFKSVGNPV
EARRWLRQARANFSAARNDLHKNANEWVCFKCYLSTKLALIAADYAVRGKSDKDVKPTALAQKIEEYSQQLEGLT
NDVHTLEAYGVDSLKTRYPDLLPFPQIPNDRFTSEVAMRVMECTACIIIKLENFMQQKV

A further example codon optimised nucleotide sequence encoding an engineered sacsin is:

(SEQ ID NO: 88)
atgGAGACCAAGGAGAACAGATGGGTACCCGTGACCGTTCTGCCCGGCTGTGTGGGCTGTCGTACGGTGGCAGCC
CTGGCAAGCTGGACCGTGAGAGACGTGAAGGAGAGAATCTTCGCCGAGACCGGCTTCCCCGTGAGCGAGCAGAGA
CTGTGGAGAGGCGGCAGAGAACTGTCCGACTGGATCAAGATCGGCGACCTGACAAGCAAGAACTGCCACCTGTTC
GTGAACCTGCAGAGCAAGGGCCTGAAGGGCGGCGGCCGGTTCGGCCAAACAACCCCGCCACTGGTGGACTTCCTG
AAGGACATCCTGAGAAGATACCCCGAGGGCGGGCAGATACTGAAAGAGCTTATTCAGAACGCCGAAGACGCCGGC
GCCACCGAGGTGAAGTTCCTGTACGACGAGACACAGTACGGCACCGAGACTCTGTGGAGCAAGGACATGGCCCCC
TACCAAGGCCCCGCCCTGTACGTGTACAACAACGCCGTGTTCACCCCCGAGGACTGGCACGGCATCCAAGAGATC
GCTAGAAGCAGAAAGAAGGACGACCCCCTGAAGGTGGGCAGATTCGGCATCGGCTTCAACAGCGTGTACCACATC
ACCGACGTGCCCTGCATCTTCAGCGGCGATCAGATCGGCATGCTGGACCCCCACCAAACACTGTTCGGCCCCCAC
GAGAGCGGGCAGTGCTGGAACCTGAAGGACGACAGCAAGGAGATCAGCGAACTGTCCGATCAGTTCGCCCCCTTC
GTGGGCATCTTCGGCAGCACCAAGGAGACCTTCATCAACGGCAACTTCCCCGGCACCTTCTTCAGATTCCCCCTG
AGACTGCAGCCTTCTCAGCTGAGCAGCAACCTGTACAACAAGCAGAAGGTGCTGGAGCTGTTCGAGAGCTTCAGA
GCCGACGCCGACACCGTGCTGCTGTTCCTGAAGAGCGTGCAAGACGTGAGCCTGTACGTGAGAGAGGCCGACGGT
ACGGAGAAACTGGTGTTCAGAGTGACAAGCAGCGAGAGCAAGGCCCTGAAGCACGAGAGACCCAACAGCATCAAG
ATCCTGGGCACCGCCATCAGCAACTACTGCAAGAAGACCCCTAGCAACAACATCACCTGCGTGACCTACCACGTG
AACATCGTGCTGGAGGAGGAGAGCACCAAGGACGCTCAGAAGACAAGCTGGCTGGTGTGCAACAGCGTGGGCGGC
AGAGGCATCAGCAGCAAGCTGGACAGCCTGGCCGACGAGCTGAAGTTCGTGCCCATCATCGGCATCGCCATGCCC
CTGAGCAGCAGAGACGACGAGGCCAAGGGCGCCACAAGCGACTTTAGTGGTAAAGCCTTCTGCTTCCTGCCACTG
CCACCCGGAGAGGAGAGCAGCACCGGCCTGCCCGTGCACATCAGCGGCTTCTTCGGCCTGACCGACAACAGAAGA
AGCATCAAGTGGAGAGAGCTGGATCAGTGGAGAGACCCCGCCGCCCTGTGGAACGAGTTCCTGGTGATGAACGTG
GTGCCCAAGGCCTACGCCACCCTGggcagcATCCTGAAAGAGGTGACGAGCGTGGTGGAGCAAGCCTGGAAGCTG
CCCGAGAGCGAGAGAAAGAAGATCATCAGAAGACTGTACCTGAAGTGGCACCCCGACAAGAACCCCGAGAACCAC
GACATCGCCAACGAGGTGTTCAAGCACCTGCAGAACGAGATCAACAGACTGGAGAAGCAAGCCTTCCTGGACCAA
AATGCCGACAGAGCAAGCAGAAGAACCTTCAGCACAAGCGCAAGCAGATTTCAGAGCGACAAGTACAGCTTTCAG
AGATTCTACACAAGCTGGAACCAAGAGGCCACAAGCCACAAGAGCGAGAGACAGCAGCAGAACAAGGAGAAGTGC
CCCCCTAGCGCCGGGCAGACCTACTCTCAGAGATTCTTCGTGCCCCCCACCTTCAAGAGCGTGGGCAACCCCGTG
GAGGCTAGAAGATGGCTGAGACAAGCTAGAGCCAACTTCAGCGCCGCTAGAAACGACCTGCACAAGAACGCCAAC
GAGTGGGTGTGCTTCAAGTGCTACCTGAGCACCAAGCTGGCCCTGATCGCCGCCGACTACGCCGTGAGAGGCAAG
AGCGACAAGGACGTGAAGCCCACCGCCCTGGCTCAGAAGATCGAGGAGTACTCTCAGCAGCTGGAGGGCCTGACC
AACGACGTGCACACCCTGGAGGCCTACGGCGTGGACAGCCTGAAGACAAGATACCCCGACCTGCTGCCCTTCCCT
CAGATCCCCAACGACAGATTCACAAGCGAGGTGGCCATGAGAGTGATGGAGTGCACCGCCTGCATCATCATCAAG
CTGGAGAACTTCATGCAGCAGAAGGTGtga

### Expression Control Sequences

The polynucleotide of the present invention may comprise one or more expression control sequence. Suitably, the nucleotide sequence encoding an engineered sacsin is operably linked to one or more expression control sequence.

As used herein an “expression control sequence” is any nucleotide sequence which controls expression of a transgene, e.g. to facilitate and/or increase expression in some cell types and/or decrease expression in other cell types.

The expression control sequence and the nucleotide sequence encoding an engineered sacsin may be in any suitable arrangement in the polynucleotide, providing that the expression control sequence is operably linked to the nucleotide sequence encoding an engineered sacsin. The term “operably linked”, as used herein, means that the parts (e.g. nucleotide sequence encoding an engineered sacsin and one or more expression control sequence) are linked together in a manner which enables both to carry out their function substantially unhindered.

The expression control sequence may be a tissue-specific expression control sequence, particularly a Purkinje cell-specific expression control sequence (e.g. such that the polynucleotide specifically expresses the nucleotide sequence encoding an engineered sacsin in Purkinje cells). Expression control sequences include promoters, enhancers, and 5′ and 3′ untranslated regions.

A “promoter” is a region of DNA that leads to initiation of transcription of a gene. Promoters are located near the transcription start sites of genes, upstream on the DNA (towards the 5′ region of the sense strand).

In some embodiments, the promoter is a constitutive promoter, a tissue-specific promoter or an inducible promoter

In some embodiments, the promoter is a CBA promoter, a CMV promoter or an L7-Pcp2 promoter. In some embodiments, the promoter is a CBA promoter.

An example CBA promoter sequence is:

(SEQ ID NO: 16)
TCGAGGTGAGCCCCACGTTCTGCTTCACTCTCCCCATCTCCCCCCCCTC
CCCACCCCCAATTTTGTATTTATTTATTTTTTAATTATTTTGTGCAGCG
ATGGGGGCGGGGGGGGGGGGGGGGCGCGCGCCAGGCGGGGCGGGGCGGG
GCGAGGGGCGGGGCGGGGCGAGGCGGAGAGGTGCGGCGGCAGCCAATCA
GAGCGGCGCGCTCCGAAAGTTTCCTTTTATGGCGAGGCGGCGGCGGCGG
CGGCCCTATAAAAAGCGAAGCGCGCGGCGGGCG

In some embodiments, the promoter comprises or consists of a nucleotide sequence having at least 70%, 80%, 90%, 95%, 96%, 97%, 98% or 99% identity to SEQ ID NO: 16. In some embodiments, the promoter comprises or consists of the nucleotide sequence of SEQ ID NO: 16.

An example CMV promoter sequence is:

(SEQ ID NO: 17)
GTGATGCGGTTTTGGCAGTACATCAATGGGCGTGGATAGCGGTTTGACT
CACGGGGATTTCCAAGTCTCCACCCCATTGACGTCAATGGGAGTTTGTT
TTGGCACCAAAATCAACGGGACTTTCCAAAATGTCGTAACAACTCCGCC
CCATTGACGCAAATGGGCGGTAGGCGTGTACGGTGGGAGGTCTATATAA
GCAGAGCT

In some embodiments, the promoter comprises or consists of a nucleotide sequence having at least 70%, 80%, 90%, 95%, 96%, 97%, 98% or 99% identity to SEQ ID NO: 17. In some embodiments, the promoter comprises or consists of the nucleotide sequence of SEQ ID NO: 17.

An example L7-Pcp2 promoter sequence is:

(SEQ ID NO: 18)
GGTTCCACCCTCATGTTGGTTGATCTTCAACATTGTCCTGACTTCTTCCCACTTGACATTCCTCAGGGTCCTGTG
ATCATGGCTGGGTCTAGTGAGGTTCAAACCTGCACTGCCCTACCCACACCCACACCCAGCTCAGCGTCAGTCAGG
ATCAACAGTTACCTAGAGATCATCTTTCTGGGGCTTAAGCATTGGTGGGAGCAGATGGGATATGAGTTGGGGATT
TGGGGATGGGGGAAGATATCTGCTCCCCCTCCCCCTACACCCTAGCCTTTTAAAAGGCCTTCTCAGGTCAGAGAC
CAGGAGAAAAGTATAGGAGAGATACACAATGGACCAGGAAGAAGAAAAGGGAGAGGGAGGCTCAGACCTTCTAGA
CAAGGTAAGAGGGCTCTGGCTGACTCCACCATCCGCTTCTTGAGGTCTCGGCACCTGTAATTGACAAGATTAATT
CATTTATAGGGCATCTAATTAGCAAGTAAGTCTCTGGAGTCCCCTGACCCAGTTACTATAACACACAGGGGGTAT
AGGTAGGGGAGTATAAGAGCCCCTCCTCAGGGCAAATGAATGGATTCTTAGTACTGTCCCCCAAGAGATAGTAGG
TACTAGGATTTAGGGGCACTTCTGAGCCCCATTTCCCTGGTAAGTGTCCCAACCCCCCAAATCAACCCAAGCCTG
GTCTCAATCTAGGACAGTGGTAGAATGCTGTCCCTAGAGTCAGTACCATGTGAAATTGTGCTGCAGGCAGGGGCC
CCAGGCTGGGAGGTGGGGGTTGGGGGAGTCAGGGGCAGGTCAGGGAAGGAGACTCAGGTTTCATTTAGAGAAATT
CTGCAGACCCGTGAGGACT

In some embodiments, the promoter comprises or consists of a nucleotide sequence having at least 70%, 80%, 90%, 95%, 96%, 97%, 98% or 99% identity to SEQ ID NO: 18. In some embodiments, the promoter comprises or consists of the nucleotide sequence of SEQ ID NO: 18.

Further example promoter sequences are:

(SEQ ID NO: 53)
CATTATGGCCTTAGGTCACTTCATCTCCATGGGGTTCTTCTTCTGATTTTCTAGAAAATGAGATGGGGGTGCAGA
GAGCTTCCTCAGTGACCTGCCCAGGGTCACATCAGAAATGTCAGAGCTAGAACTTGAACTCAGATTACTAATCTT
AAATTCCATGCCTTGGGGGCATGCAAGTACGATATACAGAAGGAGTGAACTCATTAGGGCAGATGACCAATGAGT
TTAGGAAAGAAGAGTCCAGGGCAGGGTACATCTACACCACCCGCCCAGCCCTGGGTGAGTCCAGCCACGTTCACC
TCATTATAGTTGCCTCTCTCCAGTCCTACCTTGACGGGAAGCACAAGCAGAAACTGGGACAGGAGCCCCAGGAGA
CCAAATCTTCATGGTCCCTCTGGGAGGATGGGTGGGGAGAGCTGTGGCAGAGGCCTCAGGAGGGGCCCTGCTGCT
CAGTGGTGACAGATAGGGGTGAGAAAGCAGACAGAGTCATTCCGTCAGCATTCTGGGTCTGTTTGGTACTTCTTC
TCACGCTAAGGTGGCGGTGTGATATGCACAATGGCTAAAAAGCAGGGAGAGCTGGAAAGAAACAAGGACAGAGAC
AGAGGCCAAGTCAACCAGACCAATTCCCAGAGGAAGCAAAGAAACCATTACAGAGACTACAAGGGGGAAGGGAAG
GAGAGATGAATTAGCTTCCCCTGTAAACCTTAGAACCCAGCTGTTGCCAGGGCAACGGGGCAATACCTGTCTCTT
CAGAGGAGATGAAGTTGCCAGAGTAACTACATCCTGTCTTTCTCAAGGACCATCCCAGAATGTGGCACCCACTAG
CCGTTACCATAGCAACTGCCTCTTTGCCCCACTTAATCCCATCCCGTCTGTTAAAAGGGCCCTATAGTTGGAGGT
GGGGGAGGTAGGAAGAGCGATGATCACTTGTGGACTAAGTTTGTTCGCATCCCCTTCTCCAACCCCCTCAGTACA
TCACCCTGGGGGAACAGGGTCCACTTGCTCCTGGGCCCACACAGTCCTGCAGTATTGTGTATATAAGGCCAGGGC
AAAGAGGAGCAGGTTTTAAAGTGAAAGGCAGGCAGGTGTTGGGGAGGCAGTTACCGGGGCAACGGGAACAGGGCG
TTTCGGAGGTGGTTGCCATGGGGACCTGGATGCTGACGAAGGCTCGCGAGGCTGTGAGCAGCCACAGTGCCCTGC
TCAGAAGCCCCAAGCTCGTCAGTCAAGCCGGTTCTCCGTTTGCACTCAGGAGCACGGGCAGGCGAGTGGCCCCTA
GTTCTGGGGGCAGC
(SEQ ID NO: 54)
AGTGCAAGTGGGTTTTAGGACCAGGATGAGGCGGGGTGGGGGTGCCTACCTGACGACCGACCCCGACCCACTGGA
CAAGCACCCAACCCCCATTCCCCAAATTGCGCATCCCCTATCAGAGAGGGGGAGGGGAAACAGGATGCGGCGAGG
CGCGTGCGCACTGCCAGCTTCAGCACCGCGGACAGTGCCTTCGCCCCCGCCTGGCGGCGCGCGCCACCGCCGCCT
CAGCACTGAAGGCGCGCTGACGTCACTCGCCGGTCCCCCGCAAACTCCCCTTCCCGGCCACCTTGGTCGCGTCCG
CGCCGCCGCCGGCCCAGCCGGACCGCACCACGCGAGGCGCGAGATAGGGGGGCACGGGCGCGACCATCTGCGCTG
CGGCGCCGGCGACTCAGCGCTGCCTCAGTCTGCGGTGGGCAGCGGAGGAGTCGTGTCGTGCCTGAGAGCGCAG
(SEQ ID NO: 55)
CTAGAGGATCCACAGTCTCCTGAGTAGCTGGGACTACAGGAGCTTGTTACCACACCCAGCTCCAGTTTATAAATT
CATCTCCAGTTTATAAAGGAGGAAACCGAGGTACTGAGAGGTTAAAAAACCTTCCTGCAGACACTTGTCCAGCAA
GTGGCCACTCCAGGATTTGGACCAAGGTGATGTGTCTTCAGGCTGTGTCTCTGCCACTGTGCCACGCTGCTGGGT
GGTAGGCAGCAGTGGGTGGGTGCCTGCAGTGGTCTGTAAAGACCACCTGAGATGTCCTTCCTCCTCTGTTCCACC
CTGTCCAGGTCCAAGAAGACAGTCTATGAAGAGAGAGCAGGTGTGACTCTCTCAGTGTGCTCCTCTGTGAGAAGC
AGGCTGACATCCCAAAGGGAAGGGCGGATAACAGAGACAGTGCAAGCGGAGGAGATGAGGGTGCCTCAAAGCCGG
GAGGCTGGGTGATGCAGGAGCCTGCGTGTCCCGAGGGGGGTGCTGGGCCCAGTGTGAGTACGTGTGACTGTGACT
GAGACAGTGTGACTGCTGAAGGCAGGGACACAGCAGCTCCCTGACTGGGGGCAGAAGGCGTTAACTGTGTGAAGG
CTGGTTGTGGGTGGGTGGGCTCTGGGCCTCGAACCCGGGGGCTGAGGGAGATAGTAAACAGCAGGGTGACTGACG
GGAAGATCATGTTGGTAGCCCTGCGAAGATGCTGCAGGGCTGTGGGGGTTTGTGTGACTTTGCAGTTCAACAAAT
TCAAATTCAGCCAACGCTGGCAGGGCCTGTTGTGCCAGGCAACCAGCTAGGAGGAGGAGACTCGGACCCAGCTTG
CAGCTGAAGGGCGCTGGCTGCCGGGTTCTGTGGGTTCACCTTGCGGTGTCTTCCCTTGCTAACACTGAGTCCTTA
CAATAGCCCCATCTCCAGGTTGAGGCTAGATGGAGGGGACAGAGGGAAGTGACTTGCCCAAGGTGACCCAAGCTC
CCGAGTGCCAGGGCAGGATCTGAATTCAGGCTCTCAGACTGCAGAGCCTGAGTCCCTCCCTGCCATGCCTGTGCC
AGGGTGGAAATGTCTGGTCCTGGAGGGGAGCGTGGACTCCTGGCCTTGGCTCTGGAGACATCCCCCTAGACCACG
TGGGCTCCTAACCTGTCCATGGTCACTGTGCTGAGGGGGGGGACGGTGGGTCACCCCTAGTTCTTTTTTCCCCAG
GGCCAGATTCATGGACTGAAGGGTTGCTCGGCTCTCAGAGACCCCCTAAGCGCCCCGCCCTGGCCCCAAGCCCTC
CCCCAGCTCCCGCGTCCCCCCCCTCCTGGCGCTGACTCCGGGCCAGAAGAGGAAAGGCTGTCTCCACCCACCTCT
CGCACTCTCCCTTCTCCTTTATAAAGGCCGGAACAGCTGAAAGGGTGGCAACTTCTCCTCCTGCAGCCGGGAGCG
GCCTGCCTGCCTCCCTGCGCACCCGCAGCCTCCCCCGCTGCCTCCCTAGAGTCGAGGAACTAA
(SEQ ID NO: 56)
TGGCTCCGGTGCCCGTCAGTGGGCAGAGCGCACATCGCCCACAGTCCCCGAGAAGTTGGGGGGAGGGGTCGGCAA
TTGAACCGGTGCCTAGAGAAGGTGGCGCGGGGTAAACTGGGAAAGTGATGTCGTGTACTGGCTCCGCCTTTTTCC
CGAGGGTGGGGGAGAACCGTATATAAGTGCAGTAGTCGCCGTGAACGTTCTTTTTCGCAACGGGTTTGCCGCCAG
AACACAGGTAAGTGCCGTGTGTGGTTCCCGCGGGCCTGGCCTCTTTACGGGTTATGGCCCTTGCGTGCCTTGAAT
TACTTCCACCTGGCTGCAGTACGTGATTCTTGATCCCGAGCTTCGGGTTGGAAGTGGGTGGGAGAGTTCGAGGCC
TTGCGCTTAAGGAGCCCCTTCGCCTCGTGCTTGAGTTGAGGCCTGGCCTGGGCGCTGGGGCCGCCGCGTGCGAAT
CTGGTGGCACCTTCGCGCCTGTCTCGCTGCTTTCGATAAGTCTCTAGCCATTTAAAATTTTTGATGACCTGCTGC
GACGCTTTTTTTCTGGCAAGATAGTCTTGTAAATGCGGGCCAAGATCTGCACACTGGTATTTCGGTTTTTGGGGC
CGCGGGCGGCGACGGGGCCCGTGCGTCCCAGCGCACATGTTCGGCGAGGCGGGGCCTGCGAGCGCGGCCACCGAG
AATCGGACGGGGGTAGTCTCAAGCTGGCCGGCCTGCTCTGGTGCCTGGCCTCGCGCCGCCGTGTATCGCCCCGCC
CTGGGCGGCAAGGCTGGCCCGGTCGGCACCAGTTGCGTGAGCGGAAAGATGGCCGCTTCCCGGCCCTGCTGCAGG
GAGCTCAAAATGGAGGACGCGGCGCTCGGGAGAGCGGGCGGGTGAGTCACCCACACAAAGGAAAAGGGCCTTTCC
GTCCTCAGCCGTCGCTTCATGTGACTCCACGGAGTACCGGGCGCCGTCCAGGCACCTCGATTAGTTCTCGAGCTT
TTGGAGTACGTCGTCTTTAGGTTGGGGGGAGGGGTTTTATGCGATGGAGTTTCCCCACACTGAGTGGGTGGAGAC
TGAAGTTAGGCCAGCTTGGCACTTGATGTAATTCTCCTTGGAATTTGCCCTTTTTGAGTTTGGATCTTGGTTCAT
TCTCAAGCCTCAGACAGTGGTTCAAAGTTTTTTTCTTCCATTTCAGGTGTCGTGA

In some embodiments, the promoter comprises or consists of a nucleotide sequence having at least 70%, 80%, 90%, 95%, 96%, 97%, 98% or 99% identity to any one of SEQ ID NOS: 53-56. In some embodiments, the promoter comprises or consists of the nucleotide sequence of any one of SEQ ID NOs: 53-56.

In some embodiments, the promoter is a human synapsin 1 (hSyn1) promoter or a neuronal specific enolase promoter.

An example human synapsin 1 (hSyn1) promoter sequence is:

(SEQ ID NO: 89)
agtgcaagtgggttttaggaccaggatgaggcggggtgggggtgcctacctgacgaccgaccccgacccactgga
caagcacccaacccccattccccaaattgcgcatcccctatcagagagggggaggggaaacaggatgcggcgagg
cgcgtgcgcactgccagcttcagcaccgcggacagtgccttcgcccccgcctggcggcgcgcgccaccgccgcct
cagcactgaaggcgcgctgacgtcactcgccggtcccccgcaaactccccttcccggccaccttggtcgcgtccg
cgccgccgccggcccagccggaccgcaccacgcgaggcgcgagataggggggcacgggcgcgaccatctgcgctg
cggcgccggcgactcagcgctgcctcagtctgcggtgggcagcggaggagtcgtgtcgtgcctgagagcgcag

Example neuronal specific enolase promoter sequences are:

(SEQ ID NO: 90; human)
atcgataggtaccgagctcttacgcgtgctagctgtatgcagctggacctaggagagaagcaggagaggaagatc
cagcacaaaaaatccgaagctaaaaacaggacacagagatgggggaagaaaagagggcagagtgaggcaaaaaga
gactgaagagatgagggtggccgccaggcactttagataggggagaggctttatttacctctgtttgtttttttt
tttttttttttttttttgcgaggtagtcttgcttagtctccaggctggagtgcagtggcacaatctcagctcact
gcaacttccacctcctgggttcaagcaattctcctgcctcagcctcccgagtagctgggactacaggcgcatgca
accgcgcctggctaatttttgtatttttagtagaaacggggtttcaccacgttagccaggatggtctggatctcc
tgacctcgtgatctgcccgcctccgccttccaaagtgctgggattacaggggtgagccacagcgcctggtcccta
tttacttctgtcttctacctccaggagatcaaagacgctggccttcagacctgatcagactcccaggggcagcca
ccacatgtatgacagagaacagaggatgcctgtttttgccccaaagctggaaattcatcacaacctgaggcccag
gatctgctctgtgccggtcctctgggcagtgtggggtgcagaatggggtgcctaggcctgagcgttgcctggagc
ctaggccgggggccgccctcgggcaggcgtgggtgagagccaagaccgcgtgggccgcggggtgctggtaggagt
ggttggagagacttgcgaaggcggctggggtgttcggatttccaataaagaaacagagtgatgctcctgtgtctg
accgggtttgtgagacattgaggctgtcttgggcttcactggcagtgtgggccttcgtacccgggctacaggggt
gcggctctgcctgttactgtcgagtgggtcgggccgtgggtatgagcgcttgtgtgcgctggggccaggtcgtgg
gtgcccccacccttcccccatcctcctcccttccccactccaccctcgtcggtcccccacccgcgctcgtacgtg
cgcctccgccggcagctcctgactcatcgggggctccgggtcacatgcgcccgcgcggccctataggcgcctcct
ccgcccgccgcccgggagccgcagccgccgccgccactgccactcccgctctctcagcgccgccgtcgccaccgc
caccgccaccgccactaccaccgagatctgcgatctaagtaagcttggcattccggtactgttggtaaa
(SEQ ID NO: 92; rat)
agctctgagctcctcctctgctcgcccaatccttccaaccccctatggtggtatggctgacacagaaaatgtctg
ctcctgtatgggacatttgcccctcttctccaaatataagacaggatgaggcctagcttttgctgctccaaagtt
ttaaaagaacacattgcacggcatttagggactctaaagggtggaggaggaatgagggaattgcatcatgccaag
gctggtcctcatccatcactgcttccagggcccagagtggcttccaggaagtattcttacaaaggaagcccgatc
tgtagctaacactcagagcccattttcctgcgttaacccctcccgacctcatatacaggagtaacatgatcagtg
acctgggggagctggccaaactgcgggacctgcccaagctgagggccttggtgctgctggacaacccctgtgccg
atgagactgactaccgccaggaggccctggtgcagatggcacacctagagcgcctagacaaagagtactatgagg
acgaggaccgggcagaagctgaggagatccgacagaggctgaaggaggaacaggagcaagaactcgacccggacc
aagacatggaaccgtacctcccgccaacttagtggctcctctagcctgcagggacagtaaaggtgatggcaggaa
ggcagcccccggaggtcaaaggctgggcacgcgggaggagaggccagagtcagaggctgcgggtatctcagatat
gaaggaaagatgagagaggctcaggaagaggtaagaaaagacacaagagaccagagaagggagaagaattagaga
gggaggcagaggaccgctgtctctacagacatagctggtagagactgggaggaagggatgaaccctgagcgcatg
aagggaaggaggtggctggtggtatatggaggatgtagctgggccagggaaaagatcctgcactaaaaatctgaa
gctaaaaatacaggacacggggtggagaggcgaaaggagggcagagtgaggcagagagactgagaggcctgggga
tgtgggcattccggtagggcacacagttcacttgtcttctctttttccaggaggccaaagatgctgacgtcaaga
actcataataccccagtggggaccaccgcattcatagccctgttacaagaagtgggagatgttcctttttgtccc
agactggaaatccgttacatcccgaggctcaggttctgtggtggtcatctctgtgtggcttgttctgtgggccta
cctaaagtcctaagcacagctctcaagcagatccgaggcgactaagatgctagtaggggttgtctggagagaaga
gccgaggaggtgggctgtgatggatcagttcagctttcaaataaaaaggcgtttttatattctgtgtcgagttcg
tgaacccctgtggtgggcttctccatctgtctgggttagtacctgccactatactggaataaggggacgcctgct
tccctcgagttggctggacaaggttatgagcatccgtgtacttatggggttgccagcttggtcctggatcgcccg
ggcccttcccccacccgttcggttccccaccaccacccgcgctcgtacgtgcgtctccgcctgcagctcttgact
catcggggcccccgggtcacatgcgctcgctcggctctataggcgccgccccctgcccaccccccgcccgcgctg
ggagccgcagccgccgccactcctgctctctctgcgccg

In some embodiments, the promoter comprises or consists of a nucleotide sequence having at least 70%, 80%, 90%, 95%, 96%, 97%, 98% or 99% identity to any one of SEQ ID NOs: 89, 90 or 92. In some embodiments, the promoter comprises or consists of the nucleotide sequence of any one of SEQ ID NOs: 89, 90 or 92.

The polynucleotide of the invention may also comprise one or more additional regulatory sequence which may act pre- or post-transcriptionally.

Regulatory sequences are any sequences which facilitate expression of the transgene, i.e. act to increase expression of a transcript, improve nuclear export of mRNA or enhance its stability. Such regulatory sequences include for example enhancer elements, post-transcriptional regulatory elements and polyadenylation sites. An example of a polyadenylation site is the Human or Bovine Growth Hormone poly-A signal.

The promoter, for example the CBA promoter, may optionally be used in combination with a cytomegalovirus (CMV) enhancer.

An example CMV enhancer sequence is:

(SEQ ID NO: 19)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATG

In some embodiments, the polynucleotide comprises a nucleotide sequence having at least 70%, 80%, 90%, 95%, 96%, 97%, 98% or 99% identity to SEQ ID NO: 19 operably linked to the nucleotide sequence encoding an engineered sacsin. In some embodiments, the polynucleotide comprises the nucleotide sequence of SEQ ID NO: 19 operably linked to the nucleotide sequence encoding an engineered sacsin.

In some embodiments, the nucleotide sequence encoding an engineered sacsin is operably linked to a CMV enhancer and a CBA promoter. In some embodiments, the nucleotide sequence encoding an engineered sacsin is operably linked to a CMV enhancer, a CBA promoter and a human beta globin intron.

An example human beta globin intron sequence is:

(SEQ ID NO: 52)
GGGAGTCGCTGCGACGCTGCCTTCGCCCCGTGCCCCGCTCCGCCGCCGCCTCGCGCCGCCCGCCCCGGCTCTGAC
TGACCGCGTTACTCCCACAGGTGAGCGGGCGGGACGGCCCTTCTCCTCCGGGCTGTAATTAGCCCGTTTAGTGAA
CCGTCAGATCGCCTGGAGACGCCATCCACGCTGTTTTGACCTCCATAGAAGACACCGGGACCGATCCAGCCTCCG
CGGATTCGAATCCCGGCCGGGAACGGTGCATTGGAACGCGGATTCCCCGTGCCAAGAGTGACGTAAGTACCGCCT
ATAGAGTCTATAGGCCCACAAAAAATGCTTTCTTCTTTTAATATACTTTTTTGTTTATCTTATTTCTAATACTTT
CCCTAATCTCTTTCTTTCAGGGCAATAATGATACAATGTATCATGCCTCTTTGCACCATTCTAAAGAATAACAGT
GATAATTTCTGGGTTAAGGCAATAGCAATATTTCTGCATATAAATATTTCTGCATATAAATTGTAACTGATGTAA
GAGGTTTCATATTGCTAATAGCAGCTACAATCCAGCTACCATTCTGCTTTTATTTTATGGTTGGGATAAGGCTGG
ATTATTCTGAGTCCAAGCTAGGCCCTTTTGCTAATCATGTTCATACCTCTTATCTTCCTCCCACAGCTCCTGGGC
AACGTGCTGGTCTGTGTGCTGGCCCATCACTTTGGCAAAGAATTGGGATTCGAACA

In some embodiments, the polynucleotide comprises a nucleotide sequence having at least 70%, 80%, 90%, 95%, 96%, 97%, 98% or 99% identity to SEQ ID NO: 52 operably linked to the nucleotide sequence encoding an engineered sacsin. In some embodiments, the polynucleotide comprises the nucleotide sequence of SEQ ID NO: 52 operably linked to the nucleotide sequence encoding an engineered sacsin.

In some embodiments, the nucleotide sequence encoding an engineered sacsin is operably linked to a hSyn1 promoter and a chimeric intron, optionally wherein the chimeric intron comprises or consists of a nucleotide sequence having at least 70%, 80%, 90%, 95%, 96%, 97%, 98% or 99% identity to SEQ ID NO: 91. In some embodiments, the chimeric intron comprises or consists of the nucleotide sequence of SEQ ID NO: 91.

(SEQ ID NO: 91)
gtaagtatcaaggttacaagacaggtttaaggagaccaatagaaactgg
gcttgtcgagacagagaagactcttgcgtttctgataggcacctattgg
tcttactgacatccactttgcctttctctccacag

An example human growth hormone polyA sequence is:

(SEQ ID NO: 20)
GGGTGGCATCCCTGTGACCCCTCCCCAGTGCCTCTCCTGGCCCTGGAAGTTGCCACTCCAGTGCCCACCAGCCTT
GTCCTAATAAAATTAAGTTGCATCATTTTGTCTGACTAGGTGTCCTTCTATAATATTATGGGGTGGAGGGGGGTG
GTATGGAGCAAGGGGCAAGTTGGGAAGACAACCTGTAGGGCCTGCGGGGTCTATTGGGAACCAAGCTGGAGTGCA
GTGGCACAATCTTGGCTCACTGCAATCTCCGCCTCCTGGGTTCAAGCGATTCTCCTGCCTCAGCCTCCCGAGTTG
TTGGGATTCCAGGCATGCATGACCAGGCTCAGCTAATTTTTGTTTTTTTGGTAGAGACGGGGTTTCACCATATTG
GCCAGGCTGGTCTCCAACTCCTAATCTCAGGTGATCTACCCACCTTGGCCTCCCAAATTGCTGGGATTACAGGCG
TGAACCACTGCTCCCTTCCCTGTCCTT

An example bovine growth hormone polyA sequence is:

(SEQ ID NO: 21)
CTGTGCCTTCTAGTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCTATTCTGGGGGGTG
GGGTGGGGCAGGACAGCAAGGGGGAGGATTGGGAAGACAATAGCAGGCATGCTGGGGATGCGGTGGGCTCTATGG

The polyA sequence may be an engineered polyA sequence of a small size, an example of which is the pAmin PolyA sequence:

(SEQ ID NO: 22)
AATAAAAGATCTTTATTTTCATTAGATCTGTGTGTTGGTTTTTTGTGTG

In some embodiments, the polynucleotide comprises a nucleotide sequence having at least 70%, 80%, 90%, 95%, 96%, 97%, 98% or 99% identity to SEQ ID NO: 20, 21 or 22 operably linked to the nucleotide sequence encoding an engineered sacsin. In some embodiments, the polynucleotide comprises the nucleotide sequence of SEQ ID NO: 20, 21 or 22 operably linked to the nucleotide sequence encoding an engineered sacsin.

The polynucleotide may comprise a Kozak sequence. Suitably, the protein-coding sequence is operably linked to a Kozak sequence. A Kozak sequence may be inserted before the start codon to improve the initiation of translation.

Suitable Kozak sequences will be well known to the skilled person (see, for example, Kozak, (1987) Nucleic Acids Research 15:8125-8148).

An example Kozak sequence is:

(SEQ ID NO: 23)
GCCGCCGCGATCGCC

In some embodiments, the Kozak sequence comprises or consists of a nucleotide sequence which is at least 80% identical to SEQ ID NO: 23 or a fragment thereof. In some embodiments, the Kozak sequence comprises or consists of the nucleotide sequence SEQ ID NO: 23 or a fragment thereof.

### Tags

The polynucleotide or polypeptide of the invention may further comprise a tag sequence.

Tags are typically sequences that facilitate the detection and/or isolation of the molecule to which they are attached. Tags may be particularly useful in experimental studies utilising the polynucleotides or polypeptides of the invention.

In some embodiments, the polynucleotide of the invention comprises one or more tag sequence. In some embodiments, the polynucleotide of the invention comprises one or more nucleotide sequence encoding a tag.

In some embodiments, the polypeptide of the invention comprises one or more tag sequence.

In some embodiments, the tag is a V5 tag.

An example nucleotide sequence encoding a V5 tag is

(SEQ ID NO: 24)
GGTAAGCCTATCCCTAACCCTCTCCTCGGTCTCGATTCTACG

In some embodiments, the nucleotide sequence encoding a tag comprises or consists of a sequence having at least 70%, 75%, 80%, 85%, 90%, 95%, 96%, 97%, 98% or 99% sequence identity to SEQ ID NO: 24.

In some embodiments, the nucleotide sequence encoding a tag comprises SEQ ID NO: 24.

In some embodiments, the nucleotide sequence encoding a tag consists of SEQ ID NO: 24.

### Exemplary Constructs

The polynucleotide may, for example, comprise or consist of a nucleic acid sequence that has at least 70%, 75%, 80%, 85%, 90%, 95%, 96%, 97%, 98% or 99% sequence identity to any one of SEQ ID NOs: 25-38 or 57-72 or a fragment thereof, preferably wherein the polynucleotide substantially retains the natural function of the polynucleotide of SEQ ID NO: 25-38 or 57-72, respectively.

The polynucleotide may, for example, comprise or consist of the nucleic acid sequence of any one of SEQ ID NOs: 25-38 or 57-72 or a fragment thereof.

An example nucleotide sequence encoding an engineered sacsin comprising an SRR1 domain, a CMV enhancer, a CMV promoter and bGH polyadenylation sequence is:

(SEQ ID NO: 25)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAAAGGGGGAGGTCGATTTGGTCAGACAACGCCACCACTTGTTGAT
TTTCTCAAGGACATTTTGAGAAGATATCCAGAAGGAGGACAGATTCTTAAGGAATTAATTCAGAATGCAGAAGAT
GCTGGGGCGACAGAAGTTAAATTTTTATATGATGAAACTCAATACGGAACAGAGACTCTTTGGTCAAAAGATATG
GCGCCATATCAGGGGCCAGCTCTCTATGTGTACAACAACGCGGTTTTCACCCCAGAGGACTGGCACGGCATTCAA
GAAATAGCAAGAAGCAGGAAAAAGGATGATCCTCTGAAGGTCGGAAGATTTGGAATTGGGTTTAATTCTGTCTAT
CATATAACAGATGTTCCTTGTATCTTTAGTGGTGACCAAATCGGGATGCTAGATCCTCATCAAACACTTTTTGGC
CCACATGAATCAGGCCAATGTTGGAATCTCAAAGATGACAGCAAAGAAATTAGTGAACTTTCAGACCAGTTTGCA
CCATTTGTTGGCATTTTTGGAAGCACCAAGGAAACATTTATAAACGGCAATTTTCCAGGAACATTTTTCCGTTTC
CCTCTTCGCCTACAACCTTCACAACTTAGTAGTAACCTCTACAATAAGCAGAAGGTTCTTGAGTTGTTTGAGTCT
TTTAGGGCAGATGCAGACACAGTGCTGCTCTTTCTGAAAAGTGTGCAGGATGTTTCCTTATATGTCCGAGAGGCT
GACGGAACAGAGAAACTGGTGTTTAGAGTGACTTCGAGTGAGAGTAAGGCACTGAAACATGAGCGGCCGAATTCT
ATAAAGATTCTGGGAACTGCTATAAGTAACTATTGTAAAAAGACTCCAAGCAATAACATCACCTGTGTAACATAT
CACGTAAATATTGTTTTAGAAGAGGAGAGTACTAAGGATGCACAGAAAACATCTTGGTTGGTGTGTAACAGTGTG
GGTGGGCGAGGGATCAGTAGTAAGCTTGACTCTTTAGCTGATGAACTGAAATTTGTCCCAATCATTGGAATAGCC
ATGCCTTTATCAAGCAGAGATGATGAAGCAAAAGGAGCAACGTCTGATTTCTCAGGAAAAGCATTTTGTTTCCTT
CCTTTACCACCTGGTGAGGAAAGCAGCACAGGCCTCCCAGTTCACATCAGTGGGTTCTTTGGCCTTACTGATAAC
CGCAGGAGCATAAAATGGAGAGAGCTGGACCAGTGGAGAGACCCGGCAGCCTTATGGAATGAGTTTCTTGTCATG
AATGTTGTCCCCAAAGCTTATGCTACTCTGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGTGACTCGAGTCTAGAGGGCCCGTTTAAACCCGCTGATCAGCC
TCGACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGGTGGGCTCT
ATGG

An example nucleotide sequence encoding an engineered sacsin comprising an SRR1 domain, a CMV enhancer, a CMV promoter and bGH polyadenylation sequence is:

(SEQ ID NO: 26)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAAAGGGGGAGGTCGATTTGGTCAGACAACGCCACCACTTGTTGAT
TTTCTCAAGGACATTTTGAGAAGATATCCAGAAGGAGGACAGATTCTTAAGGAATTAATTCAGAATGCAGAAGAT
GCTGGGGCGACAGAAGTTAAATTTTTATATGATGAAACTCAATACGGAACAGAGACTCTTTGGTCAAAAGATATG
GCGCCATATCAGGGGCCAGCTCTCTATGTGTACAACAACGCGGTTTTCACCCCAGAGGACTGGCACGGCATTCAA
GAAATAGCAAGAAGCAGGAAAAAGGATGATCCTCTGAAGGTCGGAAGATTTGGAATTGGGTTTAATTCTGTCTAT
CATATAACAGATGTTCCTTGTATCTTTAGTGGTGACCAAATCGGGATGCTAGATCCTCATCAAACACTTTTTGGC
CCACATGAATCAGGCCAATGTTGGAATCTCAAAGATGACAGCAAAGAAATTAGTGAACTTTCAGACCAGTTTGCA
CCATTTGTTGGCATTTTTGGAAGCACCAAGGAAACATTTATAAACGGCAATTTTCCAGGAACATTTTTCCGTTTC
CCTCTTCGCCTACAACCTTCACAACTTAGTAGTAACCTCTACAATAAGCAGAAGGTTCTTGAGTTGTTTGAGTCT
TTTAGGGCAGATGCAGACACAGTGCTGCTCTTTCTGAAAAGTGTGCAGGATGTTTCCTTATATGTCCGAGAGGCT
GACGGAACAGAGAAACTGGTGTTTAGAGTGACTTCGAGTGAGAGTAAGGCACTGAAACATGAGCGGCCGAATTCT
ATAAAGATTCTGGGAACTGCTATAAGTAACTATTGTAAAAAGACTCCAAGCAATAACATCACCTGTGTAACATAT
CACGTAAATATTGTTTTAGAAGAGGAGAGTACTAAGGATGCACAGAAAACATCTTGGTTGGTGTGTAACAGTGTG
GGTGGGCGAGGGATCAGTAGTAAGCTTGACTCTTTAGCTGATGAACTGAAATTTGTCCCAATCATTGGAATAGCC
ATGCCTTTATCAAGCAGAGATGATGAAGCAAAAGGAGCAACGTCTGATTTCTCAGGAAAAGCATTTTGTTTCCTT
CCTTTACCACCTGGTGAGGAAAGCAGCACAGGCCTCCCAGTTCACATCAGTGGGTTCTTTGGCCTTACTGATAAC
CGCAGGAGCATAAAATGGAGAGAGCTGGACCAGTGGAGAGACCCGGCAGCCTTATGGAATGAGTTTCTTGTCATG
AATGTTGTCCCCAAAGCTTATGCTACTCTGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGGGTAAGCCTATCCCTAACCCTCTCCTCGGTCTCGATTCTACG
TGACTCGAGTCTAGAGGGCCCGTTTAAACCCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGG

An example nucleotide sequence encoding an engineered sacsin comprising an SRR1 domain, a CMV enhancer, a CBA promoter, a human beta globin intron and hGH polyadenylation sequence is:

(SEQ ID NO: 27)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGCGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAAAGGGGGAGGTCGATTTGGTCAGACAACGCCACCACTT
GTTGATTTTCTCAAGGACATTTTGAGAAGATATCCAGAAGGAGGACAGATTCTTAAGGAATTAATTCAGAATGCA
GAAGATGCTGGGGCGACAGAAGTTAAATTTTTATATGATGAAACTCAATACGGAACAGAGACTCTTTGGTCAAAA
GATATGGCGCCATATCAGGGGCCAGCTCTCTATGTGTACAACAACGCGGTTTTCACCCCAGAGGACTGGCACGGC
ATTCAAGAAATAGCAAGAAGCAGGAAAAAGGATGATCCTCTGAAGGTCGGAAGATTTGGAATTGGGTTTAATTCT
GTCTATCATATAACAGATGTTCCTTGTATCTTTAGTGGTGACCAAATCGGGATGCTAGATCCTCATCAAACACTT
TTTGGCCCACATGAATCAGGCCAATGTTGGAATCTCAAAGATGACAGCAAAGAAATTAGTGAACTTTCAGACCAG
TTTGCACCATTTGTTGGCATTTTTGGAAGCACCAAGGAAACATTTATAAACGGCAATTTTCCAGGAACATTTTTC
CGTTTCCCTCTTCGCCTACAACCTTCACAACTTAGTAGTAACCTCTACAATAAGCAGAAGGTTCTTGAGTTGTTT
GAGTCTTTTAGGGCAGATGCAGACACAGTGCTGCTCTTTCTGAAAAGTGTGCAGGATGTTTCCTTATATGTCCGA
GAGGCTGACGGAACAGAGAAACTGGTGTTTAGAGTGACTTCGAGTGAGAGTAAGGCACTGAAACATGAGCGGCCG
AATTCTATAAAGATTCTGGGAACTGCTATAAGTAACTATTGTAAAAAGACTCCAAGCAATAACATCACCTGTGTA
ACATATCACGTAAATATTGTTTTAGAAGAGGAGAGTACTAAGGATGCACAGAAAACATCTTGGTTGGTGTGTAAC
AGTGTGGGTGGGCGAGGGATCAGTAGTAAGCTTGACTCTTTAGCTGATGAACTGAAATTTGTCCCAATCATTGGA
ATAGCCATGCCTTTATCAAGCAGAGATGATGAAGCAAAAGGAGCAACGTCTGATTTCTCAGGAAAAGCATTTTGT
TTCCTTCCTTTACCACCTGGTGAGGAAAGCAGCACAGGCCTCCCAGTTCACATCAGTGGGTTCTTTGGCCTTACT
GATAACCGCAGGAGCATAAAATGGAGAGAGCTGGACCAGTGGAGAGACCCGGCAGCCTTATGGAATGAGTTTCTT
GTCATGAATGTTGTCCCCAAAGCTTATGCTACTCTGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGAGAGATCTACGGGTGGCATCCCTGTGACC
CCTCCCCAGTGCCTCTCCTGGCCCTGGAAGTTGCCACTCCAGTGCCCACCAGCCTTGTCCTAATAAAATTAAGTT
GCATCATTTTGTCTGACTAGGTGTCCTTCTATAATATTATGGGGTGGAGGGGGGTGGTATGGAGCAAGGGGCAAG
TTGGGAAGACAACCTGTAGGGCCTGCGGGGTCTATTGGGAACCAAGCTGGAGTGCAGTGGCACAATCTTGGCTCA
CTGCAATCTCCGCCTCCTGGGTTCAAGCGATTCTCCTGCCTCAGCCTCCCGAGTTGTTGGGATTCCAGGCATGCA
TGACCAGGCTCAGCTAATTTTTGTTTTTTTGGTAGAGACGGGGTTTCACCATATTGGCCAGGCTGGTCTCCAACT
CCTAATCTCAGGTGATCTACCCACCTTGGCCTCCCAAATTGCTGGGATTACAGGCGTGAACCACTGCTCCCTTCC
CTGTCCTT

An example nucleotide sequence encoding an engineered sacsin comprising an SRR1 domain, a CMV enhancer, a CBA promoter, a human beta globin intron and hGH polyadenylation sequence is:

(SEQ ID NO: 28)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGGGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAAAGGGGGAGGTCGATTTGGTCAGACAACGCCACCACTT
GTTGATTTTCTCAAGGACATTTTGAGAAGATATCCAGAAGGAGGACAGATTCTTAAGGAATTAATTCAGAATGCA
GAAGATGCTGGGGCGACAGAAGTTAAATTTTTATATGATGAAACTCAATACGGAACAGAGACTCTTTGGTCAAAA
GATATGGCGCCATATCAGGGGCCAGCTCTCTATGTGTACAACAACGCGGTTTTCACCCCAGAGGACTGGCACGGC
ATTCAAGAAATAGCAAGAAGCAGGAAAAAGGATGATCCTCTGAAGGTCGGAAGATTTGGAATTGGGTTTAATTCT
GTCTATCATATAACAGATGTTCCTTGTATCTTTAGTGGTGACCAAATCGGGATGCTAGATCCTCATCAAACACTT
TTTGGCCCACATGAATCAGGCCAATGTTGGAATCTCAAAGATGACAGCAAAGAAATTAGTGAACTTTCAGACCAG
TTTGCACCATTTGTTGGCATTTTTGGAAGCACCAAGGAAACATTTATAAACGGCAATTTTCCAGGAACATTTTTC
CGTTTCCCTCTTCGCCTACAACCTTCACAACTTAGTAGTAACCTCTACAATAAGCAGAAGGTTCTTGAGTTGTTT
GAGTCTTTTAGGGCAGATGCAGACACAGTGCTGCTCTTTCTGAAAAGTGTGCAGGATGTTTCCTTATATGTCCGA
GAGGCTGACGGAACAGAGAAACTGGTGTTTAGAGTGACTTCGAGTGAGAGTAAGGCACTGAAACATGAGCGGCCG
AATTCTATAAAGATTCTGGGAACTGCTATAAGTAACTATTGTAAAAAGACTCCAAGCAATAACATCACCTGTGTA
ACATATCACGTAAATATTGTTTTAGAAGAGGAGAGTACTAAGGATGCACAGAAAACATCTTGGTTGGTGTGTAAC
AGTGTGGGTGGGCGAGGGATCAGTAGTAAGCTTGACTCTTTAGCTGATGAACTGAAATTTGTCCCAATCATTGGA
ATAGCCATGCCTTTATCAAGCAGAGATGATGAAGCAAAAGGAGCAACGTCTGATTTCTCAGGAAAAGCATTTTGT
TTCCTTCCTTTACCACCTGGTGAGGAAAGCAGCACAGGCCTCCCAGTTCACATCAGTGGGTTCTTTGGCCTTACT
GATAACCGCAGGAGCATAAAATGGAGAGAGCTGGACCAGTGGAGAGACCCGGCAGCCTTATGGAATGAGTTTCTT
GTCATGAATGTTGTCCCCAAAGCTTATGCTACTCTGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGAGAGATCTACGGGTGGCATCCCTGTGACCCCTCCCCAGTGCCTCTCCTGGCCCTGGAAGTTG
CCACTCCAGTGCCCACCAGCCTTGTCCTAATAAAATTAAGTTGCATCATTTTGTCTGACTAGGTGTCCTTCTATA
ATATTATGGGGTGGAGGGGGGTGGTATGGAGCAAGGGGCAAGTTGGGAAGACAACCTGTAGGGCCTGCGGGGTCT
ATTGGGAACCAAGCTGGAGTGCAGTGGCACAATCTTGGCTCACTGCAATCTCCGCCTCCTGGGTTCAAGCGATTC
TCCTGCCTCAGCCTCCCGAGTTGTTGGGATTCCAGGCATGCATGACCAGGCTCAGCTAATTTTTGTTTTTTTGGT
AGAGACGGGGTTTCACCATATTGGCCAGGCTGGTCTCCAACTCCTAATCTCAGGTGATCTACCCACCTTGGCCTC
CCAAATTGCTGGGATTACAGGCGTGAACCACTGCTCCCTTCCCTGTCCTT

An example nucleotide sequence encoding an engineered sacsin comprising an SRR2 domain, a CMV enhancer, a CBA promoter, a human beta globin intron and hGH polyadenylation sequence is:

(SEQ ID NO: 29)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGCGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAATAAATCCTGAAAACATGGGATTTGAGCAGTCAGGACAA
AGAGAGCCACTTACTGTAAGAATTAAAAATATTCTGGAAGAATACCCTTCAGTGTCAGATATTTTTAAAGAACTA
CTTCAAAACGCTGATGATGCAAATGCAACAGAATGCAGTTTCTTGATTGATATGAGAAGAAATATGGACATAAGA
GAGAATCTCCTAGACCCAGGGATGGCAGCTTGTCATGGACCTGCTTTGTGGTCATTCAACAATTCTCAATTCTCA
GATTCAGATTTTGTGAACATAACTAGGTTAGGAGAATCTTTAAAAAGGGGAGAAGTTGACAAAGTTGGAAAATTT
GGTCTTGGATTTAATTCTGTGTACCATATCACTGACATTCCCATCATTATGAGTCGGGAATTCATGATAATGTTC
GATCCAAACATAAATCATATCAGTAAACACATTAAAGACAAATCCAATCCTGGGATCAAAATTAATTGGAGTAAA
CAACAGAAAAGACTTAGAAAATTTCCTAATCAGTTCAAACCATTTATAGATGTATTTGGCTGTCAGTTACCTTTG
ACTGTAGAAGCACCTTACAGCTATAATGGAACCCTTTTCCGACTGTCCTTTAGAACTCAACAGGAAGCAAAAGTG
AGTGAAGTTAGTAGTACGTGCTACAATACAGCAGATATTTATTCTCTTGTGGATGAATTTAGTCTCTGTGGACAC
AGGCTTATCATTTTCACTCAGAGTGTAAAGTCAATGTATTTGAAGTACTTGAAAATTGAGGAAACCAACCCCAGT
TTAGCACAAGATACAGTAATAATTAAAAAAAAATCCTGCTCTTCCAAAGCATTGAACACACCTGTCTTAAGTGTT
TTAAAAGAGGCTGCTAAGCTCATGAAGACTTGCAGCAGCAGTAATAAAAAGCTTCCCAGTGATGAACCAAAGTCA
TCTTGCATTCTTCAGATCACAGTGGAAGAATTTCACCATGTGTTCAGAAGGATTGCTGATTTACAGTCGCCACTT
TTTAGAGGTCCAGATGATGACCCAGCTGCTCTCTTTGAAATGGCTAAGTCTGGCCAATCAAAAAAGCCATCAGAT
GAGTTGTCACAGAAAACAGTAGAGTGTACCACGTGGCTTCTGTGTACTTGCATGGACACAGGAGAGGCTCTGAAG
TTTTCCCTGAGTGAGAGTGGAAGAAGACTAGGACTGGTTCCATGTGGGGCAGTAGGAGTTCAGCTGTCAGAAATC
CAGGACCAGAAGTGGACAGTGAAACCACACATTGGAGAGGTGTTTTGCTATTTACCTTTACGAATAAAAACAGGC
TTGCCAGTTCATATCAATGGGTGCTTTGCTGTTACATCAAATAGGAAAGAAATCTGGAAAACAGATACAAAAGGA
CGATGGAATACCACGTTCATGAGACATGTTATTGTGAAAGCTTACTTACAGGTACTGAGTGTCTTACGGGACCTG
GCCACTAGTGGGGAGCTAATGGATTATACTTACTATGCAGTATGGCCCGATCCTGATTTAGTTCATGATGATTTT
TCTGTAATTTGCCAAGGATTTTATGAAGATATAGCTCATGGAGGATCCATCTTAAAAGAAGTGACATCTGTGGTG
GAGCAAGCATGGAAGCTTCCAGAATCGGAACGAAAAAAGATTATTAGGCGGTTGTATTTGAAATGGCATCCTGAC
AAAAATCCAGAGAACCATGACATTGCCAATGAAGTTTTTAAACATTTGCAGAATGAAATCAACAGATTAGAAAAA
CAGGCTTTTCTAGATCAAAATGCAGACAGGGCCTCCAGACGAACATTTTCAACCTCAGCATCCCGATTTCAGTCA
GACAAATACTCATTTCAGAGATTCTATACTTCATGGAATCAAGAAGCAACGAGCCATAAATCTGAAAGACAGCAA
CAGAACAAAGAAAAATGCCCCCCTTCAGCCGGACAGACTTACTCTCAAAGGTTCTTTGTTCCTCCCACTTTCAAG
TCGGTTGGCAATCCAGTGGAAGCACGCAGATGGCTAAGACAAGCCAGAGCAAACTTCTCAGCTGCCAGGAATGAC
CTTCATAAAAATGCCAATGAGTGGGTGTGCTTTAAATGTTACCTTTCTACCAAGTTAGCTTTGATTGCAGCTGAC
TATGCTGTGAGGGGAAAGTCTGATAAAGATGTAAAACCAACTGCACTTGCTCAGAAAATAGAGGAATATAGTCAG
CAACTTGAAGGACTGACAAATGATGTTCACACATTGGAAGCTTATGGTGTAGACAGTTTAAAAACAAGATACCCT
GATTTGCTTCCCTTTCCTCAGATCCCAAATGACAGGTTCACTTCTGAGGTTGCTATGAGGGTGATGGAATGTACT
GCCTGTATCATAATAAAACTTGAAAATTTTATGCAACAAAAAGTGTGACTCGAGAGATCTACGGGTGGCATCCCT
GTGACCCCTCCCCAGTGCCTCTCCTGGCCCTGGAAGTTGCCACTCCAGTGCCCACCAGCCTTGTCCTAATAAAAT
TAAGTTGCATCATTTTGTCTGACTAGGTGTCCTTCTATAATATTATGGGGTGGAGGGGGGTGGTATGGAGCAAGG
GGCAAGTTGGGAAGACAACCTGTAGGGCCTGCGGGGTCTATTGGGAACCAAGCTGGAGTGCAGTGGCACAATCTT
GGCTCACTGCAATCTCCGCCTCCTGGGTTCAAGCGATTCTCCTGCCTCAGCCTCCCGAGTTGTTGGGATTCCAGG
CATGCATGACCAGGCTCAGCTAATTTTTGTTTTTTTGGTAGAGACGGGGTTTCACCATATTGGCCAGGCTGGTCT
CCAACTCCTAATCTCAGGTGATCTACCCACCTTGGCCTCCCAAATTGCTGGGATTACAGGCGTGAACCACTGCTC
CCTTCCCTGTCCTT

An example nucleotide sequence encoding an engineered sacsin comprising an SRR2 domain, a CMV enhancer, a CBA promoter, a human beta globin intron and hGH polyadenylation sequence is:

(SEQ ID NO: 30)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGCGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAATAAATCCTGAAAACATGGGATTTGAGCAGTCAGGACAA
AGAGAGCCACTTACTGTAAGAATTAAAAATATTCTGGAAGAATACCCTTCAGTGTCAGATATTTTTAAAGAACTA
CTTCAAAACGCTGATGATGCAAATGCAACAGAATGCAGTTTCTTGATTGATATGAGAAGAAATATGGACATAAGA
GAGAATCTCCTAGACCCAGGGATGGCAGCTTGTCATGGACCTGCTTTGTGGTCATTCAACAATTCTCAATTCTCA
GATTCAGATTTTGTGAACATAACTAGGTTAGGAGAATCTTTAAAAAGGGGAGAAGTTGACAAAGTTGGAAAATTT
GGTCTTGGATTTAATTCTGTGTACCATATCACTGACATTCCCATCATTATGAGTCGGGAATTCATGATAATGTTC
GATCCAAACATAAATCATATCAGTAAACACATTAAAGACAAATCCAATCCTGGGATCAAAATTAATTGGAGTAAA
CAACAGAAAAGACTTAGAAAATTTCCTAATCAGTTCAAACCATTTATAGATGTATTTGGCTGTCAGTTACCTTTG
ACTGTAGAAGCACCTTACAGCTATAATGGAACCCTTTTCCGACTGTCCTTTAGAACTCAACAGGAAGCAAAAGTG
AGTGAAGTTAGTAGTACGTGCTACAATACAGCAGATATTTATTCTCTTGTGGATGAATTTAGTCTCTGTGGACAC
AGGCTTATCATTTTCACTCAGAGTGTAAAGTCAATGTATTTGAAGTACTTGAAAATTGAGGAAACCAACCCCAGT
TTAGCACAAGATACAGTAATAATTAAAAAAAAATCCTGCTCTTCCAAAGCATTGAACACACCTGTCTTAAGTGTT
TTAAAAGAGGCTGCTAAGCTCATGAAGACTTGCAGCAGCAGTAATAAAAAGCTTCCCAGTGATGAACCAAAGTCA
TCTTGCATTCTTCAGATCACAGTGGAAGAATTTCACCATGTGTTCAGAAGGATTGCTGATTTACAGTCGCCACTT
TTTAGAGGTCCAGATGATGACCCAGCTGCTCTCTTTGAAATGGCTAAGTCTGGCCAATCAAAAAAGCCATCAGAT
GAGTTGTCACAGAAAACAGTAGAGTGTACCACGTGGCTTCTGTGTACTTGCATGGACACAGGAGAGGCTCTGAAG
TTTTCCCTGAGTGAGAGTGGAAGAAGACTAGGACTGGTTCCATGTGGGGCAGTAGGAGTTCAGCTGTCAGAAATC
CAGGACCAGAAGTGGACAGTGAAACCACACATTGGAGAGGTGTTTTGCTATTTACCTTTACGAATAAAAACAGGC
TTGCCAGTTCATATCAATGGGTGCTTTGCTGTTACATCAAATAGGAAAGAAATCTGGAAAACAGATACAAAAGGA
CGATGGAATACCACGTTCATGAGACATGTTATTGTGAAAGCTTACTTACAGGTACTGAGTGTCTTACGGGACCTG
GCCACTAGTGGGGAGCTAATGGATTATACTTACTATGCAGTATGGCCCGATCCTGATTTAGTTCATGATGATTTT
TCTGTAATTTGCCAAGGATTTTATGAAGATATAGCTCATGGAGGATCCATCTTAAAAGAAGTGACATCTGTGGTG
GAGCAAGCATGGAAGCTTCCAGAATCGGAACGAAAAAAGATTATTAGGCGGTTGTATTTGAAATGGCATCCTGAC
AAAAATCCAGAGAACCATGACATTGCCAATGAAGTTTTTAAACATTTGCAGAATGAAATCAACAGATTAGAAAAA
CAGGCTTTTCTAGATCAAAATGCAGACAGGGCCTCCAGACGAACATTTTCAACCTCAGCATCCCGATTTCAGTCA
GACAAATACTCATTTCAGAGATTCTATACTTCATGGAATCAAGAAGCAACGAGCCATAAATCTGAAAGACAGCAA
CAGAACAAAGAAAAATGCCCCCCTTCAGCCGGACAGACTTACTCTCAAAGGTTCTTTGTTCCTCCCACTTTCAAG
TCGGTTGGCAATCCAGTGGAAGCACGCAGATGGCTAAGACAAGCCAGAGCAAACTTCTCAGCTGCCAGGAATGAC
CTTCATAAAAATGCCAATGAGTGGGTGTGCTTTAAATGTTACCTTTCTACCAAGTTAGCTTTGATTGCAGCTGAC
TATGCTGTGAGGGGAAAGTCTGATAAAGATGTAAAACCAACTGCACTTGCTCAGAAAATAGAGGAATATAGTCAG
CAACTTGAAGGACTGACAAATGATGTTCACACATTGGAAGCTTATGGTGTAGACAGTTTAAAAACAAGATACCCT
GATTTGCTTCCCTTTCCTCAGATCCCAAATGACAGGTTCACTTCTGAGGTTGCTATGAGGGTGATGGAATGTACT
GCCTGTATCATAATAAAACTTGAAAATTTTATGCAACAAAAAGTGGGTAAGCCTATCCCTAACCCTCTCCTCGGT
CTCGATTCTACGTGACTCGAGAGATCTACGGGTGGCATCCCTGTGACCCCTCCCCAGTGCCTCTCCTGGCCCTGG
AAGTTGCCACTCCAGTGCCCACCAGCCTTGTCCTAATAAAATTAAGTTGCATCATTTTGTCTGACTAGGTGTCCT
TCTATAATATTATGGGGTGGAGGGGGGTGGTATGGAGCAAGGGGCAAGTTGGGAAGACAACCTGTAGGGCCTGCG
GGGTCTATTGGGAACCAAGCTGGAGTGCAGTGGCACAATCTTGGCTCACTGCAATCTCCGCCTCCTGGGTTCAAG
CGATTCTCCTGCCTCAGCCTCCCGAGTTGTTGGGATTCCAGGCATGCATGACCAGGCTCAGCTAATTTTTGTTTT
TTTGGTAGAGACGGGGTTTCACCATATTGGCCAGGCTGGTCTCCAACTCCTAATCTCAGGTGATCTACCCACCTT
GGCCTCCCAAATTGCTGGGATTACAGGCGTGAACCACTGCTCCCTTCCCTGTCCTT

An example nucleotide sequence encoding an engineered sacsin comprising an SRR3 domain, a CMV enhancer, a CBA promoter, a human beta globin intron and hGH polyadenylation sequence is:

(SEQ ID NO: 31)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGCGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAATGTCTGTTTTACAACACTTGGCACAGAATTTGGGCAG
AAAGAAAAATTGACCAGCAGAATTAAGAGCATCCTTAATGCATATCCTTCTGAAAAGGAAATGTTGAAAGAGCTT
CTTCAAAATGCTGATGATGCAAAGGCGACAGAAATCTGTTTTGTGTTTGATCCTAGACAGCATCCAGTTGATAGA
ATATTTGATGATAAGTGGGCCCCATTGCAAGGGCCAGCACTTTGTGTGTACAACAACCAGCCATTTACAGAAGAT
GATGTTAGAGGAATTCAGAATCTTGGAAAAGGCACGAAAGAGGGAAATCCTTATAAAACTGGACAGTATGGAATA
GGATTCAATTCTGTGTATCATATCACAGACTGCCCATCTTTTATTTCTGGCAATGACATCCTGTGTATTTTTGAT
CCTCATGCCAGATATGCACCAGGGGCCACATCCATTAGTCCCGGACGCATGTTTAGAGATTTGGATGCAGATTTT
AGGACACAGTTCTCAGATGTTCTGGATCTTTATCTGGGAACCCATTTTAAACTGGATAATTGCACAATGTTCAGA
TTTCCTCTTCGTAATGCAGAAATGGCAAAAGTTTCGGAAATTTCGTCTGTTCCAGCATCAGACAGAATGGTCCAG
AATCTTTTGGACAAACTGCGCTCAGATGGGGCAGAACTTCTAATGTTTCTTAATCACATGGAAAAAATTTCTATT
TGTGAAATAGATAAGAGTACTGGAGCTCTAAATGTGCTGTATTCAGTAAAGGGCAAAATCACAGATGGAGACAGA
TTGAAAAGGAAACAATTTCATGCATCTGTAATTGATAGTGTTACTAAAAAGAGGCAGCTCAAAGACATACCAGTT
CAACAAATAACCTATACTATGGATACTGAGGACTCTGAAGGAAATCTTACTACGTGGCTAATTTGTAATAGATCA
GGCTTTTCAAGTATGGAGAAAGTATCTAAAAGTGTCATATCAGCTCACAAGAACCAAGATATTACTCTTTTCCCA
CGTGGTGGAGTAGCTGCCTGCATTACTCACAACTATAAAAAACCCCATAGGGCCTTCTGTTTTTTGCCTCTTTCT
TTGGAGACTGGGCTGCCATTTCATGTGAATGGCCACTTTGCACTGGATTCAGCCAGAAGGAACCTGTGGCGTGAT
GATAATGGAGTTGGTGTTCGAAGTGACTGGAATAACAGTTTAATGACAGCATTAATAGCTCCTGCATATGTTGAA
TTGCTAATACAGTTAAAAAAACGGTATTTCCCTGGTTCTGATCCAACATTATCAGTGTTACAGAACACCCCTATT
CATGTTGTAAAGGACACTTTAAAGAAGTTTTTATCGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGAGAGATCTACGGGTGGCATCCCTGTGACC
CCTCCCCAGTGCCTCTCCTGGCCCTGGAAGTTGCCACTCCAGTGCCCACCAGCCTTGTCCTAATAAAATTAAGTT
GCATCATTTTGTCTGACTAGGTGTCCTTCTATAATATTATGGGGTGGAGGGGGGTGGTATGGAGCAAGGGGCAAG
TTGGGAAGACAACCTGTAGGGCCTGCGGGGTCTATTGGGAACCAAGCTGGAGTGCAGTGGCACAATCTTGGCTCA
CTGCAATCTCCGCCTCCTGGGTTCAAGCGATTCTCCTGCCTCAGCCTCCCGAGTTGTTGGGATTCCAGGCATGCA
TGACCAGGCTCAGCTAATTTTTGTTTTTTTGGTAGAGACGGGGTTTCACCATATTGGCCAGGCTGGTCTCCAACT
CCTAATCTCAGGTGATCTACCCACCTTGGCCTCCCAAATTGCTGGGATTACAGGCGTGAACCACTGCTCCCTTCC
CTGTCCTT

An example nucleotide sequence encoding an engineered sacsin comprising an SRR3 domain, a CMV enhancer, a CBA promoter, a human beta globin intron and hGH polyadenylation sequence is:

(SEQ ID NO: 32)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGGGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAATGTCTGTTTTACAACACTTGGCACAGAATTTGGGCAG
AAAGAAAAATTGACCAGCAGAATTAAGAGCATCCTTAATGCATATCCTTCTGAAAAGGAAATGTTGAAAGAGCTT
CTTCAAAATGCTGATGATGCAAAGGCGACAGAAATCTGTTTTGTGTTTGATCCTAGACAGCATCCAGTTGATAGA
ATATTTGATGATAAGTGGGCCCCATTGCAAGGGCCAGCACTTTGTGTGTACAACAACCAGCCATTTACAGAAGAT
GATGTTAGAGGAATTCAGAATCTTGGAAAAGGCACGAAAGAGGGAAATCCTTATAAAACTGGACAGTATGGAATA
GGATTCAATTCTGTGTATCATATCACAGACTGCCCATCTTTTATTTCTGGCAATGACATCCTGTGTATTTTTGAT
CCTCATGCCAGATATGCACCAGGGGCCACATCCATTAGTCCCGGACGCATGTTTAGAGATTTGGATGCAGATTTT
AGGACACAGTTCTCAGATGTTCTGGATCTTTATCTGGGAACCCATTTTAAACTGGATAATTGCACAATGTTCAGA
TTTCCTCTTCGTAATGCAGAAATGGCAAAAGTTTCGGAAATTTCGTCTGTTCCAGCATCAGACAGAATGGTCCAG
AATCTTTTGGACAAACTGCGCTCAGATGGGGCAGAACTTCTAATGTTTCTTAATCACATGGAAAAAATTTCTATT
TGTGAAATAGATAAGAGTACTGGAGCTCTAAATGTGCTGTATTCAGTAAAGGGCAAAATCACAGATGGAGACAGA
TTGAAAAGGAAACAATTTCATGCATCTGTAATTGATAGTGTTACTAAAAAGAGGCAGCTCAAAGACATACCAGTT
CAACAAATAACCTATACTATGGATACTGAGGACTCTGAAGGAAATCTTACTACGTGGCTAATTTGTAATAGATCA
GGCTTTTCAAGTATGGAGAAAGTATCTAAAAGTGTCATATCAGCTCACAAGAACCAAGATATTACTCTTTTCCCA
CGTGGTGGAGTAGCTGCCTGCATTACTCACAACTATAAAAAACCCCATAGGGCCTTCTGTTTTTTGCCTCTTTCT
TTGGAGACTGGGCTGCCATTTCATGTGAATGGCCACTTTGCACTGGATTCAGCCAGAAGGAACCTGTGGCGTGAT
GATAATGGAGTTGGTGTTCGAAGTGACTGGAATAACAGTTTAATGACAGCATTAATAGCTCCTGCATATGTTGAA
TTGCTAATACAGTTAAAAAAACGGTATTTCCCTGGTTCTGATCCAACATTATCAGTGTTACAGAACACCCCTATT
CATGTTGTAAAGGACACTTTAAAGAAGTTTTTATCGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGAGAGATCTACGGGTGGCATCCCTGTGACCCCTCCCCAGTGCCTCTCCTGGCCCTGGAAGTTG
CCACTCCAGTGCCCACCAGCCTTGTCCTAATAAAATTAAGTTGCATCATTTTGTCTGACTAGGTGTCCTTCTATA
ATATTATGGGGTGGAGGGGGGTGGTATGGAGCAAGGGGCAAGTTGGGAAGACAACCTGTAGGGCCTGCGGGGTCT
ATTGGGAACCAAGCTGGAGTGCAGTGGCACAATCTTGGCTCACTGCAATCTCCGCCTCCTGGGTTCAAGCGATTC
TCCTGCCTCAGCCTCCCGAGTTGTTGGGATTCCAGGCATGCATGACCAGGCTCAGCTAATTTTTGTTTTTTTGGT
AGAGACGGGGTTTCACCATATTGGCCAGGCTGGTCTCCAACTCCTAATCTCAGGTGATCTACCCACCTTGGCCTC
CCAAATTGCTGGGATTACAGGCGTGAACCACTGCTCCCTTCCCTGTCCTT

An example nucleotide sequence encoding an engineered sacsin comprising an SRR1 domain, a CMV enhancer, a CBA promoter, a human beta globin intron and pAmin polyadenylation sequence is:

(SEQ ID NO: 33)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGCGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAAAGGGGGAGGTCGATTTGGTCAGACAACGCCACCACTT
GTTGATTTTCTCAAGGACATTTTGAGAAGATATCCAGAAGGAGGACAGATTCTTAAGGAATTAATTCAGAATGCA
GAAGATGCTGGGGCGACAGAAGTTAAATTTTTATATGATGAAACTCAATACGGAACAGAGACTCTTTGGTCAAAA
GATATGGCGCCATATCAGGGGCCAGCTCTCTATGTGTACAACAACGCGGTTTTCACCCCAGAGGACTGGCACGGC
ATTCAAGAAATAGCAAGAAGCAGGAAAAAGGATGATCCTCTGAAGGTCGGAAGATTTGGAATTGGGTTTAATTCT
GTCTATCATATAACAGATGTTCCTTGTATCTTTAGTGGTGACCAAATCGGGATGCTAGATCCTCATCAAACACTT
TTTGGCCCACATGAATCAGGCCAATGTTGGAATCTCAAAGATGACAGCAAAGAAATTAGTGAACTTTCAGACCAG
TTTGCACCATTTGTTGGCATTTTTGGAAGCACCAAGGAAACATTTATAAACGGCAATTTTCCAGGAACATTTTTC
CGTTTCCCTCTTCGCCTACAACCTTCACAACTTAGTAGTAACCTCTACAATAAGCAGAAGGTTCTTGAGTTGTTT
GAGTCTTTTAGGGCAGATGCAGACACAGTGCTGCTCTTTCTGAAAAGTGTGCAGGATGTTTCCTTATATGTCCGA
GAGGCTGACGGAACAGAGAAACTGGTGTTTAGAGTGACTTCGAGTGAGAGTAAGGCACTGAAACATGAGCGGCCG
AATTCTATAAAGATTCTGGGAACTGCTATAAGTAACTATTGTAAAAAGACTCCAAGCAATAACATCACCTGTGTA
ACATATCACGTAAATATTGTTTTAGAAGAGGAGAGTACTAAGGATGCACAGAAAACATCTTGGTTGGTGTGTAAC
AGTGTGGGTGGGCGAGGGATCAGTAGTAAGCTTGACTCTTTAGCTGATGAACTGAAATTTGTCCCAATCATTGGA
ATAGCCATGCCTTTATCAAGCAGAGATGATGAAGCAAAAGGAGCAACGTCTGATTTCTCAGGAAAAGCATTTTGT
TTCCTTCCTTTACCACCTGGTGAGGAAAGCAGCACAGGCCTCCCAGTTCACATCAGTGGGTTCTTTGGCCTTACT
GATAACCGCAGGAGCATAAAATGGAGAGAGCTGGACCAGTGGAGAGACCCGGCAGCCTTATGGAATGAGTTTCTT
GTCATGAATGTTGTCCCCAAAGCTTATGCTACTCTGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGACGAATTCAATAAAAGATCTTTATTTTCA
TTAGATCTGTGTGTTGGTTTTTTGTGTG

An example nucleotide sequence encoding an engineered sacsin comprising an SRR1 domain, a CMV enhancer, a CBA promoter, a human beta globin intron and pAmin polyadenylation sequence is:

(SEQ ID NO: 34)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGCGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAAAGGGGGAGGTCGATTTGGTCAGACAACGCCACCACTT
GTTGATTTTCTCAAGGACATTTTGAGAAGATATCCAGAAGGAGGACAGATTCTTAAGGAATTAATTCAGAATGCA
GAAGATGCTGGGGCGACAGAAGTTAAATTTTTATATGATGAAACTCAATACGGAACAGAGACTCTTTGGTCAAAA
GATATGGCGCCATATCAGGGGCCAGCTCTCTATGTGTACAACAACGCGGTTTTCACCCCAGAGGACTGGCACGGC
ATTCAAGAAATAGCAAGAAGCAGGAAAAAGGATGATCCTCTGAAGGTCGGAAGATTTGGAATTGGGTTTAATTCT
GTCTATCATATAACAGATGTTCCTTGTATCTTTAGTGGTGACCAAATCGGGATGCTAGATCCTCATCAAACACTT
TTTGGCCCACATGAATCAGGCCAATGTTGGAATCTCAAAGATGACAGCAAAGAAATTAGTGAACTTTCAGACCAG
TTTGCACCATTTGTTGGCATTTTTGGAAGCACCAAGGAAACATTTATAAACGGCAATTTTCCAGGAACATTTTTC
CGTTTCCCTCTTCGCCTACAACCTTCACAACTTAGTAGTAACCTCTACAATAAGCAGAAGGTTCTTGAGTTGTTT
GAGTCTTTTAGGGCAGATGCAGACACAGTGCTGCTCTTTCTGAAAAGTGTGCAGGATGTTTCCTTATATGTCCGA
GAGGCTGACGGAACAGAGAAACTGGTGTTTAGAGTGACTTCGAGTGAGAGTAAGGCACTGAAACATGAGCGGCCG
AATTCTATAAAGATTCTGGGAACTGCTATAAGTAACTATTGTAAAAAGACTCCAAGCAATAACATCACCTGTGTA
ACATATCACGTAAATATTGTTTTAGAAGAGGAGAGTACTAAGGATGCACAGAAAACATCTTGGTTGGTGTGTAAC
AGTGTGGGTGGGCGAGGGATCAGTAGTAAGCTTGACTCTTTAGCTGATGAACTGAAATTTGTCCCAATCATTGGA
ATAGCCATGCCTTTATCAAGCAGAGATGATGAAGCAAAAGGAGCAACGTCTGATTTCTCAGGAAAAGCATTTTGT
TTCCTTCCTTTACCACCTGGTGAGGAAAGCAGCACAGGCCTCCCAGTTCACATCAGTGGGTTCTTTGGCCTTACT
GATAACCGCAGGAGCATAAAATGGAGAGAGCTGGACCAGTGGAGAGACCCGGCAGCCTTATGGAATGAGTTTCTT
GTCATGAATGTTGTCCCCAAAGCTTATGCTACTCTGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGACGAATTCAATAAAAGATCTTTATTTTCATTAGATCTGTGTGTTGGTTTTTTGTGTG

An example nucleotide sequence encoding an engineered sacsin comprising an SRR2 domain, a CMV enhancer, a CBA promoter, a human beta globin intron and pAmin polyadenylation sequence is:

(SEQ ID NO: 35)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGGCGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAATAAATCCTGAAAACATGGGATTTGAGCAGTCAGGACAA
AGAGAGCCACTTACTGTAAGAATTAAAAATATTCTGGAAGAATACCCTTCAGTGTCAGATATTTTTAAAGAACTA
CTTCAAAACGCTGATGATGCAAATGCAACAGAATGCAGTTTCTTGATTGATATGAGAAGAAATATGGACATAAGA
GAGAATCTCCTAGACCCAGGGATGGCAGCTTGTCATGGACCTGCTTTGTGGTCATTCAACAATTCTCAATTCTCA
GATTCAGATTTTGTGAACATAACTAGGTTAGGAGAATCTTTAAAAAGGGGAGAAGTTGACAAAGTTGGAAAATTT
GGTCTTGGATTTAATTCTGTGTACCATATCACTGACATTCCCATCATTATGAGTCGGGAATTCATGATAATGTTC
GATCCAAACATAAATCATATCAGTAAACACATTAAAGACAAATCCAATCCTGGGATCAAAATTAATTGGAGTAAA
CAACAGAAAAGACTTAGAAAATTTCCTAATCAGTTCAAACCATTTATAGATGTATTTGGCTGTCAGTTACCTTTG
ACTGTAGAAGCACCTTACAGCTATAATGGAACCCTTTTCCGACTGTCCTTTAGAACTCAACAGGAAGCAAAAGTG
AGTGAAGTTAGTAGTACGTGCTACAATACAGCAGATATTTATTCTCTTGTGGATGAATTTAGTCTCTGTGGACAC
AGGCTTATCATTTTCACTCAGAGTGTAAAGTCAATGTATTTGAAGTACTTGAAAATTGAGGAAACCAACCCCAGT
TTAGCACAAGATACAGTAATAATTAAAAAAAAATCCTGCTCTTCCAAAGCATTGAACACACCTGTCTTAAGTGTT
TTAAAAGAGGCTGCTAAGCTCATGAAGACTTGCAGCAGCAGTAATAAAAAGCTTCCCAGTGATGAACCAAAGTCA
TCTTGCATTCTTCAGATCACAGTGGAAGAATTTCACCATGTGTTCAGAAGGATTGCTGATTTACAGTCGCCACTT
TTTAGAGGTCCAGATGATGACCCAGCTGCTCTCTTTGAAATGGCTAAGTCTGGCCAATCAAAAAAGCCATCAGAT
GAGTTGTCACAGAAAACAGTAGAGTGTACCACGTGGCTTCTGTGTACTTGCATGGACACAGGAGAGGCTCTGAAG
TTTTCCCTGAGTGAGAGTGGAAGAAGACTAGGACTGGTTCCATGTGGGGCAGTAGGAGTTCAGCTGTCAGAAATC
CAGGACCAGAAGTGGACAGTGAAACCACACATTGGAGAGGTGTTTTGCTATTTACCTTTACGAATAAAAACAGGC
TTGCCAGTTCATATCAATGGGTGCTTTGCTGTTACATCAAATAGGAAAGAAATCTGGAAAACAGATACAAAAGGA
CGATGGAATACCACGTTCATGAGACATGTTATTGTGAAAGCTTACTTACAGGTACTGAGTGTCTTACGGGACCTG
GCCACTAGTGGGGAGCTAATGGATTATACTTACTATGCAGTATGGCCCGATCCTGATTTAGTTCATGATGATTTT
TCTGTAATTTGCCAAGGATTTTATGAAGATATAGCTCATGGAGGATCCATCTTAAAAGAAGTGACATCTGTGGTG
GAGCAAGCATGGAAGCTTCCAGAATCGGAACGAAAAAAGATTATTAGGCGGTTGTATTTGAAATGGCATCCTGAC
AAAAATCCAGAGAACCATGACATTGCCAATGAAGTTTTTAAACATTTGCAGAATGAAATCAACAGATTAGAAAAA
CAGGCTTTTCTAGATCAAAATGCAGACAGGGCCTCCAGACGAACATTTTCAACCTCAGCATCCCGATTTCAGTCA
GACAAATACTCATTTCAGAGATTCTATACTTCATGGAATCAAGAAGCAACGAGCCATAAATCTGAAAGACAGCAA
CAGAACAAAGAAAAATGCCCCCCTTCAGCCGGACAGACTTACTCTCAAAGGTTCTTTGTTCCTCCCACTTTCAAG
TCGGTTGGCAATCCAGTGGAAGCACGCAGATGGCTAAGACAAGCCAGAGCAAACTTCTCAGCTGCCAGGAATGAC
CTTCATAAAAATGCCAATGAGTGGGTGTGCTTTAAATGTTACCTTTCTACCAAGTTAGCTTTGATTGCAGCTGAC
TATGCTGTGAGGGGAAAGTCTGATAAAGATGTAAAACCAACTGCACTTGCTCAGAAAATAGAGGAATATAGTCAG
CAACTTGAAGGACTGACAAATGATGTTCACACATTGGAAGCTTATGGTGTAGACAGTTTAAAAACAAGATACCCT
GATTTGCTTCCCTTTCCTCAGATCCCAAATGACAGGTTCACTTCTGAGGTTGCTATGAGGGTGATGGAATGTACT
GCCTGTATCATAATAAAACTTGAAAATTTTATGCAACAAAAAGTGTGACTCGACGAATTCAATAAAAGATCTTTA
TTTTCATTAGATCTGTGTGTTGGTTTTTTGTGTG

An example nucleotide sequence encoding an engineered sacsin comprising an SRR2 domain, a CMV enhancer, a CBA promoter, a human beta globin intron and pAmin polyadenylation sequence is:

(SEQ ID NO: 36)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGGGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAATAAATCCTGAAAACATGGGATTTGAGCAGTCAGGACAA
AGAGAGCCACTTACTGTAAGAATTAAAAATATTCTGGAAGAATACCCTTCAGTGTCAGATATTTTTAAAGAACTA
CTTCAAAACGCTGATGATGCAAATGCAACAGAATGCAGTTTCTTGATTGATATGAGAAGAAATATGGACATAAGA
GAGAATCTCCTAGACCCAGGGATGGCAGCTTGTCATGGACCTGCTTTGTGGTCATTCAACAATTCTCAATTCTCA
GATTCAGATTTTGTGAACATAACTAGGTTAGGAGAATCTTTAAAAAGGGGAGAAGTTGACAAAGTTGGAAAATTT
GGTCTTGGATTTAATTCTGTGTACCATATCACTGACATTCCCATCATTATGAGTCGGGAATTCATGATAATGTTC
GATCCAAACATAAATCATATCAGTAAACACATTAAAGACAAATCCAATCCTGGGATCAAAATTAATTGGAGTAAA
CAACAGAAAAGACTTAGAAAATTTCCTAATCAGTTCAAACCATTTATAGATGTATTTGGCTGTCAGTTACCTTTG
ACTGTAGAAGCACCTTACAGCTATAATGGAACCCTTTTCCGACTGTCCTTTAGAACTCAACAGGAAGCAAAAGTG
AGTGAAGTTAGTAGTACGTGCTACAATACAGCAGATATTTATTCTCTTGTGGATGAATTTAGTCTCTGTGGACAC
AGGCTTATCATTTTCACTCAGAGTGTAAAGTCAATGTATTTGAAGTACTTGAAAATTGAGGAAACCAACCCCAGT
TTAGCACAAGATACAGTAATAATTAAAAAAAAATCCTGCTCTTCCAAAGCATTGAACACACCTGTCTTAAGTGTT
TTAAAAGAGGCTGCTAAGCTCATGAAGACTTGCAGCAGCAGTAATAAAAAGCTTCCCAGTGATGAACCAAAGTCA
TCTTGCATTCTTCAGATCACAGTGGAAGAATTTCACCATGTGTTCAGAAGGATTGCTGATTTACAGTCGCCACTT
TTTAGAGGTCCAGATGATGACCCAGCTGCTCTCTTTGAAATGGCTAAGTCTGGCCAATCAAAAAAGCCATCAGAT
GAGTTGTCACAGAAAACAGTAGAGTGTACCACGTGGCTTCTGTGTACTTGCATGGACACAGGAGAGGCTCTGAAG
TTTTCCCTGAGTGAGAGTGGAAGAAGACTAGGACTGGTTCCATGTGGGGCAGTAGGAGTTCAGCTGTCAGAAATC
CAGGACCAGAAGTGGACAGTGAAACCACACATTGGAGAGGTGTTTTGCTATTTACCTTTACGAATAAAAACAGGC
TTGCCAGTTCATATCAATGGGTGCTTTGCTGTTACATCAAATAGGAAAGAAATCTGGAAAACAGATACAAAAGGA
CGATGGAATACCACGTTCATGAGACATGTTATTGTGAAAGCTTACTTACAGGTACTGAGTGTCTTACGGGACCTG
GCCACTAGTGGGGAGCTAATGGATTATACTTACTATGCAGTATGGCCCGATCCTGATTTAGTTCATGATGATTTT
TCTGTAATTTGCCAAGGATTTTATGAAGATATAGCTCATGGAGGATCCATCTTAAAAGAAGTGACATCTGTGGTG
GAGCAAGCATGGAAGCTTCCAGAATCGGAACGAAAAAAGATTATTAGGCGGTTGTATTTGAAATGGCATCCTGAC
AAAAATCCAGAGAACCATGACATTGCCAATGAAGTTTTTAAACATTTGCAGAATGAAATCAACAGATTAGAAAAA
CAGGCTTTTCTAGATCAAAATGCAGACAGGGCCTCCAGACGAACATTTTCAACCTCAGCATCCCGATTTCAGTCA
GACAAATACTCATTTCAGAGATTCTATACTTCATGGAATCAAGAAGCAACGAGCCATAAATCTGAAAGACAGCAA
CAGAACAAAGAAAAATGCCCCCCTTCAGCCGGACAGACTTACTCTCAAAGGTTCTTTGTTCCTCCCACTTTCAAG
TCGGTTGGCAATCCAGTGGAAGCACGCAGATGGCTAAGACAAGCCAGAGCAAACTTCTCAGCTGCCAGGAATGAC
CTTCATAAAAATGCCAATGAGTGGGTGTGCTTTAAATGTTACCTTTCTACCAAGTTAGCTTTGATTGCAGCTGAC
TATGCTGTGAGGGGAAAGTCTGATAAAGATGTAAAACCAACTGCACTTGCTCAGAAAATAGAGGAATATAGTCAG
CAACTTGAAGGACTGACAAATGATGTTCACACATTGGAAGCTTATGGTGTAGACAGTTTAAAAACAAGATACCCT
GATTTGCTTCCCTTTCCTCAGATCCCAAATGACAGGTTCACTTCTGAGGTTGCTATGAGGGTGATGGAATGTACT
GCCTGTATCATAATAAAACTTGAAAATTTTATGCAACAAAAAGTGGGTAAGCCTATCCCTAACCCTCTCCTCGGT
CTCGATTCTACGTGACTCGACGAATTCAATAAAAGATCTTTATTTTCATTAGATCTGTGTGTTGGTTTTTTGTGT
G

An example nucleotide sequence encoding an engineered sacsin comprising an SRR3 domain, a CMV enhancer, a CBA promoter, a human beta globin intron and pAmin polyadenylation sequence is:

(SEQ ID NO: 37)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGGGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAATGTCTGTTTTACAACACTTGGCACAGAATTTGGGCAG
AAAGAAAAATTGACCAGCAGAATTAAGAGCATCCTTAATGCATATCCTTCTGAAAAGGAAATGTTGAAAGAGCTT
CTTCAAAATGCTGATGATGCAAAGGCGACAGAAATCTGTTTTGTGTTTGATCCTAGACAGCATCCAGTTGATAGA
ATATTTGATGATAAGTGGGCCCCATTGCAAGGGCCAGCACTTTGTGTGTACAACAACCAGCCATTTACAGAAGAT
GATGTTAGAGGAATTCAGAATCTTGGAAAAGGCACGAAAGAGGGAAATCCTTATAAAACTGGACAGTATGGAATA
GGATTCAATTCTGTGTATCATATCACAGACTGCCCATCTTTTATTTCTGGCAATGACATCCTGTGTATTTTTGAT
CCTCATGCCAGATATGCACCAGGGGCCACATCCATTAGTCCCGGACGCATGTTTAGAGATTTGGATGCAGATTTT
AGGACACAGTTCTCAGATGTTCTGGATCTTTATCTGGGAACCCATTTTAAACTGGATAATTGCACAATGTTCAGA
TTTCCTCTTCGTAATGCAGAAATGGCAAAAGTTTCGGAAATTTCGTCTGTTCCAGCATCAGACAGAATGGTCCAG
AATCTTTTGGACAAACTGCGCTCAGATGGGGCAGAACTTCTAATGTTTCTTAATCACATGGAAAAAATTTCTATT
TGTGAAATAGATAAGAGTACTGGAGCTCTAAATGTGCTGTATTCAGTAAAGGGCAAAATCACAGATGGAGACAGA
TTGAAAAGGAAACAATTTCATGCATCTGTAATTGATAGTGTTACTAAAAAGAGGCAGCTCAAAGACATACCAGTT
CAACAAATAACCTATACTATGGATACTGAGGACTCTGAAGGAAATCTTACTACGTGGCTAATTTGTAATAGATCA
GGCTTTTCAAGTATGGAGAAAGTATCTAAAAGTGTCATATCAGCTCACAAGAACCAAGATATTACTCTTTTCCCA
CGTGGTGGAGTAGCTGCCTGCATTACTCACAACTATAAAAAACCCCATAGGGCCTTCTGTTTTTTGCCTCTTTCT
TTGGAGACTGGGCTGCCATTTCATGTGAATGGCCACTTTGCACTGGATTCAGCCAGAAGGAACCTGTGGCGTGAT
GATAATGGAGTTGGTGTTCGAAGTGACTGGAATAACAGTTTAATGACAGCATTAATAGCTCCTGCATATGTTGAA
TTGCTAATACAGTTAAAAAAACGGTATTTCCCTGGTTCTGATCCAACATTATCAGTGTTACAGAACACCCCTATT
CATGTTGTAAAGGACACTTTAAAGAAGTTTTTATCGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGACGAATTCAATAAAAGATCTTTATTTTCA
TTAGATCTGTGTGTTGGTTTTTTGTGTG

An example nucleotide sequence encoding an engineered sacsin comprising an SRR3 domain, a CMV enhancer, a CBA promoter, a human beta globin intron and pAmin polyadenylation sequence is:

(SEQ ID NO: 38)
CGTTACATAACTTACGGTAAATGGCCCGCCTGGCTGACCGCCCAACGACCCCCGCCCATTGACGTCAATAATGAC
GTATGTTCCCATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATGGCCCGCCTG
GCATTATGCCCAGTACATGACCTTATGGGACTTTCCTACTTGGCAGTACATCTACGTATTAGTCATCGCTATTAC
CATGGTCGAGGTGAGCCCCACGTTCTGCTTCACTCTCCCCATCTCCCCCCCCTCCCCACCCCCAATTTTGTATTT
ATTTATTTTTTAATTATTTTGTGCAGCGATGGGGGCGGGGGGGGGGGGGGGGCGCGCGCCAGGCGGGGCGGGGCG
GGGCGAGGGGCGGGGCGGGGCGAGGCGGAGAGGTGCGGCGGCAGCCAATCAGAGCGGCGCGCTCCGAAAGTTTCC
TTTTATGGCGAGGCGGCGGCGGCGGCGGCCCTATAAAAAGCGAAGCGCGCGGCGGGGGGGGAGTCGCTGCGACGC
TGCCTTCGCCCCGTGCCCCGCTCCGCCGCCGCCTCGCGCCGCCCGCCCCGGCTCTGACTGACCGCGTTACTCCCA
CAGGTGAGCGGGCGGGACGGCCCTTCTCCTCCGGGCTGTAATTAGCCCGTTTAGTGAACCGTCAGATCGCCTGGA
GACGCCATCCACGCTGTTTTGACCTCCATAGAAGACACCGGGACCGATCCAGCCTCCGCGGATTCGAATCCCGGC
CGGGAACGGTGCATTGGAACGCGGATTCCCCGTGCCAAGAGTGACGTAAGTACCGCCTATAGAGTCTATAGGCCC
ACAAAAAATGCTTTCTTCTTTTAATATACTTTTTTGTTTATCTTATTTCTAATACTTTCCCTAATCTCTTTCTTT
CAGGGCAATAATGATACAATGTATCATGCCTCTTTGCACCATTCTAAAGAATAACAGTGATAATTTCTGGGTTAA
GGCAATAGCAATATTTCTGCATATAAATATTTCTGCATATAAATTGTAACTGATGTAAGAGGTTTCATATTGCTA
ATAGCAGCTACAATCCAGCTACCATTCTGCTTTTATTTTATGGTTGGGATAAGGCTGGATTATTCTGAGTCCAAG
CTAGGCCCTTTTGCTAATCATGTTCATACCTCTTATCTTCCTCCCACAGCTCCTGGGCAACGTGCTGGTCTGTGT
GCTGGCCCATCACTTTGGCAAAGAATTGGGATTCGAACACCGGTCGACGAATTCGTTAACGGATCCGCTAGCGCC
GCCGCGATCGCCATGGAGACCAAGGAGAACAGGTGGGTCCCGGTGACCGTGCTCCCCGGCTGCGTGGGCTGCAGG
ACCGTCGCGGCGCTGGCGTCCTGGACCGTGCGCGATGTGAAGGAACGTATCTTCGCGGAGACTGGCTTCCCGGTG
TCGGAGCAGCGGCTGTGGCGCGGCGGCCGCGAGTTATCTGACTGGATCAAGATTGGAGATCTGACTTCCAAAAAT
TGTCATCTTTTTGTAAACCTTCAATCAAAAGGCTTAAATGTCTGTTTTACAACACTTGGCACAGAATTTGGGCAG
AAAGAAAAATTGACCAGCAGAATTAAGAGCATCCTTAATGCATATCCTTCTGAAAAGGAAATGTTGAAAGAGCTT
CTTCAAAATGCTGATGATGCAAAGGCGACAGAAATCTGTTTTGTGTTTGATCCTAGACAGCATCCAGTTGATAGA
ATATTTGATGATAAGTGGGCCCCATTGCAAGGGCCAGCACTTTGTGTGTACAACAACCAGCCATTTACAGAAGAT
GATGTTAGAGGAATTCAGAATCTTGGAAAAGGCACGAAAGAGGGAAATCCTTATAAAACTGGACAGTATGGAATA
GGATTCAATTCTGTGTATCATATCACAGACTGCCCATCTTTTATTTCTGGCAATGACATCCTGTGTATTTTTGAT
CCTCATGCCAGATATGCACCAGGGGCCACATCCATTAGTCCCGGACGCATGTTTAGAGATTTGGATGCAGATTTT
AGGACACAGTTCTCAGATGTTCTGGATCTTTATCTGGGAACCCATTTTAAACTGGATAATTGCACAATGTTCAGA
TTTCCTCTTCGTAATGCAGAAATGGCAAAAGTTTCGGAAATTTCGTCTGTTCCAGCATCAGACAGAATGGTCCAG
AATCTTTTGGACAAACTGCGCTCAGATGGGGCAGAACTTCTAATGTTTCTTAATCACATGGAAAAAATTTCTATT
TGTGAAATAGATAAGAGTACTGGAGCTCTAAATGTGCTGTATTCAGTAAAGGGCAAAATCACAGATGGAGACAGA
TTGAAAAGGAAACAATTTCATGCATCTGTAATTGATAGTGTTACTAAAAAGAGGCAGCTCAAAGACATACCAGTT
CAACAAATAACCTATACTATGGATACTGAGGACTCTGAAGGAAATCTTACTACGTGGCTAATTTGTAATAGATCA
GGCTTTTCAAGTATGGAGAAAGTATCTAAAAGTGTCATATCAGCTCACAAGAACCAAGATATTACTCTTTTCCCA
CGTGGTGGAGTAGCTGCCTGCATTACTCACAACTATAAAAAACCCCATAGGGCCTTCTGTTTTTTGCCTCTTTCT
TTGGAGACTGGGCTGCCATTTCATGTGAATGGCCACTTTGCACTGGATTCAGCCAGAAGGAACCTGTGGCGTGAT
GATAATGGAGTTGGTGTTCGAAGTGACTGGAATAACAGTTTAATGACAGCATTAATAGCTCCTGCATATGTTGAA
TTGCTAATACAGTTAAAAAAACGGTATTTCCCTGGTTCTGATCCAACATTATCAGTGTTACAGAACACCCCTATT
CATGTTGTAAAGGACACTTTAAAGAAGTTTTTATCGGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGACGAATTCAATAAAAGATCTTTATTTTCATTAGATCTGTGTGTTGGTTTTTTGTGTG

An example nucleotide sequence encoding an engineered sacsin comprising an SRR1 domain, CMV_enhancer, CMV promoter and hGh polyadenylation sequence is:

(SEQ ID NO: 57)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAAAGGGGGAGGTCGATTTGGTCAGACAACGCCACCACTTGTTGAT
TTTCTCAAGGACATTTTGAGAAGATATCCAGAAGGAGGACAGATTCTTAAGGAATTAATTCAGAATGCAGAAGAT
GCTGGGGCGACAGAAGTTAAATTTTTATATGATGAAACTCAATACGGAACAGAGACTCTTTGGTCAAAAGATATG
GCGCCATATCAGGGGCCAGCTCTCTATGTGTACAACAACGCGGTTTTCACCCCAGAGGACTGGCACGGCATTCAA
GAAATAGCAAGAAGCAGGAAAAAGGATGATCCTCTGAAGGTCGGAAGATTTGGAATTGGGTTTAATTCTGTCTAT
CATATAACAGATGTTCCTTGTATCTTTAGTGGTGACCAAATCGGGATGCTAGATCCTCATCAAACACTTTTTGGC
CCACATGAATCAGGCCAATGTTGGAATCTCAAAGATGACAGCAAAGAAATTAGTGAACTTTCAGACCAGTTTGCA
CCATTTGTTGGCATTTTTGGAAGCACCAAGGAAACATTTATAAACGGCAATTTTCCAGGAACATTTTTCCGTTTC
CCTCTTCGCCTACAACCTTCACAACTTAGTAGTAACCTCTACAATAAGCAGAAGGTTCTTGAGTTGTTTGAGTCT
TTTAGGGCAGATGCAGACACAGTGCTGCTCTTTCTGAAAAGTGTGCAGGATGTTTCCTTATATGTCCGAGAGGCT
GACGGAACAGAGAAACTGGTGTTTAGAGTGACTTCGAGTGAGAGTAAGGCACTGAAACATGAGCGGCCGAATTCT
ATAAAGATTCTGGGAACTGCTATAAGTAACTATTGTAAAAAGACTCCAAGCAATAACATCACCTGTGTAACATAT
CACGTAAATATTGTTTTAGAAGAGGAGAGTACTAAGGATGCACAGAAAACATCTTGGTTGGTGTGTAACAGTGTG
GGTGGGCGAGGGATCAGTAGTAAGCTTGACTCTTTAGCTGATGAACTGAAATTTGTCCCAATCATTGGAATAGCC
ATGCCTTTATCAAGCAGAGATGATGAAGCAAAAGGAGCAACGTCTGATTTCTCAGGAAAAGCATTTTGTTTCCTT
CCTTTACCACCTGGTGAGGAAAGCAGCACAGGCCTCCCAGTTCACATCAGTGGGTTCTTTGGCCTTACTGATAAC
CGCAGGAGCATAAAATGGAGAGAGCTGGACCAGTGGAGAGACCCGGCAGCCTTATGGAATGAGTTTCTTGTCATG
AATGTTGTCCCCAAAGCTTATGCTACTCTGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGTGACTCGAGAGATCTACGGGTGGCATCCCTGTGACCCCTCCC
CAGTGCCTCTCCTGGCCCTGGAAGTTGCCACTCCAGTGCCCACCAGCCTTGTCCTAATAAAATTAAGTTGCATCA
TTTTGTCTGACTAGGTGTCCTTCTATAATATTATGGGGTGGAGGGGGGTGGTATGGAGCAAGGGGCAAGTTGGGA
AGACAACCTGTAGGGCCTGCGGGGTCTATTGGGAACCAAGCTGGAGTGCAGTGGCACAATCTTGGCTCACTGCAA
TCTCCGCCTCCTGGGTTCAAGCGATTCTCCTGCCTCAGCCTCCCGAGTTGTTGGGATTCCAGGCATGCATGACCA
GGCTCAGCTAATTTTTGTTTTTTTGGTAGAGACGGGGTTTCACCATATTGGCCAGGCTGGTCTCCAACTCCTAAT
CTCAGGTGATCTACCCACCTTGGCCTCCCAAATTGCTGGGATTACAGGCGTGAACCACTGCTCCCTTCCCTGTCC
TT

An example nucleotide sequence encoding an engineered sacsin comprising an SRR1 domain, CMV_enhancer, CMV promoter and pAmin polyadenylation sequence is:

(SEQ ID NO: 58)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAAAGGGGGAGGTCGATTTGGTCAGACAACGCCACCACTTGTTGAT
TTTCTCAAGGACATTTTGAGAAGATATCCAGAAGGAGGACAGATTCTTAAGGAATTAATTCAGAATGCAGAAGAT
GCTGGGGCGACAGAAGTTAAATTTTTATATGATGAAACTCAATACGGAACAGAGACTCTTTGGTCAAAAGATATG
GCGCCATATCAGGGGCCAGCTCTCTATGTGTACAACAACGCGGTTTTCACCCCAGAGGACTGGCACGGCATTCAA
GAAATAGCAAGAAGCAGGAAAAAGGATGATCCTCTGAAGGTCGGAAGATTTGGAATTGGGTTTAATTCTGTCTAT
CATATAACAGATGTTCCTTGTATCTTTAGTGGTGACCAAATCGGGATGCTAGATCCTCATCAAACACTTTTTGGC
CCACATGAATCAGGCCAATGTTGGAATCTCAAAGATGACAGCAAAGAAATTAGTGAACTTTCAGACCAGTTTGCA
CCATTTGTTGGCATTTTTGGAAGCACCAAGGAAACATTTATAAACGGCAATTTTCCAGGAACATTTTTCCGTTTC
CCTCTTCGCCTACAACCTTCACAACTTAGTAGTAACCTCTACAATAAGCAGAAGGTTCTTGAGTTGTTTGAGTCT
TTTAGGGCAGATGCAGACACAGTGCTGCTCTTTCTGAAAAGTGTGCAGGATGTTTCCTTATATGTCCGAGAGGCT
GACGGAACAGAGAAACTGGTGTTTAGAGTGACTTCGAGTGAGAGTAAGGCACTGAAACATGAGCGGCCGAATTCT
ATAAAGATTCTGGGAACTGCTATAAGTAACTATTGTAAAAAGACTCCAAGCAATAACATCACCTGTGTAACATAT
CACGTAAATATTGTTTTAGAAGAGGAGAGTACTAAGGATGCACAGAAAACATCTTGGTTGGTGTGTAACAGTGTG
GGTGGGCGAGGGATCAGTAGTAAGCTTGACTCTTTAGCTGATGAACTGAAATTTGTCCCAATCATTGGAATAGCC
ATGCCTTTATCAAGCAGAGATGATGAAGCAAAAGGAGCAACGTCTGATTTCTCAGGAAAAGCATTTTGTTTCCTT
CCTTTACCACCTGGTGAGGAAAGCAGCACAGGCCTCCCAGTTCACATCAGTGGGTTCTTTGGCCTTACTGATAAC
CGCAGGAGCATAAAATGGAGAGAGCTGGACCAGTGGAGAGACCCGGCAGCCTTATGGAATGAGTTTCTTGTCATG
AATGTTGTCCCCAAAGCTTATGCTACTCTGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGTGACTCGACGAATTCAATAAAAGATCTTTATTTTCATTAGAT
CTGTGTGTTGGTTTTTTGTGTG

An example nucleotide sequence encoding an engineered sacsin comprising an SRR1 domain, CMV_enhancer, CMV promoter, and hGh polyadenylation sequence is:

(SEQ ID NO: 59)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAAAGGGGGAGGTCGATTTGGTCAGACAACGCCACCACTTGTTGAT
TTTCTCAAGGACATTTTGAGAAGATATCCAGAAGGAGGACAGATTCTTAAGGAATTAATTCAGAATGCAGAAGAT
GCTGGGGCGACAGAAGTTAAATTTTTATATGATGAAACTCAATACGGAACAGAGACTCTTTGGTCAAAAGATATG
GCGCCATATCAGGGGCCAGCTCTCTATGTGTACAACAACGCGGTTTTCACCCCAGAGGACTGGCACGGCATTCAA
GAAATAGCAAGAAGCAGGAAAAAGGATGATCCTCTGAAGGTCGGAAGATTTGGAATTGGGTTTAATTCTGTCTAT
CATATAACAGATGTTCCTTGTATCTTTAGTGGTGACCAAATCGGGATGCTAGATCCTCATCAAACACTTTTTGGC
CCACATGAATCAGGCCAATGTTGGAATCTCAAAGATGACAGCAAAGAAATTAGTGAACTTTCAGACCAGTTTGCA
CCATTTGTTGGCATTTTTGGAAGCACCAAGGAAACATTTATAAACGGCAATTTTCCAGGAACATTTTTCCGTTTC
CCTCTTCGCCTACAACCTTCACAACTTAGTAGTAACCTCTACAATAAGCAGAAGGTTCTTGAGTTGTTTGAGTCT
TTTAGGGCAGATGCAGACACAGTGCTGCTCTTTCTGAAAAGTGTGCAGGATGTTTCCTTATATGTCCGAGAGGCT
GACGGAACAGAGAAACTGGTGTTTAGAGTGACTTCGAGTGAGAGTAAGGCACTGAAACATGAGCGGCCGAATTCT
ATAAAGATTCTGGGAACTGCTATAAGTAACTATTGTAAAAAGACTCCAAGCAATAACATCACCTGTGTAACATAT
CACGTAAATATTGTTTTAGAAGAGGAGAGTACTAAGGATGCACAGAAAACATCTTGGTTGGTGTGTAACAGTGTG
GGTGGGCGAGGGATCAGTAGTAAGCTTGACTCTTTAGCTGATGAACTGAAATTTGTCCCAATCATTGGAATAGCC
ATGCCTTTATCAAGCAGAGATGATGAAGCAAAAGGAGCAACGTCTGATTTCTCAGGAAAAGCATTTTGTTTCCTT
CCTTTACCACCTGGTGAGGAAAGCAGCACAGGCCTCCCAGTTCACATCAGTGGGTTCTTTGGCCTTACTGATAAC
CGCAGGAGCATAAAATGGAGAGAGCTGGACCAGTGGAGAGACCCGGCAGCCTTATGGAATGAGTTTCTTGTCATG
AATGTTGTCCCCAAAGCTTATGCTACTCTGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGGGTAAGCCTATCCCTAACCCTCTCCTCGGTCTCGATTCTACG
TGACTCGAGAGATCTACGGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTATAATATTA
TGGGGTGGAGGGGGGTGGTATGGAGCAAGGGGCAAGTTGGGAAGACAACCTGTAGGGCCTGCGGGGTCTATTGGG
AACCAAGCTGGAGTGCAGTGGCACAATCTTGGCTCACTGCAATCTCCGCCTCCTGGGTTCAAGCGATTCTCCTGC
CTCAGCCTCCCGAGTTGTTGGGATTCCAGGCATGCATGACCAGGCTCAGCTAATTTTTGTTTTTTTGGTAGAGAC
GGGGTTTCACCATATTGGCCAGGCTGGTCTCCAACTCCTAATCTCAGGTGATCTACCCACCTTGGCCTCCCAAAT
TGCTGGGATTACAGGCGTGAACCACTGCTCCCTTCCCTGTCCTT

An example nucleotide sequence encoding an engineered sacsin comprising an SRR1 domain, a CMV_enhancer, CMV promoter, and pAmin polyadenylation sequence is:

(SEQ ID NO: 60)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAAAGGGGGAGGTCGATTTGGTCAGACAACGCCACCACTTGTTGAT
TTTCTCAAGGACATTTTGAGAAGATATCCAGAAGGAGGACAGATTCTTAAGGAATTAATTCAGAATGCAGAAGAT
GCTGGGGCGACAGAAGTTAAATTTTTATATGATGAAACTCAATACGGAACAGAGACTCTTTGGTCAAAAGATATG
GCGCCATATCAGGGGCCAGCTCTCTATGTGTACAACAACGCGGTTTTCACCCCAGAGGACTGGCACGGCATTCAA
GAAATAGCAAGAAGCAGGAAAAAGGATGATCCTCTGAAGGTCGGAAGATTTGGAATTGGGTTTAATTCTGTCTAT
CATATAACAGATGTTCCTTGTATCTTTAGTGGTGACCAAATCGGGATGCTAGATCCTCATCAAACACTTTTTGGC
CCACATGAATCAGGCCAATGTTGGAATCTCAAAGATGACAGCAAAGAAATTAGTGAACTTTCAGACCAGTTTGCA
CCATTTGTTGGCATTTTTGGAAGCACCAAGGAAACATTTATAAACGGCAATTTTCCAGGAACATTTTTCCGTTTC
CCTCTTCGCCTACAACCTTCACAACTTAGTAGTAACCTCTACAATAAGCAGAAGGTTCTTGAGTTGTTTGAGTCT
TTTAGGGCAGATGCAGACACAGTGCTGCTCTTTCTGAAAAGTGTGCAGGATGTTTCCTTATATGTCCGAGAGGCT
GACGGAACAGAGAAACTGGTGTTTAGAGTGACTTCGAGTGAGAGTAAGGCACTGAAACATGAGCGGCCGAATTCT
ATAAAGATTCTGGGAACTGCTATAAGTAACTATTGTAAAAAGACTCCAAGCAATAACATCACCTGTGTAACATAT
CACGTAAATATTGTTTTAGAAGAGGAGAGTACTAAGGATGCACAGAAAACATCTTGGTTGGTGTGTAACAGTGTG
GGTGGGCGAGGGATCAGTAGTAAGCTTGACTCTTTAGCTGATGAACTGAAATTTGTCCCAATCATTGGAATAGCC
ATGCCTTTATCAAGCAGAGATGATGAAGCAAAAGGAGCAACGTCTGATTTCTCAGGAAAAGCATTTTGTTTCCTT
CCTTTACCACCTGGTGAGGAAAGCAGCACAGGCCTCCCAGTTCACATCAGTGGGTTCTTTGGCCTTACTGATAAC
CGCAGGAGCATAAAATGGAGAGAGCTGGACCAGTGGAGAGACCCGGCAGCCTTATGGAATGAGTTTCTTGTCATG
AATGTTGTCCCCAAAGCTTATGCTACTCTGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGGGTAAGCCTATCCCTAACCCTCTCCTCGGTCTCGATTCTACG
TGACTCGACGAATTCAATAAAAGATCTTTATTTTCATTAGATCTGTGTGTTGGTTTTTTGTGTG

An example nucleotide sequence encoding an engineered sacsin comprising an SRR2 domain, a CMV enhancer, CMV promoter and bGH polyadenylation sequence is:

(SEQ ID NO: 61)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAATAAATCCTGAAAACATGGGATTTGAGCAGTCAGGACAAAGAGAG
CCACTTACTGTAAGAATTAAAAATATTCTGGAAGAATACCCTTCAGTGTCAGATATTTTTAAAGAACTACTTCAA
AACGCTGATGATGCAAATGCAACAGAATGCAGTTTCTTGATTGATATGAGAAGAAATATGGACATAAGAGAGAAT
CTCCTAGACCCAGGGATGGCAGCTTGTCATGGACCTGCTTTGTGGTCATTCAACAATTCTCAATTCTCAGATTCA
GATTTTGTGAACATAACTAGGTTAGGAGAATCTTTAAAAAGGGGAGAAGTTGACAAAGTTGGAAAATTTGGTCTT
GGATTTAATTCTGTGTACCATATCACTGACATTCCCATCATTATGAGTCGGGAATTCATGATAATGTTCGATCCA
AACATAAATCATATCAGTAAACACATTAAAGACAAATCCAATCCTGGGATCAAAATTAATTGGAGTAAACAACAG
AAAAGACTTAGAAAATTTCCTAATCAGTTCAAACCATTTATAGATGTATTTGGCTGTCAGTTACCTTTGACTGTA
GAAGCACCTTACAGCTATAATGGAACCCTTTTCCGACTGTCCTTTAGAACTCAACAGGAAGCAAAAGTGAGTGAA
GTTAGTAGTACGTGCTACAATACAGCAGATATTTATTCTCTTGTGGATGAATTTAGTCTCTGTGGACACAGGCTT
ATCATTTTCACTCAGAGTGTAAAGTCAATGTATTTGAAGTACTTGAAAATTGAGGAAACCAACCCCAGTTTAGCA
CAAGATACAGTAATAATTAAAAAAAAATCCTGCTCTTCCAAAGCATTGAACACACCTGTCTTAAGTGTTTTAAAA
GAGGCTGCTAAGCTCATGAAGACTTGCAGCAGCAGTAATAAAAAGCTTCCCAGTGATGAACCAAAGTCATCTTGC
ATTCTTCAGATCACAGTGGAAGAATTTCACCATGTGTTCAGAAGGATTGCTGATTTACAGTCGCCACTTTTTAGA
GGTCCAGATGATGACCCAGCTGCTCTCTTTGAAATGGCTAAGTCTGGCCAATCAAAAAAGCCATCAGATGAGTTG
TCACAGAAAACAGTAGAGTGTACCACGTGGCTTCTGTGTACTTGCATGGACACAGGAGAGGCTCTGAAGTTTTCC
CTGAGTGAGAGTGGAAGAAGACTAGGACTGGTTCCATGTGGGGCAGTAGGAGTTCAGCTGTCAGAAATCCAGGAC
CAGAAGTGGACAGTGAAACCACACATTGGAGAGGTGTTTTGCTATTTACCTTTACGAATAAAAACAGGCTTGCCA
GTTCATATCAATGGGTGCTTTGCTGTTACATCAAATAGGAAAGAAATCTGGAAAACAGATACAAAAGGACGATGG
AATACCACGTTCATGAGACATGTTATTGTGAAAGCTTACTTACAGGTACTGAGTGTCTTACGGGACCTGGCCACT
AGTGGGGAGCTAATGGATTATACTTACTATGCAGTATGGCCCGATCCTGATTTAGTTCATGATGATTTTTCTGTA
ATTTGCCAAGGATTTTATGAAGATATAGCTCATGGAGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGAGTCTAGAGGGCCCGTTTAAACCCGCTGA
TCAGCCTCGACTGTGCCTTCTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTGGGGATGCGGTG
GGCTCTATGG

An example nucleotide sequence encoding an engineered sacsin comprising an SRR2 domain, a CMV_enhancer, CMV promoter and a hGh polyadenylation sequence is:

(SEQ ID NO: 62)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAATAAATCCTGAAAACATGGGATTTGAGCAGTCAGGACAAAGAGAG
CCACTTACTGTAAGAATTAAAAATATTCTGGAAGAATACCCTTCAGTGTCAGATATTTTTAAAGAACTACTTCAA
AACGCTGATGATGCAAATGCAACAGAATGCAGTTTCTTGATTGATATGAGAAGAAATATGGACATAAGAGAGAAT
CTCCTAGACCCAGGGATGGCAGCTTGTCATGGACCTGCTTTGTGGTCATTCAACAATTCTCAATTCTCAGATTCA
GATTTTGTGAACATAACTAGGTTAGGAGAATCTTTAAAAAGGGGAGAAGTTGACAAAGTTGGAAAATTTGGTCTT
GGATTTAATTCTGTGTACCATATCACTGACATTCCCATCATTATGAGTCGGGAATTCATGATAATGTTCGATCCA
AACATAAATCATATCAGTAAACACATTAAAGACAAATCCAATCCTGGGATCAAAATTAATTGGAGTAAACAACAG
AAAAGACTTAGAAAATTTCCTAATCAGTTCAAACCATTTATAGATGTATTTGGCTGTCAGTTACCTTTGACTGTA
GAAGCACCTTACAGCTATAATGGAACCCTTTTCCGACTGTCCTTTAGAACTCAACAGGAAGCAAAAGTGAGTGAA
GTTAGTAGTACGTGCTACAATACAGCAGATATTTATTCTCTTGTGGATGAATTTAGTCTCTGTGGACACAGGCTT
ATCATTTTCACTCAGAGTGTAAAGTCAATGTATTTGAAGTACTTGAAAATTGAGGAAACCAACCCCAGTTTAGCA
CAAGATACAGTAATAATTAAAAAAAAATCCTGCTCTTCCAAAGCATTGAACACACCTGTCTTAAGTGTTTTAAAA
GAGGCTGCTAAGCTCATGAAGACTTGCAGCAGCAGTAATAAAAAGCTTCCCAGTGATGAACCAAAGTCATCTTGC
ATTCTTCAGATCACAGTGGAAGAATTTCACCATGTGTTCAGAAGGATTGCTGATTTACAGTCGCCACTTTTTAGA
GGTCCAGATGATGACCCAGCTGCTCTCTTTGAAATGGCTAAGTCTGGCCAATCAAAAAAGCCATCAGATGAGTTG
TCACAGAAAACAGTAGAGTGTACCACGTGGCTTCTGTGTACTTGCATGGACACAGGAGAGGCTCTGAAGTTTTCC
CTGAGTGAGAGTGGAAGAAGACTAGGACTGGTTCCATGTGGGGCAGTAGGAGTTCAGCTGTCAGAAATCCAGGAC
CAGAAGTGGACAGTGAAACCACACATTGGAGAGGTGTTTTGCTATTTACCTTTACGAATAAAAACAGGCTTGCCA
GTTCATATCAATGGGTGCTTTGCTGTTACATCAAATAGGAAAGAAATCTGGAAAACAGATACAAAAGGACGATGG
AATACCACGTTCATGAGACATGTTATTGTGAAAGCTTACTTACAGGTACTGAGTGTCTTACGGGACCTGGCCACT
AGTGGGGAGCTAATGGATTATACTTACTATGCAGTATGGCCCGATCCTGATTTAGTTCATGATGATTTTTCTGTA
ATTTGCCAAGGATTTTATGAAGATATAGCTCATGGAGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGAGAGATCTACGGGTGGCATCCCTGTGACC
CCTCCCCAGTGCCTCTCCTGGCCCTGGAAGTTGCCACTCCAGTGCCCACCAGCCTTGTCCTAATAAAATTAAGTT
GCATCATTTTGTCTGACTAGGTGTCCTTCTATAATATTATGGGGTGGAGGGGGGTGGTATGGAGCAAGGGGCAAG
TTGGGAAGACAACCTGTAGGGCCTGCGGGGTCTATTGGGAACCAAGCTGGAGTGCAGTGGCACAATCTTGGCTCA
CTGCAATCTCCGCCTCCTGGGTTCAAGCGATTCTCCTGCCTCAGCCTCCCGAGTTGTTGGGATTCCAGGCATGCA
TGACCAGGCTCAGCTAATTTTTGTTTTTTTGGTAGAGACGGGGTTTCACCATATTGGCCAGGCTGGTCTCCAACT
CCTAATCTCAGGTGATCTACCCACCTTGGCCTCCCAAATTGCTGGGATTACAGGCGTGAACCACTGCTCCCTTCC
CTGTCCTT

An example nucleotide sequence encoding an engineered sacsin comprising an SRR2 domain, a CMV_enhancer, CMV promoter and a pAmin polyadenylation sequence is:

(SEQ ID NO: 63)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAATAAATCCTGAAAACATGGGATTTGAGCAGTCAGGACAAAGAGAG
CCACTTACTGTAAGAATTAAAAATATTCTGGAAGAATACCCTTCAGTGTCAGATATTTTTAAAGAACTACTTCAA
AACGCTGATGATGCAAATGCAACAGAATGCAGTTTCTTGATTGATATGAGAAGAAATATGGACATAAGAGAGAAT
CTCCTAGACCCAGGGATGGCAGCTTGTCATGGACCTGCTTTGTGGTCATTCAACAATTCTCAATTCTCAGATTCA
GATTTTGTGAACATAACTAGGTTAGGAGAATCTTTAAAAAGGGGAGAAGTTGACAAAGTTGGAAAATTTGGTCTT
GGATTTAATTCTGTGTACCATATCACTGACATTCCCATCATTATGAGTCGGGAATTCATGATAATGTTCGATCCA
AACATAAATCATATCAGTAAACACATTAAAGACAAATCCAATCCTGGGATCAAAATTAATTGGAGTAAACAACAG
AAAAGACTTAGAAAATTTCCTAATCAGTTCAAACCATTTATAGATGTATTTGGCTGTCAGTTACCTTTGACTGTA
GAAGCACCTTACAGCTATAATGGAACCCTTTTCCGACTGTCCTTTAGAACTCAACAGGAAGCAAAAGTGAGTGAA
GTTAGTAGTACGTGCTACAATACAGCAGATATTTATTCTCTTGTGGATGAATTTAGTCTCTGTGGACACAGGCTT
ATCATTTTCACTCAGAGTGTAAAGTCAATGTATTTGAAGTACTTGAAAATTGAGGAAACCAACCCCAGTTTAGCA
CAAGATACAGTAATAATTAAAAAAAAATCCTGCTCTTCCAAAGCATTGAACACACCTGTCTTAAGTGTTTTAAAA
GAGGCTGCTAAGCTCATGAAGACTTGCAGCAGCAGTAATAAAAAGCTTCCCAGTGATGAACCAAAGTCATCTTGC
ATTCTTCAGATCACAGTGGAAGAATTTCACCATGTGTTCAGAAGGATTGCTGATTTACAGTCGCCACTTTTTAGA
GGTCCAGATGATGACCCAGCTGCTCTCTTTGAAATGGCTAAGTCTGGCCAATCAAAAAAGCCATCAGATGAGTTG
TCACAGAAAACAGTAGAGTGTACCACGTGGCTTCTGTGTACTTGCATGGACACAGGAGAGGCTCTGAAGTTTTCC
CTGAGTGAGAGTGGAAGAAGACTAGGACTGGTTCCATGTGGGGCAGTAGGAGTTCAGCTGTCAGAAATCCAGGAC
CAGAAGTGGACAGTGAAACCACACATTGGAGAGGTGTTTTGCTATTTACCTTTACGAATAAAAACAGGCTTGCCA
GTTCATATCAATGGGTGCTTTGCTGTTACATCAAATAGGAAAGAAATCTGGAAAACAGATACAAAAGGACGATGG
AATACCACGTTCATGAGACATGTTATTGTGAAAGCTTACTTACAGGTACTGAGTGTCTTACGGGACCTGGCCACT
AGTGGGGAGCTAATGGATTATACTTACTATGCAGTATGGCCCGATCCTGATTTAGTTCATGATGATTTTTCTGTA
ATTTGCCAAGGATTTTATGAAGATATAGCTCATGGAGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGTGACTCGACGAATTCAATAAAAGATCTTTATTTTCA
TTAGATCTGTGTGTTGGTTTTTTGTGTG

An example nucleotide sequence encoding an engineered sacsin comprising an SRR2 domain, a CMV_enhancer, CMV promoter and bGH polyadenylation sequence is:

(SEQ ID NO: 64)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAATAAATCCTGAAAACATGGGATTTGAGCAGTCAGGACAAAGAGAG
CCACTTACTGTAAGAATTAAAAATATTCTGGAAGAATACCCTTCAGTGTCAGATATTTTTAAAGAACTACTTCAA
AACGCTGATGATGCAAATGCAACAGAATGCAGTTTCTTGATTGATATGAGAAGAAATATGGACATAAGAGAGAAT
CTCCTAGACCCAGGGATGGCAGCTTGTCATGGACCTGCTTTGTGGTCATTCAACAATTCTCAATTCTCAGATTCA
GATTTTGTGAACATAACTAGGTTAGGAGAATCTTTAAAAAGGGGAGAAGTTGACAAAGTTGGAAAATTTGGTCTT
GGATTTAATTCTGTGTACCATATCACTGACATTCCCATCATTATGAGTCGGGAATTCATGATAATGTTCGATCCA
AACATAAATCATATCAGTAAACACATTAAAGACAAATCCAATCCTGGGATCAAAATTAATTGGAGTAAACAACAG
AAAAGACTTAGAAAATTTCCTAATCAGTTCAAACCATTTATAGATGTATTTGGCTGTCAGTTACCTTTGACTGTA
GAAGCACCTTACAGCTATAATGGAACCCTTTTCCGACTGTCCTTTAGAACTCAACAGGAAGCAAAAGTGAGTGAA
GTTAGTAGTACGTGCTACAATACAGCAGATATTTATTCTCTTGTGGATGAATTTAGTCTCTGTGGACACAGGCTT
ATCATTTTCACTCAGAGTGTAAAGTCAATGTATTTGAAGTACTTGAAAATTGAGGAAACCAACCCCAGTTTAGCA
CAAGATACAGTAATAATTAAAAAAAAATCCTGCTCTTCCAAAGCATTGAACACACCTGTCTTAAGTGTTTTAAAA
GAGGCTGCTAAGCTCATGAAGACTTGCAGCAGCAGTAATAAAAAGCTTCCCAGTGATGAACCAAAGTCATCTTGC
ATTCTTCAGATCACAGTGGAAGAATTTCACCATGTGTTCAGAAGGATTGCTGATTTACAGTCGCCACTTTTTAGA
GGTCCAGATGATGACCCAGCTGCTCTCTTTGAAATGGCTAAGTCTGGCCAATCAAAAAAGCCATCAGATGAGTTG
TCACAGAAAACAGTAGAGTGTACCACGTGGCTTCTGTGTACTTGCATGGACACAGGAGAGGCTCTGAAGTTTTCC
CTGAGTGAGAGTGGAAGAAGACTAGGACTGGTTCCATGTGGGGCAGTAGGAGTTCAGCTGTCAGAAATCCAGGAC
CAGAAGTGGACAGTGAAACCACACATTGGAGAGGTGTTTTGCTATTTACCTTTACGAATAAAAACAGGCTTGCCA
GTTCATATCAATGGGTGCTTTGCTGTTACATCAAATAGGAAAGAAATCTGGAAAACAGATACAAAAGGACGATGG
AATACCACGTTCATGAGACATGTTATTGTGAAAGCTTACTTACAGGTACTGAGTGTCTTACGGGACCTGGCCACT
AGTGGGGAGCTAATGGATTATACTTACTATGCAGTATGGCCCGATCCTGATTTAGTTCATGATGATTTTTCTGTA
ATTTGCCAAGGATTTTATGAAGATATAGCTCATGGAGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGAGTCTAGAGGGCCCGTTTAAACCCGCTGATCAGCCTCGACTGTGCCTTCTAGTTGCCAGCCA
TCTGTTGTTTGCCCCTCCCCCGTGCCTTCCTTGACCCTGGAAGGTGCCACTCCCACTGTCCTTTCCTAATAAAAT
GAGGAAATTGCATCGCATTGTCTGAGTAGGTGTCATTCTATTCTGGGGGGTGGGGTGGGGCAGGACAGCAAGGGG
GAGGATTGGGAAGACAATAGCAGGCATGCTGGGGATGCGGTGGGCTCTATGG

An example nucleotide sequence encoding an engineered sacsin comprising an SRR2 domain, a CMV_enhancer, CMV promoter and hGh polyadenylation sequence is:

(SEQ ID NO: 65)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAATAAATCCTGAAAACATGGGATTTGAGCAGTCAGGACAAAGAGAG
CCACTTACTGTAAGAATTAAAAATATTCTGGAAGAATACCCTTCAGTGTCAGATATTTTTAAAGAACTACTTCAA
AACGCTGATGATGCAAATGCAACAGAATGCAGTTTCTTGATTGATATGAGAAGAAATATGGACATAAGAGAGAAT
CTCCTAGACCCAGGGATGGCAGCTTGTCATGGACCTGCTTTGTGGTCATTCAACAATTCTCAATTCTCAGATTCA
GATTTTGTGAACATAACTAGGTTAGGAGAATCTTTAAAAAGGGGAGAAGTTGACAAAGTTGGAAAATTTGGTCTT
GGATTTAATTCTGTGTACCATATCACTGACATTCCCATCATTATGAGTCGGGAATTCATGATAATGTTCGATCCA
AACATAAATCATATCAGTAAACACATTAAAGACAAATCCAATCCTGGGATCAAAATTAATTGGAGTAAACAACAG
AAAAGACTTAGAAAATTTCCTAATCAGTTCAAACCATTTATAGATGTATTTGGCTGTCAGTTACCTTTGACTGTA
GAAGCACCTTACAGCTATAATGGAACCCTTTTCCGACTGTCCTTTAGAACTCAACAGGAAGCAAAAGTGAGTGAA
GTTAGTAGTACGTGCTACAATACAGCAGATATTTATTCTCTTGTGGATGAATTTAGTCTCTGTGGACACAGGCTT
ATCATTTTCACTCAGAGTGTAAAGTCAATGTATTTGAAGTACTTGAAAATTGAGGAAACCAACCCCAGTTTAGCA
CAAGATACAGTAATAATTAAAAAAAAATCCTGCTCTTCCAAAGCATTGAACACACCTGTCTTAAGTGTTTTAAAA
GAGGCTGCTAAGCTCATGAAGACTTGCAGCAGCAGTAATAAAAAGCTTCCCAGTGATGAACCAAAGTCATCTTGC
ATTCTTCAGATCACAGTGGAAGAATTTCACCATGTGTTCAGAAGGATTGCTGATTTACAGTCGCCACTTTTTAGA
GGTCCAGATGATGACCCAGCTGCTCTCTTTGAAATGGCTAAGTCTGGCCAATCAAAAAAGCCATCAGATGAGTTG
TCACAGAAAACAGTAGAGTGTACCACGTGGCTTCTGTGTACTTGCATGGACACAGGAGAGGCTCTGAAGTTTTCC
CTGAGTGAGAGTGGAAGAAGACTAGGACTGGTTCCATGTGGGGCAGTAGGAGTTCAGCTGTCAGAAATCCAGGAC
CAGAAGTGGACAGTGAAACCACACATTGGAGAGGTGTTTTGCTATTTACCTTTACGAATAAAAACAGGCTTGCCA
GTTCATATCAATGGGTGCTTTGCTGTTACATCAAATAGGAAAGAAATCTGGAAAACAGATACAAAAGGACGATGG
AATACCACGTTCATGAGACATGTTATTGTGAAAGCTTACTTACAGGTACTGAGTGTCTTACGGGACCTGGCCACT
AGTGGGGAGCTAATGGATTATACTTACTATGCAGTATGGCCCGATCCTGATTTAGTTCATGATGATTTTTCTGTA
ATTTGCCAAGGATTTTATGAAGATATAGCTCATGGAGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGAGAGATCTACGGGTGGCATCCCTGTGACCCCTCCCCAGTGCCTCTCCTGGCCCTGGAAGTTG
CCACTCCAGTGCCCACCAGCCTTGTCCTAATAAAATTAAGTTGCATCATTTTGTCTGACTAGGTGTCCTTCTATA
ATATTATGGGGTGGAGGGGGGTGGTATGGAGCAAGGGGCAAGTTGGGAAGACAACCTGTAGGGCCTGCGGGGTCT
ATTGGGAACCAAGCTGGAGTGCAGTGGCACAATCTTGGCTCACTGCAATCTCCGCCTCCTGGGTTCAAGCGATTC
TCCTGCCTCAGCCTCCCGAGTTGTTGGGATTCCAGGCATGCATGACCAGGCTCAGCTAATTTTTGTTTTTTTGGT
AGAGACGGGGTTTCACCATATTGGCCAGGCTGGTCTCCAACTCCTAATCTCAGGTGATCTACCCACCTTGGCCTC
CCAAATTGCTGGGATTACAGGCGTGAACCACTGCTCCCTTCCCTGTCCTT

An example nucleotide sequence encoding an engineered sacsin comprising an SRR2 domain, a CMV_enhancer, CMV promoter and pAmin polyadenylation sequence is:

(SEQ ID NO: 66)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAATAAATCCTGAAAACATGGGATTTGAGCAGTCAGGACAAAGAGAG
CCACTTACTGTAAGAATTAAAAATATTCTGGAAGAATACCCTTCAGTGTCAGATATTTTTAAAGAACTACTTCAA
AACGCTGATGATGCAAATGCAACAGAATGCAGTTTCTTGATTGATATGAGAAGAAATATGGACATAAGAGAGAAT
CTCCTAGACCCAGGGATGGCAGCTTGTCATGGACCTGCTTTGTGGTCATTCAACAATTCTCAATTCTCAGATTCA
GATTTTGTGAACATAACTAGGTTAGGAGAATCTTTAAAAAGGGGAGAAGTTGACAAAGTTGGAAAATTTGGTCTT
GGATTTAATTCTGTGTACCATATCACTGACATTCCCATCATTATGAGTCGGGAATTCATGATAATGTTCGATCCA
AACATAAATCATATCAGTAAACACATTAAAGACAAATCCAATCCTGGGATCAAAATTAATTGGAGTAAACAACAG
AAAAGACTTAGAAAATTTCCTAATCAGTTCAAACCATTTATAGATGTATTTGGCTGTCAGTTACCTTTGACTGTA
GAAGCACCTTACAGCTATAATGGAACCCTTTTCCGACTGTCCTTTAGAACTCAACAGGAAGCAAAAGTGAGTGAA
GTTAGTAGTACGTGCTACAATACAGCAGATATTTATTCTCTTGTGGATGAATTTAGTCTCTGTGGACACAGGCTT
ATCATTTTCACTCAGAGTGTAAAGTCAATGTATTTGAAGTACTTGAAAATTGAGGAAACCAACCCCAGTTTAGCA
CAAGATACAGTAATAATTAAAAAAAAATCCTGCTCTTCCAAAGCATTGAACACACCTGTCTTAAGTGTTTTAAAA
GAGGCTGCTAAGCTCATGAAGACTTGCAGCAGCAGTAATAAAAAGCTTCCCAGTGATGAACCAAAGTCATCTTGC
ATTCTTCAGATCACAGTGGAAGAATTTCACCATGTGTTCAGAAGGATTGCTGATTTACAGTCGCCACTTTTTAGA
GGTCCAGATGATGACCCAGCTGCTCTCTTTGAAATGGCTAAGTCTGGCCAATCAAAAAAGCCATCAGATGAGTTG
TCACAGAAAACAGTAGAGTGTACCACGTGGCTTCTGTGTACTTGCATGGACACAGGAGAGGCTCTGAAGTTTTCC
CTGAGTGAGAGTGGAAGAAGACTAGGACTGGTTCCATGTGGGGCAGTAGGAGTTCAGCTGTCAGAAATCCAGGAC
CAGAAGTGGACAGTGAAACCACACATTGGAGAGGTGTTTTGCTATTTACCTTTACGAATAAAAACAGGCTTGCCA
GTTCATATCAATGGGTGCTTTGCTGTTACATCAAATAGGAAAGAAATCTGGAAAACAGATACAAAAGGACGATGG
AATACCACGTTCATGAGACATGTTATTGTGAAAGCTTACTTACAGGTACTGAGTGTCTTACGGGACCTGGCCACT
AGTGGGGAGCTAATGGATTATACTTACTATGCAGTATGGCCCGATCCTGATTTAGTTCATGATGATTTTTCTGTA
ATTTGCCAAGGATTTTATGAAGATATAGCTCATGGAGGATCCATCTTAAAAGAAGTGACATCTGTGGTGGAGCAA
GCATGGAAGCTTCCAGAATCGGAACGAAAAAAGATTATTAGGCGGTTGTATTTGAAATGGCATCCTGACAAAAAT
CCAGAGAACCATGACATTGCCAATGAAGTTTTTAAACATTTGCAGAATGAAATCAACAGATTAGAAAAACAGGCT
TTTCTAGATCAAAATGCAGACAGGGCCTCCAGACGAACATTTTCAACCTCAGCATCCCGATTTCAGTCAGACAAA
TACTCATTTCAGAGATTCTATACTTCATGGAATCAAGAAGCAACGAGCCATAAATCTGAAAGACAGCAACAGAAC
AAAGAAAAATGCCCCCCTTCAGCCGGACAGACTTACTCTCAAAGGTTCTTTGTTCCTCCCACTTTCAAGTCGGTT
GGCAATCCAGTGGAAGCACGCAGATGGCTAAGACAAGCCAGAGCAAACTTCTCAGCTGCCAGGAATGACCTTCAT
AAAAATGCCAATGAGTGGGTGTGCTTTAAATGTTACCTTTCTACCAAGTTAGCTTTGATTGCAGCTGACTATGCT
GTGAGGGGAAAGTCTGATAAAGATGTAAAACCAACTGCACTTGCTCAGAAAATAGAGGAATATAGTCAGCAACTT
GAAGGACTGACAAATGATGTTCACACATTGGAAGCTTATGGTGTAGACAGTTTAAAAACAAGATACCCTGATTTG
CTTCCCTTTCCTCAGATCCCAAATGACAGGTTCACTTCTGAGGTTGCTATGAGGGTGATGGAATGTACTGCCTGT
ATCATAATAAAACTTGAAAATTTTATGCAACAAAAAGTGGGTAAGCCTATCCCTAACCCTCTCCTCGGTCTCGAT
TCTACGTGACTCGACGAATTCAATAAAAGATCTTTATTTTCATTAGATCTGTGTGTTGGTTTTTTGTGTG

An example nucleotide sequence encoding an engineered sacsin comprising an SRR3 domain, a CMV_enhancer, CMV promoter and bGH polyadenylation sequence is:

(SEQ ID NO: 67)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAATGTCTGTTTTACAACACTTGGCACAGAATTTGGGCAGAAAGAA
AAATTGACCAGCAGAATTAAGAGCATCCTTAATGCATATCCTTCTGAAAAGGAAATGTTGAAAGAGCTTCTTCAA
AATGCTGATGATGCAAAGGCGACAGAAATCTGTTTTGTGTTTGATCCTAGACAGCATCCAGTTGATAGAATATTT
GATGATAAGTGGGCCCCATTGCAAGGGCCAGCACTTTGTGTGTACAACAACCAGCCATTTACAGAAGATGATGTT
AGAGGAATTCAGAATCTTGGAAAAGGCACGAAAGAGGGAAATCCTTATAAAACTGGACAGTATGGAATAGGATTC
AATTCTGTGTATCATATCACAGACTGCCCATCTTTTATTTCTGGCAATGACATCCTGTGTATTTTTGATCCTCAT
GCCAGATATGCACCAGGGGCCACATCCATTAGTCCCGGACGCATGTTTAGAGATTTGGATGCAGATTTTAGGACA
CAGTTCTCAGATGTTCTGGATCTTTATCTGGGAACCCATTTTAAACTGGATAATTGCACAATGTTCAGATTTCCT
CTTCGTAATGCAGAAATGGCAAAAGTTTCGGAAATTTCGTCTGTTCCAGCATCAGACAGAATGGTCCAGAATCTT
TTGGACAAACTGCGCTCAGATGGGGCAGAACTTCTAATGTTTCTTAATCACATGGAAAAAATTTCTATTTGTGAA
ATAGATAAGAGTACTGGAGCTCTAAATGTGCTGTATTCAGTAAAGGGCAAAATCACAGATGGAGACAGATTGAAA
AGGAAACAATTTCATGCATCTGTAATTGATAGTGTTACTAAAAAGAGGCAGCTCAAAGACATACCAGTTCAACAA
ATAACCTATACTATGGATACTGAGGACTCTGAAGGAAATCTTACTACGTGGCTAATTTGTAATAGATCAGGCTTT
TCAAGTATGGAGAAAGTATCTAAAAGTGTCATATCAGCTCACAAGAACCAAGATATTACTCTTTTCCCACGTGGT
GGAGTAGCTGCCTGCATTACTCACAACTATAAAAAACCCCATAGGGCCTTCTGTTTTTTGCCTCTTTCTTTGGAG
ACTGGGCTGCCATTTCATGTGAATGGCCACTTTGCACTGGATTCAGCCAGAAGGAACCTGTGGCGTGATGATAAT
GGAGTTGGTGTTCGAAGTGACTGGAATAACAGTTTAATGACAGCATTAATAGCTCCTGCATATGTTGAATTGCTA
ATACAGTTAAAAAAACGGTATTTCCCTGGTTCTGATCCAACATTATCAGTGTTACAGAACACCCCTATTCATGTT
GTAAAGGACACTTTAAAGAAGTTTTTATCGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGTGACTCGAGTCTAGAGGGCCCGTTTAAACCCGCTGATCAGCC
TCGACTGTGCCTTCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGGTGGGCTCT
ATGG

An example nucleotide sequence encoding an engineered sacsin comprising an SRR3 domain, a CMV_enhancer, CMV promoter and hGH polyadenylation sequence is:

(SEQ ID NO: 68)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAATGTCTGTTTTACAACACTTGGCACAGAATTTGGGCAGAAAGAA
AAATTGACCAGCAGAATTAAGAGCATCCTTAATGCATATCCTTCTGAAAAGGAAATGTTGAAAGAGCTTCTTCAA
AATGCTGATGATGCAAAGGCGACAGAAATCTGTTTTGTGTTTGATCCTAGACAGCATCCAGTTGATAGAATATTT
GATGATAAGTGGGCCCCATTGCAAGGGCCAGCACTTTGTGTGTACAACAACCAGCCATTTACAGAAGATGATGTT
AGAGGAATTCAGAATCTTGGAAAAGGCACGAAAGAGGGAAATCCTTATAAAACTGGACAGTATGGAATAGGATTC
AATTCTGTGTATCATATCACAGACTGCCCATCTTTTATTTCTGGCAATGACATCCTGTGTATTTTTGATCCTCAT
GCCAGATATGCACCAGGGGCCACATCCATTAGTCCCGGACGCATGTTTAGAGATTTGGATGCAGATTTTAGGACA
CAGTTCTCAGATGTTCTGGATCTTTATCTGGGAACCCATTTTAAACTGGATAATTGCACAATGTTCAGATTTCCT
CTTCGTAATGCAGAAATGGCAAAAGTTTCGGAAATTTCGTCTGTTCCAGCATCAGACAGAATGGTCCAGAATCTT
TTGGACAAACTGCGCTCAGATGGGGCAGAACTTCTAATGTTTCTTAATCACATGGAAAAAATTTCTATTTGTGAA
ATAGATAAGAGTACTGGAGCTCTAAATGTGCTGTATTCAGTAAAGGGCAAAATCACAGATGGAGACAGATTGAAA
AGGAAACAATTTCATGCATCTGTAATTGATAGTGTTACTAAAAAGAGGCAGCTCAAAGACATACCAGTTCAACAA
ATAACCTATACTATGGATACTGAGGACTCTGAAGGAAATCTTACTACGTGGCTAATTTGTAATAGATCAGGCTTT
TCAAGTATGGAGAAAGTATCTAAAAGTGTCATATCAGCTCACAAGAACCAAGATATTACTCTTTTCCCACGTGGT
GGAGTAGCTGCCTGCATTACTCACAACTATAAAAAACCCCATAGGGCCTTCTGTTTTTTGCCTCTTTCTTTGGAG
ACTGGGCTGCCATTTCATGTGAATGGCCACTTTGCACTGGATTCAGCCAGAAGGAACCTGTGGCGTGATGATAAT
GGAGTTGGTGTTCGAAGTGACTGGAATAACAGTTTAATGACAGCATTAATAGCTCCTGCATATGTTGAATTGCTA
ATACAGTTAAAAAAACGGTATTTCCCTGGTTCTGATCCAACATTATCAGTGTTACAGAACACCCCTATTCATGTT
GTAAAGGACACTTTAAAGAAGTTTTTATCGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGTGACTCGAGAGATCTACGGGTGGCATCCCTGTGACCCCTCCC
CAGTGCCTCTCCTGGCCCTGGAAGTTGCCACTCCAGTGCCCACCAGCCTTGTCCTAATAAAATTAAGTTGCATCA
TTTTGTCTGACTAGGTGTCCTTCTATAATATTATGGGGTGGAGGGGGGTGGTATGGAGCAAGGGGCAAGTTGGGA
AGACAACCTGTAGGGCCTGCGGGGTCTATTGGGAACCAAGCTGGAGTGCAGTGGCACAATCTTGGCTCACTGCAA
TCTCCGCCTCCTGGGTTCAAGCGATTCTCCTGCCTCAGCCTCCCGAGTTGTTGGGATTCCAGGCATGCATGACCA
GGCTCAGCTAATTTTTGTTTTTTTGGTAGAGACGGGGTTTCACCATATTGGCCAGGCTGGTCTCCAACTCCTAAT
CTCAGGTGATCTACCCACCTTGGCCTCCCAAATTGCTGGGATTACAGGCGTGAACCACTGCTCCCTTCCCTGTCC
TT

An example nucleotide sequence encoding an engineered sacsin comprising an SRR3 domain, a CMV_enhancer, CMV promoter and pAmin polyadenylation sequence is:

(SEQ ID NO: 69)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAATGTCTGTTTTACAACACTTGGCACAGAATTTGGGCAGAAAGAA
AAATTGACCAGCAGAATTAAGAGCATCCTTAATGCATATCCTTCTGAAAAGGAAATGTTGAAAGAGCTTCTTCAA
AATGCTGATGATGCAAAGGCGACAGAAATCTGTTTTGTGTTTGATCCTAGACAGCATCCAGTTGATAGAATATTT
GATGATAAGTGGGCCCCATTGCAAGGGCCAGCACTTTGTGTGTACAACAACCAGCCATTTACAGAAGATGATGTT
AGAGGAATTCAGAATCTTGGAAAAGGCACGAAAGAGGGAAATCCTTATAAAACTGGACAGTATGGAATAGGATTC
AATTCTGTGTATCATATCACAGACTGCCCATCTTTTATTTCTGGCAATGACATCCTGTGTATTTTTGATCCTCAT
GCCAGATATGCACCAGGGGCCACATCCATTAGTCCCGGACGCATGTTTAGAGATTTGGATGCAGATTTTAGGACA
CAGTTCTCAGATGTTCTGGATCTTTATCTGGGAACCCATTTTAAACTGGATAATTGCACAATGTTCAGATTTCCT
CTTCGTAATGCAGAAATGGCAAAAGTTTCGGAAATTTCGTCTGTTCCAGCATCAGACAGAATGGTCCAGAATCTT
TTGGACAAACTGCGCTCAGATGGGGCAGAACTTCTAATGTTTCTTAATCACATGGAAAAAATTTCTATTTGTGAA
ATAGATAAGAGTACTGGAGCTCTAAATGTGCTGTATTCAGTAAAGGGCAAAATCACAGATGGAGACAGATTGAAA
AGGAAACAATTTCATGCATCTGTAATTGATAGTGTTACTAAAAAGAGGCAGCTCAAAGACATACCAGTTCAACAA
ATAACCTATACTATGGATACTGAGGACTCTGAAGGAAATCTTACTACGTGGCTAATTTGTAATAGATCAGGCTTT
TCAAGTATGGAGAAAGTATCTAAAAGTGTCATATCAGCTCACAAGAACCAAGATATTACTCTTTTCCCACGTGGT
GGAGTAGCTGCCTGCATTACTCACAACTATAAAAAACCCCATAGGGCCTTCTGTTTTTTGCCTCTTTCTTTGGAG
ACTGGGCTGCCATTTCATGTGAATGGCCACTTTGCACTGGATTCAGCCAGAAGGAACCTGTGGCGTGATGATAAT
GGAGTTGGTGTTCGAAGTGACTGGAATAACAGTTTAATGACAGCATTAATAGCTCCTGCATATGTTGAATTGCTA
ATACAGTTAAAAAAACGGTATTTCCCTGGTTCTGATCCAACATTATCAGTGTTACAGAACACCCCTATTCATGTT
GTAAAGGACACTTTAAAGAAGTTTTTATCGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGTGACTCGACGAATTCAATAAAAGATCTTTATTTTCATTAGAT
CTGTGTGTTGGTTTTTTGTGTG

An example nucleotide sequence encoding an engineered sacsin comprising an SRR3 domain, a CMV_enhancer, CMV promoter and bGH polyadenylation sequence is:

(SEQ ID NO: 70)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAATGTCTGTTTTACAACACTTGGCACAGAATTTGGGCAGAAAGAA
AAATTGACCAGCAGAATTAAGAGCATCCTTAATGCATATCCTTCTGAAAAGGAAATGTTGAAAGAGCTTCTTCAA
AATGCTGATGATGCAAAGGCGACAGAAATCTGTTTTGTGTTTGATCCTAGACAGCATCCAGTTGATAGAATATTT
GATGATAAGTGGGCCCCATTGCAAGGGCCAGCACTTTGTGTGTACAACAACCAGCCATTTACAGAAGATGATGTT
AGAGGAATTCAGAATCTTGGAAAAGGCACGAAAGAGGGAAATCCTTATAAAACTGGACAGTATGGAATAGGATTC
AATTCTGTGTATCATATCACAGACTGCCCATCTTTTATTTCTGGCAATGACATCCTGTGTATTTTTGATCCTCAT
GCCAGATATGCACCAGGGGCCACATCCATTAGTCCCGGACGCATGTTTAGAGATTTGGATGCAGATTTTAGGACA
CAGTTCTCAGATGTTCTGGATCTTTATCTGGGAACCCATTTTAAACTGGATAATTGCACAATGTTCAGATTTCCT
CTTCGTAATGCAGAAATGGCAAAAGTTTCGGAAATTTCGTCTGTTCCAGCATCAGACAGAATGGTCCAGAATCTT
TTGGACAAACTGCGCTCAGATGGGGCAGAACTTCTAATGTTTCTTAATCACATGGAAAAAATTTCTATTTGTGAA
ATAGATAAGAGTACTGGAGCTCTAAATGTGCTGTATTCAGTAAAGGGCAAAATCACAGATGGAGACAGATTGAAA
AGGAAACAATTTCATGCATCTGTAATTGATAGTGTTACTAAAAAGAGGCAGCTCAAAGACATACCAGTTCAACAA
ATAACCTATACTATGGATACTGAGGACTCTGAAGGAAATCTTACTACGTGGCTAATTTGTAATAGATCAGGCTTT
TCAAGTATGGAGAAAGTATCTAAAAGTGTCATATCAGCTCACAAGAACCAAGATATTACTCTTTTCCCACGTGGT
GGAGTAGCTGCCTGCATTACTCACAACTATAAAAAACCCCATAGGGCCTTCTGTTTTTTGCCTCTTTCTTTGGAG
ACTGGGCTGCCATTTCATGTGAATGGCCACTTTGCACTGGATTCAGCCAGAAGGAACCTGTGGCGTGATGATAAT
GGAGTTGGTGTTCGAAGTGACTGGAATAACAGTTTAATGACAGCATTAATAGCTCCTGCATATGTTGAATTGCTA
ATACAGTTAAAAAAACGGTATTTCCCTGGTTCTGATCCAACATTATCAGTGTTACAGAACACCCCTATTCATGTT
GTAAAGGACACTTTAAAGAAGTTTTTATCGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGGGTAAGCCTATCCCTAACCCTCTCCTCGGTCTCGATTCTACG
TGACTCGAGTCTAGAGGGCCCGTTTAAACCCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTTCCTAATAAAATGAGGAA
ATTGCATCGCATTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGG

An example nucleotide sequence encoding an engineered sacsin comprising an SRR3 domain, a CMV_enhancer, CMV promoter and hGH polyadenylation sequence is:

(SEQ ID NO: 71)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAATGTCTGTTTTACAACACTTGGCACAGAATTTGGGCAGAAAGAA
AAATTGACCAGCAGAATTAAGAGCATCCTTAATGCATATCCTTCTGAAAAGGAAATGTTGAAAGAGCTTCTTCAA
AATGCTGATGATGCAAAGGCGACAGAAATCTGTTTTGTGTTTGATCCTAGACAGCATCCAGTTGATAGAATATTT
GATGATAAGTGGGCCCCATTGCAAGGGCCAGCACTTTGTGTGTACAACAACCAGCCATTTACAGAAGATGATGTT
AGAGGAATTCAGAATCTTGGAAAAGGCACGAAAGAGGGAAATCCTTATAAAACTGGACAGTATGGAATAGGATTC
AATTCTGTGTATCATATCACAGACTGCCCATCTTTTATTTCTGGCAATGACATCCTGTGTATTTTTGATCCTCAT
GCCAGATATGCACCAGGGGCCACATCCATTAGTCCCGGACGCATGTTTAGAGATTTGGATGCAGATTTTAGGACA
CAGTTCTCAGATGTTCTGGATCTTTATCTGGGAACCCATTTTAAACTGGATAATTGCACAATGTTCAGATTTCCT
CTTCGTAATGCAGAAATGGCAAAAGTTTCGGAAATTTCGTCTGTTCCAGCATCAGACAGAATGGTCCAGAATCTT
TTGGACAAACTGCGCTCAGATGGGGCAGAACTTCTAATGTTTCTTAATCACATGGAAAAAATTTCTATTTGTGAA
ATAGATAAGAGTACTGGAGCTCTAAATGTGCTGTATTCAGTAAAGGGCAAAATCACAGATGGAGACAGATTGAAA
AGGAAACAATTTCATGCATCTGTAATTGATAGTGTTACTAAAAAGAGGCAGCTCAAAGACATACCAGTTCAACAA
ATAACCTATACTATGGATACTGAGGACTCTGAAGGAAATCTTACTACGTGGCTAATTTGTAATAGATCAGGCTTT
TCAAGTATGGAGAAAGTATCTAAAAGTGTCATATCAGCTCACAAGAACCAAGATATTACTCTTTTCCCACGTGGT
GGAGTAGCTGCCTGCATTACTCACAACTATAAAAAACCCCATAGGGCCTTCTGTTTTTTGCCTCTTTCTTTGGAG
ACTGGGCTGCCATTTCATGTGAATGGCCACTTTGCACTGGATTCAGCCAGAAGGAACCTGTGGCGTGATGATAAT
GGAGTTGGTGTTCGAAGTGACTGGAATAACAGTTTAATGACAGCATTAATAGCTCCTGCATATGTTGAATTGCTA
ATACAGTTAAAAAAACGGTATTTCCCTGGTTCTGATCCAACATTATCAGTGTTACAGAACACCCCTATTCATGTT
GTAAAGGACACTTTAAAGAAGTTTTTATCGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGGGTAAGCCTATCCCTAACCCTCTCCTCGGTCTCGATTCTACG
TGACTCGAGAGATCTACGGGTGGCATCCCTGTGACCCCTCCCCAGTGCCTCTCCTGGCCCTGGAAGTTGCCACTC
CAGTGCCCACCAGCCTTGTCCTAATAAAATTAAGTTGCATCATTTTGTCTGACTAGGTGTCCTTCTATAATATTA
TGGGGTGGAGGGGGGTGGTATGGAGCAAGGGGCAAGTTGGGAAGACAACCTGTAGGGCCTGCGGGGTCTATTGGG
AACCAAGCTGGAGTGCAGTGGCACAATCTTGGCTCACTGCAATCTCCGCCTCCTGGGTTCAAGCGATTCTCCTGC
CTCAGCCTCCCGAGTTGTTGGGATTCCAGGCATGCATGACCAGGCTCAGCTAATTTTTGTTTTTTTGGTAGAGAC
GGGGTTTCACCATATTGGCCAGGCTGGTCTCCAACTCCTAATCTCAGGTGATCTACCCACCTTGGCCTCCCAAAT
TGCTGGGATTACAGGCGTGAACCACTGCTCCCTTCCCTGTCCTT

An example nucleotide sequence encoding an engineered sacsin comprising an SRR3 domain, a CMV_enhancer, CMV promoter and pAmin polyadenylation sequence is:

(SEQ ID NO: 72)
GACATTGATTATTGACTAGTTATTAATAGTAATCAATTACGGGGTCATTAGTTCATAGCCCATATATGGAGTTCC
GCGTTACATAACTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACGGTAAACTGCCC
ACTTGGCAGTACATCAAGTGTATCATATGCCAAGTACGCCCCCTATTGACGTCAATGACGGTAAATGGCCCGCCT
GGCATTATGCCCAGTACATGACCTTATGGGACTTTCCTACTTGGCAGTACATCTACGTATTAGTCATCGCTATTA
CCATGGTGATGCGGTTTTGGCAGTACATCAATGGGCGTGGATAGCGGTTTGACTCACGGGGATTTCCAAGTCTCC
ACCCCATTGACGTCAATGGGAGTTTGTTTTGGCACCAAAATCAACGGGACTTTCCAAAATGTCGTAACAACTCCG
CCCCATTGACGCAAATGGGCGGTAGGCGTGTACGGTGGGAGGTCTATATAAGCAGAGCTCTCTGGCTAACTAGAG
AACCCACTGCTTACTGGCTTATCGAAATTAATACGACTCACTATAGGGAGACCCAAGCTGGCTAGCGCCGCCGCG
ATCGCCATGGAGACCAAGGAGAACAGGTGGGTCCCGGTGACCGTGCTCCCCGGCTGCGTGGGCTGCAGGACCGTC
GCGGCGCTGGCGTCCTGGACCGTGCGCGATGTGAAGGAACGTATCTTCGCGGAGACTGGCTTCCCGGTGTCGGAG
CAGCGGCTGTGGCGCGGCGGCCGCGAGTTATCTGACTGGATCAAGATTGGAGATCTGACTTCCAAAAATTGTCAT
CTTTTTGTAAACCTTCAATCAAAAGGCTTAAATGTCTGTTTTACAACACTTGGCACAGAATTTGGGCAGAAAGAA
AAATTGACCAGCAGAATTAAGAGCATCCTTAATGCATATCCTTCTGAAAAGGAAATGTTGAAAGAGCTTCTTCAA
AATGCTGATGATGCAAAGGCGACAGAAATCTGTTTTGTGTTTGATCCTAGACAGCATCCAGTTGATAGAATATTT
GATGATAAGTGGGCCCCATTGCAAGGGCCAGCACTTTGTGTGTACAACAACCAGCCATTTACAGAAGATGATGTT
AGAGGAATTCAGAATCTTGGAAAAGGCACGAAAGAGGGAAATCCTTATAAAACTGGACAGTATGGAATAGGATTC
AATTCTGTGTATCATATCACAGACTGCCCATCTTTTATTTCTGGCAATGACATCCTGTGTATTTTTGATCCTCAT
GCCAGATATGCACCAGGGGCCACATCCATTAGTCCCGGACGCATGTTTAGAGATTTGGATGCAGATTTTAGGACA
CAGTTCTCAGATGTTCTGGATCTTTATCTGGGAACCCATTTTAAACTGGATAATTGCACAATGTTCAGATTTCCT
CTTCGTAATGCAGAAATGGCAAAAGTTTCGGAAATTTCGTCTGTTCCAGCATCAGACAGAATGGTCCAGAATCTT
TTGGACAAACTGCGCTCAGATGGGGCAGAACTTCTAATGTTTCTTAATCACATGGAAAAAATTTCTATTTGTGAA
ATAGATAAGAGTACTGGAGCTCTAAATGTGCTGTATTCAGTAAAGGGCAAAATCACAGATGGAGACAGATTGAAA
AGGAAACAATTTCATGCATCTGTAATTGATAGTGTTACTAAAAAGAGGCAGCTCAAAGACATACCAGTTCAACAA
ATAACCTATACTATGGATACTGAGGACTCTGAAGGAAATCTTACTACGTGGCTAATTTGTAATAGATCAGGCTTT
TCAAGTATGGAGAAAGTATCTAAAAGTGTCATATCAGCTCACAAGAACCAAGATATTACTCTTTTCCCACGTGGT
GGAGTAGCTGCCTGCATTACTCACAACTATAAAAAACCCCATAGGGCCTTCTGTTTTTTGCCTCTTTCTTTGGAG
ACTGGGCTGCCATTTCATGTGAATGGCCACTTTGCACTGGATTCAGCCAGAAGGAACCTGTGGCGTGATGATAAT
GGAGTTGGTGTTCGAAGTGACTGGAATAACAGTTTAATGACAGCATTAATAGCTCCTGCATATGTTGAATTGCTA
ATACAGTTAAAAAAACGGTATTTCCCTGGTTCTGATCCAACATTATCAGTGTTACAGAACACCCCTATTCATGTT
GTAAAGGACACTTTAAAGAAGTTTTTATCGGGATCCATCTTAAAAGAAGTGACATCTGTGGTGGAGCAAGCATGG
AAGCTTCCAGAATCGGAACGAAAAAAGATTATTAGGCGGTTGTATTTGAAATGGCATCCTGACAAAAATCCAGAG
AACCATGACATTGCCAATGAAGTTTTTAAACATTTGCAGAATGAAATCAACAGATTAGAAAAACAGGCTTTTCTA
GATCAAAATGCAGACAGGGCCTCCAGACGAACATTTTCAACCTCAGCATCCCGATTTCAGTCAGACAAATACTCA
TTTCAGAGATTCTATACTTCATGGAATCAAGAAGCAACGAGCCATAAATCTGAAAGACAGCAACAGAACAAAGAA
AAATGCCCCCCTTCAGCCGGACAGACTTACTCTCAAAGGTTCTTTGTTCCTCCCACTTTCAAGTCGGTTGGCAAT
CCAGTGGAAGCACGCAGATGGCTAAGACAAGCCAGAGCAAACTTCTCAGCTGCCAGGAATGACCTTCATAAAAAT
GCCAATGAGTGGGTGTGCTTTAAATGTTACCTTTCTACCAAGTTAGCTTTGATTGCAGCTGACTATGCTGTGAGG
GGAAAGTCTGATAAAGATGTAAAACCAACTGCACTTGCTCAGAAAATAGAGGAATATAGTCAGCAACTTGAAGGA
CTGACAAATGATGTTCACACATTGGAAGCTTATGGTGTAGACAGTTTAAAAACAAGATACCCTGATTTGCTTCCC
TTTCCTCAGATCCCAAATGACAGGTTCACTTCTGAGGTTGCTATGAGGGTGATGGAATGTACTGCCTGTATCATA
ATAAAACTTGAAAATTTTATGCAACAAAAAGTGGGTAAGCCTATCCCTAACCCTCTCCTCGGTCTCGATTCTACG
TGACTCGACGAATTCAATAAAAGATCTTTATTTTCATTAGATCTGTGTGTTGGTTTTTTGTGTG

### Blood-Brain Barrier (BBB)

The term “blood brain barrier” (BBB) as used herein means the highly selective semi-permeable membrane barrier which separates the circulating blood from the brain and extracellular fluid in the central nervous system. It is formed by the selectivity of tight junctions between endothelial cells. The blood-brain barrier occurs along all capillaries of the brain and consists of tight junctions.

Overcoming the difficulty of delivering therapeutic agents to specific regions of the brain presents a major challenge to treatment of most brain disorders.

Preferably, the vector particle of the invention (e.g. the AAV vector particle) is adapted for crossing an intact blood brain barrier. Preferably the vector particle (e.g. the AAV vector particle) does not impair blood-brain barrier integrity and/or selectivity and/or affect permeability.

In other words, the vector particle is adapted to cross a blood-brain barrier which has not been compromised or weakened, i.e. which maintains tight junctions between endothelial cells. Methods are known in the art which can determine whether or not a blood brain barrier is intact. For example, the permeability of the blood-brain barrier can be detected by perfusion of Evan's blue dye. Alternatively a fluorescent-conjugated cadaverine dye can be used as a blood-brain barrier permeability marker, together with the AAV particle carrying a fluorescent marker.

Preferably, the vector particle (e.g. the AAV vector particle) does not cause microgliosis. Suitably, the vector particle (e.g. the AAV vector particle) does not cause sustained inflammation in the central nervous system.

The “central nervous system” as used herein means the nervous system consisting of the brain and spinal cord.

The “peripheral nervous system” as used herein means the components of the nervous system outside of the central nervous system. The peripheral nervous system consists of the nerves and ganglia outside of the brain and spinal cord.

### Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (DNA or RNA) within the cell, facilitating the replication of the vector comprising a segment of nucleic acid and/or facilitating the expression of the protein encoded by a segment of nucleic acid.

Vectors comprising polynucleotides used in the invention may be introduced into cells using a variety of techniques known in the art, such as transfection, transduction and transformation.

Transfection may refer to a general process of incorporating a nucleic acid into a cell and includes a process using a non-viral vector to deliver a polynucleotide to a cell. Transduction may refer to a process of incorporating a nucleic acid into a cell using a viral vector.

Preferably, the vectors used to transduce cells in the invention are viral vectors. The vectors of the invention are preferably adeno-associated viral (AAV) vectors, although it is contemplated that other viral vectors may be used. In some embodiments, the vector is a retroviral, lentiviral, AAV or adenoviral vector.

Preferably, the viral vector for use according to the present invention is in the form of a viral vector particle.

In one aspect the invention provides a viral vector particle adapted for crossing the blood-brain barrier, wherein the viral vector particle comprises a nucleotide sequence encoding an engineered sacsin, wherein the engineered sacsin comprises a UbL domain, an SRR domain, a DnaJ domain and an HEPN domain, and wherein the nucleotide sequence encoding the engineered sacsin is less than or equal to about 2700 bp in length.

In one embodiment the viral vector particle adapted for crossing the blood-brain barrier is a retroviral, lentiviral, adeno-associated viral (AAV) or adenoviral vector particle. Preferably, the viral vector particle is a lentiviral or AAV vector particle, more preferably an AAV vector particle.

Although, some embodiments of the invention have been described with respect to AAV vector particles, it will be appreciated that some embodiments may apply mutatis mutandis to other viral vectors disclosed herein.

### Adeno-Associated Viral (AAV) Vectors

In one aspect the invention provides a AAV vector particle, wherein the AAV vector particle comprises a nucleotide sequence encoding an engineered sacsin, wherein the engineered sacsin comprises a UbL domain, an SRR domain, a DnaJ domain and an HEPN domain, and wherein the nucleotide sequence encoding the engineered sacsin is less than or equal to about 2700 bp in length. In some embodiments, the AAV vector particle is adapted for crossing the blood-brain barrier.

Methods of preparing and modifying viral vectors and viral vector particles, such as those derived from AAV, are well known in the art.

The AAV vector may comprise an AAV genome or a fragment or derivative thereof.

An AAV genome is a polynucleotide sequence, which may encode functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, the AAV genome of the AAV vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain.

Several rAAV vectors have been reported to efficiently cross the blood-brain barrier and transduce neurons and astrocytes in the neonatal mouse central nervous system (Zhang et al., Molecular therapy vol. 19, no 8, 1440-1448).

In some embodiments, the AAV is an AAV1, AAV6, AAV6.2, AAV7, AAV9, rh10, rh39 or rh43 serotype. In some embodiments, the AAV vector particle comprises an AAV1, AAV6, AAV6.2, AAV7, AAV9, rh10, rh39 or rh43 serotype capsid protein. In some embodiments, the AAV vector particle is an AAV1, AAV6, AAV6.2, AAV7, AAV9, rh10, rh39 or rh43 vector particle.

In some embodiments, the AAV is an AAV9 PHP.eB; AAV-Se2w; AAV9; AAV9 PHP.B; or AAVrh10 serotype. In some embodiments, the AAV vector particle comprises an AAV9 PHP.eB; AAV-Se2w; AAV9; AAV9 PHP.B; or AAVrh10 serotype capsid protein.

The capsid protein may be an artificial or mutant capsid protein.

The term “artificial capsid” as used herein means that the capsid particle comprises an amino acid sequence which does not occur in nature or which comprises an amino acid sequence which has been engineered (e.g. modified) from a naturally occurring capsid amino acid sequence.

In other words the artificial capsid protein comprises a mutation or a variation in the amino acid sequence compared to the sequence of the parent capsid from which it is derived where the artificial capsid amino acid sequence and the parent capsid amino acid sequences are aligned. Methods of sequence alignment are well known in the art and referenced herein.

The term “adapted for crossing the blood brain barrier” as used herein means that the vector particle has the ability to cross the blood brain barrier, for example the vector particle may comprise a mutation or modification relative to the wild type vector particle which improves the ability to cross the blood brain barrier relative to an unmodified or wild type viral particle. Improved ability to cross the blood brain barrier may be measured for example by measuring the expression of a transgene, e.g. GFP, carried by the vector particle, wherein expression of the transgene in the brain correlates with the ability of the viral particle to cross the blood brain barrier.

In some embodiments, the AAV vector particle comprises an artificial capsid amino acid sequence which enables the viral particle to cross the blood-brain barrier.

In some embodiments, the AAV vector particle capable of crossing the blood-brain barrier comprises a VP1 capsid protein comprising an amino acid sequence comprising at least four contiguous amino acids from the sequence TLAVPFK (SEQ ID NO: 39) or KFPVALT (SEQ ID NO: 40).

In some embodiments, the AAV vector particle capable of crossing the blood-brain barrier comprises a VP1 capsid protein comprising an amino acid sequence comprising at least five contiguous amino acids from the sequence TLAVPFK (SEQ ID NO: 39) or KFPVALT (SEQ ID NO: 40).

In some embodiments, the AAV vector particle capable of crossing the blood-brain barrier comprises a VP1 capsid protein comprising an amino acid sequence comprising at least six contiguous amino acids from the sequence TLAVPFK (SEQ ID NO: 39) or KFPVALT (SEQ ID NO: 40).

In some embodiments, the AAV vector particle capable of crossing the blood-brain barrier comprises a VP1 capsid protein comprising an amino acid sequence comprising the sequence TLAVPFK (SEQ ID NO: 39) or KFPVALT (SEQ ID NO: 40).

In some embodiments, the nucleic acid sequence encoding the at least four, at least five, at least six or all seven contiguous amino acids from the sequence TLAVPFK (SEQ ID NO: 39) or KFPVALT (SEQ ID NO: 40) is inserted at a position corresponding to the position between a sequence encoding for amino acids 588 and 589 of AAV9 (SEQ ID NO: 41).

An example amino acid sequence of the (wild-type) AAV9 capsid is:

(SEQ ID NO: 41)
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAHEGCLPPFPADVFMIP
QYGYLTLNDGSQAVGRSSFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQAQAQTGWVQNQG
ILPGMVWQDRDVYLQGPIWAKIPHTDGNFHPSPLMGGFGMKHPPPQILIKNTPVPADPPTAFNKDKINSFITQYS
TGQVSVEIEWELQKENSKRWNPEIQYTSNYYKSNNVEFAVNTEGVYSEPRPIGTRYLTRNL

In some embodiments, the AAV vector particle comprises a AAV9 PHP.B capsid, preferably the AAV-PHP.B VP1 capsid protein.

In some embodiments, the AAV vector particle capable of crossing the blood-brain barrier is AAV9 PHP.B.

In some embodiments, the amino acid sequence of the AAV-PHP.B capsid VP1 protein is:

(SEQ ID NO: 42)
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSAQTLAVPFKAQAQT
GWVQNQGILPGMVWQDRDVYLQGPIWAKIPHTDGNFHPSPLMGGFGMKHPPPQILIKNTPVPADPPTAFNKDKLN
SFITQYSTGQVSVEIEWELQKENSKRWNPEIQYTSNYYKSNNVEFAVNTEGVYSEPRPIGTRYLTRNL

In some embodiments, the AAV vector particle comprises a capsid comprising an amino acid sequence that has at least 70%, 75%, 80%, 85% or 90% identity to SEQ ID NO: 42, more preferably at least 95%, 96%, 97%, 98%, 99% or 100% identity to SEQ ID NO: 42, wherein the AAV vector particle is capable of crossing the blood-brain barrier.

The AAV-PHP.B vector is described in Deverman et al. (2016) Nat Biotechnol 34:204-209 and WO 2015/038958, which are incorporated herein by reference.

In some embodiments, the AAV vector particle capable of crossing the blood-brain barrier comprises a VP1 capsid protein comprising an amino acid sequence comprising the sequence DGTLAVPFKAQ (SEQ ID NO: 43).

In some embodiments, the AAV vector particle capable of crossing the blood-brain barrier is AAV9 PHP.eB.

In some embodiments, the amino acid sequence of the AAV-PHP.eB capsid VP1 protein is:

(SEQ ID NO: 44)
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMNPLIDQYLYYLSKT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSDGTLAVPFKAQAQT
GWVQNQGILPGMVWQDRDVYLQGPIWAKIPHTDGNFHPSPLMGGFGMKHPPPQILIKNTPVPADPPTAFNKDKLN
SFITQYSTGQVSVEIEWELQKENSKRWNPEIQYTSNYYKSNNVEFAVNTEGVYSEPRPIGTRYLTRNL

In some embodiments, the AAV vector particle comprises a capsid comprising an amino acid sequence that has at least 70%, 75%, 80%, 85% or 90% identity to SEQ ID NO: 44, more preferably at least 95%, 96%, 97%, 98%, 99% or 100% identity to SEQ ID NO: 44, wherein the AAV vector particle is capable of crossing the blood-brain barrier.

The AAV-PHP.eB vector is described in WO 2017/100671, which is incorporated herein by reference.

In some embodiments, the AAV vector particle capable of crossing the blood-brain barrier is AAV-Se2w.

In some embodiments, the amino acid sequence of the AAV-Se2w capsid VP1 protein is:

(SEQ ID NO: 45)
MAADGYLPDWLEDNLSEGIREWWALKPGAPQPKANQQHQDNARGLVLPGYKYLGPGNGLDKGEPVNAADAAALEH
DKAYDQQLKAGDNPYLKYNHADAEFQERLKEDTSFGGNLGRAVFQAKKRLLEPLGLVEEAAKTAPGKKRPVEQSP
QEPDSSAGIGKSGAQPAKKRLNFGQTGDTESVPDPQPIGEPPAAPSGVGSLTMASGGGAPVADNNEGADGVGSSS
GNWHCDSQWLGDRVITTSTRTWALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLGSAHEGCLPPFPADVEMIP
QYGYLTLNDGSQAVGRSSFYCLEYFPSQMLRTGNNFQFSYEFENVPFHSSYAHSQSLDRLMNPLIDQYLYYLSRT
INGSGQNQQTLKFSVAGPSNMAVQGRNYIPGPSYRQQRVSTTVTQNNNSEFAWPGASSWALNGRNSLMNPGPAMA
SHKEGEDRFFPLSGSLIFGKQGTGRDNVDADKVMITNEEEIKTTNPVATESYGQVATNHQSGQAGPGVPGRFAQA
ATGWVQNQGILPGMVWQDRDVYLQGPIWAKIPHTDGNFHPSPLMGGFGMKHPPPQILIKNTPVPADPPTAFNKDK
LNSFITQYSTGQVSVEIEWELQKENSKRWNPEIQYTSNYYKSNNVEFAVNTEGVYSEPRPIGTRYLTRNL

In some embodiments, the AAV vector particle comprises a capsid comprising an amino acid sequence that has at least 70%, 75%, 80%, 85% or 90% identity to SEQ ID NO: 45, more preferably at least 95%, 96%, 97%, 98%, 99% or 100% identity to SEQ ID NO: 45, wherein the AAV vector particle is capable of crossing the blood-brain barrier.

Reviews of AAV serotypes may be found in Choi et al. (2005) Curr. Gene Ther. 5:299-310 and Wu et al. (2006) Molecular Therapy 14:316-27. The sequences of AAV genomes or of elements of AAV genomes including ITR sequences, rep or cap genes for use in the invention may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof. Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, clade, clone or isolate of AAV for use in the invention on the basis of their common general knowledge.

The AAV serotype determines the tissue specificity of infection (or tropism) of an AAV virus.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. In preferred embodiments, one or more ITR sequences flank the nucleotide sequence encoding an engineered sacsin. The AAV genome may also comprise packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle.

A promoter will be operably linked to each of the packaging genes. Specific examples of such promoters include the p5, p19 and p40 promoters (Laughlin et al. (1979) Proc. Natl. Acad. Sci. USA 76:5567-5571). For example, the p5 and p19 promoters are generally used to express the rep gene, while the p40 promoter is generally used to express the cap gene.

As discussed above, the AAV genome used in the AAV vector of the invention may therefore be the full genome of a naturally occurring AAV. For example, a vector comprising a full AAV genome may be used to prepare an AAV vector or vector particle in vitro. However, while such a vector may in principle be administered to patients, this will rarely be done in practice. Preferably the AAV genome will be derivatised for the purpose of administration to patients. Such derivatisation is standard in the art and the invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4:99, and in Choi et al, and Wu et al., referenced above.

Derivatives of an AAV genome include any truncated or modified forms of an AAV genome which allow for expression of a transgene from an AAV vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

In some embodiments, the AAV vector comprises at least one, such as two, AAV1, 2, 3, 4, 5, 6, 7, 8, 9, 10, or 11 ITRs. In some embodiments, the AAV vector comprises at least one AAV9 ITR.

In some embodiments, the AAV vector comprises two AAV9 ITRs.

The one or more ITRs will preferably flank the nucleotide sequence encoding an engineered sacsin at either end. The inclusion of one or more ITRs is preferred to aid concatamer formation of the vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector). Thus, in one embodiment the AAV vector is in the form of a pseudotyped AAV vector particle.

Chimeric, shuffled or capsid-modified derivatives will be typically selected to provide one or more desired functionalities for the AAV vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form. Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population (e.g. to Purkinje cells). The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

The AAV vector of the invention may take the form of a nucleotide sequence comprising an AAV genome or derivative thereof and a nucleotide sequence encoding an engineered sacsin or derivatives thereof.

The AAV particles of the invention include transcapsidated forms wherein an AAV genome or derivative having an ITR of one serotype is packaged in the capsid of a different serotype. The AAV particles of the invention also include mosaic forms wherein a mixture of unmodified capsid proteins from two or more different serotypes makes up the viral capsid. The AAV particle also includes chemically modified forms bearing ligands adsorbed to the capsid surface. For example, such ligands may include antibodies for targeting a particular cell surface receptor.

Thus, for example, the AAV particles of the invention include those with an AAV2 genome and AAV9 capsid proteins (AAV2/9), or AAV9 PHP.B, PHP.eB or AAV-Se2w capsid proteins.

The AAV vector may comprise multiple copies (e.g., 2, 3 etc.) of the nucleotide sequence referred to herein.

### Exemplary Vectors

In one aspect, the invention provides a vector comprising or consisting of a nucleotide sequence having at least 70%, 75%, 80%, 85%, 90%, 95%, 96%, 97%, 98% or 99% sequence identity to any one of SEQ ID NOs: 73-80.

In some embodiments, the vector comprises or consists of the nucleotide sequence of any one of SEQ ID NOs: 73-80.

An exemplary vector comprising a CMV enhancer, CBA promoter, β-globin intron, minisacsin with SRR1 and hGh polyadenylation sequence is:

(SEQ ID NO: 73)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
ACGCGCTAGTTATTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGGTCGA
GGTGAGCCCCACGTTCTGCTTCACTCTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGCGGGGAGTCGCTGCGACGCTGCCTTCGC
CCCGTGCCCCGCTCCGCCGCCGCCTCGCGCCGCCCGCCCCGGCTCTGACTGACCGCGTTACTCCCACAGGTGAGC
GGGCGGGACGGCCCTTCTCCTCCGGGCTGTAATTAGCCCGTTTAGTGAACCGTCAGATCGCCTGGAGACGCCATC
CACGCTGTTTTGACCTCCATAGAAGACACCGGGACCGATCCAGCCTCCGCGGATTCGAATCCCGGCCGGGAACGG
TGCATTGGAACGCGGATTCCCCGTGCCAAGAGTGACGTAAGTACCGCCTATAGAGTCTATAGGCCCACAAAAAAT
GCTTTCTTCTTTTAATATACTTTTTTGTTTATCTTATTTCTAATACTTTCCCTAATCTCTTTCTTTCAGGGCAAT
AATGATACAATGTATCATGCCTCTTTGCACCATTCTAAAGAATAACAGTGATAATTTCTGGGTTAAGGCAATAGC
AATATTTCTGCATATAAATATTTCTGCATATAAATTGTAACTGATGTAAGAGGTTTCATATTGCTAATAGCAGCT
ACAATCCAGCTACCATTCTGCTTTTATTTTATGGTTGGGATAAGGCTGGATTATTCTGAGTCCAAGCTAGGCCCT
TTTGCTAATCATGTTCATACCTCTTATCTTCCTCCCACAGCTCCTGGGCAACGTGCTGGTCTGTGTGCTGGCCCA
TCACTTTGGCAAAGAATTGGGATTCGAACACCGGTCGACGAATTCGTTAACGGATCCGCTAGCGCCGCCGCGATC
GCCATGGAGACCAAGGAGAACAGGTGGGTCCCGGTGACCGTGCTCCCCGGCTGCGTGGGCTGCAGGACCGTCGCG
GCGCTGGCGTCCTGGACCGTGCGCGATGTGAAGGAACGTATCTTCGCGGAGACTGGCTTCCCGGTGTCGGAGCAG
CGGCTGTGGCGCGGCGGCCGCGAGTTATCTGACTGGATCAAGATTGGAGATCTGACTTCCAAAAATTGTCATCTT
TTTGTAAACCTTCAATCAAAAGGCTTAAAAGGGGGAGGTCGATTTGGTCAGACAACGCCACCACTTGTTGATTTT
CTCAAGGACATTTTGAGAAGATATCCAGAAGGAGGACAGATTCTTAAGGAATTAATTCAGAATGCAGAAGATGCT
GGGGCGACAGAAGTTAAATTTTTATATGATGAAACTCAATACGGAACAGAGACTCTTTGGTCAAAAGATATGGCG
CCATATCAGGGGCCAGCTCTCTATGTGTACAACAACGCGGTTTTCACCCCAGAGGACTGGCACGGCATTCAAGAA
ATAGCAAGAAGCAGGAAAAAGGATGATCCTCTGAAGGTCGGAAGATTTGGAATTGGGTTTAATTCTGTCTATCAT
ATAACAGATGTTCCTTGTATCTTTAGTGGTGACCAAATCGGGATGCTAGATCCTCATCAAACACTTTTTGGCCCA
CATGAATCAGGCCAATGTTGGAATCTCAAAGATGACAGCAAAGAAATTAGTGAACTTTCAGACCAGTTTGCACCA
TTTGTTGGCATTTTTGGAAGCACCAAGGAAACATTTATAAACGGCAATTTTCCAGGAACATTTTTCCGTTTCCCT
CTTCGCCTACAACCTTCACAACTTAGTAGTAACCTCTACAATAAGCAGAAGGTTCTTGAGTTGTTTGAGTCTTTT
AGGGCAGATGCAGACACAGTGCTGCTCTTTCTGAAAAGTGTGCAGGATGTTTCCTTATATGTCCGAGAGGCTGAC
GGAACAGAGAAACTGGTGTTTAGAGTGACTTCGAGTGAGAGTAAGGCACTGAAACATGAGCGGCCGAATTCTATA
AAGATTCTGGGAACTGCTATAAGTAACTATTGTAAAAAGACTCCAAGCAATAACATCACCTGTGTAACATATCAC
GTAAATATTGTTTTAGAAGAGGAGAGTACTAAGGATGCACAGAAAACATCTTGGTTGGTGTGTAACAGTGTGGGT
GGGCGAGGGATCAGTAGTAAGCTTGACTCTTTAGCTGATGAACTGAAATTTGTCCCAATCATTGGAATAGCCATG
CCTTTATCAAGCAGAGATGATGAAGCAAAAGGAGCAACGTCTGATTTCTCAGGAAAAGCATTTTGTTTCCTTCCT
TTACCACCTGGTGAGGAAAGCAGCACAGGCCTCCCAGTTCACATCAGTGGGTTCTTTGGCCTTACTGATAACCGC
AGGAGCATAAAATGGAGAGAGCTGGACCAGTGGAGAGACCCGGCAGCCTTATGGAATGAGTTTCTTGTCATGAAT
GTTGTCCCCAAAGCTTATGCTACTCTGGGATCCATCTTAAAAGAAGTGACATCTGTGGTGGAGCAAGCATGGAAG
CTTCCAGAATCGGAACGAAAAAAGATTATTAGGCGGTTGTATTTGAAATGGCATCCTGACAAAAATCCAGAGAAC
CATGACATTGCCAATGAAGTTTTTAAACATTTGCAGAATGAAATCAACAGATTAGAAAAACAGGCTTTTCTAGAT
CAAAATGCAGACAGGGCCTCCAGACGAACATTTTCAACCTCAGCATCCCGATTTCAGTCAGACAAATACTCATTT
CAGAGATTCTATACTTCATGGAATCAAGAAGCAACGAGCCATAAATCTGAAAGACAGCAACAGAACAAAGAAAAA
TGCCCCCCTTCAGCCGGACAGACTTACTCTCAAAGGTTCTTTGTTCCTCCCACTTTCAAGTCGGTTGGCAATCCA
GTGGAAGCACGCAGATGGCTAAGACAAGCCAGAGCAAACTTCTCAGCTGCCAGGAATGACCTTCATAAAAATGCC
AATGAGTGGGTGTGCTTTAAATGTTACCTTTCTACCAAGTTAGCTTTGATTGCAGCTGACTATGCTGTGAGGGGA
AAGTCTGATAAAGATGTAAAACCAACTGCACTTGCTCAGAAAATAGAGGAATATAGTCAGCAACTTGAAGGACTG
ACAAATGATGTTCACACATTGGAAGCTTATGGTGTAGACAGTTTAAAAACAAGATACCCTGATTTGCTTCCCTTT
CCTCAGATCCCAAATGACAGGTTCACTTCTGAGGTTGCTATGAGGGTGATGGAATGTACTGCCTGTATCATAATA
AAACTTGAAAATTTTATGCAACAAAAAGTGTGACTCGAGAGATCTACGGGTGGCATCCCTGTGACCCCTCCCCAG
TGCCTCTCCTGGCCCTGGAAGTTGCCACTCCAGTGCCCACCAGCCTTGTCCTAATAAAATTAAGTTGCATCATTT
TGTCTGACTAGGTGTCCTTCTATAATATTATGGGGTGGAGGGGGGTGGTATGGAGCAAGGGGCAAGTTGGGAAGA
CAACCTGTAGGGCCTGCGGGGTCTATTGGGAACCAAGCTGGAGTGCAGTGGCACAATCTTGGCTCACTGCAATCT
CCGCCTCCTGGGTTCAAGCGATTCTCCTGCCTCAGCCTCCCGAGTTGTTGGGATTCCAGGCATGCATGACCAGGC
TCAGCTAATTTTTGTTTTTTTGGTAGAGACGGGGTTTCACCATATTGGCCAGGCTGGTCTCCAACTCCTAATCTC
AGGTGATCTACCCACCTTGGCCTCCCAAATTGCTGGGATTACAGGCGTGAACCACTGCTCCCTTCCCTGTCCTTC
TGATTTTGTAGGTAACCACGTGCGGACCGAGCGGCCGCAGGAACCCCTAGTGATGGAGTTGGCCACTCCCTCTCT
GCGCGCTCGCTCGCTCACTGAGGCCGGGCGACCAAAGGTCGCCCGACGCCCGGGCTTTGCCCGGGCGGCCTCAGT
GAGCGAGCGAGCGCGCAGCTGCCTGCAGG

An exemplary vector comprising a CMV enhancer, CBA promoter, β-globin intron, minisacsin with SRR1 and pAmin polyadenylation sequence is:

(SEQ ID NO: 74)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
ACGCGCTAGTTATTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGGTCGA
GGTGAGCCCCACGTTCTGCTTCACTCTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGGGGGGAGTCGCTGCGACGCTGCCTTCGC
CCCGTGCCCCGCTCCGCCGCCGCCTCGCGCCGCCCGCCCCGGCTCTGACTGACCGCGTTACTCCCACAGGTGAGC
GGGCGGGACGGCCCTTCTCCTCCGGGCTGTAATTAGCCCGTTTAGTGAACCGTCAGATCGCCTGGAGACGCCATC
CACGCTGTTTTGACCTCCATAGAAGACACCGGGACCGATCCAGCCTCCGCGGATTCGAATCCCGGCCGGGAACGG
TGCATTGGAACGCGGATTCCCCGTGCCAAGAGTGACGTAAGTACCGCCTATAGAGTCTATAGGCCCACAAAAAAT
GCTTTCTTCTTTTAATATACTTTTTTGTTTATCTTATTTCTAATACTTTCCCTAATCTCTTTCTTTCAGGGCAAT
AATGATACAATGTATCATGCCTCTTTGCACCATTCTAAAGAATAACAGTGATAATTTCTGGGTTAAGGCAATAGC
AATATTTCTGCATATAAATATTTCTGCATATAAATTGTAACTGATGTAAGAGGTTTCATATTGCTAATAGCAGCT
ACAATCCAGCTACCATTCTGCTTTTATTTTATGGTTGGGATAAGGCTGGATTATTCTGAGTCCAAGCTAGGCCCT
TTTGCTAATCATGTTCATACCTCTTATCTTCCTCCCACAGCTCCTGGGCAACGTGCTGGTCTGTGTGCTGGCCCA
TCACTTTGGCAAAGAATTGGGATTCGAACACCGGTCGACGAATTCGTTAACGGATCCGCTAGCGCCGCCGCGATC
GCCATGGAGACCAAGGAGAACAGGTGGGTCCCGGTGACCGTGCTCCCCGGCTGCGTGGGCTGCAGGACCGTCGCG
GCGCTGGCGTCCTGGACCGTGCGCGATGTGAAGGAACGTATCTTCGCGGAGACTGGCTTCCCGGTGTCGGAGCAG
CGGCTGTGGCGCGGCGGCCGCGAGTTATCTGACTGGATCAAGATTGGAGATCTGACTTCCAAAAATTGTCATCTT
TTTGTAAACCTTCAATCAAAAGGCTTAAAAGGGGGAGGTCGATTTGGTCAGACAACGCCACCACTTGTTGATTTT
CTCAAGGACATTTTGAGAAGATATCCAGAAGGAGGACAGATTCTTAAGGAATTAATTCAGAATGCAGAAGATGCT
GGGGCGACAGAAGTTAAATTTTTATATGATGAAACTCAATACGGAACAGAGACTCTTTGGTCAAAAGATATGGCG
CCATATCAGGGGCCAGCTCTCTATGTGTACAACAACGCGGTTTTCACCCCAGAGGACTGGCACGGCATTCAAGAA
ATAGCAAGAAGCAGGAAAAAGGATGATCCTCTGAAGGTCGGAAGATTTGGAATTGGGTTTAATTCTGTCTATCAT
ATAACAGATGTTCCTTGTATCTTTAGTGGTGACCAAATCGGGATGCTAGATCCTCATCAAACACTTTTTGGCCCA
CATGAATCAGGCCAATGTTGGAATCTCAAAGATGACAGCAAAGAAATTAGTGAACTTTCAGACCAGTTTGCACCA
TTTGTTGGCATTTTTGGAAGCACCAAGGAAACATTTATAAACGGCAATTTTCCAGGAACATTTTTCCGTTTCCCT
CTTCGCCTACAACCTTCACAACTTAGTAGTAACCTCTACAATAAGCAGAAGGTTCTTGAGTTGTTTGAGTCTTTT
AGGGCAGATGCAGACACAGTGCTGCTCTTTCTGAAAAGTGTGCAGGATGTTTCCTTATATGTCCGAGAGGCTGAC
GGAACAGAGAAACTGGTGTTTAGAGTGACTTCGAGTGAGAGTAAGGCACTGAAACATGAGCGGCCGAATTCTATA
AAGATTCTGGGAACTGCTATAAGTAACTATTGTAAAAAGACTCCAAGCAATAACATCACCTGTGTAACATATCAC
GTAAATATTGTTTTAGAAGAGGAGAGTACTAAGGATGCACAGAAAACATCTTGGTTGGTGTGTAACAGTGTGGGT
GGGCGAGGGATCAGTAGTAAGCTTGACTCTTTAGCTGATGAACTGAAATTTGTCCCAATCATTGGAATAGCCATG
CCTTTATCAAGCAGAGATGATGAAGCAAAAGGAGCAACGTCTGATTTCTCAGGAAAAGCATTTTGTTTCCTTCCT
TTACCACCTGGTGAGGAAAGCAGCACAGGCCTCCCAGTTCACATCAGTGGGTTCTTTGGCCTTACTGATAACCGC
AGGAGCATAAAATGGAGAGAGCTGGACCAGTGGAGAGACCCGGCAGCCTTATGGAATGAGTTTCTTGTCATGAAT
GTTGTCCCCAAAGCTTATGCTACTCTGGGATCCATCTTAAAAGAAGTGACATCTGTGGTGGAGCAAGCATGGAAG
CTTCCAGAATCGGAACGAAAAAAGATTATTAGGCGGTTGTATTTGAAATGGCATCCTGACAAAAATCCAGAGAAC
CATGACATTGCCAATGAAGTTTTTAAACATTTGCAGAATGAAATCAACAGATTAGAAAAACAGGCTTTTCTAGAT
CAAAATGCAGACAGGGCCTCCAGACGAACATTTTCAACCTCAGCATCCCGATTTCAGTCAGACAAATACTCATTT
CAGAGATTCTATACTTCATGGAATCAAGAAGCAACGAGCCATAAATCTGAAAGACAGCAACAGAACAAAGAAAAA
TGCCCCCCTTCAGCCGGACAGACTTACTCTCAAAGGTTCTTTGTTCCTCCCACTTTCAAGTCGGTTGGCAATCCA
GTGGAAGCACGCAGATGGCTAAGACAAGCCAGAGCAAACTTCTCAGCTGCCAGGAATGACCTTCATAAAAATGCC
AATGAGTGGGTGTGCTTTAAATGTTACCTTTCTACCAAGTTAGCTTTGATTGCAGCTGACTATGCTGTGAGGGGA
AAGTCTGATAAAGATGTAAAACCAACTGCACTTGCTCAGAAAATAGAGGAATATAGTCAGCAACTTGAAGGACTG
ACAAATGATGTTCACACATTGGAAGCTTATGGTGTAGACAGTTTAAAAACAAGATACCCTGATTTGCTTCCCTTT
CCTCAGATCCCAAATGACAGGTTCACTTCTGAGGTTGCTATGAGGGTGATGGAATGTACTGCCTGTATCATAATA
AAACTTGAAAATTTTATGCAACAAAAAGTGTGACTCGACGAATTCAATAAAAGATCTTTATTTTCATTAGATCTG
TGTGTTGGTTTTTTGTGTGCGGCCGCAGGAACCCCTAGTGATGGAGTTGGCCACTCCCTCTCTGCGCGCTCGCTC
GCTCACTGAGGCCGGGCGACCAAAGGTCGCCCGACGCCCGGGCTTTGCCCGGGCGGCCTCAGTGAGCGAGCGAGC
GCGCAGCTGCCTGCAGG

An exemplary vector comprising a Pcp2-L7 promoter and minisacsin with SRR1 is:

(SEQ ID NO: 75)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTTCTAGACAA
CTTTGTATAGAAAAGTTGGGTTCCACCCTCATGTTGGTTGATCTTCAACATTGTCCTGACTTCTTCCCACTTGAC
ATTCCTCAGGGTCCTGTGATCATGGCTGGGTCTAGTGAGGTTCAAACCTGCACTGCCCTACCCACACCCACACCC
AGCTCAGCGTCAGTCAGGATCAACAGTTACCTAGAGATCATCTTTCTGGGGCTTAAGCATTGGTGGGAGCAGATG
GGATATGAGTTGGGGATTTGGGGATGGGGGAAGATATCTGCTCCCCCTCCCCCTACACCCTAGCCTTTTAAAAGG
CCTTCTCAGGTCAGAGACCAGGAGAAAAGTATAGGAGAGATACACAATGGACCAGGAAGAAGAAAAGGGAGAGGG
AGGCTCAGACCTTCTAGACAAGGTAAGAGGGCTCTGGCTGACTCCACCATCCGCTTCTTGAGGTCTCGGCACCTG
TAATTGACAAGATTAATTCATTTATAGGGCATCTAATTAGCAAGTAAGTCTCTGGAGTCCCCTGACCCAGTTACT
ATAACACACAGGGGGTATAGGTAGGGGAGTATAAGAGCCCCTCCTCAGGGCAAATGAATGGATTCTTAGTACTGT
CCCCCAAGAGATAGTAGGTACTAGGATTTAGGGGCACTTCTGAGCCCCATTTCCCTGGTAAGTGTCCCAACCCCC
CAAATCAACCCAAGCCTGGTCTCAATCTAGGACAGTGGTAGAATGCTGTCCCTAGAGTCAGTACCATGTGAAATT
GTGCTGCAGGCAGGGGCCCCAGGCTGGGAGGTGGGGGTTGGGGGAGTCAGGGGCAGGTCAGGGAAGGAGACTCAG
GTTTCATTTAGAGAAATTCTGCAGACCCGTGAGGACTCAAGTTTGTACAAAAAAGCAGGCTGCCACCATGGAGAC
CAAGGAGAACAGGTGGGTCCCGGTGACCGTGCTCCCCGGCTGCGTGGGCTGCAGGACCGTCGCGGCGCTGGCGTC
CTGGACCGTGCGCGATGTGAAGGAACGTATCTTCGCGGAGACTGGCTTCCCGGTGTCGGAGCAGCGGCTGTGGCG
CGGCGGCCGCGAGTTATCTGACTGGATCAAGATTGGAGATCTGACTTCCAAAAATTGTCATCTTTTTGTAAACCT
TCAATCAAAAGGCTTAAAAGGGGGAGGTCGATTTGGTCAGACAACGCCACCACTTGTTGATTTTCTCAAGGACAT
TTTGAGAAGATATCCAGAAGGAGGACAGATTCTTAAGGAATTAATTCAGAATGCAGAAGATGCTGGGGCGACAGA
AGTTAAATTTTTATATGATGAAACTCAATACGGAACAGAGACTCTTTGGTCAAAAGATATGGCGCCATATCAGGG
GCCAGCTCTCTATGTGTACAACAACGCGGTTTTCACCCCAGAGGACTGGCACGGCATTCAAGAAATAGCAAGAAG
CAGGAAAAAGGATGATCCTCTGAAGGTCGGAAGATTTGGAATTGGGTTTAATTCTGTCTATCATATAACAGATGT
TCCTTGTATCTTTAGTGGTGACCAAATCGGGATGCTAGATCCTCATCAAACACTTTTTGGCCCACATGAATCAGG
CCAATGTTGGAATCTCAAAGATGACAGCAAAGAAATTAGTGAACTTTCAGACCAGTTTGCACCATTTGTTGGCAT
TTTTGGAAGCACCAAGGAAACATTTATAAACGGCAATTTTCCAGGAACATTTTTCCGTTTCCCTCTTCGCCTACA
ACCTTCACAACTTAGTAGTAACCTCTACAATAAGCAGAAGGTTCTTGAGTTGTTTGAGTCTTTTAGGGCAGATGC
AGACACAGTGCTGCTCTTTCTGAAAAGTGTGCAGGATGTTTCCTTATATGTCCGAGAGGCTGACGGAACAGAGAA
ACTGGTGTTTAGAGTGACTTCGAGTGAGAGTAAGGCACTGAAACATGAGCGGCCGAATTCTATAAAGATTCTGGG
AACTGCTATAAGTAACTATTGTAAAAAGACTCCAAGCAATAACATCACCTGTGTAACATATCACGTAAATATTGT
TTTAGAAGAGGAGAGTACTAAGGATGCACAGAAAACATCTTGGTTGGTGTGTAACAGTGTGGGTGGGCGAGGGAT
CAGTAGTAAGCTTGACTCTTTAGCTGATGAACTGAAATTTGTCCCAATCATTGGAATAGCCATGCCTTTATCAAG
CAGAGATGATGAAGCAAAAGGAGCAACGTCTGATTTCTCAGGAAAAGCATTTTGTTTCCTTCCTTTACCACCTGG
TGAGGAAAGCAGCACAGGCCTCCCAGTTCACATCAGTGGGTTCTTTGGCCTTACTGATAACCGCAGGAGCATAAA
ATGGAGAGAGCTGGACCAGTGGAGAGACCCGGCAGCCTTATGGAATGAGTTTCTTGTCATGAATGTTGTCCCCAA
AGCTTATGCTACTCTGGGATCCATCTTAAAAGAAGTGACATCTGTGGTGGAGCAAGCATGGAAGCTTCCAGAATC
GGAACGAAAAAAGATTATTAGGCGGTTGTATTTGAAATGGCATCCTGACAAAAATCCAGAGAACCATGACATTGC
CAATGAAGTTTTTAAACATTTGCAGAATGAAATCAACAGATTAGAAAAACAGGCTTTTCTAGATCAAAATGCAGA
CAGGGCCTCCAGACGAACATTTTCAACCTCAGCATCCCGATTTCAGTCAGACAAATACTCATTTCAGAGATTCTA
TACTTCATGGAATCAAGAAGCAACGAGCCATAAATCTGAAAGACAGCAACAGAACAAAGAAAAATGCCCCCCTTC
AGCCGGACAGACTTACTCTCAAAGGTTCTTTGTTCCTCCCACTTTCAAGTCGGTTGGCAATCCAGTGGAAGCACG
CAGATGGCTAAGACAAGCCAGAGCAAACTTCTCAGCTGCCAGGAATGACCTTCATAAAAATGCCAATGAGTGGGT
GTGCTTTAAATGTTACCTTTCTACCAAGTTAGCTTTGATTGCAGCTGACTATGCTGTGAGGGGAAAGTCTGATAA
AGATGTAAAACCAACTGCACTTGCTCAGAAAATAGAGGAATATAGTCAGCAACTTGAAGGACTGACAAATGATGT
TCACACATTGGAAGCTTATGGTGTAGACAGTTTAAAAACAAGATACCCTGATTTGCTTCCCTTTCCTCAGATCCC
AAATGACAGGTTCACTTCTGAGGTTGCTATGAGGGTGATGGAATGTACTGCCTGTATCATAATAAAACTTGAAAA
TTTTATGCAACAAAAAGTGTGAACCCAGCTTTCTTGTACAAAGTGGGAATTCCGATAATCAACCTCTGGATTACA
AAATTTGTGAAAGATTGACTGGTATTCTTAACTATGTTGCTCCTTTTACGCTATGTGGATACGCTGCTTTAATGC
CTTTGTATCATGCTATTGCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCTGACGCAACCCCCACTGGTT
GGGGCATTGCCACCACCTGTCAGCTCCTTTCCGGGACTTTCGCTTTCCCCCTCCCTATTGCCACGGCGGAACTCA
TCGCCGCCTGCCTTGCCCGCTGCTGGACAGGGGCTCGGCTGTTGGGCACTGACAATTCCGTGGTGTTGTCGGGGA
AGCTGACGTCCTTTCCATGGCTGCTCGCCTGTGTTGCCACCTGGATTCTGCGCGGGACGTCCTTCTGCTACGTCC
CTTCGGCCCTCAATCCAGCGGACCTTCCTTCCCGCGGCCTGCTGCCGGCTCTGCGGCCTCTTCCGCGTCTTCGCC
TTCGCCCTCAGACGAGTCGGATCTCCCTTTGGGCCGCCTCCCCGCATCGGGAATTCCTAGAGCTCGCTGATCAGC
CTCGACTGTGCCTTCTAGTTGCCAGCCATCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTCATTCTATTCTGGG
GGGTGGGGTGGGGCAGGACAGCAAGGGGGAGGATTGGGAAGAGAATAGCAGGCATGCTGGGGAGGGCCGCAGGAA
CCCCTAGTGATGGAGTTGGCCACTCCCTCTCTGCGCGCTCGCTCGCTCACTGAGGCCGGGCGACCAAAGGTCGCC
CGACGCCCGGGCTTTGCCCGGGCGGCCTCAGTGAGCGAGCGAGCGCGCAGCTGCCTGCAGG

An exemplary vector comprising a human neuronal synapsin1 promoter and minisacsin with SRR1 is:

(SEQ ID NO: 76)
CCTGCAGGCAGCTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCACGCGTGTGTCT
AGACTGCAGAGGGCCCTGCGTATGAGTGCAAGTGGGTTTTAGGACCAGGATGAGGCGGGGTGGGGGTGCCTACCT
GACGACCGACCCCGACCCACTGGACAAGCACCCAACCCCCATTCCCCAAATTGCGCATCCCCTATCAGAGAGGGG
GAGGGGAAACAGGATGCGGCGAGGCGCGTGCGCACTGCCAGCTTCAGCACCGCGGACAGTGCCTTCGCCCCCGCC
TGGCGGCGCGCGCCACCGCCGCCTCAGCACTGAAGGCGCGCTGACGTCACTCGCCGGTCCCCCGCAAACTCCCCT
TCCCGGCCACCTTGGTCGCGTCCGCGCCGCCGCCGGCCCAGCCGGACCGCACCACGCGAGGCGCGAGATAGGGGG
GCACGGGCGCGACCATCTGCGCTGCGGCGCCGGCGACTCAGCGCTGCCTCAGTCTGCGGTGGGCAGCGGAGGAGT
CGTGTCGTGCCTGAGAGCGCAGTCGAGAAGGTACCATGGAGACCAAGGAGAACAGGTGGGTCCCGGTGACCGTGC
TCCCCGGCTGCGTGGGCTGCAGGACCGTCGCGGCGCTGGCGTCCTGGACCGTGCGCGATGTGAAGGAACGTATCT
TCGCGGAGACTGGCTTCCCGGTGTCGGAGCAGCGGCTGTGGCGCGGCGGCCGCGAGTTATCTGACTGGATCAAGA
TTGGAGATCTGACTTCCAAAAATTGTCATCTTTTTGTAAACCTTCAATCAAAAGGCTTAAAAGGGGGAGGTCGAT
TTGGTCAGACAACGCCACCACTTGTTGATTTTCTCAAGGACATTTTGAGAAGATATCCAGAAGGAGGACAGATTC
TTAAGGAATTAATTCAGAATGCAGAAGATGCTGGGGCGACAGAAGTTAAATTTTTATATGATGAAACTCAATACG
GAACAGAGACTCTTTGGTCAAAAGATATGGCGCCATATCAGGGGCCAGCTCTCTATGTGTACAACAACGCGGTTT
TCACCCCAGAGGACTGGCACGGCATTCAAGAAATAGCAAGAAGCAGGAAAAAGGATGATCCTCTGAAGGTCGGAA
GATTTGGAATTGGGTTTAATTCTGTCTATCATATAACAGATGTTCCTTGTATCTTTAGTGGTGACCAAATCGGGA
TGCTAGATCCTCATCAAACACTTTTTGGCCCACATGAATCAGGCCAATGTTGGAATCTCAAAGATGACAGCAAAG
AAATTAGTGAACTTTCAGACCAGTTTGCACCATTTGTTGGCATTTTTGGAAGCACCAAGGAAACATTTATAAACG
GCAATTTTCCAGGAACATTTTTCCGTTTCCCTCTTCGCCTACAACCTTCACAACTTAGTAGTAACCTCTACAATA
AGCAGAAGGTTCTTGAGTTGTTTGAGTCTTTTAGGGCAGATGCAGACACAGTGCTGCTCTTTCTGAAAAGTGTGC
AGGATGTTTCCTTATATGTCCGAGAGGCTGACGGAACAGAGAAACTGGTGTTTAGAGTGACTTCGAGTGAGAGTA
AGGCACTGAAACATGAGCGGCCGAATTCTATAAAGATTCTGGGAACTGCTATAAGTAACTATTGTAAAAAGACTC
CAAGCAATAACATCACCTGTGTAACATATCACGTAAATATTGTTTTAGAAGAGGAGAGTACTAAGGATGCACAGA
AAACATCTTGGTTGGTGTGTAACAGTGTGGGTGGGCGAGGGATCAGTAGTAAGCTTGACTCTTTAGCTGATGAAC
TGAAATTTGTCCCAATCATTGGAATAGCCATGCCTTTATCAAGCAGAGATGATGAAGCAAAAGGAGCAACGTCTG
ATTTCTCAGGAAAAGCATTTTGTTTCCTTCCTTTACCACCTGGTGAGGAAAGCAGCACAGGCCTCCCAGTTCACA
TCAGTGGGTTCTTTGGCCTTACTGATAACCGCAGGAGCATAAAATGGAGAGAGCTGGACCAGTGGAGAGACCCGG
CAGCCTTATGGAATGAGTTTCTTGTCATGAATGTTGTCCCCAAAGCTTATGCTACTCTGGGATCCATCTTAAAAG
AAGTGACATCTGTGGTGGAGCAAGCATGGAAGCTTCCAGAATCGGAACGAAAAAAGATTATTAGGCGGTTGTATT
TGAAATGGCATCCTGACAAAAATCCAGAGAACCATGACATTGCCAATGAAGTTTTTAAACATTTGCAGAATGAAA
TCAACAGATTAGAAAAACAGGCTTTTCTAGATCAAAATGCAGACAGGGCCTCCAGACGAACATTTTCAACCTCAG
CATCCCGATTTCAGTCAGACAAATACTCATTTCAGAGATTCTATACTTCATGGAATCAAGAAGCAACGAGCCATA
AATCTGAAAGACAGCAACAGAACAAAGAAAAATGCCCCCCTTCAGCCGGACAGACTTACTCTCAAAGGTTCTTTG
TTCCTCCCACTTTCAAGTCGGTTGGCAATCCAGTGGAAGCACGCAGATGGCTAAGACAAGCCAGAGCAAACTTCT
CAGCTGCCAGGAATGACCTTCATAAAAATGCCAATGAGTGGGTGTGCTTTAAATGTTACCTTTCTACCAAGTTAG
CTTTGATTGCAGCTGACTATGCTGTGAGGGGAAAGTCTGATAAAGATGTAAAACCAACTGCACTTGCTCAGAAAA
TAGAGGAATATAGTCAGCAACTTGAAGGACTGACAAATGATGTTCACACATTGGAAGCTTATGGTGTAGACAGTT
TAAAAACAAGATACCCTGATTTGCTTCCCTTTCCTCAGATCCCAAATGACAGGTTCACTTCTGAGGTTGCTATGA
GGGTGATGGAATGTACTGCCTGTATCATAATAAAACTTGAAAATTTTATGCAACAAAAAGTGTGAGAATTCGATA
TCAAGCTTATCGATAATCAACCTCTGGATTACAAAATTTGTGAAAGATTGACTGGTATTCTTAACTATGTTGCTC
CTTTTACGCTATGTGGATACGCTGCTTTAATGCCTTTGTATCATGCTATTGCTTCCCGTATGGCTTTCATTTTCT
CCTCCTTGTATAAATCCTGGTTGCTGTCTCTTTATGAGGAGTTGTGGCCCGTTGTCAGGCAACGTGGCGTGGTGT
GCACTGTGTTTGCTGACGCAACCCCCACTGGTTGGGGCATTGCCACCACCTGTCAGCTCCTTTCCGGGACTTTCG
CTTTCCCCCTCCCTATTGCCACGGCGGAACTCATCGCCGCCTGCCTTGCCCGCTGCTGGACAGGGGCTCGGCTGT
TGGGCACTGACAATTCCGTGGTGTTGTCGGGGAAATCATCGTCCTTTCCTTGGCTGCTCGCCTATGTTGCCACCT
GGATTCTGCGCGGGACGTCCTTCTGCTACGTCCCTTCGGCCCTCAATCCAGCGGACCTTCCTTCCCGCGGCCTGC
TGCCGGCTCTGCGGCCTCTTCCGCGTCTTCGCCTTCGCCCTCAGACGAGTCGGATCTCCCTTTGGGCCGCCTCCC
CGCATCGATACCGAGCGCTGCTCGAGAGATCTACGGGTGGCATCCCTGTGACCCCTCCCCAGTGCCTCTCCTGGC
CCTGGAAGTTGCCACTCCAGTGCCCACCAGCCTTGTCCTAATAAAATTAAGTTGCATCATTTTGTCTGACTAGGT
GTCCTTCTATAATATTATGGGGTGGAGGGGGGTGGTATGGAGCAAGGGGCAAGTTGGGAAGACAACCTGTAGGGC
CTGCGGGGTCTATTGGGAACCAAGCTGGAGTGCAGTGGCACAATCTTGGCTCACTGCAATCTCCGCCTCCTGGGT
TCAAGCGATTCTCCTGCCTCAGCCTCCCGAGTTGTTGGGATTCCAGGCATGCATGACCAGGCTCAGCTAATTTTT
GTTTTTTTGGTAGAGACGGGGTTTCACCATATTGGCCAGGCTGGTCTCCAACTCCTAATCTCAGGTGATCTACCC
ACCTTGGCCTCCCAAATTGCTGGGATTACAGGCGTGAACCACTGCTCCCTTCCCTGTCCTTCTGATTTTGTAGGT
AACCACGTGCGGACCGAGCGGCCGCAGGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTCAGTGAGCGAGCGAGCG
CGCAGCTGCCTGCAGG

An exemplary vector comprising a CMV enhancer, CBA promoter, β-globin intron, minisacsin with SRR1 and hGh polyadenylation sequence is:

(SEQ ID NO: 77)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
ACGCGCTAGTTATTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGGTCGA
GGTGAGCCCCACGTTCTGCTTCACTCTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGCCAGGCGGGGCGGGGCGGGGCGAGGG
GCGGGGCGGGGCGAGGCGGAGAGGTGCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGCGGGGAGTCGCTGCGACGCTGCCTTCGC
CCCGTGCCCCGCTCCGCCGCCGCCTCGCGCCGCCCGCCCCGGCTCTGACTGACCGCGTTACTCCCACAGGTGAGC
GGGCGGGACGGCCCTTCTCCTCCGGGCTGTAATTAGCCCGTTTAGTGAACCGTCAGATCGCCTGGAGACGCCATC
CACGCTGTTTTGACCTCCATAGAAGACACCGGGACCGATCCAGCCTCCGCGGATTCGAATCCCGGCCGGGAACGG
TGCATTGGAACGCGGATTCCCCGTGCCAAGAGTGACGTAAGTACCGCCTATAGAGTCTATAGGCCCACAAAAAAT
GCTTTCTTCTTTTAATATACTTTTTTGTTTATCTTATTTCTAATACTTTCCCTAATCTCTTTCTTTCAGGGCAAT
AATGATACAATGTATCATGCCTCTTTGCACCATTCTAAAGAATAACAGTGATAATTTCTGGGTTAAGGCAATAGC
AATATTTCTGCATATAAATATTTCTGCATATAAATTGTAACTGATGTAAGAGGTTTCATATTGCTAATAGCAGCT
ACAATCCAGCTACCATTCTGCTTTTATTTTATGGTTGGGATAAGGCTGGATTATTCTGAGTCCAAGCTAGGCCCT
TTTGCTAATCATGTTCATACCTCTTATCTTCCTCCCACAGCTCCTGGGCAACGTGCTGGTCTGTGTGCTGGCCCA
TCACTTTGGCAAAGAATTGGGATTCGAACACCGGTCGACGAATTCGTTAACGGATCCGCTAGCGCCGCCGCGATC
GCCATGGAGACCAAGGAGAACAGGTGGGTCCCGGTGACCGTGCTCCCCGGCTGCGTGGGCTGCAGGACCGTCGCG
GCGCTGGCGTCCTGGACCGTGCGCGATGTGAAGGAACGTATCTTCGCGGAGACTGGCTTCCCGGTGTCGGAGCAG
CGGCTGTGGCGCGGCGGCCGCGAGTTATCTGACTGGATCAAGATTGGAGATCTGACTTCCAAAAATTGTCATCTT
TTTGTAAACCTTCAATCAAAAGGCTTAAAAGGGGGAGGTCGATTTGGTCAGACAACGCCACCACTTGTTGATTTT
CTCAAGGACATTTTGAGAAGATATCCAGAAGGAGGACAGATTCTTAAGGAATTAATTCAGAATGCAGAAGATGCT
GGGGCGACAGAAGTTAAATTTTTATATGATGAAACTCAATACGGAACAGAGACTCTTTGGTCAAAAGATATGGCG
CCATATCAGGGGCCAGCTCTCTATGTGTACAACAACGCGGTTTTCACCCCAGAGGACTGGCACGGCATTCAAGAA
ATAGCAAGAAGCAGGAAAAAGGATGATCCTCTGAAGGTCGGAAGATTTGGAATTGGGTTTAATTCTGTCTATCAT
ATAACAGATGTTCCTTGTATCTTTAGTGGTGACCAAATCGGGATGCTAGATCCTCATCAAACACTTTTTGGCCCA
CATGAATCAGGCCAATGTTGGAATCTCAAAGATGACAGCAAAGAAATTAGTGAACTTTCAGACCAGTTTGCACCA
TTTGTTGGCATTTTTGGAAGCACCAAGGAAACATTTATAAACGGCAATTTTCCAGGAACATTTTTCCGTTTCCCT
CTTCGCCTACAACCTTCACAACTTAGTAGTAACCTCTACAATAAGCAGAAGGTTCTTGAGTTGTTTGAGTCTTTT
AGGGCAGATGCAGACACAGTGCTGCTCTTTCTGAAAAGTGTGCAGGATGTTTCCTTATATGTCCGAGAGGCTGAC
GGAACAGAGAAACTGGTGTTTAGAGTGACTTCGAGTGAGAGTAAGGCACTGAAACATGAGCGGCCGAATTCTATA
AAGATTCTGGGAACTGCTATAAGTAACTATTGTAAAAAGACTCCAAGCAATAACATCACCTGTGTAACATATCAC
GTAAATATTGTTTTAGAAGAGGAGAGTACTAAGGATGCACAGAAAACATCTTGGTTGGTGTGTAACAGTGTGGGT
GGGCGAGGGATCAGTAGTAAGCTTGACTCTTTAGCTGATGAACTGAAATTTGTCCCAATCATTGGAATAGCCATG
CCTTTATCAAGCAGAGATGATGAAGCAAAAGGAGCAACGTCTGATTTCTCAGGAAAAGCATTTTGTTTCCTTCCT
TTACCACCTGGTGAGGAAAGCAGCACAGGCCTCCCAGTTCACATCAGTGGGTTCTTTGGCCTTACTGATAACCGC
AGGAGCATAAAATGGAGAGAGCTGGACCAGTGGAGAGACCCGGCAGCCTTATGGAATGAGTTTCTTGTCATGAAT
GTTGTCCCCAAAGCTTATGCTACTCTGGGATCCATCTTAAAAGAAGTGACATCTGTGGTGGAGCAAGCATGGAAG
CTTCCAGAATCGGAACGAAAAAAGATTATTAGGCGGTTGTATTTGAAATGGCATCCTGACAAAAATCCAGAGAAC
CATGACATTGCCAATGAAGTTTTTAAACATTTGCAGAATGAAATCAACAGATTAGAAAAACAGGCTTTTCTAGAT
CAAAATGCAGACAGGGCCTCCAGACGAACATTTTCAACCTCAGCATCCCGATTTCAGTCAGACAAATACTCATTT
CAGAGATTCTATACTTCATGGAATCAAGAAGCAACGAGCCATAAATCTGAAAGACAGCAACAGAACAAAGAAAAA
TGCCCCCCTTCAGCCGGACAGACTTACTCTCAAAGGTTCTTTGTTCCTCCCACTTTCAAGTCGGTTGGCAATCCA
GTGGAAGCACGCAGATGGCTAAGACAAGCCAGAGCAAACTTCTCAGCTGCCAGGAATGACCTTCATAAAAATGCC
AATGAGTGGGTGTGCTTTAAATGTTACCTTTCTACCAAGTTAGCTTTGATTGCAGCTGACTATGCTGTGAGGGGA
AAGTCTGATAAAGATGTAAAACCAACTGCACTTGCTCAGAAAATAGAGGAATATAGTCAGCAACTTGAAGGACTG
ACAAATGATGTTCACACATTGGAAGCTTATGGTGTAGACAGTTTAAAAACAAGATACCCTGATTTGCTTCCCTTT
CCTCAGATCCCAAATGACAGGTTCACTTCTGAGGTTGCTATGAGGGTGATGGAATGTACTGCCTGTATCATAATA
AAACTTGAAAATTTTATGCAACAAAAAGTGGGTAAGCCTATCCCTAACCCTCTCCTCGGTCTCGATTCTACGTGA
CTCGAGAGATCTACGGGTGGCATCCCTGTGACCCCTCCCCAGTGCCTCTCCTGGCCCTGGAAGTTGCCACTCCAG
TGCCCACCAGCCTTGTCCTAATAAAATTAAGTTGCATCATTTTGTCTGACTAGGTGTCCTTCTATAATATTATGG
GGTGGAGGGGGGTGGTATGGAGCAAGGGGCAAGTTGGGAAGACAACCTGTAGGGCCTGCGGGGTCTATTGGGAAC
CAAGCTGGAGTGCAGTGGCACAATCTTGGCTCACTGCAATCTCCGCCTCCTGGGTTCAAGCGATTCTCCTGCCTC
AGCCTCCCGAGTTGTTGGGATTCCAGGCATGCATGACCAGGCTCAGCTAATTTTTGTTTTTTTGGTAGAGACGGG
GTTTCACCATATTGGCCAGGCTGGTCTCCAACTCCTAATCTCAGGTGATCTACCCACCTTGGCCTCCCAAATTGC
TGGGATTACAGGCGTGAACCACTGCTCCCTTCCCTGTCCTTCTGATTTTGTAGGTAACCACGTGCGGACCGAGCG
GCCGCAGGAACCCCTAGTGATGGAGTTGGCCACTCCCTCTCTGCGCGCTCGCTCGCTCACTGAGGCCGGGCGACC
AAAGGTCGCCCGACGCCCGGGCTTTGCCCGGGCGGCCTCAGTGAGCGAGCGAGCGCGCAGCTGCCTGCAGG

An exemplary vector comprising a CMV enhancer, CBA promoter, β-globin intron, minisacsin with SRR1 and pAmin polyadenylation sequence is:

(SEQ ID NO: 78)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTGCGGCCGCA
ACGCGCTAGTTATTAATAGTAATCAATTACGGGGTCATTAGTTCATAGCCCATATATGGAGTTCCGCGTTACATA
ACTTACGGTAAATGGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGAGTATTTACGGTAAACTGCCCACTTGGCAGT
ACATCAAGTGTATCATATGCCAAGTACGCCCCCTATTGACGTCAATGACGGTAAATGGCCCGCCTGGCATTATGC
CCAGTACATGACCTTATGGGACTTTCCTACTTGGCAGTACATCTACGTATTAGTCATCGCTATTACCATGGTCGA
GGTGAGCCCCACGTTCTGCTTCACTCTCCCCATCTCCCCCCCCTCCCCACCCCCAATTTTGTATTTATTTATTTT
TTAATTATTTTGTGCAGCGATGGGGGCGGGGGGGGGGGGGGGGCGCGCGCCAGGCGGGGGGGGGCGGGGCGAGGG
GCGGGGCGGGGCGAGGCGGAGAGGTGCGGCGGCAGCCAATCAGAGCGGCGCGCTCCGAAAGTTTCCTTTTATGGC
GAGGCGGCGGCGGCGGCGGCCCTATAAAAAGCGAAGCGCGCGGCGGGGGGGGAGTCGCTGCGACGCTGCCTTCGC
CCCGTGCCCCGCTCCGCCGCCGCCTCGCGCCGCCCGCCCCGGCTCTGACTGACCGCGTTACTCCCACAGGTGAGC
GGGCGGGACGGCCCTTCTCCTCCGGGCTGTAATTAGCCCGTTTAGTGAACCGTCAGATCGCCTGGAGACGCCATC
CACGCTGTTTTGACCTCCATAGAAGACACCGGGACCGATCCAGCCTCCGCGGATTCGAATCCCGGCCGGGAACGG
TGCATTGGAACGCGGATTCCCCGTGCCAAGAGTGACGTAAGTACCGCCTATAGAGTCTATAGGCCCACAAAAAAT
GCTTTCTTCTTTTAATATACTTTTTTGTTTATCTTATTTCTAATACTTTCCCTAATCTCTTTCTTTCAGGGCAAT
AATGATACAATGTATCATGCCTCTTTGCACCATTCTAAAGAATAACAGTGATAATTTCTGGGTTAAGGCAATAGC
AATATTTCTGCATATAAATATTTCTGCATATAAATTGTAACTGATGTAAGAGGTTTCATATTGCTAATAGCAGCT
ACAATCCAGCTACCATTCTGCTTTTATTTTATGGTTGGGATAAGGCTGGATTATTCTGAGTCCAAGCTAGGCCCT
TTTGCTAATCATGTTCATACCTCTTATCTTCCTCCCACAGCTCCTGGGCAACGTGCTGGTCTGTGTGCTGGCCCA
TCACTTTGGCAAAGAATTGGGATTCGAACACCGGTCGACGAATTCGTTAACGGATCCGCTAGCGCCGCCGCGATC
GCCATGGAGACCAAGGAGAACAGGTGGGTCCCGGTGACCGTGCTCCCCGGCTGCGTGGGCTGCAGGACCGTCGCG
GCGCTGGCGTCCTGGACCGTGCGCGATGTGAAGGAACGTATCTTCGCGGAGACTGGCTTCCCGGTGTCGGAGCAG
CGGCTGTGGCGCGGCGGCCGCGAGTTATCTGACTGGATCAAGATTGGAGATCTGACTTCCAAAAATTGTCATCTT
TTTGTAAACCTTCAATCAAAAGGCTTAAAAGGGGGAGGTCGATTTGGTCAGACAACGCCACCACTTGTTGATTTT
CTCAAGGACATTTTGAGAAGATATCCAGAAGGAGGACAGATTCTTAAGGAATTAATTCAGAATGCAGAAGATGCT
GGGGCGACAGAAGTTAAATTTTTATATGATGAAACTCAATACGGAACAGAGACTCTTTGGTCAAAAGATATGGCG
CCATATCAGGGGCCAGCTCTCTATGTGTACAACAACGCGGTTTTCACCCCAGAGGACTGGCACGGCATTCAAGAA
ATAGCAAGAAGCAGGAAAAAGGATGATCCTCTGAAGGTCGGAAGATTTGGAATTGGGTTTAATTCTGTCTATCAT
ATAACAGATGTTCCTTGTATCTTTAGTGGTGACCAAATCGGGATGCTAGATCCTCATCAAACACTTTTTGGCCCA
CATGAATCAGGCCAATGTTGGAATCTCAAAGATGACAGCAAAGAAATTAGTGAACTTTCAGACCAGTTTGCACCA
TTTGTTGGCATTTTTGGAAGCACCAAGGAAACATTTATAAACGGCAATTTTCCAGGAACATTTTTCCGTTTCCCT
CTTCGCCTACAACCTTCACAACTTAGTAGTAACCTCTACAATAAGCAGAAGGTTCTTGAGTTGTTTGAGTCTTTT
AGGGCAGATGCAGACACAGTGCTGCTCTTTCTGAAAAGTGTGCAGGATGTTTCCTTATATGTCCGAGAGGCTGAC
GGAACAGAGAAACTGGTGTTTAGAGTGACTTCGAGTGAGAGTAAGGCACTGAAACATGAGCGGCCGAATTCTATA
AAGATTCTGGGAACTGCTATAAGTAACTATTGTAAAAAGACTCCAAGCAATAACATCACCTGTGTAACATATCAC
GTAAATATTGTTTTAGAAGAGGAGAGTACTAAGGATGCACAGAAAACATCTTGGTTGGTGTGTAACAGTGTGGGT
GGGCGAGGGATCAGTAGTAAGCTTGACTCTTTAGCTGATGAACTGAAATTTGTCCCAATCATTGGAATAGCCATG
CCTTTATCAAGCAGAGATGATGAAGCAAAAGGAGCAACGTCTGATTTCTCAGGAAAAGCATTTTGTTTCCTTCCT
TTACCACCTGGTGAGGAAAGCAGCACAGGCCTCCCAGTTCACATCAGTGGGTTCTTTGGCCTTACTGATAACCGC
AGGAGCATAAAATGGAGAGAGCTGGACCAGTGGAGAGACCCGGCAGCCTTATGGAATGAGTTTCTTGTCATGAAT
GTTGTCCCCAAAGCTTATGCTACTCTGGGATCCATCTTAAAAGAAGTGACATCTGTGGTGGAGCAAGCATGGAAG
CTTCCAGAATCGGAACGAAAAAAGATTATTAGGCGGTTGTATTTGAAATGGCATCCTGACAAAAATCCAGAGAAC
CATGACATTGCCAATGAAGTTTTTAAACATTTGCAGAATGAAATCAACAGATTAGAAAAACAGGCTTTTCTAGAT
CAAAATGCAGACAGGGCCTCCAGACGAACATTTTCAACCTCAGCATCCCGATTTCAGTCAGACAAATACTCATTT
CAGAGATTCTATACTTCATGGAATCAAGAAGCAACGAGCCATAAATCTGAAAGACAGCAACAGAACAAAGAAAAA
TGCCCCCCTTCAGCCGGACAGACTTACTCTCAAAGGTTCTTTGTTCCTCCCACTTTCAAGTCGGTTGGCAATCCA
GTGGAAGCACGCAGATGGCTAAGACAAGCCAGAGCAAACTTCTCAGCTGCCAGGAATGACCTTCATAAAAATGCC
AATGAGTGGGTGTGCTTTAAATGTTACCTTTCTACCAAGTTAGCTTTGATTGCAGCTGACTATGCTGTGAGGGGA
AAGTCTGATAAAGATGTAAAACCAACTGCACTTGCTCAGAAAATAGAGGAATATAGTCAGCAACTTGAAGGACTG
ACAAATGATGTTCACACATTGGAAGCTTATGGTGTAGACAGTTTAAAAACAAGATACCCTGATTTGCTTCCCTTT
CCTCAGATCCCAAATGACAGGTTCACTTCTGAGGTTGCTATGAGGGTGATGGAATGTACTGCCTGTATCATAATA
AAACTTGAAAATTTTATGCAACAAAAAGTGGGTAAGCCTATCCCTAACCCTCTCCTCGGTCTCGATTCTACGTGA
CTCGACGAATTCAATAAAAGATCTTTATTTTCATTAGATCTGTGTGTTGGTTTTTTGTGTGCGGCCGCAGGAACC
CCTAGTGATGGAGTTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGCTGCCTGCAGG

An exemplary vector comprising a Pcp2-L7 promoter and minisacsin with SRR1 is:

(SEQ ID NO: 79)
CCTGCAGGCAGCTGCGCGCTCGCTCGCTCACTGAGGCCGCCCGGGCAAAGCCCGGGCGTCGGGCGACCTTTGGTC
GCCCGGCCTCAGTGAGCGAGCGAGCGCGCAGAGAGGGAGTGGCCAACTCCATCACTAGGGGTTCCTTCTAGACAA
CTTTGTATAGAAAAGTTGGGTTCCACCCTCATGTTGGTTGATCTTCAACATTGTCCTGACTTCTTCCCACTTGAC
ATTCCTCAGGGTCCTGTGATCATGGCTGGGTCTAGTGAGGTTCAAACCTGCACTGCCCTACCCACACCCACACCC
AGCTCAGCGTCAGTCAGGATCAACAGTTACCTAGAGATCATCTTTCTGGGGCTTAAGCATTGGTGGGAGCAGATG
GGATATGAGTTGGGGATTTGGGGATGGGGGAAGATATCTGCTCCCCCTCCCCCTACACCCTAGCCTTTTAAAAGG
CCTTCTCAGGTCAGAGACCAGGAGAAAAGTATAGGAGAGATACACAATGGACCAGGAAGAAGAAAAGGGAGAGGG
AGGCTCAGACCTTCTAGACAAGGTAAGAGGGCTCTGGCTGACTCCACCATCCGCTTCTTGAGGTCTCGGCACCTG
TAATTGACAAGATTAATTCATTTATAGGGCATCTAATTAGCAAGTAAGTCTCTGGAGTCCCCTGACCCAGTTACT
ATAACACACAGGGGGTATAGGTAGGGGAGTATAAGAGCCCCTCCTCAGGGCAAATGAATGGATTCTTAGTACTGT
CCCCCAAGAGATAGTAGGTACTAGGATTTAGGGGCACTTCTGAGCCCCATTTCCCTGGTAAGTGTCCCAACCCCC
CAAATCAACCCAAGCCTGGTCTCAATCTAGGACAGTGGTAGAATGCTGTCCCTAGAGTCAGTACCATGTGAAATT
GTGCTGCAGGCAGGGGCCCCAGGCTGGGAGGTGGGGGTTGGGGGAGTCAGGGGCAGGTCAGGGAAGGAGACTCAG
GTTTCATTTAGAGAAATTCTGCAGACCCGTGAGGACTCAAGTTTGTACAAAAAAGCAGGCTGCCACCATGGAGAC
CAAGGAGAACAGGTGGGTCCCGGTGACCGTGCTCCCCGGCTGCGTGGGCTGCAGGACCGTCGCGGCGCTGGCGTC
CTGGACCGTGCGCGATGTGAAGGAACGTATCTTCGCGGAGACTGGCTTCCCGGTGTCGGAGCAGCGGCTGTGGCG
CGGCGGCCGCGAGTTATCTGACTGGATCAAGATTGGAGATCTGACTTCCAAAAATTGTCATCTTTTTGTAAACCT
TCAATCAAAAGGCTTAAAAGGGGGAGGTCGATTTGGTCAGACAACGCCACCACTTGTTGATTTTCTCAAGGACAT
TTTGAGAAGATATCCAGAAGGAGGACAGATTCTTAAGGAATTAATTCAGAATGCAGAAGATGCTGGGGCGACAGA
AGTTAAATTTTTATATGATGAAACTCAATACGGAACAGAGACTCTTTGGTCAAAAGATATGGCGCCATATCAGGG
GCCAGCTCTCTATGTGTACAACAACGCGGTTTTCACCCCAGAGGACTGGCACGGCATTCAAGAAATAGCAAGAAG
CAGGAAAAAGGATGATCCTCTGAAGGTCGGAAGATTTGGAATTGGGTTTAATTCTGTCTATCATATAACAGATGT
TCCTTGTATCTTTAGTGGTGACCAAATCGGGATGCTAGATCCTCATCAAACACTTTTTGGCCCACATGAATCAGG
CCAATGTTGGAATCTCAAAGATGACAGCAAAGAAATTAGTGAACTTTCAGACCAGTTTGCACCATTTGTTGGCAT
TTTTGGAAGCACCAAGGAAACATTTATAAACGGCAATTTTCCAGGAACATTTTTCCGTTTCCCTCTTCGCCTACA
ACCTTCACAACTTAGTAGTAACCTCTACAATAAGCAGAAGGTTCTTGAGTTGTTTGAGTCTTTTAGGGCAGATGC
AGACACAGTGCTGCTCTTTCTGAAAAGTGTGCAGGATGTTTCCTTATATGTCCGAGAGGCTGACGGAACAGAGAA
ACTGGTGTTTAGAGTGACTTCGAGTGAGAGTAAGGCACTGAAACATGAGCGGCCGAATTCTATAAAGATTCTGGG
AACTGCTATAAGTAACTATTGTAAAAAGACTCCAAGCAATAACATCACCTGTGTAACATATCACGTAAATATTGT
TTTAGAAGAGGAGAGTACTAAGGATGCACAGAAAACATCTTGGTTGGTGTGTAACAGTGTGGGTGGGCGAGGGAT
CAGTAGTAAGCTTGACTCTTTAGCTGATGAACTGAAATTTGTCCCAATCATTGGAATAGCCATGCCTTTATCAAG
CAGAGATGATGAAGCAAAAGGAGCAACGTCTGATTTCTCAGGAAAAGCATTTTGTTTCCTTCCTTTACCACCTGG
TGAGGAAAGCAGCACAGGCCTCCCAGTTCACATCAGTGGGTTCTTTGGCCTTACTGATAACCGCAGGAGCATAAA
ATGGAGAGAGCTGGACCAGTGGAGAGACCCGGCAGCCTTATGGAATGAGTTTCTTGTCATGAATGTTGTCCCCAA
AGCTTATGCTACTCTGGGATCCATCTTAAAAGAAGTGACATCTGTGGTGGAGCAAGCATGGAAGCTTCCAGAATC
GGAACGAAAAAAGATTATTAGGCGGTTGTATTTGAAATGGCATCCTGACAAAAATCCAGAGAACCATGACATTGC
CAATGAAGTTTTTAAACATTTGCAGAATGAAATCAACAGATTAGAAAAACAGGCTTTTCTAGATCAAAATGCAGA
CAGGGCCTCCAGACGAACATTTTCAACCTCAGCATCCCGATTTCAGTCAGACAAATACTCATTTCAGAGATTCTA
TACTTCATGGAATCAAGAAGCAACGAGCCATAAATCTGAAAGACAGCAACAGAACAAAGAAAAATGCCCCCCTTC
AGCCGGACAGACTTACTCTCAAAGGTTCTTTGTTCCTCCCACTTTCAAGTCGGTTGGCAATCCAGTGGAAGCACG
CAGATGGCTAAGACAAGCCAGAGCAAACTTCTCAGCTGCCAGGAATGACCTTCATAAAAATGCCAATGAGTGGGT
GTGCTTTAAATGTTACCTTTCTACCAAGTTAGCTTTGATTGCAGCTGACTATGCTGTGAGGGGAAAGTCTGATAA
AGATGTAAAACCAACTGCACTTGCTCAGAAAATAGAGGAATATAGTCAGCAACTTGAAGGACTGACAAATGATGT
TCACACATTGGAAGCTTATGGTGTAGACAGTTTAAAAACAAGATACCCTGATTTGCTTCCCTTTCCTCAGATCCC
AAATGACAGGTTCACTTCTGAGGTTGCTATGAGGGTGATGGAATGTACTGCCTGTATCATAATAAAACTTGAAAA
TTTTATGCAACAAAAAGTGGGTAAGCCTATCCCTAACCCTCTCCTCGGTCTCGATTCTACGTGAACCCAGCTTTC
TTGTACAAAGTGGGAATTCCGATAATCAACCTCTGGATTACAAAATTTGTGAAAGATTGACTGGTATTCTTAACT
ATGTTGCTCCTTTTACGCTATGTGGATACGCTGCTTTAATGCCTTTGTATCATGCTATTGCTTCCCGTATGGCTT
TCATTTTCTCCTCCTTGTATAAATCCTGGTTGCTGTCTCTTTATGAGGAGTTGTGGCCCGTTGTCAGGCAACGTG
GCGTGGTGTGCACTGTGTTTGCTGACGCAACCCCCACTGGTTGGGGCATTGCCACCACCTGTCAGCTCCTTTCCG
GGACTTTCGCTTTCCCCCTCCCTATTGCCACGGCGGAACTCATCGCCGCCTGCCTTGCCCGCTGCTGGACAGGGG
CTCGGCTGTTGGGCACTGACAATTCCGTGGTGTTGTCGGGGAAGCTGACGTCCTTTCCATGGCTGCTCGCCTGTG
TTGCCACCTGGATTCTGCGCGGGACGTCCTTCTGCTACGTCCCTTCGGCCCTCAATCCAGCGGACCTTCCTTCCC
GCGGCCTGCTGCCGGCTCTGCGGCCTCTTCCGCGTCTTCGCCTTCGCCCTCAGACGAGTCGGATCTCCCTTTGGG
CCGCCTCCCCGCATCGGGAATTCCTAGAGCTCGCTGATCAGCCTCGACTGTGCCTTCTAGTTGCCAGCCATCTGT
TGTTTGCCCCTCCCCCGTGCCTTCCTTGACCCTGGAAGGTGCCACTCCCACTGTCCTTTCCTAATAAAATGAGGA
AATTGCATCGCATTGTCTGAGTAGGTGTCATTCTATTCTGGGGGGTGGGGTGGGGCAGGACAGCAAGGGGGAGGA
TTGGGAAGAGAATAGCAGGCATGCTGGGGAGGGCCGCAGGAACCCCTAGTGATGGAGTTGGCCACTCCCTCTCTG
CGCGCTCGCTCGCTCACTGAGGCCGGGCGACCAAAGGTCGCCCGACGCCCGGGCTTTGCCCGGGCGGCCTCAGTG
AGCGAGCGAGCGCGCAGCTGCCTGCAGG

An exemplary vector comprising a human neuronal synapsin1 promoter and minisacsin with SRR1 is:

(SEQ ID NO: 80)
CCTGCAGGCAGCTGCGCGCTCGCTCGCTCACTGAGGCCGCCCGGGCGTCGGGCGACCTTTGGTCGCCCGGCCTCA
GTGAGCGAGCGAGCGCGCAGAGAGGGAGTGGCCAACTCCATCACTAGGGGTTCCTGCGGCCGCACGCGTGTGTCT
AGACTGCAGAGGGCCCTGCGTATGAGTGCAAGTGGGTTTTAGGACCAGGATGAGGCGGGGTGGGGGTGCCTACCT
GACGACCGACCCCGACCCACTGGACAAGCACCCAACCCCCATTCCCCAAATTGCGCATCCCCTATCAGAGAGGGG
GAGGGGAAACAGGATGCGGCGAGGCGCGTGCGCACTGCCAGCTTCAGCACCGCGGACAGTGCCTTCGCCCCCGCC
TGGCGGCGCGCGCCACCGCCGCCTCAGCACTGAAGGCGCGCTGACGTCACTCGCCGGTCCCCCGCAAACTCCCCT
TCCCGGCCACCTTGGTCGCGTCCGCGCCGCCGCCGGCCCAGCCGGACCGCACCACGCGAGGCGCGAGATAGGGGG
GCACGGGCGCGACCATCTGCGCTGCGGCGCCGGCGACTCAGCGCTGCCTCAGTCTGCGGTGGGCAGCGGAGGAGT
CGTGTCGTGCCTGAGAGCGCAGTCGAGAAGGTACCATGGAGACCAAGGAGAACAGGTGGGTCCCGGTGACCGTGC
TCCCCGGCTGCGTGGGCTGCAGGACCGTCGCGGCGCTGGCGTCCTGGACCGTGCGCGATGTGAAGGAACGTATCT
TCGCGGAGACTGGCTTCCCGGTGTCGGAGCAGCGGCTGTGGCGCGGCGGCCGCGAGTTATCTGACTGGATCAAGA
TTGGAGATCTGACTTCCAAAAATTGTCATCTTTTTGTAAACCTTCAATCAAAAGGCTTAAAAGGGGGAGGTCGAT
TTGGTCAGACAACGCCACCACTTGTTGATTTTCTCAAGGACATTTTGAGAAGATATCCAGAAGGAGGACAGATTC
TTAAGGAATTAATTCAGAATGCAGAAGATGCTGGGGCGACAGAAGTTAAATTTTTATATGATGAAACTCAATACG
GAACAGAGACTCTTTGGTCAAAAGATATGGCGCCATATCAGGGGCCAGCTCTCTATGTGTACAACAACGCGGTTT
TCACCCCAGAGGACTGGCACGGCATTCAAGAAATAGCAAGAAGCAGGAAAAAGGATGATCCTCTGAAGGTCGGAA
GATTTGGAATTGGGTTTAATTCTGTCTATCATATAACAGATGTTCCTTGTATCTTTAGTGGTGACCAAATCGGGA
TGCTAGATCCTCATCAAACACTTTTTGGCCCACATGAATCAGGCCAATGTTGGAATCTCAAAGATGACAGCAAAG
AAATTAGTGAACTTTCAGACCAGTTTGCACCATTTGTTGGCATTTTTGGAAGCACCAAGGAAACATTTATAAACG
GCAATTTTCCAGGAACATTTTTCCGTTTCCCTCTTCGCCTACAACCTTCACAACTTAGTAGTAACCTCTACAATA
AGCAGAAGGTTCTTGAGTTGTTTGAGTCTTTTAGGGCAGATGCAGACACAGTGCTGCTCTTTCTGAAAAGTGTGC
AGGATGTTTCCTTATATGTCCGAGAGGCTGACGGAACAGAGAAACTGGTGTTTAGAGTGACTTCGAGTGAGAGTA
AGGCACTGAAACATGAGCGGCCGAATTCTATAAAGATTCTGGGAACTGCTATAAGTAACTATTGTAAAAAGACTC
CAAGCAATAACATCACCTGTGTAACATATCACGTAAATATTGTTTTAGAAGAGGAGAGTACTAAGGATGCACAGA
AAACATCTTGGTTGGTGTGTAACAGTGTGGGTGGGCGAGGGATCAGTAGTAAGCTTGACTCTTTAGCTGATGAAC
TGAAATTTGTCCCAATCATTGGAATAGCCATGCCTTTATCAAGCAGAGATGATGAAGCAAAAGGAGCAACGTCTG
ATTTCTCAGGAAAAGCATTTTGTTTCCTTCCTTTACCACCTGGTGAGGAAAGCAGCACAGGCCTCCCAGTTCACA
TCAGTGGGTTCTTTGGCCTTACTGATAACCGCAGGAGCATAAAATGGAGAGAGCTGGACCAGTGGAGAGACCCGG
CAGCCTTATGGAATGAGTTTCTTGTCATGAATGTTGTCCCCAAAGCTTATGCTACTCTGGGATCCATCTTAAAAG
AAGTGACATCTGTGGTGGAGCAAGCATGGAAGCTTCCAGAATCGGAACGAAAAAAGATTATTAGGCGGTTGTATT
TGAAATGGCATCCTGACAAAAATCCAGAGAACCATGACATTGCCAATGAAGTTTTTAAACATTTGCAGAATGAAA
TCAACAGATTAGAAAAACAGGCTTTTCTAGATCAAAATGCAGACAGGGCCTCCAGACGAACATTTTCAACCTCAG
CATCCCGATTTCAGTCAGACAAATACTCATTTCAGAGATTCTATACTTCATGGAATCAAGAAGCAACGAGCCATA
AATCTGAAAGACAGCAACAGAACAAAGAAAAATGCCCCCCTTCAGCCGGACAGACTTACTCTCAAAGGTTCTTTG
TTCCTCCCACTTTCAAGTCGGTTGGCAATCCAGTGGAAGCACGCAGATGGCTAAGACAAGCCAGAGCAAACTTCT
CAGCTGCCAGGAATGACCTTCATAAAAATGCCAATGAGTGGGTGTGCTTTAAATGTTACCTTTCTACCAAGTTAG
CTTTGATTGCAGCTGACTATGCTGTGAGGGGAAAGTCTGATAAAGATGTAAAACCAACTGCACTTGCTCAGAAAA
TAGAGGAATATAGTCAGCAACTTGAAGGACTGACAAATGATGTTCACACATTGGAAGCTTATGGTGTAGACAGTT
TAAAAACAAGATACCCTGATTTGCTTCCCTTTCCTCAGATCCCAAATGACAGGTTCACTTCTGAGGTTGCTATGA
GGGTGATGGAATGTACTGCCTGTATCATAATAAAACTTGAAAATTTTATGCAACAAAAAGTGGGTAAGCCTATCC
CTAACCCTCTCCTCGGTCTCGATTCTACGTGAGAATTCGATATCAAGCTTATCGATAATCAACCTCTGGATTACA
AAATTTGTGAAAGATTGACTGGTATTCTTAACTATGTTGCTCCTTTTACGCTATGTGGATACGCTGCTTTAATGC
CTTTGTATCATGCTATTGCTTCCCGTATGGCTTTCATTTTCTCCTCCTTGTATAAATCCTGGTTGCTGTCTCTTT
ATGAGGAGTTGTGGCCCGTTGTCAGGCAACGTGGCGTGGTGTGCACTGTGTTTGCTGACGCAACCCCCACTGGTT
GGGGCATTGCCACCACCTGTCAGCTCCTTTCCGGGACTTTCGCTTTCCCCCTCCCTATTGCCACGGCGGAACTCA
TCGCCGCCTGCCTTGCCCGCTGCTGGACAGGGGCTCGGCTGTTGGGCACTGACAATTCCGTGGTGTTGTCGGGGA
AATCATCGTCCTTTCCTTGGCTGCTCGCCTATGTTGCCACCTGGATTCTGCGCGGGACGTCCTTCTGCTACGTCC
CTTCGGCCCTCAATCCAGCGGACCTTCCTTCCCGCGGCCTGCTGCCGGCTCTGCGGCCTCTTCCGCGTCTTCGCC
TTCGCCCTCAGACGAGTCGGATCTCCCTTTGGGCCGCCTCCCCGCATCGATACCGAGCGCTGCTCGAGAGATCTA
CGGGTGGCATCCCTGTGACCCCTCCCCAGTGCCTCTCCTGGCCCTGGAAGTTGCCACTCCAGTGCCCACCAGCCT
TGTCCTAATAAAATTAAGTTGCATCATTTTGTCTGACTAGGTGTCCTTCTATAATATTATGGGGTGGAGGGGGGT
GGTATGGAGCAAGGGGCAAGTTGGGAAGACAACCTGTAGGGCCTGCGGGGTCTATTGGGAACCAAGCTGGAGTGC
AGTGGCACAATCTTGGCTCACTGCAATCTCCGCCTCCTGGGTTCAAGCGATTCTCCTGCCTCAGCCTCCCGAGTT
GTTGGGATTCCAGGCATGCATGACCAGGCTCAGCTAATTTTTGTTTTTTTGGTAGAGACGGGGTTTCACCATATT
GGCCAGGCTGGTCTCCAACTCCTAATCTCAGGTGATCTACCCACCTTGGCCTCCCAAATTGCTGGGATTACAGGC
GTGAACCACTGCTCCCTTCCCTGTCCTTCTGATTTTGTAGGTAACCACGTGCGGACCGAGCGGCCGCAGGAACCC
CTAGTGATGGAGTTGGCCACTCCCTCTCTGCGCGCTCGCTCGCTCACTGAGGCCGGGCGACCAAAGGTCGCCCGA
CGCCCGGGCTTTGCCCGGGCGGCCTCAGTGAGCGAGCGAGCGCGCAGCTGCCTGCAGG

### Variants, Derivatives, Analogues, Homologues and Fragments

In addition to the specific proteins and nucleotides mentioned herein, the invention also encompasses variants, derivatives, analogues, homologues and fragments thereof.

In the context of the invention, a “variant” of any given sequence is a sequence in which the specific sequence of residues (whether amino acid or nucleic acid residues) has been modified in such a manner that the polypeptide or polynucleotide in question retains at least one of its endogenous functions. A variant sequence can be obtained by addition, deletion, substitution, modification, replacement and/or variation of at least one residue present in the naturally occurring polypeptide or polynucleotide.

The term “derivative” as used herein in relation to proteins or polypeptides of the invention includes any substitution of, variation of, modification of, replacement of, deletion of and/or addition of one (or more) amino acid residues from or to the sequence, providing that the resultant protein or polypeptide retains at least one of its endogenous functions.

The term “analogue” as used herein in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3, to 10 or 20 substitutions, provided that the modified sequence retains the required activity or ability. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homologue” as used herein means an entity having a certain homology with the wild type amino acid sequence or the wild type nucleotide sequence. The term “homology” can be equated with “identity”.

In the present context, a homologous sequence is taken to include an amino acid sequence which may be at least 50%, 55%, 65%, 75%, 85% or 90% identical, preferably at least 95%, 96% or 97% or 98% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, a homologous sequence is taken to include a nucleotide sequence which may be at least 50%, 55%, 65%, 75%, 85% or 90% identical, preferably at least 95%, 96% or 97% or 98% or 99% identical to the subject sequence. Although homology can also be considered in terms of similarity, in the context of the present invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye, or more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the amino acid or nucleotide sequence may cause the following residues or codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids or nucleotides, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, USA; Devereux et al. (1984) Nucleic Acids Research 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, BLAST 2 Sequences, is also available for comparing protein and nucleotide sequences (FEMS Microbiol. Lett. (1999) 174 (2): 247-50; FEMS Microbiol. Lett. (1999) 177 (1): 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix (the default matrix for the BLAST suite of programs). GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Codon Optimisation

The polynucleotides used in the invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Codon usage tables are known in the art for mammalian cells, as well as for a variety of other organisms.

### Method of Treatment

All references herein to treatment include curative, palliative and prophylactic treatment. The treatment of mammals, particularly humans, is preferred. Both human and veterinary treatments are within the scope of the invention.

In one aspect, the invention provides a method of treating a cerebellar ataxia comprising administering the polynucleotide, vector, polypeptide or cell of the invention to a subject in need thereof.

In one aspect, the invention provides a method of treating autosomal recessive spastic ataxia of Charlevoix-Saguenay (ARSACS) comprising administering the polynucleotide, vector, polypeptide or cell of the invention to a subject in need thereof.

In some embodiments, the method of treatment provides the engineered sacsin to the central nervous system of a subject.

In some embodiments, the method of treatment provides the engineered sacsin to Purkinje cells (PCs).

In some embodiments, the method of treatment provides a decrease in neurofilament heavy subunit accumulation and/or neurofilament bundle volume.

In some embodiments, the method of treatment provides an improvement in motor function in a subject. Methods for measuring motor function are known to those skilled in the art, for example, the beam balance test.

Advantageously, the present invention provides a method for treatment by systemically administering the vector particle of the invention.

### Pharmaceutical Compositions and Injected Solutions

Although the agents for use in the invention can be administered alone, they will generally be administered in admixture with a pharmaceutical carrier, excipient or diluent, particularly for human therapy.

The medicaments, for example vector particles, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. intravenous or intra-arterial.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

### Administration

In some embodiments, the polynucleotide, vector, polypeptide or cell is administered to a subject systemically.

In some embodiments, the polynucleotide, vector, polypeptide or cell is administered to a subject locally.

In some embodiments, the polynucleotide, vector, polypeptide or cell is administered to a subject intracranially, intracerebrally or intraparenchymally.

The term “systemic delivery” or “systemic administration” as used herein means that the agent of the invention is administered into the circulatory system, for example to achieve broad distribution of the agent. In contrast, topical or local administration restricts the delivery of the agent to a localised area e.g. intracerebral administration entails direct injection into the brain.

In some embodiments, the polynucleotide, vector, polypeptide or cell is administered intravascularly, intravenously or intra-arterially.

Suitably, in some embodiments the polynucleotide, vector, polypeptide or cell is administered to the internal carotid artery.

### Dosage

The skilled person can readily determine an appropriate dose of an agent of the invention to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is human.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J.O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

### Material and Methods

### Plasmid Cloning and Cell Transfection

Minisacsin was cloned inside pcDNA3.1™ (+) (Invitrogen V790-20), between NheI and XhoI sites. Human cDNAs corresponding to sacsin human protein aa 1-508 and aa 4306-4579 (Uniprot Q9NZJ4) were cloned and linked together with a BamHI site in frame, which allowed also for the insertion of a GS-linker. V5 tag was cloned after codon 4579 before the STOP codon and the XhoI site. A 10 bp Kozak sequence was cloned between NheI and the first codon. Minisacsin-V5 was inserted into pAAV vectors by enzymatic digestions, between CBA universal promoter and either human growth hormone terminator sequence (hGh) or a shorter PolyA terminator signal (pAmin). The final constructs were all subjected to Sanger sequencing with primers spanning all the construct length. For Western blot (WB) analysis, SH-SY5Y cells or HEK293T cells were transfected with using Lipofectamine3000 (Invitrogen L3000015) and collected after 48 h; for immunofluorescence analysis, cells were transfected with either minisacsin-V5 pr GFP-V5 as a control and fixed in 4% paraformaldehyde after 48 h.

### Western Blot

For Western blot (WB), cells were homogenized in 100 mM Tris-HCl (pH 7.4), 1 mM EDTA (pH 8), 1% Triton X-100 and 150 mM NaCl supplemented with Protease Inhibitor Cocktail (PIC, Sigma-Aldrich, Merck KGaA) and incubated for 30′ on ice. Cell debris were discarded by centrifugation at 8000 g for 10′ at 4° C. Commercially available antibody was used for the detection of V5-tag (Invitrogen, MA515253). For detection of sacsin N-terminal region, AbN was produced according to Longo (2021) Neurology 97: E2315-E2327. Secondary antibodies included Horseradish Peroxidase (HRP)-conjugated anti-mouse and anti-rabbit IgG (Amersham Bioscience).

### Immunofluorescence and NFH Imaging Analysis

Fixed cells with PFA 4% were blocked and permeabilized with 10% goat serum and 0.5% Triton X-100 in PBS 1× solution (1 h, RT), and then incubated in 5% goat serum and 0.1% Triton X-100 in PBS 1× solution with primary antibodies (over/night, 4° C.): V5-tag (Invitrogene MA515253) and NFH (Millipore AB1989). Secondary antibodies conjugated with Alexa 488 and Alexa 596 (Invitrogen) were added in 5% goat serum and 0.2% Triton X-100 in PBS 1× solution (2 h, RT). Stacks of consecutive images were taken at 0.2 μm intervals using the widefield deconvolution microscopy (60×) Deltavision (GE Healthcare) or the FluoVIEW FV3000RS (Olympus) for confocal imaging. Analyses of NFH bundling was done with Arivis Vision 4D software, using an automatic pipeline.

### Minisacsin AAV Production and Purification

Recombinant AAV vectors were produced by transfection of HEK293T cells. Three plasmids were transfected by using polyethylenimine (PEI) (Polyscience): the transgene-containing plasmid, the plasmid coding for the AAV packaging proteins and the pHelper. Five days after, cells were lysed in hypertonic buffer and the supernatant was concentrated (8% PEG8000). A discontinuous iodixanol gradient was prepared to isolate the viral particles. Upon ultracentrifugation, the purified library particles were aspirated from the layer containing 40% iodixanol and dialyzed against phosphate buffer solution (PBS). The virus titer was determined using AAVpro© Titration Kit Ver2 (TakaRa).

### Primary PCs Derivation and Viral Infection

Primary PCs were derived as described in Maltecca et al. (2015) J. Clin. Invest. doi: 10.1172/JCI74770. AAV PHP.eB vector carrying GFP under CBA promoter was derived as described in Luoni et al. (2020) Elife 9:1-30. Lentiviral and AAV PHP.eB vectors carrying GFP under L7 promoter were produced by VectorBuilder. All viruses were delivered to PCs at DIVO prior to plating; a total amount of 10\hat{ }8 vg was delivered for each PCs preparation, in 500 μL final volume medium.

### Mice Treatment

Adult (1 month old) wild-type mice were warmed under a lamp and then put inside a restrainer for intra-tail injection in the caudal vein. A total of 10\hat{ }11 vg per mouse was injected, in a volume of 100 μL PBS 1×. Mice were sacrificed 1 month after injection for tissue collection and analysis.

### Cerebellar Sections Immunostaining

Mice at two months of age were sacrificed in the presence of anaesthesia (2,2,2-Tribromoethanol, Sigma-Aldrich, Merck KGaA). Transcardially perfusion was performed and then the brain was isolated. Tissues were fixed in 4% paraformaldehyde (2 h, 4° C.), then dehydrated in 30% sucrose solution (over/night, 4° C.) and finally included in OCT solution. Cryostat sagittal slices were cut at the thickness of 20 μm and conserved at −80° C. Immunofluorescence was performed as described for fixed primary PCs. Images were taken at FluoVIEW FV3000RS Confocal (Olympus) at 63× magnification and analyzed with FIJI.

### Results

### Minisacsin Gene Expression and Functionality in Human Neuronal Like Cells

For our gene replacement approach in ARSACS, we designed a recombinant sacsin protein (minisacsin), that combines different sacsin domains, with suitable size for packaging into AAV vectors, whose limit is 4.8 kb. Minisacsin consists of cDNA encoding the N-terminal region of human sacsin (aa 1-508), carrying the Ubl and SRR1 domains, linked in frame to cDNA of human sacsin C-terminal region (aa 4306-4579), carrying the DnaJ and HEPN domains. Although SIRPT1 was reported to recover neurofilaments (NFs) accumulation when expressed alone in motor neurons (Gentil et al. (2019) FASEB J. 33:2982-2994), its size (3.8 kb) would prevent insertion of other domains and regulatory sequences for AAV-based delivery, so we opted for the shorter SRR1 domain. We put a V5-tag in frame at the C-terminus of minisacsin, as V5 works efficiently in immunofluorescence. Minisacsin-V5 cDNA was cloned into pcDNA3.1 plasmid, between CMV promoter and BGH polyadenylation signal for mammalian expression. The length of minisacsin-V5 coding sequence is 2.397 kb and with its regulatory elements is 3.346 kb (FIG. 1A).

We transfected minisacsin in neuronal-like SH-SY5Y wild-type cells and in two SH-SY5Y SACS^{-/-} clones we previously generated by CRISPR/Cas9 (Longo et al. (2021) Neurology 97: E2315-E2327). Minisacsin lacks the epitope of the most efficient sacsin commercial antibody (AbC) (aa 4200-4300), but we had generated and validated in the lab a polyclonal Ab recognizing sacsin N-terminal region (AbN; aa 1-728) 1 (FIG. 1A). In WB, a unique band is detected only in transfected cells both with sacsin-specific AbN and with the C-terminal V5-tag Ab (FIG. 1B) around the expected molecular weight (˜90 kDa).

In immunofluorescence, both wild-type and SACS/transfected cells showed stable and high expression of minisacsin, revealed with anti V5-tag Ab; minisacsin is widely diffused in the cytosol, as expected from sacsin full length distribution, with no abnormal localization—e.g. in the nucleus—or aggregates formation. (FIG. 1C).

We evaluated if minisacsin reverts NFs accumulation in SACS^{-/-} SH-SY5Y cells. This neuronal-like model recapitulates well the NF heavy subunit (NFH) accumulation seen in Sacs^{-/-} PCs, with the formation of a perinuclear bundle (FIG. 1C). In most of the untransfected or GFP-control transfected SACS^{-/-} cells, NFH distribution was strikingly remodelled, presenting as a perinuclear densely packed bundle contrasting with the diffuse and filamentous distribution observed in wild-type cells. Many of the SACS−/− cells transfected with minisacsin had instead a decrease of NFH intensity or decreased volume of the dense bundle (FIG. 1 C-D). Furthermore, wild-type cells transfected with minisacsin did not show any differences in respect to control cells in terms of NFH distribution. We repeated this test by adding a second SACS^{-/-} SH-SY5Y CRISPR/Cas9 clone. Measurement of the volume of the largest NFH object in each cell, a parameter indicative of bundling, confirmed again a statistically significant reduction of NF bundle in each clone, while wild-type cells transfected with minisacsin did not display any significant alteration in NFH volume distribution when compared to control cells (FIG. 1E). Overall, this data confirms the strategy reliability and tool suitability for the gene replacement mediated by AAV vectors in ARSACS.

### Minisacsin AAV-Mediated Expression in Cerebellum and PCs

We infected primary PCs from wild-type mice at DIVO with PHP.eB carrying GFP gene (FIG. 2A). GFP under universal Chicken Beta-Actin (CBA) promoter showed higher expression in PCs compared to other cells. We also produced a PHP.eB-GFP under a shorter, AAV-suitable, version of the PC-specific L7-Pcp2 promoter 20, which effectively and specifically transduced PCs (FIG. 2A).

We next cloned minisacsin sequence into pAAV plasmids for AAV packaging and production. Minisacsin was put under strong universal CBA promoter. Two PolyA tail sequences for transcriptional termination were proofed: the terminator of human growth hormone (hGh terminator—477 bp) and a much shorter terminator based on a PolyA terminator signal (pAmin terminator—49 bp). In vitro expression of all minisacsin-carrying plasmids in immortalized human cells confirmed strong and stable expression of minisacsin, regardless of the nature of promoter and PolyA signal (FIG. 2B). We therefore opted for the pAAV encoding minisacsin-pAmin for AAV production, as the shorter final sequence allows higher AAV production yield. This vector was used both to produce AAV-PHP.eB and AAV-Se2w viruses carrying CBA-minisacsin-V5-pAmin gene.

AAV-mediated minisacsin expression in the sites of interest, cerebellum and PCs, is assessed, both in vivo, by intra-tail injection in the caudal vein of adult wild-type mice, and ex vivo, by transducing at DIVO PCs cultures obtained from wild-type mice at P0. Data show that PCs transduced with a very low amount of AAV-Se2w carrying minisacsin (10\hat{ }8 vg for a single PCs preparation) showed good and specific expression of minisacsin, as revealed by anti V5 antibody in immunofluorescence post fixation at DIV11 (FIG. 2C). We repeated the test using the AAV-PHP.eB capsid to transduce minisacsin into PCs cultures. The expression in these cells was strong and without any abnormal subcellular localization or aggregation signs by confocal microscopy (FIG. 2D), at 10\hat{ }10 vg dosage. Also, the cells did not have signs of toxicity. With the universal CBA promoter, the signal in PCs was stronger than in other neurons in the culture, which confirms the high tropism of AAVs for PCs observed in the literature.

Moreover, minisacsin expression pattern looked very similar to the endogenous sacsin staining pattern in PCs, displaying a dotted appearance throughout the cytosol and dendrites (FIG. 2E). In a subsequent experiment, we evaluated if minisacsin reverts NFs accumulation in Sacs^{-/-} PCs. We transduced Sacs-PCs cells using AAV-PHP.eB carrying either minisacsin-V5 or EGFP-V5, with a dosage of 10\hat{ }10 vg. NF bundles were present in proximal dendrites of Sacs^{-/-} PCs treated with PHP.eB-EGFP, but they were notably absent in those treated with PHP.eB-minisacsin, by confocal microscopy (FIG. 2F). Quantification of the NFH signal along dendrites confirmed this observation as significant (FIG. 2G).

Expression of minisacsin in PCs in vivo in adult wild-type mice was evaluated using immunofluorescence on brain sections. Mice were systemically injected at one month of age with either PHP.eB-minisacsin or PHP.eB-EGFP at a dose of 10\hat{ }11 vg into the caudal vein, while another mouse remained uninjected for additional control. Mice were then sacrificed a month later. The PCs from the minisacsin-injected mice showed specific and good expression of minisacsin when compared to the uninjected mice. However, the expression was slightly lower than the expression of EGFP observed in the control mice (FIG. 2H), likely due to low systemic dosage at this age and extended analysis time frame.

### The Codon-Optimized Version of Minisacsin Exhibits Stability and High Expression In Vitro

To enhance therapeutic applications, we recoded the minisacsin CDS via codon-optimization. This process increases expression levels in humans without altering the amino acid sequence, by increasing mRNA stability and translation efficiency. An external industry performed the recoding using proprietary codon optimization algorithms (Azenta Life Sciences). We then transfected HeLa cells with the recoded construct, cloned under the CBA promoter with an hGh polyA tail. Importantly, the expression stability and level of the recoded construct were maintained, at a level similar to the original coding sequences with the same regulatory genetic elements (FIG. 3A).

### DISCUSSION

ARSACS is a rare cerebellar ataxia for which no cure is available and no clinical trials are ongoing. Since we have previously demonstrated that ARSACS is a pure loss-of-function disease, due to striking reduction of sacsin protein regardless of the mutation in charge of the SACS gene, this disease is suitable for therapeutic gene replacement strategy. Gene replacement mediated by AAV vectors is gaining increasingly interest due to its safety profile, the fact that doesn't integrate in the genome and its ability to target non-dividing mature cells as neurons. Currently, multiple clinical and preclinical trials are ongoing for the treatment of different neurodegenerative disease targeting neurons in the central nervous system; also the cerebellum is regarded as a suitable target, for the treatment of genetic ataxia, due to high tropism observed for different AAVs in this site and in particular in PCs.

However, a major hurdle for translating this strategy to ARSACS is the large size of sacsin protein. Indeed, the only coding sequence of sacsin is 13.73 kb, much larger than the limit of packaging size of AAV which is ˜4.8 kb. For this reason, we generated a miniaturized version of sacsin protein, by combining different sacsin domains. Previous reports have showed solely the effect of transfection of single sacsin domains in dissociated motoneurons cultures (Gentil et al. (2019) FASEB J. 33:2982-2994). We decided to include all in silico identified sacsin domains and a single copy of the three SRR1 (Ménade et al. (2018) J. Biol. Chem. 293:12832-12842); Anderson et al. (2010) J. Mol. Biol. 400:665-674). Although SRR are shorter than SIRPTs, each SIRPT is too large to allow suitable AAV production. The final construct cloned under CMV promoter and with BGH polyA tail has a length of ˜3.3 kb, well suitable for AAV packaging and which allows for the future also the insertion of other regulatory sequences and the changing with other promoters, including an ˜800 bp version of PCs specific promoter to drive selective expression in this cell type.

Our recombinant minisacsin protein proved to be stable, highly expressed and not toxic in vitro in neuronal like SH-SY5Y cells which recapitulate ARSACS main hallmark in the absence of sacsin, i.e. NFs accumulation and bundling. Minisacsin was also able to reduce NFs bundling in transfected SH-SY5Y SACS^{-/-} cells. The outcome of this analysis was limited by the few number of cells which are transfected (a limit intrinsic to SH-SY5Y model), but those cells that get transfected show high expression as well as normal localization of minisacsin, with complete resolution or decrease of the NF bundle intensity and size in SACS/cells. No toxicity and altered NFS distribution were observed as well in wild-type cells, in line with minisacsin distribution.

Our results demonstrate that minisacsin is stable and not toxic, with different combinations of promoters and transcription termination signals; minisacsin also reverts NFH accumulation in neuronal-like cells in vitro. We have confirmed the ability of AAV PHP.eB vectors to infect PCs in vitro, thus confirming that it is a valid tool for preclinical cytopathological studies too in our ARSACS model disease. Mice injected with both AAV-PHP.eB and AAV-Se2w carrying minisacsin, did not show any sign of toxicity and had normal weight course as monitored after injection, suggesting that also in vivo in preclinical models minisacsin is not toxic. Moreover, expression of minisacsin in ex vivo PCs was also assessed: minisacsin expression was detectable and specific and primary PCs cultures did not suffer from toxicity. Also, minisacsin expression appeared to be much higher in PCs compared to other cell types, suggesting a strong tropism of AAV vectors for PCs and demonstrating that minisacsin is stable in PCs, the main target of our strategy.

All publications mentioned in the above specification are herein incorporated by reference. Various modifications and variations of the disclosed polynucleotides, polypeptides, vectors, cells, compositions, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

### EMBODIMENTS

Various preferred features and embodiments of the present invention will now be described with reference to the following numbered paragraphs (paras).
1. A polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof, and wherein the nucleotide sequence encoding the engineered sacsin is less than or equal to about 4000 bp in length.
2. The polynucleotide of para 1, wherein the SRR domain is an SRR1 domain, an SRR2 domain or an SRR3 domain, optionally wherein the SRR domain is an SRR1 domain.
3. The polynucleotide of para 1 or 2, wherein the engineered sacsin comprises: (a) a UbL domain, an SRR1 domain, a DnaJ domain and an HEPN domain; (b) a UbL domain, an SRR2 domain, a DnaJ domain and an HEPN domain; or (c) a UbL domain, an SRR3 domain, a DnaJ domain and an HEPN domain.
4. The polynucleotide of any preceding para, wherein the nucleotide sequence encoding the SRR domain comprises a nucleotide sequence having at least 70% sequence identity to SEQ ID NO: 1, 2 or 3.
5. The polynucleotide of any preceding para, wherein the nucleotide sequence encoding the UbL domain comprises a nucleotide sequence having at least 70% sequence identity to SEQ ID NO: 4.
6. The polynucleotide of any preceding para, wherein the nucleotide sequence encoding the DnaJ domain comprises a nucleotide sequence having at least 70% sequence identity to SEQ ID NO: 5.
7. The polynucleotide of any preceding para, wherein the nucleotide sequence encoding the HEPN domain comprises a nucleotide sequence having at least 70% sequence identity to SEQ ID NO: 6.
8. The polynucleotide of any preceding para, wherein the nucleotide sequence encoding an engineered sacsin comprises or consists of a nucleotide sequence having at least 70% sequence identity to SEQ ID NO: 8, 10, 12 or 14.
9. The polynucleotide of any preceding para, wherein the polynucleotide is less than or equal to about 5200 bp in length.
10. The polynucleotide of any preceding para, wherein the nucleotide sequence encoding an engineered sacsin is operably linked to one or more promoter, optionally wherein the promoter is a CBA promoter, a CMV promoter or an L7-Pcp2 promoter.
11. The polynucleotide of any preceding para, wherein the nucleotide sequence encoding an engineered sacsin is operably linked to one or more polyadenylation sequence, optionally wherein the polyadenylation sequence is a human growth hormone (hGh) polyadenylation sequence, bovine growth hormone (bGH) polyadenylation sequence, a SV40 polyadenylation sequence, or a rabbit beta-globin polyadenylation sequence.
12. The polynucleotide of any preceding para, wherein the polynucleotide comprises or consists of a nucleotide sequence having at least 70% sequence identity to any one of SEQ ID NOs: 25-38.
13. A polypeptide encoded by the polynucleotide of any one of paras 1-12.
14. A polypeptide that comprises or consists of an amino acid sequence having at least 70% sequence identity to any one of SEQ ID NOs: 9, 11, 13 or 15.
15. A vector comprising the polynucleotide of any one of paras 1-12.
16. The vector of para 15, wherein the vector is a viral vector, optionally wherein the vector is an AAV vector.
17. The vector of para 15 or 16, wherein the vector comprises or consists of a nucleotide sequence having at least 70% sequence identity to any one of SEQ ID NOs: 73-80.
18 The vector of any one of paras 15-17, wherein the vector is in the form of a viral vector particle, optionally wherein the vector is in the form of an AAV vector particle.
19. The vector of any one of paras 15-18, wherein the viral vector particle comprises a capsid selected from the group consisting of an AAV9 PHP.eB; AAV-Se2w; AAV9; AAV9 PHP.B; and AAVrh10 capsid.
20. A cell comprising the polynucleotide or vector of any one of paras 1-12 or 15-19.
21. A pharmaceutical composition comprising the polynucleotide, polypeptide, vector or cell of any preceding para and a pharmaceutically-acceptable carrier, diluent or excipient.
22. The polynucleotide, vector, polypeptide, cell or pharmaceutical composition of any preceding para for use in medicine.
23. The polynucleotide, vector, polypeptide, cell or pharmaceutical composition of any one of paras 1-21 for use in treating autosomal recessive spastic ataxia of Charlevoix-Saguenay (ARSACS).

### Further Embodiments

Various further preferred features and embodiments of the present invention will now be described with reference to the following numbered paragraphs (paras).
1. A polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof, and wherein the nucleotide sequence encoding the engineered sacsin is less than or equal to about 4000 bp in length.
2. The polynucleotide of para 1, wherein the SRR domain is an SRR1 domain, an SRR2 domain or an SRR3 domain, optionally wherein the SRR domain is an SRR1 domain.
3. The polynucleotide of para 1 or 2, wherein the engineered sacsin comprises: (a) a UbL domain, an SRR1 domain, a DnaJ domain and an HEPN domain; (b) a UbL domain, an SRR2 domain, a DnaJ domain and an HEPN domain; or (c) a UbL domain, an SRR3 domain, a DnaJ domain and an HEPN domain.
4. The polynucleotide of any preceding para, wherein:
   (a) the nucleotide sequence encoding the SRR domain comprises a nucleotide sequence having at least 70% sequence identity to SEQ ID NO: 1, 2 or 3;
   (b) the nucleotide sequence encoding the UbL domain comprises a nucleotide sequence having at least 70% sequence identity to SEQ ID NO: 4;
   (c) the nucleotide sequence encoding the DnaJ domain comprises a nucleotide sequence having at least 70% sequence identity to SEQ ID NO: 5;
   (d) the nucleotide sequence encoding the HEPN domain comprises a nucleotide sequence having at least 70% sequence identity to SEQ ID NO: 6; and/or
   (e) the nucleotide sequence encoding an engineered sacsin comprises or consists of a nucleotide sequence having at least 70% sequence identity to SEQ ID NO: 8, 10, 12 or 14
5. The polynucleotide of any preceding para, wherein the polynucleotide is less than or equal to about 5200 bp in length.
6. The polynucleotide of any preceding para, wherein the nucleotide sequence encoding an engineered sacsin is operably linked to:
   (a) one or more promoter, optionally wherein the promoter is a CBA promoter, a CMV promoter or an L7-Pcp2 promoter; and/or
   (b) one or more polyadenylation sequence, optionally wherein the polyadenylation sequence is a human growth hormone (hGh) polyadenylation sequence, bovine growth hormone (bGH) polyadenylation sequence, a SV40 polyadenylation sequence, or a rabbit beta-globin polyadenylation sequence.
7. The polynucleotide of any preceding para, wherein the polynucleotide comprises or consists of a nucleotide sequence having at least 70% sequence identity to any one of SEQ ID NOs: 25-38.
8. A polypeptide, wherein the polypeptide:
   (a) is encoded by the polynucleotide of any one of paras 1-7; and/or
   (b) comprises or consists of an amino acid sequence having at least 70% sequence identity to any one of SEQ ID NOs: 9, 11, 13 or 15.
9. A vector comprising the polynucleotide of any one of paras 1-7, optionally wherein the vector is a viral vector, optionally wherein the vector is an AAV vector.
10. The vector of para 9, wherein the vector comprises or consists of a nucleotide sequence having at least 70% sequence identity to any one of SEQ ID NOs: 73-80.
11. The vector of para 9 or 10, wherein the vector is in the form of a viral vector particle, optionally wherein the vector is in the form of an AAV vector particle, optionally wherein the viral vector particle comprises a capsid selected from the group consisting of an AAV9 PHP.eB; AAV-Se2w; AAV9; AAV9 PHP.B; and AAVrh10 capsid.
12. A cell comprising the polynucleotide or vector of any one of paras 1-7 or 9-11.
13. A pharmaceutical composition comprising the polynucleotide, polypeptide, vector or cell of any preceding para and a pharmaceutically-acceptable carrier, diluent or excipient.
14. The polynucleotide, vector, polypeptide, cell or pharmaceutical composition of any preceding para for use in medicine.
15. The polynucleotide, vector, polypeptide, cell or pharmaceutical composition of any one of paras 1-13 for use in treating autosomal recessive spastic ataxia of Charlevoix-Saguenay (ARSACS).

1. A polynucleotide comprising a nucleotide sequence encoding an engineered sacsin, wherein the engineered sacsin comprises a UbL domain or variant thereof, an SRR domain or variant thereof, a DnaJ domain or variant thereof and an HEPN domain or variant thereof, and wherein the nucleotide sequence encoding the engineered sacsin is less than or equal to about 4000 bp in length.

2. The polynucleotide of claim 1, wherein the SRR domain is an SRR1 domain, an SRR2 domain or an SRR3 domain, optionally wherein the SRR domain is an SRR1 domain.

3. The polynucleotide of claim 1 or 2, wherein the engineered sacsin comprises: (a) a UbL domain, an SRR1 domain, a DnaJ domain and an HEPN domain; (b) a UbL domain, an SRR2 domain, a DnaJ domain and an HEPN domain; or (c) a UbL domain, an SRR3 domain, a DnaJ domain and an HEPN domain.

4. The polynucleotide of any preceding claim, wherein:
(a) the nucleotide sequence encoding the SRR domain comprises a nucleotide sequence having at least 70% sequence identity to SEQ ID NO: 1, 84, 2 or 3;
(b) the nucleotide sequence encoding the UbL domain comprises a nucleotide sequence having at least 70% sequence identity to SEQ ID NO: 4 or 85;
(c) the nucleotide sequence encoding the DnaJ domain comprises a nucleotide sequence having at least 70% sequence identity to SEQ ID NO: 5 or 86;
(d) the nucleotide sequence encoding the HEPN domain comprises a nucleotide sequence having at least 70% sequence identity to SEQ ID NO: 6 or 87; and/or
(e) the nucleotide sequence encoding an engineered sacsin comprises or consists of a nucleotide sequence having at least 70% sequence identity to SEQ ID NO: 8, 10, 12, 14 or 88.

5. The polynucleotide of any preceding claim, wherein the polynucleotide is less than or equal to about 5200 bp in length.

6. The polynucleotide of any preceding claim, wherein the nucleotide sequence encoding an engineered sacsin is operably linked to:
(a) one or more promoter, optionally wherein the promoter is a CBA promoter, a CMV promoter, CAG promoter, an L7-Pcp2 promoter, a human synapsin 1 (hSyn1) promoter or a neuronal specific enolase promoter; and/or
(b) one or more polyadenylation sequence, optionally wherein the polyadenylation sequence is a human growth hormone (hGh) polyadenylation sequence, bovine growth hormone (bGH) polyadenylation sequence, a SV40 polyadenylation sequence, or a rabbit beta-globin polyadenylation sequence.

7. The polynucleotide of any preceding claim, wherein the polynucleotide comprises or consists of a nucleotide sequence having at least 70% sequence identity to any one of SEQ ID NOs: 25-38.

8. A polypeptide, wherein the polypeptide:
(a) is encoded by the polynucleotide of any one of claims 1-7; and/or
(b) comprises or consists of an amino acid sequence having at least 70% sequence identity to any one of SEQ ID NOs: 9, 11, 13 or 15.

9. A vector comprising the polynucleotide of any one of claims 1-7, optionally wherein the vector is a viral vector, optionally wherein the vector is an AAV vector.

10. The vector of claim 9, wherein the vector comprises or consists of a nucleotide sequence having at least 70% sequence identity to any one of SEQ ID NOs: 73-80.

11. The vector of claim 9 or 10, wherein the vector is in the form of a viral vector particle, optionally wherein the vector is in the form of an AAV vector particle, optionally wherein the viral vector particle comprises a capsid selected from the group consisting of an AAV9 PHP.eB; AAV-Se2w; AAV9; AAV9 PHP.B; and AAVrh10 capsid.

12. A cell comprising the polynucleotide or vector of any one of claim 1-7 or 9-11.

13. A pharmaceutical composition comprising the polynucleotide, polypeptide, vector or cell of any preceding claim and a pharmaceutically-acceptable carrier, diluent or excipient.

14. The polynucleotide, vector, polypeptide, cell or pharmaceutical composition of any preceding claim for use in medicine.

15. The polynucleotide, vector, polypeptide, cell or pharmaceutical composition of any one of claims 1-13 for use in treating autosomal recessive spastic ataxia of Charlevoix-Saguenay (ARSACS).


SEQUENCE LISTING (92 sequences)

SEQ ID NO: 1 - DNA, 1275 bp, synthetic construct
  source 1..1275: mol_type = other DNA
aaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctg

SEQ ID NO: 2 - DNA, 1581 bp, synthetic construct
  source 1..1581: mol_type = other DNA
ataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatgga

SEQ ID NO: 3 - DNA, 1350 bp, synthetic construct
  source 1..1350: mol_type = other DNA
aatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcg

SEQ ID NO: 4 - DNA, 249 bp, synthetic construct
  source 1..249: mol_type = other DNA
atggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggctta

SEQ ID NO: 5 - DNA, 264 bp, synthetic construct
  source 1..264: mol_type = other DNA
atcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactca

SEQ ID NO: 6 - DNA, 417 bp, synthetic construct
  source 1..417: mol_type = other DNA
gttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtg

SEQ ID NO: 7 - skipped

SEQ ID NO: 8 - DNA, 2352 bp, synthetic construct
  source 1..2352: mol_type = other DNA
atggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtg

SEQ ID NO: 9 - protein, 784 aa, synthetic construct
METKENRWVPVTVLPGCVGCRTVAALASWTVRDVKERIFAETGFPVSEQRLWRGGRELSDWIKIGDLTSKNCHLFVNLQSKGLKGGGRFGQTTPPLVDFLKDILRRYPEGGQILKELIQNAEDAGATEVKFLYDETQYGTETLWSKDMAPYQGPALYVYNNAVFTPEDWHGIQEIARSRKKDDPLKVGRFGIGFNSVYHITDVPCIFSGDQIGMLDPHQTLFGPHESGQCWNLKDDSKEISELSDQFAPFVGIFGSTKETFINGNFPGTFFRFPLRLQPSQLSSNLYNKQKVLELFESFRADADTVLLFLKSVQDVSLYVREADGTEKLVFRVTSSESKALKHERPNSIKILGTAISNYCKKTPSNNITCVTYHVNIVLEEESTKDAQKTSWLVCNSVGGRGISSKLDSLADELKFVPIIGIAMPLSSRDDEAKGATSDFSGKAFCFLPLPPGEESSTGLPVHISGFFGLTDNRRSIKWRELDQWRDPAALWNEFLVMNVVPKAYATLGSILKEVTSVVEQAWKLPESERKKIIRRLYLKWHPDKNPENHDIANEVFKHLQNEINRLEKQAFLDQNADRASRRTFSTSASRFQSDKYSFQRFYTSWNQEATSHKSERQQQNKEKCPPSAGQTYSQRFFVPPTFKSVGNPVEARRWLRQARANFSAARNDLHKNANEWVCFKCYLSTKLALIAADYAVRGKSDKDVKPTALAQKIEEYSQQLEGLTNDVHTLEAYGVDSLKTRYPDLLPFPQIPNDRFTSEVAMRVMECTACIIIKLENFMQQKV

SEQ ID NO: 10 - DNA, 2352 bp, synthetic construct
  source 1..2352: mol_type = other DNA
atggaaactaaggagaatagatgggttcctgtgactgtcctcccgggatgcgttgggtgtaggacagtggctgccttggcgagttggacggttcgggatgtaaaggagagaatattcgcggaaactgggttccccgttagtgagcaacgattgtggagaggtggaagggagctgagtgattggatcaagataggggatcttacttccaaaaactgtcatcttttcgtaaatctgcagtctaaaggtctcaaaggcggtgggcgcttcggtcaaacgacgcctccgctcgttgattttctcaaagacatacttcgcagataccctgaaggagggcagatcctcaaggaacttatccaaaatgccgaggacgctggcgctactgaggtaaaattcctttatgatgaaacacagtatggcaccgaaaccctgtggagcaaggatatggctccctatcagggcccggctctctacgtctataacaacgcggttttcaccccggaagactggcatggtatccaggaaatagcgagatccaggaaaaaggacgatccgctgaaggttgggcggttcggaataggttttaatagcgtttatcatattactgatgtgccgtgcatcttctctggcgaccagataggtatgctggacccccatcagaccctgttcggtccccacgagtctgggcagtgttggaacttgaaagatgatagtaaggaaataagtgagttgagcgaccaatttgcgccattcgtaggtatatttggctcaacgaaagagacctttattaacggaaacttccccggtaccttcttccgattccctctccgactccaaccctcacagttgagtagcaatctgtacaataaacagaaggtattggaacttttcgaatcctttcgagcagatgcggacactgtgcttttgttcctcaagagcgtacaagatgtctcactgtatgtacgagaggccgatggcactgagaaattggtatttagagtaaccagctccgaatctaaagcgctcaaacatgagcgacccaacagtattaagattcttgggacggcgatttcaaactactgcaaaaagaccccttccaacaacataacttgtgtaacctatcatgttaacattgttcttgaggaggaaagtaccaaggacgctcaaaagacgtcttggctggtctgcaactctgtcggcgggcgaggcatcagctcaaaactggactccttggcggacgagctcaaattcgtccccataatcgggatcgcaatgcccctgagttcccgggatgatgaggcaaagggcgccacaagtgactttagcggaaaagcattttgctttcttccgctgccacctggagaagaatcatctacaggtctcccagtacacatatcagggttttttggcctcacggataatagaaggtccataaagtggcgagaactggaccaatggcgggatccggctgcattgtggaacgagttcctggtaatgaatgtggtaccaaaagcttacgccacgctggggtcaatactcaaggaggtaacttctgtagtagagcaagcgtggaagcttccggaatctgaaagaaagaaaatcatacgacgactgtatctcaaatggcatcccgataaaaaccctgagaaccatgatatcgcgaatgaagtgttcaagcatctgcaaaacgagattaaccgactcgaaaagcaagcttttctcgaccaaaatgcagatcgcgcatctcggaggacctttagcacctctgccagtcgctttcagtccgacaagtactcattccaacgattttatacatcctggaatcaagaggcaacatcccataaatcagagagacagcaacagaataaagaaaagtgcccacctagcgcgggacagacgtatagtcaacgcttttttgttccacctacattcaagtctgttggtaacccagtggaggcccgaagatggctgaggcaagctcgcgcaaattttagtgctgcacgcaatgaccttcacaagaatgccaatgagtgggtgtgttttaaatgctatttgagtacaaaactcgcgttgattgcggctgactatgcagttcgcggcaaaagtgacaaggatgttaagcctacggctttggcgcagaagatagaagaatactcccaacaacttgagggtctcactaatgatgttcacaccttggaagcgtatggggtagatagccttaaaacgagataccctgatctgttgccattccctcaaattccgaacgaccgcttcactagtgaggtcgcaatgagggtaatggagtgcaccgcgtgtatcattataaagttggagaattttatgcaacagaaggta

SEQ ID NO: 11 - protein, 784 aa, synthetic construct; identical to SEQ ID NO: 9

SEQ ID NO: 12 - DNA, 2658 bp, synthetic construct
  source 1..2658: mol_type = other DNA
atggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtg

SEQ ID NO: 13 - protein, 886 aa, synthetic construct
METKENRWVPVTVLPGCVGCRTVAALASWTVRDVKERIFAETGFPVSEQRLWRGGRELSDWIKIGDLTSKNCHLFVNLQSKGLINPENMGFEQSGQREPLTVRIKNILEEYPSVSDIFKELLQNADDANATECSFLIDMRRNMDIRENLLDPGMAACHGPALWSFNNSQFSDSDFVNITRLGESLKRGEVDKVGKFGLGFNSVYHITDIPIIMSREFMIMFDPNINHISKHIKDKSNPGIKINWSKQQKRLRKFPNQFKPFIDVFGCQLPLTVEAPYSYNGTLFRLSFRTQQEAKVSEVSSTCYNTADIYSLVDEFSLCGHRLIIFTQSVKSMYLKYLKIEETNPSLAQDTVIIKKKSCSSKALNTPVLSVLKEAAKLMKTCSSSNKKLPSDEPKSSCILQITVEEFHHVFRRIADLQSPLFRGPDDDPAALFEMAKSGQSKKPSDELSQKTVECTTWLLCTCMDTGEALKFSLSESGRRLGLVPCGAVGVQLSEIQDQKWTVKPHIGEVFCYLPLRIKTGLPVHINGCFAVTSNRKEIWKTDTKGRWNTTFMRHVIVKAYLQVLSVLRDLATSGELMDYTYYAVWPDPDLVHDDFSVICQGFYEDIAHGGSILKEVTSVVEQAWKLPESERKKIIRRLYLKWHPDKNPENHDIANEVFKHLQNEINRLEKQAFLDQNADRASRRTFSTSASRFQSDKYSFQRFYTSWNQEATSHKSERQQQNKEKCPPSAGQTYSQRFFVPPTFKSVGNPVEARRWLRQARANFSAARNDLHKNANEWVCFKCYLSTKLALIAADYAVRGKSDKDVKPTALAQKIEEYSQQLEGLTNDVHTLEAYGVDSLKTRYPDLLPFPQIPNDRFTSEVAMRVMECTACIIIKLENFMQQKV

SEQ ID NO: 14 - DNA, 2427 bp, synthetic construct
  source 1..2427: mol_type = other DNA
atggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtg

SEQ ID NO: 15 - protein, 809 aa, synthetic construct
METKENRWVPVTVLPGCVGCRTVAALASWTVRDVKERIFAETGFPVSEQRLWRGGRELSDWIKIGDLTSKNCHLFVNLQSKGLNVCFTTLGTEFGQKEKLTSRIKSILNAYPSEKEMLKELLQNADDAKATEICFVFDPRQHPVDRIFDDKWAPLQGPALCVYNNQPFTEDDVRGIQNLGKGTKEGNPYKTGQYGIGFNSVYHITDCPSFISGNDILCIFDPHARYAPGATSISPGRMFRDLDADFRTQFSDVLDLYLGTHFKLDNCTMFRFPLRNAEMAKVSEISSVPASDRMVQNLLDKLRSDGAELLMFLNHMEKISICEIDKSTGALNVLYSVKGKITDGDRLKRKQFHASVIDSVTKKRQLKDIPVQQITYTMDTEDSEGNLTTWLICNRSGFSSMEKVSKSVISAHKNQDITLFPRGGVAACITHNYKKPHRAFCFLPLSLETGLPFHVNGHFALDSARRNLWRDDNGVGVRSDWNNSLMTALIAPAYVELLIQLKKRYFPGSDPTLSVLQNTPIHVVKDTLKKFLSGSILKEVTSVVEQAWKLPESERKKIIRRLYLKWHPDKNPENHDIANEVFKHLQNEINRLEKQAFLDQNADRASRRTFSTSASRFQSDKYSFQRFYTSWNQEATSHKSERQQQNKEKCPPSAGQTYSQRFFVPPTFKSVGNPVEARRWLRQARANFSAARNDLHKNANEWVCFKCYLSTKLALIAADYAVRGKSDKDVKPTALAQKIEEYSQQLEGLTNDVHTLEAYGVDSLKTRYPDLLPFPQIPNDRFTSEVAMRVMECTACIIIKLENFMQQKV

SEQ ID NO: 16 - DNA, 278 bp, synthetic construct
  source 1..278: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 17 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 18 - DNA, 844 bp, synthetic construct
  source 1..844: mol_type = other DNA
ggttccaccctcatgttggttgatcttcaacattgtcctgacttcttcccacttgacattcctcagggtcctgtgatcatggctgggtctagtgaggttcaaacctgcactgccctacccacacccacacccagctcagcgtcagtcaggatcaacagttacctagagatcatctttctggggcttaagcattggtgggagcagatgggatatgagttggggatttggggatgggggaagatatctgctccccctccccctacaccctagccttttaaaaggccttctcaggtcagagaccaggagaaaagtataggagagatacacaatggaccaggaagaagaaaagggagagggaggctcagaccttctagacaaggtaagagggctctggctgactccaccatccgcttcttgaggtctcggcacctgtaattgacaagattaattcatttatagggcatctaattagcaagtaagtctctggagtcccctgacccagttactataacacacagggggtataggtaggggagtataagagcccctcctcagggcaaatgaatggattcttagtactgtcccccaagagatagtaggtactaggatttaggggcacttctgagccccatttccctggtaagtgtcccaaccccccaaatcaacccaagcctggtctcaatctaggacagtggtagaatgctgtccctagagtcagtaccatgtgaaattgtgctgcaggcaggggccccaggctgggaggtgggggttgggggagtcaggggcaggtcagggaaggagactcaggtttcatttagagaaattctgcagacccgtgaggact

SEQ ID NO: 19 - DNA, 304 bp, synthetic construct
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 20 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 21 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 22 - DNA, 49 bp, synthetic construct
  source 1..49: mol_type = other DNA
aataaaagatctttattttcattagatctgtgtgttggttttttgtgtg

SEQ ID NO: 23 - DNA, 15 bp, synthetic construct
  source 1..15: mol_type = other DNA
gccgccgcgatcgcc

SEQ ID NO: 24 - DNA, 42 bp, synthetic construct
  source 1..42: mol_type = other DNA
ggtaagcctatccctaaccctctcctcggtctcgattctacg

SEQ ID NO: 25 - DNA, 3304 bp, synthetic construct
  source 1..3304: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 26 - DNA, 3346 bp, synthetic construct
  source 1..3346: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 27 - DNA, 4208 bp, synthetic construct
  source 1..4208: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 28 - DNA, 4250 bp, synthetic construct
  source 1..4250: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 29 - DNA, 4514 bp, synthetic construct
  source 1..4514: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 30 - DNA, 4556 bp, synthetic construct
  source 1..4556: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 31 - DNA, 4283 bp, synthetic construct
  source 1..4283: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 32 - DNA, 4325 bp, synthetic construct
  source 1..4325: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 33 - DNA, 3778 bp, synthetic construct
  source 1..3778: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtg

SEQ ID NO: 34 - DNA, 3820 bp, synthetic construct
  source 1..3820: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtg

SEQ ID NO: 35 - DNA, 4084 bp, synthetic construct
  source 1..4084: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtg

SEQ ID NO: 36 - DNA, 4126 bp, synthetic construct
  source 1..4126: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtg

SEQ ID NO: 37 - DNA, 3853 bp, synthetic construct
  source 1..3853: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtg

SEQ ID NO: 38 - DNA, 3895 bp, synthetic construct
  source 1..3895: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtg

SEQ ID NO: 39 - protein, 7 aa, synthetic construct
TLAVPFK

SEQ ID NO: 40 - protein, 7 aa, synthetic construct
KFPVALT

SEQ ID NO: 41 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 42 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 43 - protein, 11 aa, synthetic construct
DGTLAVPFKAQ

SEQ ID NO: 44 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 45 - protein, 745 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSGQAGPGVPGRFAQAATGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - protein, 5 aa, synthetic construct
GGGGS

SEQ ID NO: 47 - protein, 16 aa, synthetic construct
AEAAAKALEAEAAAKA

SEQ ID NO: 48 - DNA, 15 bp, synthetic construct
  source 1..15: mol_type = other DNA
gggggtgggggcagt

SEQ ID NO: 49 - DNA, 48 bp, synthetic construct
  source 1..48: mol_type = other DNA
gcggaagctgcggctaaagccctcgaagctgaggctgctgccaaagcc

SEQ ID NO: 50 - protein, 47 aa, synthetic construct
FQRFYTSWNQEATSHKSERQQQNKEKCPPSAGQTYSQRFFVPPTFKS

SEQ ID NO: 51 - DNA, 141 bp, synthetic construct
  source 1..141: mol_type = other DNA
tttcagagattctatacttcatggaatcaagaagcaacgagccataaatctgaaagacagcaacagaacaaagaaaaatgccccccttcagccggacagacttactctcaaaggttctttgttcctcccactttcaagtcg

SEQ ID NO: 52 - DNA, 731 bp, synthetic construct
  source 1..731: mol_type = other DNA
gggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaaca

SEQ ID NO: 53 - DNA, 1289 bp, synthetic construct
  source 1..1289: mol_type = other DNA
cattatggccttaggtcacttcatctccatggggttcttcttctgattttctagaaaatgagatgggggtgcagagagcttcctcagtgacctgcccagggtcacatcagaaatgtcagagctagaacttgaactcagattactaatcttaaattccatgccttgggggcatgcaagtacgatatacagaaggagtgaactcattagggcagatgaccaatgagtttaggaaagaagagtccagggcagggtacatctacaccacccgcccagccctgggtgagtccagccacgttcacctcattatagttgcctctctccagtcctaccttgacgggaagcacaagcagaaactgggacaggagccccaggagaccaaatcttcatggtccctctgggaggatgggtggggagagctgtggcagaggcctcaggaggggccctgctgctcagtggtgacagataggggtgagaaagcagacagagtcattccgtcagcattctgggtctgtttggtacttcttctcacgctaaggtggcggtgtgatatgcacaatggctaaaaagcagggagagctggaaagaaacaaggacagagacagaggccaagtcaaccagaccaattcccagaggaagcaaagaaaccattacagagactacaagggggaagggaaggagagatgaattagcttcccctgtaaaccttagaacccagctgttgccagggcaacggggcaatacctgtctcttcagaggagatgaagttgccagagtaactacatcctgtctttctcaaggaccatcccagaatgtggcacccactagccgttaccatagcaactgcctctttgccccacttaatcccatcccgtctgttaaaagggccctatagttggaggtgggggaggtaggaagagcgatgatcacttgtggactaagtttgttcgcatccccttctccaaccccctcagtacatcaccctgggggaacagggtccacttgctcctgggcccacacagtcctgcagtattgtgtatataaggccagggcaaagaggagcaggttttaaagtgaaaggcaggcaggtgttggggaggcagttaccggggcaacgggaacagggcgtttcggaggtggttgccatggggacctggatgctgacgaaggctcgcgaggctgtgagcagccacagtgccctgctcagaagccccaagctcgtcagtcaagccggttctccgtttgcactcaggagcacgggcaggcgagtggcccctagttctgggggcagc

SEQ ID NO: 54 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 55 - DNA, 1488 bp, synthetic construct
  source 1..1488: mol_type = other DNA
ctagaggatccacagtctcctgagtagctgggactacaggagcttgttaccacacccagctccagtttataaattcatctccagtttataaaggaggaaaccgaggtactgagaggttaaaaaaccttcctgcagacacttgtccagcaagtggccactccaggatttggaccaaggtgatgtgtcttcaggctgtgtctctgccactgtgccacgctgctgggtggtaggcagcagtgggtgggtgcctgcagtggtctgtaaagaccacctgagatgtccttcctcctctgttccaccctgtccaggtccaagaagacagtctatgaagagagagcaggtgtgactctctcagtgtgctcctctgtgagaagcaggctgacatcccaaagggaagggcggataacagagacagtgcaagcggaggagatgagggtgcctcaaagccgggaggctgggtgatgcaggagcctgcgtgtcccgaggggggtgctgggcccagtgtgagtacgtgtgactgtgactgagacagtgtgactgctgaaggcagggacacagcagctccctgactgggggcagaaggcgttaactgtgtgaaggctggttgtgggtgggtgggctctgggcctcgaacccgggggctgagggagatagtaaacagcagggtgactgacgggaagatcatgttggtagccctgcgaagatgctgcagggctgtgggggtttgtgtgactttgcagttcaacaaattcaaattcagccaacgctggcagggcctgttgtgccaggcaaccagctaggaggaggagactcggacccagcttgcagctgaagggcgctggctgccgggttctgtgggttcaccttgcggtgtcttcccttgctaacactgagtccttacaatagccccatctccaggttgaggctagatggaggggacagagggaagtgacttgcccaaggtgacccaagctcccgagtgccagggcaggatctgaattcaggctctcagactgcagagcctgagtccctccctgccatgcctgtgccagggtggaaatgtctggtcctggaggggagcgtggactcctggccttggctctggagacatccccctagaccacgtgggctcctaacctgtccatggtcactgtgctgaggggcgggacggtgggtcacccctagttcttttttccccagggccagattcatggactgaagggttgctcggctctcagagaccccctaagcgccccgccctggccccaagccctcccccagctcccgcgtcccccccctcctggcgctgactccgggccagaagaggaaaggctgtctccacccacctctcgcactctcccttctcctttataaaggccggaacagctgaaagggtggcaacttctcctcctgcagccgggagcggcctgcctgcctccctgcgcacccgcagcctcccccgctgcctccctagagtcgaggaactaa

SEQ ID NO: 56 - DNA, 1180 bp, synthetic construct
  source 1..1180: mol_type = other DNA
tggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 57 - DNA, 3527 bp, synthetic construct
  source 1..3527: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 58 - DNA, 3097 bp, synthetic construct
  source 1..3097: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtg

SEQ ID NO: 59 - DNA, 3569 bp, synthetic construct
  source 1..3569: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 60 - DNA, 3139 bp, synthetic construct
  source 1..3139: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtg

SEQ ID NO: 61 - DNA, 3610 bp, synthetic construct
  source 1..3610: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 62 - DNA, 3833 bp, synthetic construct
  source 1..3833: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 63 - DNA, 3403 bp, synthetic construct
  source 1..3403: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtg

SEQ ID NO: 64 - DNA, 3652 bp, synthetic construct
  source 1..3652: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 65 - DNA, 3875 bp, synthetic construct
  source 1..3875: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 66 - DNA, 3445 bp, synthetic construct
  source 1..3445: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtg

SEQ ID NO: 67 - DNA, 3379 bp, synthetic construct
  source 1..3379: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 68 - DNA, 3602 bp, synthetic construct
  source 1..3602: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 69 - DNA, 3172 bp, synthetic construct
  source 1..3172: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtg

SEQ ID NO: 70 - DNA, 3421 bp, synthetic construct
  source 1..3421: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 71 - DNA, 3644 bp, synthetic construct
  source 1..3644: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 72 - DNA, 3214 bp, synthetic construct
  source 1..3214: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtg

SEQ ID NO: 73 - DNA, 4604 bp, synthetic construct
  source 1..4604: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 74 - DNA, 4142 bp, synthetic construct
  source 1..4142: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgactcgacgaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 75 - DNA, 4411 bp, synthetic construct
  source 1..4411: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggttccaccctcatgttggttgatcttcaacattgtcctgacttcttcccacttgacattcctcagggtcctgtgatcatggctgggtctagtgaggttcaaacctgcactgccctacccacacccacacccagctcagcgtcagtcaggatcaacagttacctagagatcatctttctggggcttaagcattggtgggagcagatgggatatgagttggggatttggggatgggggaagatatctgctccccctccccctacaccctagccttttaaaaggccttctcaggtcagagaccaggagaaaagtataggagagatacacaatggaccaggaagaagaaaagggagagggaggctcagaccttctagacaaggtaagagggctctggctgactccaccatccgcttcttgaggtctcggcacctgtaattgacaagattaattcatttatagggcatctaattagcaagtaagtctctggagtcccctgacccagttactataacacacagggggtataggtaggggagtataagagcccctcctcagggcaaatgaatggattcttagtactgtcccccaagagatagtaggtactaggatttaggggcacttctgagccccatttccctggtaagtgtcccaaccccccaaatcaacccaagcctggtctcaatctaggacagtggtagaatgctgtccctagagtcagtaccatgtgaaattgtgctgcaggcaggggccccaggctgggaggtgggggttgggggagtcaggggcaggtcagggaaggagactcaggtttcatttagagaaattctgcagacccgtgaggactcaagtttgtacaaaaaagcaggctgccaccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 76 - DNA, 4291 bp, synthetic construct
  source 1..4291: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 77 - DNA, 4646 bp, synthetic construct
  source 1..4646: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 78 - DNA, 4184 bp, synthetic construct
  source 1..4184: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcaacgcgctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcccgtttagtgaaccgtcagatcgcctggagacgccatccacgctgttttgacctccatagaagacaccgggaccgatccagcctccgcggattcgaatcccggccgggaacggtgcattggaacgcggattccccgtgccaagagtgacgtaagtaccgcctatagagtctataggcccacaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagaattgggattcgaacaccggtcgacgaattcgttaacggatccgctagcgccgccgcgatcgccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgactcgacgaattcaataaaagatctttattttcattagatctgtgtgttggttttttgtgtgcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 79 - DNA, 4453 bp, synthetic construct
  source 1..4453: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttctagacaactttgtatagaaaagttgggttccaccctcatgttggttgatcttcaacattgtcctgacttcttcccacttgacattcctcagggtcctgtgatcatggctgggtctagtgaggttcaaacctgcactgccctacccacacccacacccagctcagcgtcagtcaggatcaacagttacctagagatcatctttctggggcttaagcattggtgggagcagatgggatatgagttggggatttggggatgggggaagatatctgctccccctccccctacaccctagccttttaaaaggccttctcaggtcagagaccaggagaaaagtataggagagatacacaatggaccaggaagaagaaaagggagagggaggctcagaccttctagacaaggtaagagggctctggctgactccaccatccgcttcttgaggtctcggcacctgtaattgacaagattaattcatttatagggcatctaattagcaagtaagtctctggagtcccctgacccagttactataacacacagggggtataggtaggggagtataagagcccctcctcagggcaaatgaatggattcttagtactgtcccccaagagatagtaggtactaggatttaggggcacttctgagccccatttccctggtaagtgtcccaaccccccaaatcaacccaagcctggtctcaatctaggacagtggtagaatgctgtccctagagtcagtaccatgtgaaattgtgctgcaggcaggggccccaggctgggaggtgggggttgggggagtcaggggcaggtcagggaaggagactcaggtttcatttagagaaattctgcagacccgtgaggactcaagtttgtacaaaaaagcaggctgccaccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgaacccagctttcttgtacaaagtgggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 80 - DNA, 4333 bp, synthetic construct
  source 1..4333: mol_type = other DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaaggtaccatggagaccaaggagaacaggtgggtcccggtgaccgtgctccccggctgcgtgggctgcaggaccgtcgcggcgctggcgtcctggaccgtgcgcgatgtgaaggaacgtatcttcgcggagactggcttcccggtgtcggagcagcggctgtggcgcggcggccgcgagttatctgactggatcaagattggagatctgacttccaaaaattgtcatctttttgtaaaccttcaatcaaaaggcttaaaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgggatccatcttaaaagaagtgacatctgtggtggagcaagcatggaagcttccagaatcggaacgaaaaaagattattaggcggttgtatttgaaatggcatcctgacaaaaatccagagaaccatgacattgccaatgaagtttttaaacatttgcagaatgaaatcaacagattagaaaaacaggcttttctagatcaaaatgcagacagggcctccagacgaacattttcaacctcagcatcccgatttcagtcagacaaatactcatttcagagattctatacttcatggaatcaagaagcaacgagccataaatctgaaagacagcaacagaacaaagaaaaatgccccccttcagccggacagacttactctcaaaggttctttgttcctcccactttcaagtcggttggcaatccagtggaagcacgcagatggctaagacaagccagagcaaacttctcagctgccaggaatgaccttcataaaaatgccaatgagtgggtgtgctttaaatgttacctttctaccaagttagctttgattgcagctgactatgctgtgaggggaaagtctgataaagatgtaaaaccaactgcacttgctcagaaaatagaggaatatagtcagcaacttgaaggactgacaaatgatgttcacacattggaagcttatggtgtagacagtttaaaaacaagataccctgatttgcttccctttcctcagatcccaaatgacaggttcacttctgaggttgctatgagggtgatggaatgtactgcctgtatcataataaaacttgaaaattttatgcaacaaaaagtgggtaagcctatccctaaccctctcctcggtctcgattctacgtgagaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctatgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 81 - DNA, 3873 bp, synthetic construct
  source 1..3873: mol_type = other DNA
aaagggggaggtcgatttggtcagacaacgccaccacttgttgattttctcaaggacattttgagaagatatccagaaggaggacagattcttaaggaattaattcagaatgcagaagatgctggggcgacagaagttaaatttttatatgatgaaactcaatacggaacagagactctttggtcaaaagatatggcgccatatcaggggccagctctctatgtgtacaacaacgcggttttcaccccagaggactggcacggcattcaagaaatagcaagaagcaggaaaaaggatgatcctctgaaggtcggaagatttggaattgggtttaattctgtctatcatataacagatgttccttgtatctttagtggtgaccaaatcgggatgctagatcctcatcaaacactttttggcccacatgaatcaggccaatgttggaatctcaaagatgacagcaaagaaattagtgaactttcagaccagtttgcaccatttgttggcatttttggaagcaccaaggaaacatttataaacggcaattttccaggaacatttttccgtttccctcttcgcctacaaccttcacaacttagtagtaacctctacaataagcagaaggttcttgagttgtttgagtcttttagggcagatgcagacacagtgctgctctttctgaaaagtgtgcaggatgtttccttatatgtccgagaggctgacggaacagagaaactggtgtttagagtgacttcgagtgagagtaaggcactgaaacatgagcggccgaattctataaagattctgggaactgctataagtaactattgtaaaaagactccaagcaataacatcacctgtgtaacatatcacgtaaatattgttttagaagaggagagtactaaggatgcacagaaaacatcttggttggtgtgtaacagtgtgggtgggcgagggatcagtagtaagcttgactctttagctgatgaactgaaatttgtcccaatcattggaatagccatgcctttatcaagcagagatgatgaagcaaaaggagcaacgtctgatttctcaggaaaagcattttgtttccttcctttaccacctggtgaggaaagcagcacaggcctcccagttcacatcagtgggttctttggccttactgataaccgcaggagcataaaatggagagagctggaccagtggagagacccggcagccttatggaatgagtttcttgtcatgaatgttgtccccaaagcttatgctactctgatcttagattcaataaaacgtctggagatggaaaagagctctgatttccccttgtcagttgatgttatctataagctttggccggaggcgagcaaagtcaaggtgcactggcaaccggtgttagagcctctattcagcgagctgttgcagaatgcagtgatttattcaattagctgtgactgggtcaggttggagcaggtgtacttctcagaacttgatgaaaatttagaatacacaaaaactgtgctcaactacctccagagctcagggaagcagattgccaaggtaccagggaatgtggatgctgctgttcagctcacagctgcctctggcacaacacctgtgaggaaggtgacgcccgcgtgggtgcggcaggtgctgcggaagtgtgcacacctgggctgtgctgaagaaaagcttcaccttctagaatttgtgctttctgaccaagcctacagtgagctgcttgggctggagctgctccctttacaaaatggcaattttgtccccttctcctcatctgtatcagaccaagatgtcatttatattacctcagcagaatatccaaggtcccttttcccaagtcttgaaggaagatttattttggataacttgaaacctcaccttgtggctgctttaaaggaagctgcccaaacccgaggaagaccatgtactcagctgcagcttctaaatccagaacgatttgcacgtcttatcaaggaagtaatgaatacattctggcctggcagagaattgattgttcaatggtatccatttgatgaaaacagaaatcacccatctgtttcatggcttaagatggtttggaaaaatctttatatacatttttcagaggatttgactttatttgatgagatgccacttatccccagaactatactagaggaaggtcagacatgtgtggaactcattagactcaggattccatcgttagtcattttagacgatgaatctgaagcacagcttccagaatttttagcagacattgtacaaaaacttggagggtttgtccttaaaaaattagatgcatctatacaacatccgcttattaaaaaatatattcattcaccattaccaagtgctgttttgcagataatggagaagatgccattgcagaaattgtgtaatcaaataacttcgctacttccaacacacaaagatgccctgaggaagttcttggctagtttaaccgatagcagtgagaaagagaaaagaattattcaagaattggcaatattcaagcgcattaaccattcttctgatcagggaatttcctcttatacaaaattgaaaggttgtaaagtcttacaccatactgccaaactcccagcagatctgcgactttctatttcagtaatagacagtagtgatgaagctactattcgtctggcaaacatgttgaaaatagaacagttaaagaccactagctgcttaaagcttgttttaaaagatattgaaaatgcattttattcacatgaagaggtaacacagcttatgttatgggtccttgagaatctatcttctcttaaaaatgagaatccaaatgtgcttgagtggttaacaccattaaaattcatccagatatcacaggaacagatggtatcagctggtgaactctttgaccctgatatagaagtactaaaggatctcttttgtaatgaagaaggaacctatttcccaccctcagtttttacctcaccagatattcttcactccttaagacagattggtttaaaaaacgaagccagtctcaaagaaaaggatgttgtgcaagtggcaaaaaaaattgaagccttacaggtcggtgcttgtcctgatcaagatgttcttctgaagaaagccaaaaccctcttactggttttaaataagaatcacacactgttgcaatcatctgaaggaaagatgacattgaagaaaataaaatgggttccagcctgcaaggaaaggcctccaaattatccaggctctttggtctggaaaggagatctctgtaatctctgtgcaccaccagatatgtgtgatgtaggccatgcaattctcattggctcctcacttcctcttgttgaaagtatccatgtaaacctggaaaaagcattagggatcttcacaaaacctagccttagtgctgtcttaaaacactttaaaattgttgttgattggtattcttcaaaaacctttagtgatgaagactactatcaattccagcatattttgcttgagatttacggattcatgcatgatcatctaaatgaagggaaagattcttttagagccttaaaatttccatgggtttggactggcaaaaagttttgtccacttgcccaggctgtgattaaaccaatccatgatcttgaccttcagccttatttgcataatgtacctaaaaccatggcaaaattccaccaactatttaaggtctgtggttcaatagaggagttgacatcagatcatatttccatggttattcagaagatatatctcaaaagtgaccaagatctcagtgaacaagaaagcaaacaaaatcttcatcttatgttgaatattatcagatggctgtatagcaatcagattccagcaagc

SEQ ID NO: 82 - DNA, 3000 bp, synthetic construct
  source 1..3000: mol_type = other DNA
ataaatcctgaaaacatgggatttgagcagtcaggacaaagagagccacttactgtaagaattaaaaatattctggaagaatacccttcagtgtcagatatttttaaagaactacttcaaaacgctgatgatgcaaatgcaacagaatgcagtttcttgattgatatgagaagaaatatggacataagagagaatctcctagacccagggatggcagcttgtcatggacctgctttgtggtcattcaacaattctcaattctcagattcagattttgtgaacataactaggttaggagaatctttaaaaaggggagaagttgacaaagttggaaaatttggtcttggatttaattctgtgtaccatatcactgacattcccatcattatgagtcgggaattcatgataatgttcgatccaaacataaatcatatcagtaaacacattaaagacaaatccaatcctgggatcaaaattaattggagtaaacaacagaaaagacttagaaaatttcctaatcagttcaaaccatttatagatgtatttggctgtcagttacctttgactgtagaagcaccttacagctataatggaacccttttccgactgtcctttagaactcaacaggaagcaaaagtgagtgaagttagtagtacgtgctacaatacagcagatatttattctcttgtggatgaatttagtctctgtggacacaggcttatcattttcactcagagtgtaaagtcaatgtatttgaagtacttgaaaattgaggaaaccaaccccagtttagcacaagatacagtaataattaaaaaaaaatcctgctcttccaaagcattgaacacacctgtcttaagtgttttaaaagaggctgctaagctcatgaagacttgcagcagcagtaataaaaagcttcccagtgatgaaccaaagtcatcttgcattcttcagatcacagtggaagaatttcaccatgtgttcagaaggattgctgatttacagtcgccactttttagaggtccagatgatgacccagctgctctctttgaaatggctaagtctggccaatcaaaaaagccatcagatgagttgtcacagaaaacagtagagtgtaccacgtggcttctgtgtacttgcatggacacaggagaggctctgaagttttccctgagtgagagtggaagaagactaggactggttccatgtggggcagtaggagttcagctgtcagaaatccaggaccagaagtggacagtgaaaccacacattggagaggtgttttgctatttacctttacgaataaaaacaggcttgccagttcatatcaatgggtgctttgctgttacatcaaataggaaagaaatctggaaaacagatacaaaaggacgatggaataccacgttcatgagacatgttattgtgaaagcttacttacaggtactgagtgtcttacgggacctggccactagtggggagctaatggattatacttactatgcagtatggcccgatcctgatttagttcatgatgatttttctgtaatttgccaaggattttatgaagatatagctcatggaaaagggaaagaactgaccaaagtcttctctgatggatctacttgggtttccatgaagaacgtaagatttctagatgactctatacttaaaagaagagatgttggttcagcagccttcaagatatttttgaaatacctcaagaagactgggtccaaaaacctttgtgctgttgaacttccttcttcggtaaaattaggatttgaagaagctggctgcaaacagatactacttgaaaacacattttcagagaaacagtttttttctgaagtgttttttccaaatattcaagaaattgaagcagaacttagagatcctttaatgatctttgttctaaatgaaaaagttgatgagttctcgggagttcttcgtgttactccatgtattccttgttccttggaggggcatcctttggttttgccatcaagattgatccaccccgaaggacgagttgcaaagttatttgatattaaagatgggagattcccttatggttctactcaggattatctcaatcctattattttgattaaactagttcagttaggtatggcaaaagatgatattttatgggatgatatgctagaacgtgcagtgtcagtagctgaaattaataaaagtgatcatgttgctgcatgcctaagaagtagtatcttattgagtcttatcgatgagaaactaaaaataagggatcctagagcaaaggattttgctgcaaaatatcaaacaatccgcttccttccatttctgacaaaaccagcaggtttttctttggactggaaaggcaacagttttaagcctgaaaccatgtttgcagcaactgacctttatacagctgaacatcaagatatagtttgtcttttgcaaccaattctaaatgaaaattcccattcttttagaggttgtggttcagtgtcattggctgttaaagagtttttgggattactcaagaagccaacagttgatctggttataaaccaattgaaagaagtagcaaaatcagttgatgatggaattacactgtaccaggagaatatcaccaatgcttgctacaaataccttcatgaagccttgatgcaaaatgaaatcactaagatgtcaattattgataagttaaaaccctttagcttcattctagttgagaatgcatatgttgactcagaaaaggtttcttttcatttaaattttgaggcggcaccatacctttatcagttgcctaataagtataaaaataatttccgcgaactttttgaaaccgtgggtgtgaggcagtcatgcactgttgaagattttgctcttgttttggaatctattgatcaagaaagaggaacaaagcaaataacagaagagaattttcagctttgccgacgaataatcagtgaaggaatatggagtctcattagagaaaagaaacaagaattt

SEQ ID NO: 83 - DNA, 4155 bp, synthetic construct
  source 1..4155: mol_type = other DNA
aatgtctgttttacaacacttggcacagaatttgggcagaaagaaaaattgaccagcagaattaagagcatccttaatgcatatccttctgaaaaggaaatgttgaaagagcttcttcaaaatgctgatgatgcaaaggcgacagaaatctgttttgtgtttgatcctagacagcatccagttgatagaatatttgatgataagtgggccccattgcaagggccagcactttgtgtgtacaacaaccagccatttacagaagatgatgttagaggaattcagaatcttggaaaaggcacgaaagagggaaatccttataaaactggacagtatggaataggattcaattctgtgtatcatatcacagactgcccatcttttatttctggcaatgacatcctgtgtatttttgatcctcatgccagatatgcaccaggggccacatccattagtcccggacgcatgtttagagatttggatgcagattttaggacacagttctcagatgttctggatctttatctgggaacccattttaaactggataattgcacaatgttcagatttcctcttcgtaatgcagaaatggcaaaagtttcggaaatttcgtctgttccagcatcagacagaatggtccagaatcttttggacaaactgcgctcagatggggcagaacttctaatgtttcttaatcacatggaaaaaatttctatttgtgaaatagataagagtactggagctctaaatgtgctgtattcagtaaagggcaaaatcacagatggagacagattgaaaaggaaacaatttcatgcatctgtaattgatagtgttactaaaaagaggcagctcaaagacataccagttcaacaaataacctatactatggatactgaggactctgaaggaaatcttactacgtggctaatttgtaatagatcaggcttttcaagtatggagaaagtatctaaaagtgtcatatcagctcacaagaaccaagatattactcttttcccacgtggtggagtagctgcctgcattactcacaactataaaaaaccccatagggccttctgttttttgcctctttctttggagactgggctgccatttcatgtgaatggccactttgcactggattcagccagaaggaacctgtggcgtgatgataatggagttggtgttcgaagtgactggaataacagtttaatgacagcattaatagctcctgcatatgttgaattgctaatacagttaaaaaaacggtatttccctggttctgatccaacattatcagtgttacagaacacccctattcatgttgtaaaggacactttaaagaagtttttatcgtttttcccagttaaccgtcttgatctacagccagatttatattgtctagtgaaagcactttacaattgcattcacgaagacatgaaacgtcttttacctgttgtgcgggctccaaatattgatggctctgacttgcactctgcagttataattacttggatcaatatgtctacttctaataaaactagaccattttttgacaatttactacaggatgaattacaacaccttaaaaatgcagattataatatcaccacacgcaaaacagtagcagagaatgtctataggctgaaacatctccttttagaaattggtttcaacttggtttataactgtgatgaaactgctaatctttaccactgtcttatagatgcagatattcctgttagttatgtgacccctgctgatatcagatcttttttaatgacattttcctctcctgacactaattgccatattgggaagctgccttgtcgtctgcagcagactaatctaaaactttttcatagtttaaaacttttagttgattattgttttaaagatgcagaagaaaatgagattgaagttgagggattgccccttctcatcacactggacagtgttttgcaaacttttgatgcaaaacgacccaagtttctaacaacatatcatgaattgattccatcccgcaaagacttgtttatgaatacattatatttgaaatatagtaatattttattgaactgtaaagttgcaaaagtgtttgacatttccagctttgctgatttgttatcctctgtgttgcctcgagaatataagaccaaaagttgcacaaagtggaaagacaattttgcaagtgagtcttggcttaagaatgcatggcattttattagtgaatctgtaagtgtgaaagaagatcaggaagaaacaaaaccaacatttgacattgttgttgatactctaaaagactgggcattgcttccaggaacaaagtttactgtttcagccaaccagcttgtggttcctgaaggagatgttctgcttcctctcagccttatgcacattgcagtttttccaaatgcccagagtgataaagtttttcatgctctaatgaaagctggctgtattcagcttgctttgaacaaaatctgttccaaagacagtgcatttgttcctttgttgtcatgtcacacagcaaatatagagagccccacaagcatcttgaaggctctacattatatggtccaaacttcaacatttagagcagaaaaattagtagaaaatgattttgaggcacttttgatgtatttcaactgcaatttgaatcatttgatgtcccaagatgatataaaaattctaaagtcacttccgtgctataaatccatcagtggccgctatgtaagcattggaaaatttggaacatgctacgtacttacaaaaagtatcccttcagctgaagtggagaaatggacacagtcatcatcatctgcatttcttgaagaaaaaatacacttaaaagaactatatgaggtgattggttgtgtacctgtagatgatcttgaggtatatttgaaacacctcttaccaaaaattgaaaatctctcttatgatgcaaaattagagcacttgatctaccttaagaatagattatcaagtgctgaggaattatcagagattaaggaacaactttttgaaaaactggaaagtttattgataatccatgatgctaacagtagactaaagcaagcaaagcatttctatgatagaactgtgagagtttttgaagttatgcttcctgaaaaattgtttattcctaatgatttctttaagaaattggaacaacttataaaacccaaaaatcatgttacatttatgacatcctgggtggaattcttaagaaatattggactaaaatacatactttctcagcagcagttgttacagtttgctaaggaaatcagtgtgagggctaatacagaaaactggtccaaagaaacattgcaaaatacagttgatatccttctgcatcatatattccaagaacgaatggatttgttatctggaaattttctgaaagaactatctttaataccattcttatgtcctgagcgggcccccgcggaattcattagatttcatcctcaatatcaagaggtaaatggaacacttcctcttataaagttcaatggagcacaggtaaatccaaaattcaagcaatgtgatgtactccagctgttatggacatcctgccctattcttccagagaaagctacacccttaagcattaaagaacaagaaggtagtgaccttggtccacaagaacagcttgaacaagttttaaatatgcttaatgttaacctggatcctcctcttgataaggtaatcaataactgcagaaacatatgcaacataacgacgttggatgaagaaatggtaaaaactagagcaaaagtcttaaggagcatatatgaattcctcagtgcagaaaaaagggaatttcgttttcagttgcgaggggttgcttttgtgatggtagaagatggttggaaacttctgaagcctgaggaggtagtcataaacctagaatatgaatctgattttaaaccttatttgtacaagctacctttagaacttggcacatttcaccagttgttcaaacacttaggtactgaagatattatttcaactaagcaatatgttgaagtgttgagccgcatatttaaaaattctgagggcaaacaattagatcctaatgaaatgcgtacagttaagagagtagtttctggtctgttcaggagtctacagaatgattcagtcaaggtgagg

SEQ ID NO: 84 - DNA, 1275 bp, synthetic construct
  source 1..1275: mol_type = other DNA
aagggcggcggccggttcggccaaacaaccccgccactggtggacttcctgaaggacatcctgagaagataccccgagggcgggcagatactgaaagagcttattcagaacgccgaagacgccggcgccaccgaggtgaagttcctgtacgacgagacacagtacggcaccgagactctgtggagcaaggacatggccccctaccaaggccccgccctgtacgtgtacaacaacgccgtgttcacccccgaggactggcacggcatccaagagatcgctagaagcagaaagaaggacgaccccctgaaggtgggcagattcggcatcggcttcaacagcgtgtaccacatcaccgacgtgccctgcatcttcagcggcgatcagatcggcatgctggacccccaccaaacactgttcggcccccacgagagcgggcagtgctggaacctgaaggacgacagcaaggagatcagcgaactgtccgatcagttcgcccccttcgtgggcatcttcggcagcaccaaggagaccttcatcaacggcaacttccccggcaccttcttcagattccccctgagactgcagccttctcagctgagcagcaacctgtacaacaagcagaaggtgctggagctgttcgagagcttcagagccgacgccgacaccgtgctgctgttcctgaagagcgtgcaagacgtgagcctgtacgtgagagaggccgacggtacggagaaactggtgttcagagtgacaagcagcgagagcaaggccctgaagcacgagagacccaacagcatcaagatcctgggcaccgccatcagcaactactgcaagaagacccctagcaacaacatcacctgcgtgacctaccacgtgaacatcgtgctggaggaggagagcaccaaggacgctcagaagacaagctggctggtgtgcaacagcgtgggcggcagaggcatcagcagcaagctggacagcctggccgacgagctgaagttcgtgcccatcatcggcatcgccatgcccctgagcagcagagacgacgaggccaagggcgccacaagcgactttagtggtaaagccttctgcttcctgccactgccacccggagaggagagcagcaccggcctgcccgtgcacatcagcggcttcttcggcctgaccgacaacagaagaagcatcaagtggagagagctggatcagtggagagaccccgccgccctgtggaacgagttcctggtgatgaacgtggtgcccaaggcctacgccaccctg

SEQ ID NO: 85 - DNA, 246 bp, synthetic construct
  source 1..246: mol_type = other DNA
gagaccaaggagaacagatgggtacccgtgaccgttctgcccggctgtgtgggctgtcgtacggtggcagccctggcaagctggaccgtgagagacgtgaaggagagaatcttcgccgagaccggcttccccgtgagcgagcagagactgtggagaggcggcagagaactgtccgactggatcaagatcggcgacctgacaagcaagaactgccacctgttcgtgaacctgcagagcaagggcctg

SEQ ID NO: 86 - DNA, 264 bp, synthetic construct
  source 1..264: mol_type = other DNA
atcctgaaagaggtgacgagcgtggtggagcaagcctggaagctgcccgagagcgagagaaagaagatcatcagaagactgtacctgaagtggcaccccgacaagaaccccgagaaccacgacatcgccaacgaggtgttcaagcacctgcagaacgagatcaacagactggagaagcaagccttcctggaccaaaatgccgacagagcaagcagaagaaccttcagcacaagcgcaagcagatttcagagcgacaagtacagc

SEQ ID NO: 87 - DNA, 417 bp, synthetic construct
  source 1..417: mol_type = other DNA
gtgggcaaccccgtggaggctagaagatggctgagacaagctagagccaacttcagcgccgctagaaacgacctgcacaagaacgccaacgagtgggtgtgcttcaagtgctacctgagcaccaagctggccctgatcgccgccgactacgccgtgagaggcaagagcgacaaggacgtgaagcccaccgccctggctcagaagatcgaggagtactctcagcagctggagggcctgaccaacgacgtgcacaccctggaggcctacggcgtggacagcctgaagacaagataccccgacctgctgcccttccctcagatccccaacgacagattcacaagcgaggtggccatgagagtgatggagtgcaccgcctgcatcatcatcaagctggagaacttcatgcagcagaaggtg

SEQ ID NO: 88 - DNA, 2355 bp, synthetic construct
  source 1..2355: mol_type = other DNA
atggagaccaaggagaacagatgggtacccgtgaccgttctgcccggctgtgtgggctgtcgtacggtggcagccctggcaagctggaccgtgagagacgtgaaggagagaatcttcgccgagaccggcttccccgtgagcgagcagagactgtggagaggcggcagagaactgtccgactggatcaagatcggcgacctgacaagcaagaactgccacctgttcgtgaacctgcagagcaagggcctgaagggcggcggccggttcggccaaacaaccccgccactggtggacttcctgaaggacatcctgagaagataccccgagggcgggcagatactgaaagagcttattcagaacgccgaagacgccggcgccaccgaggtgaagttcctgtacgacgagacacagtacggcaccgagactctgtggagcaaggacatggccccctaccaaggccccgccctgtacgtgtacaacaacgccgtgttcacccccgaggactggcacggcatccaagagatcgctagaagcagaaagaaggacgaccccctgaaggtgggcagattcggcatcggcttcaacagcgtgtaccacatcaccgacgtgccctgcatcttcagcggcgatcagatcggcatgctggacccccaccaaacactgttcggcccccacgagagcgggcagtgctggaacctgaaggacgacagcaaggagatcagcgaactgtccgatcagttcgcccccttcgtgggcatcttcggcagcaccaaggagaccttcatcaacggcaacttccccggcaccttcttcagattccccctgagactgcagccttctcagctgagcagcaacctgtacaacaagcagaaggtgctggagctgttcgagagcttcagagccgacgccgacaccgtgctgctgttcctgaagagcgtgcaagacgtgagcctgtacgtgagagaggccgacggtacggagaaactggtgttcagagtgacaagcagcgagagcaaggccctgaagcacgagagacccaacagcatcaagatcctgggcaccgccatcagcaactactgcaagaagacccctagcaacaacatcacctgcgtgacctaccacgtgaacatcgtgctggaggaggagagcaccaaggacgctcagaagacaagctggctggtgtgcaacagcgtgggcggcagaggcatcagcagcaagctggacagcctggccgacgagctgaagttcgtgcccatcatcggcatcgccatgcccctgagcagcagagacgacgaggccaagggcgccacaagcgactttagtggtaaagccttctgcttcctgccactgccacccggagaggagagcagcaccggcctgcccgtgcacatcagcggcttcttcggcctgaccgacaacagaagaagcatcaagtggagagagctggatcagtggagagaccccgccgccctgtggaacgagttcctggtgatgaacgtggtgcccaaggcctacgccaccctgggcagcatcctgaaagaggtgacgagcgtggtggagcaagcctggaagctgcccgagagcgagagaaagaagatcatcagaagactgtacctgaagtggcaccccgacaagaaccccgagaaccacgacatcgccaacgaggtgttcaagcacctgcagaacgagatcaacagactggagaagcaagccttcctggaccaaaatgccgacagagcaagcagaagaaccttcagcacaagcgcaagcagatttcagagcgacaagtacagctttcagagattctacacaagctggaaccaagaggccacaagccacaagagcgagagacagcagcagaacaaggagaagtgcccccctagcgccgggcagacctactctcagagattcttcgtgccccccaccttcaagagcgtgggcaaccccgtggaggctagaagatggctgagacaagctagagccaacttcagcgccgctagaaacgacctgcacaagaacgccaacgagtgggtgtgcttcaagtgctacctgagcaccaagctggccctgatcgccgccgactacgccgtgagaggcaagagcgacaaggacgtgaagcccaccgccctggctcagaagatcgaggagtactctcagcagctggagggcctgaccaacgacgtgcacaccctggaggcctacggcgtggacagcctgaagacaagataccccgacctgctgcccttccctcagatccccaacgacagattcacaagcgaggtggccatgagagtgatggagtgcaccgcctgcatcatcatcaagctggagaacttcatgcagcagaaggtgtga

SEQ ID NO: 89 - DNA, 448 bp, synthetic construct; identical to SEQ ID NO: 54
  source 1..448: mol_type = other DNA

SEQ ID NO: 90 - DNA, 1344 bp, synthetic construct
  source 1..1344: mol_type = other DNA
atcgataggtaccgagctcttacgcgtgctagctgtatgcagctggacctaggagagaagcaggagaggaagatccagcacaaaaaatccgaagctaaaaacaggacacagagatgggggaagaaaagagggcagagtgaggcaaaaagagactgaagagatgagggtggccgccaggcactttagataggggagaggctttatttacctctgtttgtttttttttttttttttttttttttgcgaggtagtcttgcttagtctccaggctggagtgcagtggcacaatctcagctcactgcaacttccacctcctgggttcaagcaattctcctgcctcagcctcccgagtagctgggactacaggcgcatgcaaccgcgcctggctaatttttgtatttttagtagaaacggggtttcaccacgttagccaggatggtctggatctcctgacctcgtgatctgcccgcctccgccttccaaagtgctgggattacaggggtgagccacagcgcctggtccctatttacttctgtcttctacctccaggagatcaaagacgctggccttcagacctgatcagactcccaggggcagccaccacatgtatgacagagaacagaggatgcctgtttttgccccaaagctggaaattcatcacaacctgaggcccaggatctgctctgtgccggtcctctgggcagtgtggggtgcagaatggggtgcctaggcctgagcgttgcctggagcctaggccgggggccgccctcgggcaggcgtgggtgagagccaagaccgcgtgggccgcggggtgctggtaggagtggttggagagacttgcgaaggcggctggggtgttcggatttccaataaagaaacagagtgatgctcctgtgtctgaccgggtttgtgagacattgaggctgtcttgggcttcactggcagtgtgggccttcgtacccgggctacaggggtgcggctctgcctgttactgtcgagtgggtcgggccgtgggtatgagcgcttgtgtgcgctggggccaggtcgtgggtgcccccacccttcccccatcctcctcccttccccactccaccctcgtcggtcccccacccgcgctcgtacgtgcgcctccgccggcagctcctgactcatcgggggctccgggtcacatgcgcccgcgcggccctataggcgcctcctccgcccgccgcccgggagccgcagccgccgccgccactgccactcccgctctctcagcgccgccgtcgccaccgccaccgccaccgccactaccaccgagatctgcgatctaagtaagcttggcattccggtactgttggtaaa

SEQ ID NO: 91 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 92 - DNA, 1764 bp, synthetic construct
  source 1..1764: mol_type = other DNA
agctctgagctcctcctctgctcgcccaatccttccaaccccctatggtggtatggctgacacagaaaatgtctgctcctgtatgggacatttgcccctcttctccaaatataagacaggatgaggcctagcttttgctgctccaaagttttaaaagaacacattgcacggcatttagggactctaaagggtggaggaggaatgagggaattgcatcatgccaaggctggtcctcatccatcactgcttccagggcccagagtggcttccaggaagtattcttacaaaggaagcccgatctgtagctaacactcagagcccattttcctgcgttaacccctcccgacctcatatacaggagtaacatgatcagtgacctgggggagctggccaaactgcgggacctgcccaagctgagggccttggtgctgctggacaacccctgtgccgatgagactgactaccgccaggaggccctggtgcagatggcacacctagagcgcctagacaaagagtactatgaggacgaggaccgggcagaagctgaggagatccgacagaggctgaaggaggaacaggagcaagaactcgacccggaccaagacatggaaccgtacctcccgccaacttagtggctcctctagcctgcagggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaatacaggacacggggtggagaggcgaaaggagggcagagtgaggcagagagactgagaggcctggggatgtgggcattccggtagggcacacagttcacttgtcttctctttttccaggaggccaaagatgctgacgtcaagaactcataataccccagtggggaccaccgcattcatagccctgttacaagaagtgggagatgttcctttttgtcccagactggaaatccgttacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccg